(19) United States
(12) Patent Application Publication
(10) Pub. No.: US 2026/0337565 A1
(43) Pub. Date: Oct. 29, 2026

(54) COMPOSITIONS AND METHODS OF USE OF T CELLS IN IMMUNOTHERAPY

(71) Applicants: The Broad Institute, Inc., Cambridge, MA (US); Massachusetts Institute of Technology, Cambridge, MA (US)

(72) Inventors: Steven Carr, Cambridge, MA (US); Mirco Julian Friedrich, Cambridge, MA (US); Gabrielle Hernandez, Cambridge, MA (US); Jennifer Abelin, Cambridge, MA (US)

(21) Appl. No.: 19/661,903

(22) Filed: Apr. 29, 2026

Related U.S. Application Data
(63) Continuation of application No. PCT/US2024/054265, filed on Nov. 1, 2024.
(60) Provisional application No. 63/595,737, filed on Nov. 2, 2023.
(60) Provisional application No. 63/682,327, filed on Aug. 12, 2024.

(51) Int. Cl. C12N 5/0783; A61K 39/00; A61K 40/11; A61K 40/32; A61K 40/42; A61P 35/00; A61P 35/02; C07K 14/705; C12Q 1/6869; G01N 33/68

(52) U.S. Cl. CPC C12N 5/0636; A61K 39/001102; A61K 40/11; A61K 40/32; A61K 40/4202; A61P 35/00; A61P 35/02; C07K 14/7051; C12Q 1/6869; G01N 33/6863; G01N 33/6866; G01N 33/6869; A61K 2039/53; C12N 2510/00; G01N 2333/525; G01N 2333/55; G01N 2333/57

(57) ABSTRACT

The present disclosure relates in some aspects to methods, cells, and compositions for preparing isolated engineered immune cells comprising T cell receptors (TCRs) capable of recognizing a disease-associated antigen. In some aspects, the immune cells are T cells for use in immunotherapy. Provided In an embodiment are T cell preparation methods, including isolation, processing, incubation, and genetic engineering of cells and populations of cells. Also provided are the isolated engineered T cells and compositions produced by the methods in the present disclosure. In some aspects, the methods prepare T cells for adoptive therapy. In an embodiment, the disease-associated antigen is a cancer-associated antigen.

### CROSS REFERENCE TO RELATED APPLICATIONS

This application is a continuation application of International Patent Application No. PCT/US2024/054265 filed Nov. 1, 2024, which claims the benefit of U.S. Provisional Application Nos. 63/595,737 filed Nov. 2, 2023 and 63/682,327 filed Aug. 12, 2024. The entire contents of the above-identified applications are hereby fully incorporated herein by reference.

### REFERENCE TO AN ELECTRONIC SEQUENCE LISTING

The contents of the electronic sequence listing (“BROD-5940US_ST26.xml”; Size is 35,616,963 bytes and it was created on Apr. 22, 2026) is herein incorporated by reference in its entirety.

### TECHNICAL FIELD

The subject matter disclosed herein is generally directed to T cell receptors (TCRs) capable of recognizing antigens that are shared across multiple patients in a particular disease context and their use in adoptive cell therapies and vaccine compositions.

### BACKGROUND

Approximately 1.6 million Americans are diagnosed with neoplasia every year, and approximately 580,000 people in the United States are expected to die of the disease in 2013. Over the past few decades there been significant improvements in the detection, diagnosis, and treatment of neoplasia, which have significantly increased the survival rate for many types of neoplasia. However, only about 60% of people diagnosed with neoplasia are still alive 5 years after the onset of treatment, which makes neoplasia the second leading cause of death in the United States.

Multiple myeloma (MM), also known as plasma cell myeloma, myelomatosis, Kahler's, is a cancer of plasma cells, a type of white blood cell normally responsible for producing antibodies in which collections of the neoplastic plasma cells accumulate in the bone marrow. It is the second most common hematologic cancer as it accounts for 10% of all hematologic malignancies and represents 1% of all cancer diagnosis and 2% of all cancer deaths. MM leads to bone lesions with 80% of patients developing osteoporosis, lytic bone lesions, or fractures during the course of the disease. MM treatments with alkylating agents, corticosteroids, proteasome inhibitors, and immunomodulatory drugs have resulted in significant survival benefits, however relapse is inevitable and disease remains incurable with a median survival of 5 years.

Acute myeloid leukemia (AML) is a heterogeneous hematologic disorder characterized by clonal expansion of myeloid blasts in bone marrow, peripheral blood, and other tissues. Despite recent progress, current treatment of AML remains unsatisfactory with a 5-year relapse-free survival rate lower than 30%.

Various strategies are available for producing and administering engineered cells for adoptive therapy. Some available strategies include engineering immune cells expressing genetically engineered antigen receptors, such as CARs, and for suppression or repression of gene expression in the cells. Improved strategies are needed, for example, to provide a wider range of target antigens and diseases that may be treated using such cells, to improve specificity or selectivity of the cells, e.g., to avoid off-target effects, and to improve efficacy of the cells, for example, by avoiding suppression of effector functions and improving the activity and/or survival of the cells upon administration to subjects. Provided are methods, cells, compositions, kits, and systems that meet such needs.

Citation or identification of any document in this application is not an admission that such a document is available as prior art to the present invention.

### SUMMARY

In an embodiment, the techniques described herein relate to an isolated engineered immune cell including a T cell receptor (TCR) capable of recognizing a disease-associated antigen.

In an embodiment, the techniques described herein relate to a cell, wherein the disease-associated antigen is a virus-associated antigen. In an embodiment, the techniques described herein relate to a cell, wherein the disease-associated antigen is a cancer-associated antigen.

In an embodiment, the techniques described herein relate to a cell, wherein the cancer-associated antigens are associated with one or more hematological malignancies. In an embodiment, the techniques described herein relate to a cell, wherein the hematological malignancy is multiple myeloma (MM). In an embodiment, the techniques described herein relate to a cell, wherein the hematological malignancy is acute myeloid leukemia (AML). In an embodiment, the techniques described herein relate to a cell, wherein the hematological malignancy is chronic lymphocytic leukemia (CLL).

In an embodiment, the techniques described herein relate to a cell, wherein the disease-associated antigen is selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

In an embodiment, the techniques described herein relate to a cell, wherein the TCR includes SEQ ID NOs: 1-121, and/or a TCR alpha chain CDR3 sequence selected from SEQ ID NO: 1-62 or 41855-41902 or TCR beta chain CDR3 sequence selected from SEQ ID NO: 63-121 or 41903-41948.

In an embodiment, the techniques described herein relate to a cell, wherein the cell is a CD8 T cell. In an embodiment, the techniques described herein relate to a cell, wherein the CD8 T cell is isolated from a subject to be treated.

In an embodiment, the techniques described herein relate to a cell, wherein the cell includes one or more modifications to one or more genes that modify an immune reactivity of the cell.

In an embodiment, a method of treating cancer comprises administering the engineered immune cell to a subject in need thereof. In an embodiment, the subject suffers from a cancer that is a hematological malignancy. In an embodiment, wherein the hematological malignancy is MM, AML, or CLL.

In an embodiment, the techniques described herein relate to a vaccine including a cancer-associated antigen. In an embodiment, the techniques described herein relate to a vaccine, wherein the antigen is recognized by a TCR selected from SEQ ID NOs: 1-121 and/or a TCR alpha chain CDR3 sequence selected from SEQ ID NO: 1-62 or 41855-41902 or TCR beta chain CDR3 sequence selected from SEQ ID NO: 63-121 or 41903-41948

In an embodiment, the techniques described herein relate to a vaccine, wherein the antigen is selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

In an embodiment, the vaccine includes a polynucleotide encoding the conserved cancer antigen. In an embodiment, the polynucleotide is mRNA.

In an embodiment, wherein the vaccine includes the antigen and optionally a carrier or adjuvant.

In an embodiment, a method of treating cancer comprises administering the vaccine to a subject in need thereof. In an embodiment, the subject suffers from a hematological malignancy. In an embodiment, the hematological malignancy is multiple myeloma, acute myeloid leukemia, or chronic lymphocytic leukemia.

In an embodiment, the techniques described herein relate to a method for detecting tumor-reactive T-cell receptors (TCRs): (a) characterizing the phenotype and clonality of a population of isolated T cells to define a baseline transcriptional state; (b) segregating single isolated T cells from the population of isolated T cells into individual discrete volumes and exposing the single isolated T cells to a tumor cell; (c) identifying and retrieving single isolated T cells from the individual discrete volumes and conducting TCR alpha and beta chain sequencing; and (d) identifying antigen-reactive T cells by matching each TCR to its baseline transcriptional state using the CDR3 amino acid sequence as an endogenous barcode of each TCR.

In an embodiment, the techniques described herein relate to a method, wherein step (b) further includes capture beads to detect T cell-derived cytokines and wherein single isolated T cells are retrieved for step (c) if T cell cytokines are detected.

In an embodiment, the techniques described herein relate to a method, wherein the T cell-derived cytokines include interleukin-2 (IL-2), interferon-gamma, and tumor necrosis factor (TNF).

In an embodiment, the techniques described herein relate to a method, wherein step (b) further includes assaying for expression of surface 4-IBB as an indicator of an antigen-activated T cell.

In an embodiment, the techniques described herein relate to a method, further includes exposing a subset of the population of isolated T cells to stimulation with tumor or viral antigens and obtaining TCR sequencing TCRs using TCRV (Beta)-seq, and integrating the TCRV (beta)-seq with the baseline transcriptional state using the CDR3 amino acid sequence.

In an embodiment, the techniques described herein relate to a method further including defining an antigen-reactive TCR signature based on the identified baseline transcriptional state.

In an embodiment, the techniques described herein relate to a method, wherein characterizing the phenotype and clonality of the cells includes using high-throughput single-cell RNA sequencing (scRNA-seq), single-cell TCR sequencing (scTCR-seq) coupled with the detection of surface proteins using cellular indexing of transcriptomes and epitopes by sequencing (CITE-seq).

In an embodiment, the techniques described herein relate to a method, wherein determining one or more epitopes on the cells to define the clonotype includes using high-throughput single-cell RNA sequencing (scRNA) and single-cell TCR sequencing.

In an embodiment, the techniques described herein relate to a method, wherein determining one or more epitopes on the cells includes using cellular indexing of the transcriptomes and epitopes by sequencing (CITE-seq).

In an embodiment, the techniques described herein relate to a method, wherein step (b) further includes optical screening to quantify T cell activation and cytokine production.

In an embodiment, the techniques described herein relate to a method, further including expanding the identified antigen-specific T cells in a cell population and delivering the cell population to a subject in need thereof.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1H show the transcriptomic landscape of clonal T cells in the diseased bone marrow. FIG. 1A is a graphical overview of TCR discovery platform. To characterize the phenotype and clonality of bone marrow resident T cells (BMR-T), Applicants used high-throughput single-cell RNA sequencing (scRNA-seq) and single-cell TCR sequencing (scTCR-seq) coupled with the detection of surface proteins (that is, cellular indexing of transcriptomes and epitopes by sequencing (CITE-seq), thereby defining the in vivo transcriptional state as baseline (Step 1). Functionally tumor reactive TCRs were identified in parallel using 1), a microfluidics-based forward screening approach of single BMR-T exposed to single autologous tumor cells and 2) the MANA functional expansion of specific T cells assay (MANAFEST) on BMR-T of all individuals (Step 2). Functionally screened TCRs were retrieved from their microfluidic reaction chambers and subjected to combined TCR-alpha and beta-chain sequencing (TCRA/B-seq), while MANAFEST-cultures were sequenced after 4 weeks in total with combined scRNA/TCR-seq (Step 3). The data of both assays was then used to identify and phenotypically map antigen reactive T cells by matching each TCR to its baseline transcriptional state using the CDR3 nucleotide sequence as unique barcode of a given clone A signature of tumor reactivity was established based on the transcriptional features of tumor reactive TCRs in the establishment cohort (n=6 NDMM patients) and tested for sensitivity and specificity to prospectively identify tumor reactive TCRs in an independent validation cohort (n=6 NDMM patients) (Step 4). Lastly, the clonal dynamics and clinical relevance of tumor reactive TCRs were explored in two clinical trial cohorts with longitudinal bone marrow biopsies: newly diagnosed multiple myeloma patients undergoing induction immunochemotherapy followed by autologous stem cell transplant (ASCT; n=14 patients) and relapsed/refractory multiple patients undergoing bispecific BCMAxCD3 antibody treatment (bsAb, n=16 patients) (Step 5). FIG. 1B is a UMAP of T cell subtypes with productive TCR identified in the NDMM establishment cohort (N=6 patients, n=101,210 cells, n=81,122 TCRs post QC). FIG. 1C is a stacked bar chart of single cell count and the respective cluster annotation per TCR-clonotype. Top 50 clonotypes per patient shown. FIG. 1D is a UMAP depicting expansion of BMR-T clonotypes (large=0.01-1; medium=0.001-0.001; small=0.0001-0.001; rare=0-0.0001). FIG. 1E shows the relative abundance of expansion-categories within cells of each patient. FIG. 1F shows the average clonotype proportion in sample as dot size by T cell subtype within each patient. FIG. 1G shows the T cell subtype composition in expanded clones (Proportion in bone marrow >0.01) and non-expanded (Proportion in bone marrow <0.01) clones. FIG. 1H shows the gini clonality and inversed Simpson diversity indices per transcriptionally defined BMR-T cluster. N=6 NDMM patients.

FIGS. 2A-2L shows phenotype and specificity of bone-marrow associated T cells. FIG. 2A shows representative images of microfluidics based forward TCR screening approach. Single BMR-T were co-cultured with autologous myeloma cells for 16 h. Each microfluidic reaction chamber further contained capture beads to detect the T cell-derived cytokines Interleukin-2 (IL-2), Interferon-gamma (IFN-γ) and Tumor necrosis factor (TNF) and was observed for surface 4-1BB (CD137) protein expression. If one or more signals of tumor reactivity were detected, this T cell was retrieved from its reaction chamber and subjected to TCRA/B-seq (Methods). FIG. 2B is a venn diagram outlining characteristics of n=413 TCR: Tumor recognition events. BMR-T that secreted any cytokine together with 4-1BB surface protein expression were classified as polyfunctional. FIG. 2C (Left) shows a stacked bar chart outlining distribution of TCR: Tumor recognition events in each run of the microfluidics-based forward screening assay. n=26 assay runs. Definition of BMR-T function as in b). FIG. 2C (Right) Summary statistics of n=1,243 TCR: Tumor recognition events. Definition of BMR-T function as in b). FIG. 2D shows summary statistics of n=1,243 TCR: Tumor recognition events split by CD4+ or CD8+ subtype. Definition of BMR-T function as in b). Statistical significance of enrichment was determined by mixed effects analysis with Bonferroni post-hoc test for multiple hypothesis testing correction. FIG. 2E shows functional validation of cloned and mRNA transfected TCRs derived from the microfluidics-based forward screening assay. T cells were transfected with cloned TCRA/B fragments and murine TRAC/TRBC chains and co-cultured with patient-autologous multiple myeloma cells. CD69 surface expression was measured by flow cytometry. TCRs derived from N=3 patients, n=3 experimental replicates per patient. Statistical significance was determined by two-way ANOVA with Tukey post-hoc test for multiple hypothesis testing correction. FIG. 2F shows a UMAP of T cells in the establishment cohort colored by recognized antigen (Myeloma, SARS-CoV-2, Influenza-A, CMV, EBV and bystander (non-reactive). TCRs derived from N=6 patients, n=81,122 total TCRs post QC. (FIG. 2G) T cell subtype composition of BMR-T reactive to the outlined antigens. Total number of cells per antigen indicated. TCRs derived from N=6 patients, n=81,122 total TCRs post QC. (FIG. 2H) Scaled average transcript expression heatmap of top 10 differentially expressed genes per recognized antigen (Myeloma, SARS-CoV-2, Influenza-A, CMV, EBV and bystander (non-reactive). TCRs derived from N=6 patients, n=81,122 total TCRs post QC. (FIG. 2I) Scaled average expression heatmap of selected marker genes per recognized antigen. (FIG. 2J) X-Y scatter of average expression of cytotoxicity and dysfunction module scores in BMR-T color-coded by antigen-recognizing TCR. Bystander T cells colored in grey. TCRs derived from N=6 patients, n=81,122 total TCRs post QC. FIG. 2K is a violin plot depicting cell-wise expression of cytotoxicity signature split by antigen reactivity. Statistical significance was determined by two-way ANOVA with Tukey post-hoc test for multiple hypothesis testing correction. FIG. 2L shows a 2MHC-I blocking experiment. TCR-transgenic T cells were tested against autologous MM cells as outlined in FIG. 2E and CD137 and CD69 expression were measured by flow cytometry. MM cells were pre-incubated with either HLA-ABC blocking antibody or isotype control for 1 h before co-culture. Statistical significance was determined by two-way ANOVA with Dunnet post-hoc test for multiple hypothesis testing correction. N=3 experimental replicates per TCR. Irrelevant TCRs were used as negative controls for MHC-I blocking (C1, C2).

FIGS. 3A-3G show conserved transcriptional signatures of tumor reactive BMR-T. FIG. 3A shows scaled average expression heatmap of top differentially expressed genes used to define the tumor reactive TCR transcriptional signature (MM-TCR). FIG. 3B shows a ridge plot of selected marker genes per reactivity group (Myeloma, Virus (SARS-CoV-2, Influenza-A, CMV, EBV), and bystander (non-reactive) in signature establishment cohort. TCRs derived from N=6 patients, n=81,122 total TCRs post QC. FIG. 3C is a UMAP of subsetted validated tumor reactive clonotypes (N=6 patients, n=938 cells). FIG. 3D is a UMAPs overlaid with gene-weighted density of indicated genes. FIG. 3E is a violin plot indicating ITGB1 gene expression in antigen-specific BMR-T color-coded by antigen reactivity. Statistical analysis for enrichment was performed by hypergeometric testing. FIG. 3F shows B16 gp100-expressing and MC38 OVA-expressing tumor cells were injected into C57BL/6J animals followed by intravenous adoptive transfer of 50:50 pmel:OT-I transgenic CD90.1:CD45.1 T cells. Adoptively transferred T cells were detected 7 days post-injection in blood, tumors and tumor-draining lymph nodes (TDLN) by CD90.1 (pmel) or CD45.1 (OT-I) and subjected to flow cytometry analysis of phenotype and CD29 expression. FIG. 3G (Left) shows flow cytometry analysis of CD29 surface protein expression on homed TCR-transgenic T cells in TDLN from k) (Left). FIG. 3G (Right) shows flow cytometry analysis of CD44/CD62L surface protein expression on homed TCR-transgenic T cells in TDLN from k). Statistical significance was determined by two-way ANOVA with Tukey post-hoc test for multiple hypothesis testing correction.

FIGS. 4A-4J show the clinical relevance of tumor reactive T cells in multiple myeloma. FIG. 4A shows N=6 NDMM patients (Tumor reactivity signature validation cohort) were profiled by scRNA/TCR sequencing of bone marrow and peripheral blood. TCRs were classified based on MM-TCR signature expression, followed by microfluidics tumor reactivity screening of BMR-T and autologous tumor cells. Stacked bar chart of single cell count and the respective cluster annotation per TCR-clonotype. Top 50 clonotypes per patient shown. FIG. 4B shows a UMAP of T cells in the validation cohort colored by recognized antigen (Myeloma, SARS-CoV-2, Influenza-A, CMV, EBV and bystander (non-reactive). TCRs derived from N=6 patients, n=61,459 total TCRs post QC. FIG. 4C shows prospective area under the curve (AUC) of receiver operator characteristic (ROC) shown (Methods). AUROC curves of MM-TCR (AUC: 0.9452), MANA_Caushi⁵ (AUC: 0.8119) NeoTCR_8 (AUC: 0.8762) and NeoTCR_4 (AUC: 0.5530)⁷ signature scores to predict tumor reactivity in validation cohort TCRs (n=85,268 cells, n=3,885 validated myeloma reactive BMR-T). FIG. 4D shows a scatter plot depicting frequency of tumor reactive TCRs in the bone marrow and tumor immunogenicity metrics: tumor mutational burden (TMB in mut/MB; right y-axis) and resulting neoantigen load (total count) as per neoepitope prediction using WGS and RNA-seq of tumor cells and germline controls (Methods; left y-axis). FIG. 4E shows a graphical overview of patient cohort and procedure. N=14 NDMM patients were profiled by deep TCR sequencing (TCR-seq) of the alpha and beta TCR chain at baseline (pre-treatment) and 100 days post ASCT. N=6 NDMM patients (Tumor reactivity signature validation cohort) were profiled by scRNA/TCR sequencing of bone marrow and peripheral blood. FIG. 4F shows TCR clonality (see Methods) for each patient at each timepoint for each TCR chain. FIG. 4G shows TCR clonal dynamics over time for three donors with large increases in clonality following ASCT. Each bar represents a single beta chain TCR clone. The height of each bar at Baseline or Post-therapy represents the proportion of the total repertoire each clone occupied at that timepoint. Only clones that occupied >0.002% of the repertoire at either timepoint are shown. Bar color represents the temporal behavior of a TCR over therapy: expanded and contracted TCRs increased or decreased in size after therapy, respectively; while novel or disappeared TCRs were only observed post-therapy or at baseline, respectively. FIG. 4H shows a UMAPs depicting n=3,829 BMR-T (initial diagnosis; top) and n=4,188 BMR-T (d100 post ASCT; bottom) color-coded by MM-TCR signature scored anti-tumor reactivity (predicted reactive=MM-TCR signature score >0.42; predicted not reactive=MM-TCR signature score <0.42). FIG. 4I show TCR clonal dynamics over time for one NDMM patient with following ASCT. Each bar represents a single TCR clone determined by scTCR-seq. Bar color represents the anti-tumor reactivity based on MM-TCR signature score. Log 2 fold change (ASCT/initial diagnosis) shown. FIG. 4J shows a bar chart depicting average count of tumor reactive TCRs detected in the bone marrow of NDMM patients at initial diagnosis split by clinical IMWG consensus response category after induction (immuno-) chemotherapy. N=12 patients (Pt-01 to Pt-12; Table 2). Statistical significance between response groups was determined by one-way ANOVA with Tukey post-hoc test for multiple hypothesis testing correction. Statistical significance for linear trend with increasing response depth (PR-VGPR-nCR-CR MRD-) was determined by one-way ANOVA with Dunn's post-hoc test for multiple hypothesis testing correction. PR, partial response; VGPR, very good partial response; CR, complete response; MRD, minimal residual disease.

FIGS. 5A-5I shows myeloma reactive T cells target shared cancer antigens. FIG. 5A shows total T cell counts (top) and TCR clonotype counts (bottom) retrieved from combined scRNA/TCR-seq of matching bone marrow (BM) biopsies and peripheral blood (PB) samples taken at initial diagnosis of multiple myeloma. N=6 patients. Statistical significance was determined by a two-tailed paired t-test. FIG. 5B shows T cell antigen specificity of antigen-validated (‘deorphanized’) PB-derived (top) and BM-derived (bottom) T cells. Total number of TCRs per antigen indicated. TCR clonotypes were classified as shared between tissues if there was a fully matching CDR3 amino acid sequence across samples. TCRs derived from N=6 patients, n=45,777 (PB), n=45,713 (BM) total TCRs post QC. FIG. 5C shows cells per clonotype in BM and PB averaged across all patients with matching BM and PB tissue and annotated by experimentally validated or VDJb-derived TCR antigen specificity. N=6 biological replicates (matched patient-individual BM-PB datasets), n=45,777 (PB), n=45,713 (BM) total TCRs post QC. FIG. 5D shows distribution of peptide amino acid (AA) length detected by immunoprecipitation of HLA class I:peptide complexes from patient-autologous CD138+ multiple myeloma cell fraction followed by LC-MS/MS analysis (N=6 NDMM patients). FIG. 5E shows distribution of epitope calls and the non-healthy protein families they are derived from as detected by immunoprecipitation of HLA class I:peptide complexes from patient-autologous CD138+ multiple myeloma cell fraction followed by LC-MS/MS analysis (N=6 NDMM patients). CAAs, cancer-associated antigens; nuORFs, novel or unannotated open reading frames. FIG. 5F shows bar charts depicting tumoral MHC class I-derived antigens and the number of patients each antigen was detected in. Peptides eluted from MHC class I molecules of CD138+ multiple myeloma cell fractions derived from N=6 NDMM patients. Only proteins with detection in >1 patient shown. CAAs, cancer-associated antigens; 5′ uORF, 5′ upstream open reading frame; 3′ dORF, 3′ downstream open reading frame; OOF, out-of-frame; ncRNA, non-coding RNA. FIG. 5G is a heatmap showing pairwise similarities of TCR TRA-TRB sequences based on scaled BLOSUM45-similarity. Only values above the 95% bootstrapping threshold as established by background distributions are displayed. TCRs are annotated by the respective patient of origin and clustered across all patients (N=12 NDMM patients). FIG. 5H (SEQ ID NO: 122-123) shows peptide-loaded MHC class I tetramer flow cytometry staining of TCR1-expressing Pt-08 T cells (Methods). Tetramers were loaded with identified epitopes in f). Full experimental data found in Extended Data FIG. 14b. FIG. 5I (SEQ ID NO: 122-127) shows mRNA transfection and functional testing of transgenic TCR1-expressing Pt-08 T cells. Tumor necrosis factor (TNF) was stained in TCR-transgenic T cells expressing the detected shared TCR in g) that were co-cultured with peptide-pulsed PBMCs (Methods). CEFT, pool of SARS-CoV-2 spike and nucleoproteins, major histocompatibility complex (MHC) class I-restricted cytomegalovirus (CMV), Epstein-Barr virus (EBV) and influenza virus epitopes; MM, autologous multiple myeloma cells. N=2 independent co-cultures.

FIGS. 6A-6I show MM-TCR signature identifies TCRs responsive to bispecific antibodies. FIG. 6A provides a graphical overview of patient cohort and procedure. N=18 RRMM patients were profiled by single-cell RNA-seq and single-cell TCR-seq baseline (pre-treatment), post cycle 1 of bispecific BCMA x CD3 antibody treatment and post cycle 3 of bispecific BCMA x CD3 antibody treatment or at relapse. Patient-derived BMR-T were classified at baseline and clonal dynamics of each TCR traced over time. FIG. 6B (Left) shows a UMAP of T cell subtypes with productive TCR identified in the RRMM cohort (N=18 patients at 3 timepoints, n=245,817 cells, n=62,273 TCRs post QC); FIG. 6B (Right) shows a UMAP and bar chart of MM-TCR signature predictions in RRMM BMR-T at indicated timepoints (predicted reactive=MM-TCR signature score >0.42; predicted not reactive=MM-TCR signature score <0.42). Statistical significance between antigen specificity groups was determined by one-way ANOVA with Tukey post-hoc test for multiple hypothesis testing correction. FIG. 6C is a dot plot indicating the proportion of antigen-reactive or bystander BMR-T pre- and post-bispecific BCMAxCD3 antibody treatment, split by clinical response (N=18 patients). Statistical significance was determined by repeated-measures ANOVA with Tukey post-hoc test for multiple hypothesis testing correction. FIG. 6D is a dot plot indicating the proportion of ITGB1+ predicted tumor reactive or non-tumor reactive BMR-T pre- and post-bispecific BCMAxCD3 antibody treatment (N=18 patients). Statistical significance was determined by repeated-measures ANOVA with Tukey post-hoc test for multiple hypothesis testing correction. FIGS. 6E-6F show TCR clonal dynamics over time for representative RRMM patients following 3 cycles of bispecific BCMAxCD3 antibody treatment. Each bar represents a single TCR clone determined by scTCR-seq. The height of each bar at baseline or post-therapy represents the proportion of the total repertoire each clone occupied at that timepoint. Only clones that occupied >0.002% of the repertoire at either timepoint are shown. Bar color represents the anti-tumor reactivity based on MM-TCR signature score. FIGS. 6G-6H show bone marrow counts of T cells with TCRs detected among tumor reactive CD8+ BMR-T classified as effector-memory (EM; FIG. 6G) or progenitor-exhausted (PEX; FIG. 6H). Samples were collected from 18 patients with RRMM who experienced clinical remission (orange; n=12) or poor clinical outcome (grey; n=4) after 3 months of bsAb treatment. Patients with poor clinical outcome were further divided into those who did (n=2) or did not experience (n=4) immediate disease progression on-treatment. Single dots show values for patients with a single time point available. FIG. 6I is violin plot of MM-TCR signature expression per cell split by clinical response to bispecific BCMAxCD3 antibody treatment (n_{NR}=51,123 cells, n_{R}=64,615 cells). Statistical significance was determined by a two-tailed unpaired t-test.

FIGS. 7A-7L show the expansion of tumor reactive BMR-T underlies response to immune checkpoint inhibition in AML. FIG. 7A is a graphical overview of patient cohort and procedure. N=8 relapsed/refractory acute myeloid leukemia (R/R AML) patients were profiled by single-cell RNA-seq and single-cell TCR-seq baseline (pre-treatment), at response assessment/remission and at relapse. Patient-derived BMR-T were classified at baseline and clonal dynamics of each TCR traced over time. FIG. 7B is a UMAP of T cell subtypes with productive TCR identified in the R/R AML cohort (N=8 patients at 3 timepoints, n=21,708 cells, n=11,300 TCRs post QC). FIG. 7C is a UMAP of TCR_BM classifier predictions in R/R MM BMR-T at indicated timepoints (predicted reactive=TCR_BM signature score >0.42; predicted not reactive=TCR_BM signature score <0.42). FIG. 7D shows TCR_BM signature score in cells with a TCR exclusive pre-therapy, exclusive post-therapy or overlapping between pre- and post-therapy (n=8 patients, 131,469 cells with productive TCR). Statistical significance was determined by one-way ANOVA with Tukey post hoc test for multiple hypothesis testing correction. FIG. 7E is a dot plot indicating the proportion of ITGB1+ antigen-reactive or bystander BMR-T pre- and post azacytidine+nivolumab (N=8 patients). Statistical significance was determined by repeated-measures ANOVA with Turkey post-hoc test for multiple hypothesis testing correction. (FIG. 7F) UMAP depicting expansion of BMR-T clonotypes in AML patients (large=0.01-1; medium=0.001-0.001; small=0.0001-0.001; rare=0-0.0001). FIG. 7G shows relative abundance of expansion-categories within T cell clones of each AML patient on azacytidine+nivolumab grouped by clinical response category. FIG. 7H is a scatter plot indicating frequency among BMR-T of single T cell clones pre- and post-therapy with azacytidine+nivolumab aggregated across AML patients. The best clinical response of the patient each analyzed TCR is derived from is indicated by color. TCR classifier output for each clone indicated by shape. FIG. 7I-7K show TCR clonal dynamics over time for representative R/R AML patients on-treatment with azacytidine+nivolumab. Each bar represents a single TCR clone determined by scTCR-seq. The height of each bar at baseline or post-therapy represents the proportion of the total repertoire each clone occupied at that timepoint. Only clones that occupied >0.002% of the repertoire at either timepoint are shown. Bar color represents the anti-tumor reactivity based on TCR_BM classifier score. FIG. 7L is a box plot of TCR_BM signature expression per cell split by clinical response and clinical sampling timepoint (diagnosis, remission, relapse). Statistical significance was determined by one-way ANOVA with Tukey post hoc test for multiple hypothesis testing correction.

FIG. 8A-8F show identification of antigen-specific bone-marrow associated T cells using MHC immunopeptidomes. FIG. 8A shows distribution of peptide amino acid (AA) length detected by immunoprecipitation of HLA class I:peptide complexes from patient-autologous CD138+ multiple myeloma cell fraction followed by LC-MS/MS analysis (N=10 NDMM patients). FIG. 8B shows distribution of epitope calls and the non-healthy protein families they are derived from as detected by immunoprecipitation of HLA class I:peptide complexes from patient-autologous CD138+ multiple myeloma cell fraction followed by LC-MS/MS analysis (N=10 NDMM patients). CAAs, cancer-associated antigens; nuORFs, novel or unannotated open reading frames. FIG. 8C shows bar charts depicting shared tumoral MHC class I-derived antigens and the number of patients each antigen was detected in. Peptides eluted from MHC class I molecules of CD138+ multiple myeloma cell fractions derived from N=10 NDMM patients. Only proteins with detection in >2 patient shown. CAAs, cancer-associated antigens; 5′ uORF, 5′ upstream open reading frame; 3′ dORF, 3′ downstream open reading frame; OOF, out-of-frame; ncRNA, non-coding RNA. FIG. 8D shows the results MANAFEST assay in BMTC cultures of N=9 NDMM patients exposed to the indicated antigen pools. Normalized clonal expansion for each TCR across conditions shown. Only significantly enriched TCRs as per FEST analysis are plotted. FIG. 8E shows a UMAP of identified tumor-reactive T cells in the full multiple myeloma cohort colored by screening technology. TCRs derived from N=15 patients (Pts 01-15, n=174,131 cells from 128,302 TCRs post QC). FIG. 8F shows stacked bar charts summarizing identified TCRs with tumor or virus specificities across all patients. Statistical significance was determined by two-way ANOVA with Tukey post-hoc test for multiple hypothesis testing correction.

FIGS. 9A-9I show myeloma reactive T cells target public or immunoglobulin-derived antigens. FIG. 9A (Left) shows a UMAP of BMTCs in the full multiple myeloma cohort colored by reactivity (Myeloma, SARS-CoV-2, Influenza-A, CMV, EBV, ambiguous (tumor/virus-reactive), and bystander (non-reactive). TCRs derived from N=15 patients, n=128,302 total TCRs post QC. FIG. 9A (Right) shows a UMAP of BMTCs in the full multiple myeloma cohort colored by recognized antigen. T cells responsive to shared MANA pool peptides are highlighted in yellow. TCRs derived from N=15 patients, n=128,302 total TCRs post QC. FIG. 9B shows T cell antigen specificity of antigen-validated (‘deorphanized’) T cells in peripheral blood (PB) and bone marrow (BM). Total number of TCRs per antigen indicated. TCR clonotypes were classified as shared between tissues if there was a fully matching CDR3 amino acid sequence across samples. TCRs derived from N=6 patients, n=45,777 (PB), n=45,713 (BM) total TCRs post QC. FIG. 9C shows clonality (1/Shannon diversity) of bone marrow TCRs split by reactivity. Statistical significance was determined by one-way ANOVA with Tukey post-hoc test for multiple hypothesis testing correction. FIG. 9D shows a X-Y scatter of average expression of cytotoxicity and dysfunction module scores in BMTC color-coded by antigen-recognizing TCR. Bystander T cells colored in grey. TCRs derived from N=15 patients, n=174,131 cells from n=128,302 TCRs post QC. FIG. 9E shows a violin plot depicting cell-wise expression of cytotoxicity score split by antigen reactivity. Statistical significance was determined by two-way ANOVA with Tukey post-hoc test for multiple hypothesis testing correction. FIG. 9F shows a violin plot depicting cell-wise expression of dysfunction score split by antigen reactivity. Statistical significance was determined by two-way ANOVA with Tukey post-hoc test for multiple hypothesis testing correction. FIG. 9G shows a heatmap showing pairwise similarities of TCR TRA-TRB sequences based on scaled BLOSUM45-similarity. Only values above the 95% bootstrapping threshold as established by background distributions are displayed. TCRs are annotated by the respective patient of origin and clustered across all patients (N=15 NDMM patients). FIGS. 9H-9I show peptide-loaded MHC class I tetramer flow cytometry staining of BMTCs of various HLA-haplotypes (Methods). MHC-specific tetramers were loaded with the CTAG2₈₆₋₉₄ (RLLELHITM (SEQ ID NO: 128)) epitope for h) and 6 shared epitopes for i).

FIGS. 10A-10G show conserved transcriptional signatures of tumor-reactive BMTCs. FIG. 10A shows scaled average expression heatmap of top differentially expressed genes per reactivity category derived from N=15 patients (Pts 01-15). FIG. 10B shows a ridge plot of selected marker genes per reactivity group (Myeloma, Virus (SARS-CoV-2, Influenza-A, CMV, EBV), ambiguous (tumor/virus-reactive), and bystander (non-reactive) in signature establishment cohort. TCRs derived from N=15 patients, n=174.131 cells post QC. FIG. 10C shows a UMAP of subsetted validated tumor-reactive clonotypes (N=15 patients, n=938 cells). FIG. 10D shows UMAPs overlaid with gene-weighted density of indicated genes. FIG. 10E shows a dot plot outlining the average expression of MM-TCR signature marker genes between bone marrow TCRs of indicated specificities in the establishment cohort. FIG. 10F shows prospective area under the curve (AUC) of receiver operator characteristic (ROC) shown (Methods). AUROC curves of MM-TCR (AUC: 0.895), MANA_Caushi⁵ (AUC: 0.810) NeoTCR_8 (AUC: 0.812) and NeoTCR_4 (AUC: 0.563)⁷ signature scores to predict tumor reactivity in validation cohort TCRs (N=9 patients (Pts 07-15), n=101,256 cells, n=1302 validated myeloma reactive BMTCs). FIG. 10G shows a bar chart depicting average count of tumor-reactive TCRs detected in the bone marrow of NDMM patients at initial diagnosis (left) or frequency of tumor-reactive BMTCs per MM-TCR signature (right) split by remission status after induction (immuno-) chemotherapy. N=14 patients (Pt-01 to Pt-15). Induction therapy response for Pt-08 was not available. Statistical significance between response groups was determined by unpaired t-test with Welch's correction. CR, complete response.

FIGS. 11A-11I show MM-TCR signature identifies TCRs responsive to bispecific antibodies. FIG. 11A shows a graphical overview of patient cohort and procedure. N=18 RRMM patients were profiled by single-cell RNA-seq and single-cell TCR-seq baseline (pre-treatment), post cycle 1 of bispecific BCMAxCD3 antibody treatment and post cycle 3 of bispecific BCMA x CD3 antibody treatment or at relapse. Patient-derived BMR-T were classified at baseline and clonal dynamics of each TCR traced over time. FIG. 11B (Left) shows a UMAP of T cell subtypes with productive TCR identified in the RRMM cohort (N=18 patients at 3 timepoints, n=245,817 cells, n=62,273 TCRs post QC). FIG. 11B (Right) shows a UMAP of MM-TCR signature predictions in RRMM BMR-T at indicated timepoints (predicted reactive=MM-TCR signature score >0.42; predicted not reactive=MM-TCR signature score <0.42). FIG. 11C shows a dot plot indicating the proportion of antigen-reactive or bystander BMR-T pre- and post bispecific BCMAxCD3 antibody treatment, split by clinical response (N=18 patients). Statistical significance was determined by repeated-measures ANOVA with Tukey post-hoc test for multiple hypothesis testing correction. FIG. 11D shows TCR clonal dynamics over time for representative RRMM patients following 3 cycles of bispecific BCMAxCD3 antibody treatment. Each bar represents a single TCR clone determined by scTCR-seq. The height of each bar at baseline or post-therapy represents the proportion of the total repertoire each clone occupied at that timepoint. Only clones that occupied >0.002% of the repertoire at either timepoint are shown. Bar color represents the anti-tumor reactivity based on MM-TCR signature score. FIG. 11E shows TCR clonal dynamics over time for representative RRMM patients following 3 cycles of bispecific BCMAxCD3 antibody treatment. Each bar represents a single TCR clone determined by scTCR-seq. The height of each bar at baseline or post-therapy represents the proportion of the total repertoire each clone occupied at that timepoint. Only clones that occupied >0.002% of the repertoire at either timepoint are shown. Bar color represents the anti-tumor reactivity based on MM-TCR signature score. FIG. 11F shows bone marrow counts of T cells with TCRs detected among tumor reactive CD8+ BMTC classified as effector-memory (EM) Samples were collected from 18 patients with RRMM who experienced clinical remission (orange; n=12) or poor clinical outcome (grey; n=4) after 3 months of bsAb treatment. Patients with poor clinical outcome were further divided into those who did (n=2) or did not experience (n=4) immediate disease progression on-treatment. Single dots show values for patients with a single time point available. FIG. 11G shows bone marrow counts of T cells with TCRs detected among tumor reactive CD8+ BMTC classified as progenitor-exhausted (PEX; FIG. 11H). Samples were collected from 18 patients with RRMM who experienced clinical remission (orange; n=12) or poor clinical outcome (grey; n=4) after 3 months of bsAb treatment. Patients with poor clinical outcome were further divided into those who did (n=2) or did not experience (n=4) immediate disease progression on-treatment. Single dots show values for patients with a single time point available. FIG. 11H shows a violin plot of MM-TCR signature expression per cell split by clinical response to bispecific BCMAxCD3 antibody treatment (n_{NR}=51,123 cells, n_{R}=64,615 cells). Statistical significance was determined by a two-tailed unpaired t-test. FIG. 11I is shows clinical response status.

FIGS. 12A-12L show transfer of tumor-reactive TCRs by autologous stem cell transplantation. FIG. 12A shows TCR clonality (1/Shannon diversity) for N=14 NDMM patients profiled by TRVα/β-seq at baseline (initial diagnosis) and after 100 days post ASCT for each TCR chain. FIG. 12B shows TCR clonal dynamics over time for one NDMM patient with following ASCT. Each area in the alluvial plot represents a single TCR clone determined by scTCR-seq. The height of each bar at baseline or post-therapy represents the proportion of the total repertoire each clone occupied at that timepoint. Color shows, if the clone was found in the PBSC product. Anti-tumor reactivity is based on the previously defined signature, calculated on a per-clone level at diagnosis. (tumor-reactive=clone-aggregated signature score >0.55; not reactive=clone-aggregated signature score <0.55). FIG. 12C shows clonal dynamics of N=9 patients with paired single-cell and VDJ data at diagnosis and post-TPL. Significant clonotype dynamics were determined by a bootstrapping approach stratified to number of cells at each time point followed by Benjamini-Hochberg correction for multiple testing. Reactivity was determined per clone with the previously established signature. FIG. 12D shows representative gating strategy of PBSC samples subjected to multiparametric flow cytometry. FIG. 12E shows frequency of CD45 hi CD3+ T cells and Lin-CD34+ CD38− HSCs/MPPs in PBSC samples from N=19 multiple myeloma patients. FIG. 12F shows single-cell RNA and VDJ-sequencing data of PBSC products of 5 patients. UMAP of T cell subtypes with productive TCR. FIG. 12G shows TCR clonal dynamics over time for the patient shown in FIG. 12H after the transplantation. Each area in the alluvial plot represents a single TCR clone determined by scTCR-seq. The height of each bar at baseline or post-therapy represents the proportion of the total repertoire each clone occupied at either +100 or +360 days after stem cell transplant. Color shows if the clone was found in the PBSC product. Anti-tumor reactivity is based on the previously defined signature, calculated on a per-clone level at diagnosis. FIG. 12I shows clonal dynamics of predicted tumor-reactive clones after the autologous stem-cell transplantation. Color shows the different previously established expansion categories as described in the methods. Clone frequencies are separately compared for clones in the PBSC and predicted reactive. FIG. 12J shows differential expression analysis of clones at diagnosis based on if the clones were found in PBSC. Differential expression was assessed by DESeq2 based on patient-level pseudo-bulk aggregation of expression. Visualized as volcano plot using the R package EnhancedVolcano. FIG. 12K shows linear mixed-effects logistic regression analysis for identifying factors predicting likelihood of apheresis on a TCR-clone-level. Figure shows a forest plot of odds ratios with 95% confidence intervals, highlighting significance of tumor-reactive signature, broad cell type classification and previously established expansion characteristics. FIG. 12L shows summarized alluvial of T-cell subtype fractions of shared and unshared clones across the time course of diagnosis, PBSC and +100 and +360 days after transplantation. Figure is split into shared and unshared clonotypes, as defined by not found in the PBSC product.

Analysis of fractions of exhausted-like (PEX) T-cells throughout the time course. Comparison of fractions of shared and not-transplanted T-cells before and after the transplantation per patient (N=5). Statistical significance was determined by repeated-measures two-way ANOVA followed by Sidak's test for multiple hypothesis testing correction.

FIGS. 13A-13D shows profiling of BMR-T in newly diagnosed multiple myeloma. FIG. 13A show a representative gating strategy used for purification of CD45+ and CD3+ cells by fluorescence-activated cell sorting (FACS). Sorted populations were then processed using the 10× Genomics 5′ single-cell sequencing strategy (methods). FIG. 13B show a Uniform Manifold Approximation and Projection (UMAP) map of T cells. Overlay highlights the average expression of indicated canonical T cell surface proteins detected by CITE-seq. EXT. FIG. 13C-13D a dot plots indicating expression of canonical marker genes across CD8+ (c) and CD4+ (d) clusters. Marker gene lists derived from Zheng et al., Science 202147, Cohen et al., Nat Cancer 202248, and Andreatta et al., Nat Commun. 202149.

FIGS. 14A-14B show bone marrow immune repertoire composition in establishment patient cohort. FIG. 14A show a Uniform Manifold Approximation and Projection (UMAP) map of reference-mapped and subsetted T cells post integration and QC split by patient and color-coded for annotated transcriptional clusters. FIG. 14B show a proportion of T cell subtypes in individual patient bone marrow samples evaluated by scRNA-seq.

FIGS. 15-16 show fluorescence imaging of BMR-T identified in establishment NDMM cohort by microfluidics-based forward tumor reactivity screening. Myeloma reactive T cells were detected among BMR-T from bone marrow biopsies of NDMM patients. Reactive T cells were identified upon detection of secreted cytokines IFN-γ, IL-2, TNF (yellow) and surface expression of 4-1BB protein (CD137; blue). Per experimental run, approximately 1,400 individual CD8+ T cells were co-cultured with CD138+ autologous plasma cells after magnetic bead-based isolation from patient bone marrow samples. NEG: A reaction chamber containing a single T cell+cytokine capture beads only. POS: A reaction chamber containing a single T cell plus human αCD3/αCD28 T cell activation beads. +, positive; (+), dim positive; (−), negative for cytokine secretion or 4-1BB expression; ND, due to a non-loaded cytokine capture bead, the respective cytokine could not be determined.

FIGS. 17A-17D show phenotypes of TCRs recovered from patient-derived tumor reactive T cells. FIG. 17A amplified V(D)J regions of TCR chains are visible at 500 to 700 bp. TCR alpha and beta chains are similar in length and therefore mostly visible as a single band. Due to alternative splicing, double bands can be generated in some cases. 5% agarose gels in TBE shown. FIG. 17B show transcriptional cluster composition of each successfully to scRNA/TCR-seq mapped CD4+ and CD8+ T cell clonotype. Relative abundance of cells in each cluster per clonotype shown. FIG. 17C show linearized DNA templates used for in vitro transcription (IVT) of retrieved and inserted TCRA/B V(D)J sequences together with murine TRAC or TRBC chains. mRNA products were then transfected into primary T cells for functional testing of transgenic TCR recognition. FIG. 17D show the representative flow cytometry gating strategy to identify T cells expressing transfected transgenic TCRs by detection of the murine TRBC chain (mTRBC). Activation state of these cells was then measured by CD69 or CD137 surface protein expression.

FIGS. 18A-18H show functional expansion of tumor reactive T cells on BMR-T in establishment cohort. FIG. 18A show absolute T cell count in assay at baseline (d0) and post expansion (d28) per patient. FIG. 18B show a UMAP highlighting expanded (proportion >0.01) clones and not expanded (proportion <0.01) clones. Cluster phenotype annotation as in FIG. 1b. FIG. 18C a UMAP of BMR-T clonal expansion categories split by patient. FIG. 18D shows a TCR clonal homeostasis per patient at baseline input of BMNC expansion culture (do). FIG. 18E shows TCR clonal homeostasis per patient after BMNC expansion culture (d28). EXT. FIG. 18F shows Shannon diversity index of TCRs sequenced in BMR-T cultures at do and d28 of BMNC expansion culture. Statistical significance was determined by a two-tailed paired t-test. FIG. 18G shows a bar chart of T cell subtype composition of large (proportion >0.01 in bone marrow) clones and small (proportion <0.01 in bone marrow) clones by patient. FIG. 18H shows a heatmap of scaled average expression of top 20 marker genes of small (proportion >0.01) non-reactive T cell clones, large (proportion >0.01) non-reactive T cell clones, small (proportion >0.01) reactive T cell clones, large (proportion >0.01) reactive T cell clones.

FIGS. 19A-19B shows retrospective and prospective TCR signature benchmarking of MM-TCR signature versus published signatures of tumor-infiltrating lymphocytes. FIG. 19A) Heatmap depicting AUROC values of published and generated signatures to predict tumor reactive TCRs in BMR-T of establishment NDMM cohort (retrospective). N=6 patients. FIG. 19B shows a heatmap depicting AUROC values of published and generated signature sig9=MM-TCR to predict tumor reactive TCRs in BMR-T of validation NDMM cohort (prospective). N=6 patients.

FIGS. 20A-20F shows trajectory and fate mapping of tumor reactive and bystander BMR-T using RNA velocities and CellRank. FIGS. 20A-20B shows assessment of a) average and b) patient-wise spliced versus unspliced mRNA ratio detected by 5′ scRNA-seq of primary BMR-T. FIG. 20C shows a UMAP of subclustered CD8+ T cells colored according to original cluster annotations overlaid by RNA velocities as computed by CellRank scVelo algorithm. FIG. 20D shows density plots indicating module scores of cytotoxicity (left) and dysfunction (right) signatures overlaid on UMAP from c. Functional signatures derived from Li et al., Cell 2019a27. FIG. 20E shows module scores for T cell cytotoxicity (top) and dysfunction (bottom) for each cluster. Functional signatures derived from Li et al., Cell 2019a27. FIG. 20F shows heatmap visualizing lineage drivers computed for tumor reactivity. Smooth gene expression for the putative tumor reactivity driver genes in latent time, using as cell-level weights the Alpha fate probabilities. Genes sorted according to their peak in latent time (proportion of cells contributing to each bin shown at the bottom), thus revealing a cascade of gene expression events.

FIGS. 21A-21E shows profiling and tumor reactivity classification of BMR-T in validation NDMM cohort. FIG. 21A shows a UMAP of T cell subtypes with productive TCR identified in the NDMM validation cohort (N=6 patients, n=101,210 cells, n=81, 122 TCRs post QC). FIG. 21B shows a UMAP depicting expansion of BMR-T clonotypes (large=0.01-1; medium=0.001-0.001; small=0.0001-0.001; rare=0-0.0001). FIG. 21C shows relative abundance of expansion-categories within cells of each patient. FIG. 21D shows a UMAPs depicting BMR-T per validation cohort patient color-coded by MM-TCR signature scored anti-tumor reactivity (predicted reactive=MM-TCR signature score >0.42; predicted not reactive=MM-TCR signature score <0.42). FIG. 21E shows average clonotype proportion in sample as dot size by T cell subtype within each patient.

FIGS. 22A-22B shows tumor reactive BMR-T identified in validation NDMM cohort by microfluidics-based forward tumor reactivity screening. FIG. 22A shows myeloma reactive T cells were detected among BMR-T from bone marrow biopsies of NDMM patients. Reactive T cells were identified upon detection of secreted cytokines IFN-γ, IL-2, TNF (yellow) and surface expression of 4-1BB protein (CD137; blue). Per experimental run, approximately 1,400 individual CD8+ T cells were co-cultured with CD138+ autologous plasma cells after magnetic bead-based isolation from patient bone marrow samples. NEG: A reaction chamber containing a single T cell+cytokine capture beads only. POS: A reaction chamber containing a single T cell plus human αCD3/αCD28 T cell activation beads. +, positive; (+), dim positive; (−), negative for cytokine secretion or 4-1BB expression; ND, due to a non-loaded cytokine capture bead, the respective cytokine could not be determined. FIG. 22B shows amplified V(D)J regions of TCR chains are visible at 500 to 700 bp. TCR alpha and beta chains are similar in length and therefore mostly visible as a single band. Due to alternative splicing, double bands can be generated in some cases. 5% agarose gels in TBE shown.

FIGS. 23A-23M shows tumor reactive T cells expand upon autologous stem cell transplantation. FIG. 23A shows the number of total TCR counts fit to log 10 for each patient's repertoire at each timepoints for each TCR chain (ASCT cohort, N=14 patients). FIG. 23B shows TCR clonality quantified using the Rényi Entropy from order 0 to infinity for each patient, with the average of the timepoints for each treatment arm and TCR chain overlayed in bold. FIG. 23C shows the individual total TCR counts fit for each patient at each timepoint for each TCR chain. FIG. 23D (SEQ ID NO: 129-187) shows heatmap depicting longitudinal changes of TCR frequency in bone morrow between initial diagnosis and day 100 post-ASCT. TCRs classified and color-coded by MM-TCR signature score for anti-tumor reactivity (predicted reactive=MM-TCR signature score >0.42; predicted not reactive=MM-TCR signature score <0.42). FIG. 23E shows a Uniform Manifold Approximation and Projection (UMAP) map of reference-mapped and subsetted T cells post integration and QC split by time point (initial diagnosis (a) and day 100 post-ASCT (b)). Cluster phenotype annotation as in FIG. 1b. FIG. 23F shows a T cell subtype composition in clones at initial diagnosis and post ASCT that were either classified as anti-myeloma reactive (blue) or non-reactive bystander (grey). FIG. 23G (top) shows a graphical overview of patient Pt-07 and procedure. A 57-year-old male with NDMM underwent bone marrow biopsy, followed by prospective prediction of reactive BMR-T TCRs using the MM-TCR classifier. BMR-T were then tested using the microfluidics-based forward screening assay and outcomes compared on a per-clone basis between anti-tumor reactivity prediction and measured reactivity. Prospective sensitivity and reactivity of the MM-TCR classifier was then compared to published tumor reactive TCR signatures. FIG. 23G (bottom) shows a UMAPs depicting n=9,148 BMR-T at initial diagnosis color-coded by TCR_BM classifier scored anti-tumor reactivity (predicted reactive=TCR_BM signature score >0.42; predicted not reactive=TCR_BM signature score <0.42). N=9148 BMR-T cells. FIG. 23H representative results of microfluidics-based forward screening assay of BMR-T isolated from Pt-07 in e). FIG. 23I UMAPs depicting n=9,148 BMR-T at initial diagnosis color-coded by validated anti-tumor reactivity. Primary transcriptional phenotype of each detected TCR annotated. FIG. 23J shows counts of tumor reactive T cells (Part of TCR1 and TCR2 clonotypes) in bone marrow and peripheral blood of Pt-07. FIG. 23K shows prospective area under the curve (AUC) of receiver operator characteristic (ROC) shown (Methods). AUROC curves of MM-TCR (AUC: 0.9845), MANA_Caushi5 (AUC: 0.9184) NeoTCR_8 (AUC: 0.9431) and NeoTCR_4 (AUC: 0.6067) 7 signature scores to predict tumor reactivity in Pt-07 TCRs (n=9,148 cells, n=478 validated myeloma reactive BMR-T). FIG. 23L shows summary statistics of MM-TCR classifier per detected TCR in Pt-08 (n=9,148 cells, n=7,548 TCRs). FIG. 23M shows blood serum IgG and M protein concentrations [g/L] in Pt-07 over time. Clinical response assessment results according to IMWG response criteria at indicated timepoints post diagnosis shown.

FIGS. 24A-24E shows compartment tracing of antigen-specific patient TCRs and tumor-associated antigens detected by MHC class I immunoprecipitation. FIGS. 24A-24C show UMAPs depicting T cells in bone marrow and peripheral blood at initial diagnosis color-coded by transcriptional phenotype (a), overlap between both compartments (b), or tumor reactivity status (c). Primary transcriptional phenotype of each detected T cell annotated as in FIG. 1b. FIG. 24D shows a dot plot indicating number of T cells (left) and TCR clonotypes (right) and their reactivity status in each analyzed NDMM patient with available matching bone marrow and peripheral blood. BMNCs, bone marrow mononuclear cells; PBMCs peripheral-blood mononuclear cells. FIG. 24E show bar charts depicting tumoral MHC class I-derived antigens and the number of patients each antigen was detected in. Peptides eluted from MHC class I molecules of CD138+multiple myeloma cell fractions derived from N=6 NDMM patients. CAAs, cancer-associated antigens; 5′ uORF, 5′ upstream open reading frame; 3′ dORF, 3′ downstream open reading frame; ncRNA, non-coding RNA; lincRNA, Long intergenic non-coding RNA.

FIG. 25 shows TCR sequence sharing in tumor and virus reactive BMR-T.

Heatmaps showing pairwise similarities of TCR TRA-TRB sequences split by tested antigen recognition and based on scaled BLOSUM45-similarity (Methods). TCRs are annotated by the respective patient of origin and clustered across all patients (N=12 NDMM patients).

FIGS. 26A-26C show epitope validation of a tumor reactive TCR shared by three NDMM patients. FIG. 26A (SEQ ID NO: 188-227) shows a network diagram of similar tumor reactive CDR3 sequences. Pairwise similarities of TCR TRA-TRB sequence are based on scaled BLOSUM45-similarity. Only events above the 95% bootstrapping threshold as established by background distributions are displayed. TCRs are annotated by the respective patient of origin and clustered across all patients (N=12 NDMM patients). Consensus TRA and TRB sequences for TCR tested in b-c) shown on the left. FIG. 26B (SEQ ID NO: 228-233) shows MHC class I-derived peptide-loaded MHC tetramer flow cytometry staining of autologous BMR-T (methods). Epitope sequences of tested tumor antigens found in Pt-08 by MHC class I immunoprecipitation indicated. FIG. 26C (SEQ ID NO: 228-233) shows fold change (FC) clonal expansion of Pt-08 BMR-T in antigen-specific T cell expansion assay from do to d28 shown as determined by longitudinal TCR sequencing. Irradiated autologous PBMCs loaded with indicated epitopes of tumor antigens found in Pt-08 by MHC class I immunoprecipitation.

FIGS. 27A-27C show tumor reactive T cells expand upon autologous stem cell transplantation. FIG. 27A shows MM-TCR signature score in cells with a TCR exclusive pre bsAb therapy, exclusive post bsAb therapy or overlapping between pre- and post bsAb therapy (n=16 patients, 131,469 cells with productive TCR). Statistical significance was determined by one-way ANOVA with Tukey post hoc test for multiple hypothesis testing correction. FIG. 27B shows a dot plot indicating the proportion of antigen-reactive or orphan BMR-T pre- and post bsAb treatment, split by clinical response (N=18 patients). Statistical significance was determined by repeated-measures ANOVA with Turkey post-hoc test for multiple hypothesis testing correction. FIG. 27C shows a UMAPs and bar charts indicating the proportion of predicted tumor reactive, virus reactivity and orphan TCRs among RRMM patient BMR-T at initial diagnosis (predicted reactive=MM-TCR signature score >0.42; predicted not reactive=MM-TCR signature score <0.42). N=18 patients treated with bispecific BCMAxCD3 antibodies.

FIG. 28 shows fluorescence imaging of BMTCs targeting multiple myeloma by antigen-agnostic microfluidics screening.

FIGS. 29 and 30 show amplified TCRs from tumor-reactive BMTCs retrieved from antigen-agnostic microfluidics screening.

FIGS. 31A-31G show phenotype composition and cloning of TCRs targeting multiple myeloma retrieved from antigen-agnostic microfluidics screening.

FIGS. 32A-32D, 33A-33D, and 34 show tumor specificity validation of TCRs targeting multiple myeloma retrieved from antigen-agnostic microfluidics screening.

FIG. 35A-35D shows MHC class I blocking experiments of TCRs targeting multiple myeloma retrieved from antigen-agnostic microfluidics screening.

FIGS. 36A-36C shows fluorescence imaging of peripheral blood T cells targeting acute myeloid leukemia by antigen-agnostic microfluidics screening.

FIGS. 37A-37C show fluorescence imaging of peripheral blood T cells targeting chronic lymphocytic leukemia by antigen-agnostic microfluidics screening.

FIGS. 38A-38F show compartment tracing of antigen-specific TCRs in multiple myeloma patients.

FIGS. 39A and 39B show TCR sequence similarities in tumor-reactive BMTCs in multiple myeloma patients.

FIGS. 40A and 40B show TCR sequence similarities in virus-specific and random BMTCs in multiple myeloma patients.

FIGS. 41A-41C show epitope mapping of a tumor-reactive TCR shared by three multiple myeloma patients (41A-SEQ ID NO: 234-280) (41B-SEQ ID NO: 228-233), (41C-SEQ ID NO: 228-233).

FIGS. 42A, 42B (SEQ ID NO: 281-286), and 43A-43E (43A-SEQ ID NO: 287-303) show bone marrow reactivity screening against personalized and shared antigens identified in multiple myeloma immunopeptidomes.

FIGS. 44A and 44B show retrospective and prospective TCR signature benchmarking of MM-TCR signature versus published signatures of tumor-infiltrating lymphocytes.

FIGS. 45A-45F show CD29 (ITGB1) as marker gene of tumor specific T cells.

FIGS. 46A-46F show clinical trial cohort of TCRVβ multiple myeloma patients undergoing ASCT.

FIGS. 47A and 47B show tumor-reactive T cells expand upon ASCT.

FIG. 48 shows transfer of tumor-reactive T cells with ASCT.

FIG. 49 shows persistence of tumor-reactive T cells one year after ASCT.

The figures herein are for illustrative purposes only and are not necessarily drawn to scale.

### DETAILED DESCRIPTION OF THE EXAMPLE EMBODIMENTS

### General Definitions

Unless defined otherwise, technical, and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a,” “an,” and “the” include both singular and plural referents unless the context clearly dictates otherwise.

The term “optional” or “optionally” means that the subsequent described event, circumstance, or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The term “about” or “approximately,” as used herein when referring to a measurable value such as a parameter, an amount, a temporal duration, and the like, is meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Examples of subjects/patients include humans and non-human mammals, e.g., non-human primates, dogs, cows, horses, pigs, sheep, goats, cats, mice, rabbits, rats, and transgenic non-human animals. Mammals include, but are not limited to, murines, simians, humans, farm animals, sport animals, and pets. Tissues, cells and their progeny of a biological entity obtained in vivo or cultured in vitro are also encompassed. In specific embodiments, the subject is a human.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure, or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

The “tumor infiltrating lymphocyte (TIL)” used herein means a lymphocyte infiltrating cancer tissue or a tumor microenvironment (TME) after moving from the bloodstream to the site of tumor tissue.

All publications, published patent documents, and patent applications cited herein are hereby incorporated by reference to the same extent as though each individual publication, published patent document, or patent application was specifically and individually indicated as being incorporated by reference.

### OVERVIEW

The present disclosure relates to a platform that may be used to identify and enrich for disease-reactive T cells in a particular disease context, for example cancer-reactive T cells. The platform enables the identification of gene expression profiles that characterize the reactive T cells allowing the disease-reactive T cells to be cloned and further characterized. These gene expression profiles may also be used as a prognostic marker to improve treatment outcomes and to select patients that would most benefit from the therapeutic modalities discussed herein. For example, as further detailed herein, the gene expressions profiles can predict a consistent response to cell based therapies, antibody based therapeutics, including bi-specific antibodies, and disease-specific vaccines. In one aspect, the embodiments disclosed herein are directed to T cell receptors from the identified cancer-reactive T cells and their use in preparing engineered cell therapy products. The present disclosure also relates to methods for identifying the specific antigens recognized by the disease-reactive T cells. As detailed further herein, the methods enable the identification of antigens that are found across multiple patients in a given disease setting leading to a convergence of shared immune responses and the potential for off-the-shelf cell therapeutics comprising T cell receptors targeting such antigens, and more effective vaccines comprising such antigens.

### Engineered Immune Cells

In one aspect, embodiments disclosed herein are directed to engineered immune cells comprising the disease-reactive antigen receptors identified using the methods disclosed herein. The engineered immune cell may be a CD4+ T cell, a CD8+ T cell, or a natural killer (NK) T cell. The immune cell may be autologous or allogenic. The immune cell may be a chimeric antigen receptor (CAR) T cell, wherein the CAR comprises all or an antigen-binding portion of a TCR identified using the methods disclosed herein. The engineered immune cell may be a tumor-infiltrating lymphocyte (TIL) identified as comprising or engineered to comprise TCRs identified using the methods disclosed herein and expanded ex vivo before being administered to a patient in need thereof. The ex vivo expansion may include culturing the TIL in specific culture conditions that modify a phenotype or gene expression profile of the TIL from its natural state. The TIL may also be formulated in a composition that comprises additional molecules, such as cytokines, to enhance TIL cell acceptance by a patient and/or TIL activity. The engineered immune cell may further comprise one or more modifications, for example one or more gene modifications to modify antigen processing by the cell. The one or more modifications may comprise editing to knock-out or knock-down expression of B2M, human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI) (see WO2016/011210). In an embodiment, the T cells are edited ex vivo by CRISPR to knock-out or knock down the expression of an antigen selected from B cell maturation antigen (BCMA), transmembrane activator and CAML Interactor (TACI), or B-cell activating factor receptor (BAFF-R), CD38, CD138, CS-1, CD33, CD26, CD30, CD53, CD92, CD100, CD148, CD150, CD200, CD261, CD262, or CD362.

In one embodiment, the engineered immune cell comprises a TCR capable of recognizing a cancer-associated antigen. In one embodiment, the cancer-associated antigen is an antigen associated with a hematological malignancy. The hematological malignancy may be a leukemia, a lymphoma, a myeloma, myelodysplastic syndrome, a myeloproliferative neoplasm, a histocytic disorder. The leukemia may be acute lymphoblastic leukemia, chronic lymphoblastic leukemia, acute myeloid leukemia, chronic myeloid leukemia, acute promyelocytic leukemia. The lymphoma may be a Non-Hodgkin's lymphoma or Hodgkin's lymphoma. The Non-Hodgkin lymphoma may be diffuse large B-cell lymphoma, follicular lymphoma, mantle cell lymphoma, marginal zone lymphoma, Burkitt lymphoma, T-cell lymphoma, or Waldenström's macroglobulinemia. The myeloma may be multiple myeloma or light chain amyloidosis myeloma. In one embodiment, the hematological malignancy is multiple myeloma. In another embodiment, the hematological malignancy is a leukemia. In one embodiment, the leukemia is acute myeloid leukemia.

In one embodiment, the engineered immune cell comprises a TCR capable of recognizing a microbial-associated antigen including virus-associated antigens, bacteria-associated antigens, fungal-associated antigens, and parasite-associated antigens.

### TCRs

Provided herein are TCRs or antigen-binding fragment thereof, comprising an alpha chain comprising a variable alpha region and a beta chain comprising a variable beta region. The variable regions include a complementary determining region 1 (CDR-1), a complementary determining region 2 (CDR-2), and a complementary determining region 3 (CDR-3). The TCR is a heterodimer composed of two different protein chains. The highly polymorphic TCR is generated by joining of non-contiguous gene segments (Vβ, Dβ, Jβ for TCRβ and Vα, Jα for TCRα) together with deletion/insertion of random sequences at junctions and Recombination Signal Sequences (RSS) to form the highly variable CDR3 regions. The recognition of MHC-bound peptide by the combined TCRβ and TCRα proteins occurs primarily by the CDR3 regions (see e.g., Robins H S, Srivastava S K, Campregher P V, et al. Overlap and effective size of the human CD8+ T cell receptor repertoire. Sci Transl Med. 2010; 2(47):47ra64). In most T cells (about 95%), these two protein chains are termed the alpha (α) and beta (β) chains. However, in a small percentage of T cells (about 5%), these two protein chains are termed the gamma and delta (γ/δ) chains. The ratio of TCRs comprised of α/β chains versus γ/δ chains may change during a diseased state. When the TCR engages with antigenic peptide and MHC (peptide/MHC), the T lymphocyte is activated through a series of biochemical events mediated by associated enzymes, co-receptors, specialized adaptor molecules, and activated or released transcription factors.

In one embodiment, the TCR or antigen-binding fragment thereof binds to or recognizes one or more peptide epitopes. In an embodiment, the TCR or antigen-binding fragment thereof, when expressed on the surface of a T cell, stimulates cytotoxic activity against a target cell. In an embodiment, the target cell is a cancer cell.

In an embodiment, the TCR is encoded by a nucleotide sequence that has been codon-optimized. In an embodiment, the alpha and/or beta chain further comprise a signal peptide. In particular embodiments, the TCR is isolated or purified or is recombinant. In an embodiment, the TCR is human. In an embodiment the TCR is monoclonal. In an embodiment, the TCR is single-chain. In an embodiment the TCR comprises two chains.

Provided herein are nucleic acid molecules encoding any of the provided TCRs, or an alpha or beta chain thereof. In one embodiment the nucleotide sequence is codon-optimized. Provided herein is a vector comprising a nucleic acid of any provided herein. In an embodiment, the vector is an expression vector. In particular embodiments, the vector is a viral vector. Provided herein is an engineered cell comprising the nucleic acid molecule of any provided herein or vector of any provided herein. Also provided herein is an engineered cell, including the TCR of any provided herein. In an embodiment the TCR is heterologous to the cell. In an embodiment, the engineered cell is a cell line. In particular embodiments, the engineered cell is a primary cell obtained from a subject. In an embodiment, the subject is a mammalian subject. In an embodiment, the subject is human. In particular embodiments, the engineered cell is a T cell. In an embodiment, the T cell is CD8+. In an embodiment, the T cell is CD4+.

In an embodiment, TCRs are identified that recognize a tumor antigen. The term “tumor antigen” as used throughout this specification refers to an antigen that is uniquely or differentially expressed by a tumor cell, whether intracellular or on the tumor cell surface (preferably on the tumor cell surface), compared to a normal or non-neoplastic cell. By means of example, a tumor antigen may be present in or on a tumor cell and not typically in or on normal cells or non-neoplastic cells (e.g., only expressed by a restricted number of normal tissues, such as testis and/or placenta), or a tumor antigen may be present in or on a tumor cell in greater amounts than in or on normal or non-neoplastic cells, or a tumor antigen may be present in or on tumor cells in a different form than that found in or on normal or non-neoplastic cells. The term thus includes tumor-specific antigens (TSA), including tumor-specific membrane antigens, tumor-associated antigens (TAA), including tumor-associated membrane antigens, embryonic antigens on tumors, growth factor receptors, growth factor ligands, etc.

In one embodiment, the engineered immune cell comprises a TCR capable of recognizing an antigen in SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

In one embodiment, the engineered immune cell comprises a TCR comprising a TCR alpha chain CDR3 sequence selected from SEQ ID NO: 1-62, 41855-41902 or a TCR beta chain CDR3 sequence selected from SEQ ID NO: 63-121 or 41903-41948. In one embodiment, the TCR comprise recognizes CTAG2 or IGKV. In an embodiment, TCR comprises an alpha or beta chain CDR3 sequences of TCR No. 11729 or 15343 from Table 8.

### CARS

In some aspects, the genetically engineered antigen receptor is a T cell receptor (TCR) or a functional non-TCR antigen recognition receptor. In an embodiment, it is a chimeric antigen receptor (CAR), such as an activating or stimulatory CAR, an inhibitory CAR and/or a costimulatory CAR. Among the CARs are those with an extracellular antigen-recognition domain that specifically binds to the target antigen and an intracellular signaling domain comprising an ITAM, such as an intracellular domain of a CD3-zeta (CD35) chain those that further comprise a costimulatory signaling region, such as a signaling domain of CD28 or 41BB. In an embodiment, the CAR comprises an extracellular antigen-recognition domain that specifically binds to the target antigen and an intracellular signaling domain that comprises a signaling portion of an immune checkpoint molecule, such as PD-1 or CTLA4.

In an embodiment, the engineered antigen receptors include chimeric antigen receptors (CARs), including activating or stimulatory CARs, costimulatory CARs (see WO2014/055668), and/or inhibitory CARs (iCARs, see Fedorov et al., Sci. Transl. Medicine, 5 (215) (December 2013). The CARs generally include an extracellular antigen (or ligand) binding domain linked to one or more intracellular signaling components, in an embodiment, via linkers and/or transmembrane domain(s). Such molecules typically mimic or approximate a signal through a natural antigen receptor, a signal through such a receptor in combination with a costimulatory receptor, and/or a signal through a costimulatory receptor alone.

In an embodiment, CAR is constructed with specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen-binding molecules, such as one or more antigen-binding fragments, domains, or portions, or one or more antibody variable domains and/or antibody molecules. In an embodiment, the CAR includes an antigen-binding portion or portions of an antibody molecule, such as a single-chain antibody fragment (scFv) derived from the variable heavy (VH) and variable light (VL) chains of a monoclonal antibody (mAb).

In an embodiment, the CAR contains an antibody or an antigen-binding fragment (e.g., scFv) that specifically recognizes an antigen, such as an intact antigen, expressed on the surface of a cell.

In an embodiment, the CAR contains a TCR-like antibody, such as an antibody or an antigen-binding fragment (e.g. scFv) that specifically recognizes an intracellular antigen, such as a tumor-associated antigen, presented on the cell surface as an MHC-peptide complex. In an embodiment, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

In one embodiment, the engineered immune cell comprises a CAR comprising a sequence comprising a TCR alpha chain CDR3 sequence selected from SEQ ID NO: 1-62, 41855-41902 or a TCR beta chain CDR3 sequence selected from SEQ ID NO: 63-121 or 41903-41948.

### Methods for Making Engineered Cells

Provided herein is a method for producing a cell of any of the provided embodiments, including introducing any of the provided vectors into a cell in vitro or ex vivo. In an embodiment, the introduction is carried out by transduction. In particular embodiments, the method further includes introducing into the cell one or more agent, wherein each of the one or more agent is independently capable of inducing genetic disruption of a T cell receptor alpha or beta chain gene. In an embodiment, the one or more agents capable of inducing a genetic disruption comprises a DNA binding protein or DNA-binding nucleic acid that specifically binds to or hybridizes to the target site.

In embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse, or remove cells sensitive to particular reagents. In some examples, cells are separated based on one or more property, such as density, adherent properties, size, sensitivity, and/or resistance to particular components.

In some examples, cells from the circulating blood of a subject are obtained, e.g., by apheresis or leukapheresis. The samples, in an embodiment, contain lymphocytes, including T cells, monocytes, granulocytes, B cells, other nucleated white blood cells, red blood cells, and/or platelets, and in an embodiment contains cells other than red blood cells and platelets.

In an embodiment, the blood cells collected from the subject are washed, e.g., to remove the plasma fraction and to place the cells in an appropriate buffer or media for subsequent processing steps. In an embodiment, the cells are washed with phosphate buffered saline (PBS). In an embodiment, the wash solution lacks calcium and/or magnesium and/or many or all divalent cations. In an embodiment, a washing step is accomplished a semi-automated “flow-through” centrifuge (for example, the Cobe 2991 cell processor, Baxter) according to the manufacturer's instructions. In an embodiment, a washing step is accomplished by tangential flow filtration (TFF) according to the manufacturer's instructions. In an embodiment, the cells are resuspended in a variety of biocompatible buffers after washing, such as, for example, Ca++/Mg++ free PBS. In an embodiment, components of a blood cell sample are removed, and the cells directly resuspended in culture media.

In an embodiment, the methods include density-based cell separation methods, such as the preparation of white blood cells from peripheral blood by lysing the red blood cells and centrifugation through a Percoll or Ficoll gradient.

### Methods of Use in Adoptive Cell Therapy

In embodiments disclosed herein, the engineered cells described above may be used in novel therapeutic approaches for treating cancer. These engineered immune cells can be utilized to target hematological malignancies including MM, AML, and CLL. The engineered T cells may be autologous or allogeneic and may include modifications to further enhance their therapeutic efficacy. The engineered immune cells may comprise one or more modifications to enhance their immune reactivity, longevity, and anti-tumor effects. These modifications may include, but are not limited to, gene editing to knock out inhibitory receptors, enhance expression of co-stimulatory molecules, or secrete therapeutic cytokines. Additionally, the immune cells may include engineered receptors, such as chimeric antigen receptors (CARs) or specific T cell receptors (TCRs), to target cancer cells.

In example embodiments, identified antigen-activated T cell receptor (TCR) disclosed herein are used in constructing cells for adoptive cell transfer. In example embodiments, TCRs that are clonal or specific to an antigen are identified. In example embodiment, the TCR CDR3 is used to generate a chimeric antigen receptor. As used herein, “ACT,” “adoptive cell therapy,” and “adoptive cell transfer” are used interchangeably. In an embodiment, adoptive cell therapy (ACT) refers to the transfer of cells to a patient with the goal of transferring the functionality and characteristics into the new host by engraftment of the cells (see, e.g., Mettananda et al., Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells (e.g., T cells or NK cells), back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer, and colorectal carcinoma, as well as patients with CD19-expressing hematologic malignancies (Kalos et al., (2011) Science Translational Medicine 3 (95): 95ra73). In an embodiment, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

In an embodiment, an antigen to be targeted in adoptive cell therapy (such as particularly CAR or TCR T cell therapy) of a disease (such as particularly of tumor or cancer) is a tumor-specific antigen (TSA).

In an embodiment, an antigen to be targeted in adoptive cell therapy (such as particularly CAR or TCR T cell therapy) of a disease (such as particularly of tumor or cancer) is a neoantigen.

In an embodiment, an antigen to be targeted in adoptive cell therapy (such as particularly CAR or TCR T cell therapy) of a disease (such as particularly of tumor or cancer) is a tumor-associated antigen (TAA) or cancer-associated antigen (CAA).

In an embodiment, an antigen to be targeted in adoptive cell therapy (such as particularly CAR or TCR T cell therapy) of a disease (such as particularly of tumor or cancer) is a universal tumor antigen. In certain preferred embodiments, the universal tumor antigen is selected from the group consisting of: a human telomerase reverse transcriptase (hTERT), urviving, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (DI), and any combinations thereof.

In an embodiment, an antigen (such as a tumor antigen) to be targeted in adoptive cell therapy (such as particularly CAR or TCR T cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin's lymphoma, indolent non-Hodgkin's lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells or natural killer cells (NK), specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target (see, e.g., Gong Y, Klein Wolterink R G J, Wang J, Bos G M J, Germeraad W T V. Chimeric antigen receptor natural killer (CAR-NK) cell design and engineering for cancer therapy. J Hematol Oncol. 2021; 14(1):73; Guedan S, Calderon H, Posey A D Jr, Maus M V. Engineering and Design of Chimeric Antigen Receptors. Mol Ther Methods Clin Dev. 2018; 12:145-156; Petersen C T, Krenciute G. Next Generation CAR T Cells for the Immunotherapy of High-Grade Glioma. Front Oncol. 2019; 9:69; and Lu H, Zhao X, Li Z, Hu Y, Wang H. From CAR-T Cells to CAR-NK Cells: A Developing Immunotherapy Method for Hematological Malignancies. Front Oncol. 2021). While the antigen-binding domain of a CAR is often an antibody or antibody fragment (e.g., a single chain variable fragment, scFv), the binding domain is not particularly limited so long as it results in specific recognition of a target. For example, In an embodiment, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified to prevent off-target binding by FcRs or other potential interfering objects. For example, the hinge may comprise an IgG4 Fc domain with or without a S228P, L235E, and/or N297Q mutation (according to Kabat numbering) to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In an embodiment, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In an embodiment, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In an embodiment, the one or more costimulatory signaling domains comprise a functional signaling domain of a protein selected, each independently, from the group consisting of: 4-1BB, CD27, and CD28. In an embodiment, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, include the transmembrane and signaling domains of CD28 (such as amino acid residues 114-220 of SEQ ID NO: 10, full sequence shown in SEQ ID NO: 6 of U.S. Pat. No. 7,446,190; these include the following portion of CD28 as set forth in Genbank identifier NM 006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ. ID NO: 304). Alternatively, when the zeta sequence lies between the CD28 sequence and the antigen-binding element, intracellular domain of CD28 is used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen to be activated and expanded following engagement of their native αβTCR, for example by antigen on professional antigen-presenting cells, with attendant costimulation. In addition, additional engineered receptors may be provided on the immunoresponsive cells, for example to improve targeting of a T cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34:1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. ID NO: 305) and continuing all the way to the carboxy-terminus of the protein. To encode the anti-CD19 scFv component of the vector, the authors designed a DNA sequence which was based on a portion of a previously published CAR (Cooper et al., (2003) Blood 101:1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16:457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20:70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, In an embodiment, cells intended for adoptive cell therapies, more particularly immunoresponsive cells such as T cells, may express the FMC63-28Z CAR as described by Kochenderfer et al. (supra). Hence, In an embodiment, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 305) and continuing all the way to the carboxy-terminus of the protein. The sequence is reproduced herein:

(SEQ ID NO: 304)
IEVMYPPPYLDNEKSNGTIIHVKGKHLCPSPLFPGPSKPFWVL
VVVGGVLACYSLLVTVAFIIFWVRSKRSRLLHSDYMNMTPRRP
GPTRKHYQPYAPPRDFAAYRS.

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FceRI gamma chain; or CD28-FceRI gamma chain) were disclosed. Hence, In an embodiment, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T cell signaling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In an embodiment, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., Oral Oncol. 2018 March; 78:145-150; and Jin et al., Neuro Oncol. 2018 Jan. 10; 20(1):55-65). CD70 is expressed by diffuse large B-cell and follicular lymphoma and also by the malignant cells of Hodgkin's lymphoma, Waldenstrom's macroglobulinemia and multiple myeloma, and by HTLV-1- and EBV-associated malignancies. (Agathanggelou et al. Am. J. Pathol. 1995; 147:1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In an embodiment, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In an embodiment, the chimeric inhibitory receptor comprises an extracellular antigen-binding element (or portion or domain) configured to specifically bind to a target antigen, a transmembrane domain, and an intracellular immunosuppressive or repressive signaling domain. In an embodiment, the second target antigen is an antigen that is not expressed on the surface of a cancer cell or infected cell or the expression of which is downregulated on a cancer cell or an infected cell. In an embodiment, the second target antigen is an MHC-class I molecule. In an embodiment, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T cells expressing CARs may be further modified to reduce or eliminate expression of endogenous TCRs to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, In an embodiment, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR also may comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T cell that expresses the CAR can be administered to a subject, but the CAR cannot bind its target antigen until the second composition comprising an antigen-specific binding domain is administered.

Alternative switch mechanisms include CARs that require multimerization to activate their signaling function (see, e.g., US 2015/0368342, US 2016/0175359, US 2015/0368360) and/or an exogenous signal, such as a small molecule drug (US 2016/0166613, Yung et al., Science, 2015), to elicit a T cell response. Some CARs may also comprise a “suicide switch” to induce cell death of the CAR T cells following treatment (Buddee et al., PLOS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids, or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV. In an embodiment, inducible gene switches are used to regulate expression of a CAR or TCR (see, e.g., Chakravarti, Deboki et al. “Inducible Gene Switches with Memory in Human T Cells for Cellular Immunotherapy.” ACS synthetic biology vol. 8,8 (2019): 1744-1754).

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T cells may be expanded, for example by co-culture on AaPC in presence of soluble factors, such as IL-2 and IL-21. This expansion may for example be carried out to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In an embodiment, ACT includes co-transferring CD4+ Th1 cells and CD8+ CTLs to induce a synergistic antitumour response (see, e.g., Li et al., Clin Transl Immunology. 2017 October; 6(10): e160).

In an embodiment, antigen specificity can be conferred to Tregs by engineering the expression of transgenic T cell receptor (TCR) or chimeric antigen receptor (CAR), such as to modulate immune responses in organ transplant and autoimmune diseases (see, e.g., Arjomandnejad M, Kopec A L, Keeler A M. Biomedicines. 2022; 10(2):287). Regulatory T cells (Tregs) are a T cell subset known for their immunomodulatory function. Expression of CD4, CD25, and the master transcription factor, forkhead box P3 (FOXP3), are the main characteristic markers of conventional Tregs. However, other regulatory immune cells with different properties such as CD8+ Tregs, or type 1 regulatory T cells (Tr1) have been described. Id. Tregs are divided into “natural” Tregs that develop in the thymus or “induced” Tregs that are generated in the periphery. Id. Regulatory T cells suppress immune responses through multiple mechanisms including direct interaction with other immune cells or by producing immunosuppressive cytokines such as interleukin-10 (IL-10) and Transforming growth factor beta (TGF-β). Id. Directing Tregs towards a desired antigen may boost the overall response and lower the risk of broad and systemic immunosuppression or generation of an inflammatory response. Id.

In an embodiment, Th17 cells are transferred to a subject in need thereof. Th17 cells have been reported to directly eradicate melanoma tumors in mice to a greater extent than Th1 cells (Muranski P, et al., Blood. 2008 Jul. 15; 112(2):362-73; and Martin-Orozco N, et al., Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In an embodiment, ACT may include autologous iPSC-based vaccines, such as irradiated iPSCs in autologous anti-tumor vaccines (see e.g., Kooreman, Nigel G. et al., Cell Stem Cell 22, 1-13, 2018).

Unlike T cell receptors (TCRs) that are MHC restricted, CARs can potentially bind any cell surface-expressed antigen and can thus be more universally used to treat patients (see Irving et al., Front. Immunol., 3 Apr. 2017). In an embodiment, in the absence of endogenous T cell infiltrate (e.g., due to aberrant antigen processing and presentation), which precludes the use of TIL therapy and immune checkpoint blockade, the transfer of CAR T cells may be used to treat patients (see, e.g., Hinrichs C S, Rosenberg S A. Immunol Rev (2014) 257(1):56-71).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In an embodiment, the treatment is administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., Cancer Immunol Res (2015) 3(10):1115-22; and Kamta et al., Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment is administrated into patients undergoing an immunosuppressive treatment (e.g., glucocorticoid treatment). The cells, or population of cells, may be made resistant to at least one immunosuppressive agent due to the inactivation of a gene encoding a receptor for such immunosuppressive agent. In an embodiment, the immunosuppressive treatment provides for the selection and expansion of the immunoresponsive T cells within the patient.

In an embodiment, the treatment is administered before primary treatment (e.g., surgery or radiation therapy) to shrink a tumor before the primary treatment. In another embodiment, the treatment is administered after primary treatment to remove any remaining cancer cells.

In an embodiment, immunometabolic barriers are targeted therapeutically prior to and/or during ACT to enhance responses to ACT or CAR T cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation, or transplantation. The cells or population of cells can be administered to a patient subcutaneously, intradermally, intratumorally, intranodally, intramedullary, intramuscularly, intrathecally, by intravenous or intralymphatic injection, or intraperitoneally. In an embodiment, the disclosed CARs are delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells comprises administering 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the ordinary skill of one in the art. An effective amount means an amount that provides a therapeutic or prophylactic benefit. The dosage administrated will be dependent upon the age, health and weight of the recipient, kind of concurrent treatment, if any, frequency of treatment and the nature of the effect desired.

In an example embodiment, the effective amount of cells can be any amount ranging from about 1 or 2 cells to 1×101 cells/mL, 1×1020 cells/mL or more, such as about 1×101 cells/mL, 1×102 cells/mL, 1×103 cells/mL, 1×104 cells/mL, 1×105 cells/mL, 1×106 cells/mL, 1×107 cells/mL, 1×108 cells/mL, 1×109 cells/mL, 1×1010 cells/mL, 1×1011 cells/mL, 1×1012 cells/mL, 1×1013 cells/mL, 1×1014 cells/mL, 1×1015 cells/mL, 1×1016 cells/mL, 1×1017 cells/mL, 1×1018 cells/mL, 1×1019 cells/mL, to/or about 1×1020/cells/mL or any numerical value or subrange within any of these ranges.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Front. Pharmacol. 2015; 6:95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; Georgiadis et al., Mol Ther. 2018 May 2; 26(5):1215-1227; and Roth, T. L. Curr Hematol Malig Rep 15, 235-240 (2020)). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T cells (see PCT Patent Publications: WO2013176915, WO2014059173, WO2014172606, WO2014184744, and WO2014191128).

In an embodiment, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In an embodiment, homology directed repair (HDR) is used to concurrently inactivate a gene (e.g., TRAC) and insert an endogenous TCR or CAR into the inactivated locus.

Hence, In an embodiment, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T cell receptor, such as T cell receptor alpha locus (TRA) or T cell receptor beta locus (TRB), for example T cell receptor alpha constant (TRAC) locus, T cell receptor beta constant 1 (TRBC1) locus or T cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543:113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, In an embodiment, editing of cells (such as by CRISPR/Cas), particularly cells intended for adoptive cell therapies, more particularly immunoresponsive cells such as T cells, is performed to knock-out or knock-down expression of an endogenous TCR in a cell. For example, NHEJ-based or HDR-based gene editing approaches are employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid, or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In an embodiment, editing of cells (such as by CRISPR/Cas), particularly cells intended for adoptive cell therapies, more particularly immunoresponsive cells such as T cells, is performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In an embodiment, the immune checkpoint targeted is the programmed death-1 (PD-1 or CD279) gene (PDCD1) (see, e.g., Rupp L J, Schumann K, Roybal K T, et al. CRISPR/Cas9-mediated PD-1 disruption enhances anti-tumor efficacy of human chimeric antigen receptor T cells. Sci Rep. 2017; 7(1):737). In other embodiments, the immune checkpoint targeted is cytotoxic T lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., Biochem Soc Trans. 2016 Apr. 15; 44(2):356-62). SHP-1 is a widely expressed inhibitory protein tyrosine phosphatase (PTP). In T cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Front. Immunol. 6:418).

WO2014172606 relates to the use of MT1 and/or MT2 inhibitors to increase proliferation and/or activity of exhausted CD8+ T cells and to decrease CD8+ T cell exhaustion (e.g., decrease functionally exhausted or unresponsive CD8+ immune cells). In an embodiment, metallothioneins are targeted by gene editing in adoptively transferred T cells.

In an embodiment, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAGI, SITI, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In an embodiment, cells are engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In an embodiment, editing of cells (such as by CRISPR/Cas), particularly cells intended for adoptive cell therapies, more particularly immunoresponsive cells such as T cells, is performed to knock-out or knock-down expression of an endogenous gene in a cell, said endogenous gene encoding an antigen targeted by an exogenous CAR or TCR, thereby reducing the likelihood of targeting of the engineered cells. In an embodiment, the targeted antigen is one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In an embodiment, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M are knocked-out or knocked-down. Preferably, B2M is knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In an embodiment, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method comprises obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. In a preferred embodiment, the tumor sample is obtained from a human. In an embodiment, the tumor sample is obtained from a subject to be treated.

T cells can be obtained from a number of sources, including peripheral blood mononuclear cells (PBMC), bone marrow, lymph node tissue, spleen tissue, and tumors. In an embodiment of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis are washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or many, if not all, divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells can be resuspended in a variety of biocompatible buffers, such as, for example, Ca-free, Mg-free PBS. Alternatively, undesirable components of the apheresis sample can be removed, and the cells can be directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for an incubation time sufficient for positive selection of the desired T cells. In one embodiment, the period is about 30 minutes. In a further embodiment, the incubation time ranges from 30 minutes to 36 hours or longer and all integer values there between. In a further embodiment, the incubation time is at least 1, 2, 3, 4, 5, or 6 hours. In yet another preferred embodiment, the incubation time is from 10 hours to 24 hours. In one preferred embodiment, the incubation time is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In an embodiment, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In an embodiment, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In an embodiment, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml are used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/mL. In other embodiments, the concentration used is from about 1×105/ml to 1×106/mL, and any integer value in between.

T cells can also be frozen. Without wishing to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° C. per minute and stored in the vapor phase of a liquid nitrogen storage tank. Other methods of controlled freezing may be used, such as uncontrolled freezing immediately at −20° C. or in liquid nitrogen.

T cells for use in the present invention also may be antigen-specific T cells. For example, tumor-specific T cells can be used. In an embodiment, antigen-specific T cells are isolated from a patient of interest, such as a patient afflicted with a cancer or an infectious disease. In one embodiment, neoepitopes are determined for a subject, and T cells specific to these antigens are isolated. Antigen-specific cells for use in expansion may also be generated in vitro using any number of methods known in the art (e.g., as described in U.S. Patent Publication No. US 20040224402 and U.S. Pat. No. 6,040,177). Antigen-specific cells for use in the present invention also may be generated using any number of methods known in the art (e.g., as described in Current Protocols in Immunology and Current Protocols in Cell Biology, both published by John Wiley & Sons, Inc., Boston, Mass).

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 125I labeled β2-microglobulin (β2m) into MHC class I/β2m/peptide heterotrimeric complexes (see Parker et al., J. Immunol. 152:163, 1994).

In one embodiment, cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or at least about 20-, at least about 30-, at least about 40-, at least about 50-, at least about 60-, at least about 70-, at least about 80-, or at least about 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment, both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In an embodiment, T cells comprising a CAR or an exogenous TCR, may be manufactured as described in WO2015120096, by a method comprising: enriching a population of lymphocytes obtained from a donor subject; stimulating the population of lymphocytes with one or more T cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In an embodiment, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In an embodiment, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In an embodiment, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m2/day.

In an embodiment, a patient in need of adoptive cell transfer may be administered a TLR agonist to enhance anti-tumor immunity (see, e.g., Urban-Wojciuk, et al., Front Immunol. 2019; 10:2388; and Kaczanowska et al., J Leukoc Biol. 2013 June; 93 (6): 847-863). In an embodiment, TLR agonists are delivered in a nanoparticle system (see, e.g., Buss and Bhatia, Proc Natl Acad Sci.

### Methods of Use in Stem Cell Transplant

Autologous stem cell transplantation (ASCT) represents a therapeutic approach for treating hematological malignancies such as multiple myeloma (MM), acute myeloid leukemia (AML), and chronic lymphocytic leukemia (CLL). This method involves the collection and reinfusion of hematopoietic stem cells (HSCs) into patients following high-dose chemotherapy or radiation therapy designed to eradicate malignant cells. The reinfused HSCs subsequently repopulate the bone marrow, facilitating the recovery of the patient's hematopoietic system. As disclosed herein, the engineered cells may be included in compositions used for ASCT. The antigen-activated TCRs disclosed herein may be engineered into patient derived T cells and included in compositions used for ASCT. As discussed in further detail in the Examples section below, the antigen-activated TCRs may enhance patient response to ASCT therapy and help sustain cancer remission. The identification, characterization, and utilization of these tumor-reactive TCRs represent a targeted therapeutic strategy that holds promise for improving patient prognosis and achieving durable remissions.

### Immunogenic Compositions

Described In an embodiment herein are immunogenic compositions that can contain one or more disease associated antigens, e.g. cancer associated antigens (CAAs) and/or one or more polynucleotides encoding the one or more CAAs. In an embodiment, the cancer associated antigen is a conserved cancer antigen. In this context herein, “conserved cancer antigen” refers to a cancer associated antigen of a cancer cell that is recognized by a TCR comprising a conserved cancer gene signature. In an embodiment, the CAA (including but not limited to, a conserved cancer antigen) is a peptide or polypeptide antigen found in SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC. In an embodiment, the CAA (including but not limited to, a conserved cancer antigen) is a polynucleotide. In an embodiment, the CAA (including but not limited to, a conserved cancer antigen) is recognized by a TCR. In an embodiment, the CAA (including but not limited to, a conserved cancer antigen) is capable of presentation in an MHC I (HLA I) or MCH II (HLA II) molecule on a cancer cell. In an embodiment, the CAA (including but not limited to, a conserved cancer antigen) is capable of presentation in an MHC I (HLA I) or MCH II (HLA II) molecule as identified in SEQ ID NO: 29988-41854.

In an embodiment the CAA is selected from SEQ ID NOs: 325-4747, SEQ ID NOs: 4748-4778, SEQ ID NO:s 4779-4902, SEQ ID NO:s 4903-4927, SEQ ID NOs: 4928-26232, SEQ ID NO: 26233-26364, SEQ ID NO: 26365-26738, SEQ ID NO: 26739-28624, SEQ ID NOs: 26825-28633, SEQ ID NOs: 28634-28675, SEQ ID NOs: 28676-29125, SEQ ID NOs: 29126-29987, or SEQ ID NO: 29988-41854.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In an embodiment, MHC class I molecules deliver peptides originating in the cytosol to the cell surface, where a MHC-peptide complex is recognized by T cells, such as generally CD8+ T cells, but in some cases CD4+ T cells. In an embodiment, MHC class II molecules deliver peptides originating in the vesicular system to the cell surface, where they are typically recognized by CD4+ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In an embodiment, the MHC-peptide complex is present or displayed on the surface of cells. In an embodiment, the MHC-peptide complex can be specifically recognized by an antigen receptor, such as a TCR, TCR-like CAR or antigen-binding portions thereof.

In an embodiment, the CAA(s) (including but not limited to, a conserved cancer antigen(s)) are selected from a peptide selected from or are encoded by a polynucleotide selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC. In an embodiment, the conserved cancer antigens are or are encoded by a polynucleotide selected from a target sequence of SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC. In an embodiment, the conserved cancer antigens are selected from a peptide selected from or are encoded by a polynucleotide selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC. In an embodiment, the conserved cancer antigens are or are encoded by a polynucleotide selected from a target sequence of SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC. In an embodiment, the one or more polynucleotides encoding the one or more CAAs (including but not limited to, conserved cancer antigens) is DNA. In an embodiment, the one or more polynucleotides encoding the one or more CAAs (including but not limited to, conserved cancer antigens) is RNA. In an embodiment, the one or more polynucleotides encoding the one or more CAAs (including but not limited to, conserved cancer antigens) is mRNA.

In an embodiment, the immunogenic composition can stimulate an immune response in a subject to which it is administered. In an embodiment, the immune response is a cell-mediated immune response. In an embodiment, the immune response is a humoral immune response. In an embodiment, the immune response includes B-cell, plasma cell, and/or antibody production (collectively referred to as a B-cell response). In an embodiment, the immune response includes a T-cell production (also referred to as a T-cell response). In an embodiment, the T-cell response includes CD 4+ T-cell production, CD8+ T cell production, or both. In an embodiment, the immune response includes both a B-cell and T-cell response.

In an embodiment, the immunogenic composition is formulated as a vaccine. In an embodiment, the immunogenic composition is formulated as a protein or peptide vaccine. In an embodiment, the immunogenic composition is formulated as a DNA vaccine. In an embodiment, the immunogenic composition is formulated as an RNA, such as an mRNA, vaccine. In an embodiment, the immunogenic composition or formulation thereof is a cancer vaccine. In other words, In an embodiment, the immunogenic composition can stimulate an immune response against a cancer. In an embodiment, the immune response stimulated by the cancer vaccine is effective to reduce or eliminate the cancer in subject. In an embodiment, the cancer is a blood cancer. In an embodiment, the cancer is a white blood cell cancer. In an embodiment, the cancer is multiple myeloma.

In an embodiment, the immunogenic compositions may be combined with one or more antigenic components and/or anti-viral therapeutics, anti-proliferative therapeutics, anti-neoplastic therapeutics, and/or chemotherapeutics. In some examples, such combination may elicit cellular and/or antibody-mediated immune response, e.g., production or activation of antibodies, B cells, helper T cells, suppressor T cells, and/or cytotoxic T cells and/or gamma-delta T cells. These and other embodiments are described in greater detail elsewhere herein.

### Cancer Associated Antigen Polynucleotides and Polypeptides

In an embodiment, the CAA (including but not limited to, conserved cancer antigen) is a peptide or a polypeptide or a polynucleotide encoding said peptide or polypeptide. In an embodiment the CAA (including but not limited to, conserved cancer antigen) is recognized by a TCR or component thereof. In an embodiment, the TCR that recognizes a CAA (including but not limited to, conserved cancer antigen) described comprises a TCR alpha CDR3 sequence selected from SEQ ID NOs: 1-62, 41855-41902, or a TCR beta CDR3 sequence selected from SEQ ID NO: 63-121 [or 41903-41948.

In an embodiment the CAA is peptide selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC, or a combination thereof. In an embodiment, the polynucleotides are codon optimized for expression in humans or non-human animals. In an embodiment, the one or more CAA polynucleotides or encoding polynucleotides (including, but not limited to, one or more conserved antigen polynucleotides or conserved antigen encoding polynucleotides) has a sequence corresponding to a (a) an annotated region of a genome; (b) an unannotated region of a genome; (c) a mutation; (d) a 5′UTR; (e) a 3′UTR; (f) an open reading frame; (g) a non-canonical open reading frames (nuORFs), or (h) any combination thereof. In an embodiment, the one or more CAA polynucleotides or encoding polynucleotides (including, but not limited to, one or more conserved antigen polynucleotides or conserved antigen encoding polynucleotides) has a sequence corresponding to a (a) an annotated region of a cancer cell genome; (b) an unannotated region of a cancer cell genome; (c) a mutation; (d) a 5′UTR of a cancer cell; (e) a 3′UTR or a cancer cell; (f) an open reading frame of a cancer cell; (g) a non-canonical open reading frames (nuORFs) of a cancer cell, or (h) any combination thereof. In an embodiment, the cancer cell is a blood cancer cell. In an embodiment, the cancer cell is a white blood cell cancer cell. In an embodiment the cancer cell is a plasma cell. In an embodiment, the cancer is multiple myeloma, and the cancer cell is a multiple myeloma cell.

In an embodiment, a CAA antigen or antigen encoding polynucleotide (including, but not limited to, a conserved cancer antigen or conserved antigen encoding polynucleotide) of the present invention has 50-100% identity a peptide of SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC. In an embodiment, a CAA antigen or antigen encoding polynucleotide (including, but not limited to, a conserved cancer antigen or conserved antigen encoding polynucleotide) of the present invention of the present invention has 50%, to/or 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identity to a peptide of SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

The terms “percent (%) sequence identity”, and the like, generally refer to the degree of identity or correspondence between different nucleotide sequences of nucleic acid molecules or amino acid sequences of polypeptides that may or may not share a common evolutionary origin. Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc.

### Sizes of Polynucleotides and Polypeptides

In an embodiment, the polynucleotides may be any length reasonable to encode an epitope. In an embodiment, the polynucleotides range in length from about 10 to about 200 or more polynucleotides. In an embodiment, the polynucleotides in length from 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, to/or 200 nucleotides in length.

In an embodiment, the polypeptides may be any length that is reasonable for an epitope. For example, the polypeptides may have a size of from 5 to 30 or more, e.g., from 5 to 25, from 5 to 20, from 5 to 15, from 5 to 10, from 6 to 10, from 7 to 9, or from 8 to 9 amino acids. For example, the polypeptides may have 5, 6, 7, 8, 9, 10, 11, 12, 13, 14, or 15 amino acids. In an embodiment, the optimal length of a polypeptide may be determined based the immunogenicity of the polypeptides of different lengths when introduced to a cell or subject.

### Modifications on Polypeptides

In an embodiment, polypeptides of the present invention herein may comprise one or more modifications (e.g., post-translational modifications). In some cases, the polypeptides may comprise cysteinylated Cysteine. Other examples of modifications include ubiquitination, phosphorylation, sulfonation, glycosylation, acetylation, methylation, ADP-ribosylation, methionine oxidation, cysteine oxidation, cysteine lipidation, farnesylation, geranylation, pyroglutamation, and deamidation. In an embodiment, the polypeptide comprises one or more (e.g., 1, 2, 3, 4, 5, 6, 7, 8, 9, 10, or more) amino acids that are each independently modified with an ubiquitination, phosphorylation, sulfonation, glycosylation, acetylation, methylation, ADP-ribosylation, methionine oxidation, cysteine oxidation, cysteine lipidation, farnesylation, geranylation, pyroglutamation, or deamidation.

Synthetic mRNA

In an embodiment, the CAA polynucleotide of the present invention is mRNA, e.g., synthetic mRNA. In an embodiment, the synthetic mRNA may comprise coding sequence(s) for one or more CAA polypeptides herein. In an embodiment, the synthetic mRNA is or is encoded by a CAA encoding polynucleotide described elsewhere herein.

A synthetic mRNA may be an mRNA produced through an in vitro transcription reaction or through artificial (non-natural) chemical synthesis or through a combination thereof. In an embodiment, the synthetic mRNA further comprises a poly A tail, a Kozak sequence, a 3′ untranslated region, a 5′ untranslated region, or any combination thereof. Poly A tails in particular can be added to a synthetic RNA using a variety of art-recognized techniques, e.g., using poly A polymerase, using transcription directly from PCR products, or by ligating to the 3′ end of a synthetic RNA with RNA ligase.

The synthetic mRNA may comprise one or more stabilizing elements that maintain or enhance the stabilities of mRNA, e.g., reducing or preventing degradation of the mRNA. Examples of stabilizing elements include untranslated regions (UTR) at their 5′-end (5′UTR) and/or at their 3′-end (3′UTR), in addition to other structural features, such as a 5′-cap structure or a 3′-poly(A) tail. The stabilizing elements may be a histone stem-loop, e.g., a histone stem loop added by a stem-loop binding protein (SLBP).

### Delivery Vehicles

The CAA polynucleotides and/or peptides of the present invention can be incorporated into a delivery vehicle. The delivery vehicles can be used to deliver a CAA polynucleotide and/or peptide of the present invention to a cell. Thus, also described in certain example embodiments herein delivery vehicles, including but not limited to, vectors and virus particles that can deliver a CAA polynucleotide and/or polypeptide of the present invention, which is also generally referred to as “cargo” in this context. It will be appreciated that other molecules can also be included as cargo. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles, and other delivery reagents described herein.

The delivery vehicles described herein can have a greatest dimension or greatest average dimension (e.g., diameter or greatest average diameter) of less than 100 microns (μm). In an embodiment, the delivery vehicles have a greatest dimension or greatest average dimension of less than 10 μm. In an embodiment, the delivery vehicles may have a greatest dimension or greatest average dimension of less than 2000 nanometers (nm). In an embodiment, the delivery vehicles may have a greatest dimension or greatest average dimension of less than 1000 nanometers (nm). In an embodiment, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In an embodiment, the delivery vehicles may have a greatest dimension or greatest average dimension ranging between 25 nm and 200 nm.

In an embodiment, the delivery vehicles may be or comprise particles. For example, the delivery vehicle may be or comprise nanoparticles (e.g., particles with a greatest dimension or greatest average dimension (e.g., diameter or greatest average diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

In an embodiment, the delivery vehicles are nanoparticles. Exemplary nanoparticles are described in WO 2008042156, US20130185823, and WO2015089419. In general, a “nanoparticle” refers to any particle having a diameter of less than 1000 nm. In an embodiment, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi: 10.1038/nnano.2014.84, describing particles, methods of making and using them and measurements thereof.

### Vectors and Vector Systems

Also provided herein are vectors that can contain one or more of the CAA polynucleotides of the present invention described elsewhere herein. In an embodiment, the vector can contain one or more polynucleotides encoding one or more polypeptides, such as a CAA polypeptide, of the present invention described elsewhere herein. The vectors can be useful in producing bacterial, fungal, yeast, plant cells, animal cells, and transgenic animals that can express one or more CAA polynucleotides and/or polypeptides of the present invention described elsewhere herein. Within the scope of this disclosure are vectors containing one or more of the polynucleotide sequences described herein. The vectors and/or vector systems can be used, for example, to express one or more of the polynucleotides in a cell, such as a producer cell, to produce virus particles containing one or more polynucleotide(s) of the present invention described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In an embodiment, the vector can be a viral vector. In an embodiment, the viral vector is an is an adeno-associated virus (AAV), adenovirus vector, a retroviral vector, or lentiviral vector. These and others are further detailed and described elsewhere herein.

### Cell-Based Vector Amplification and Expression

Vectors may be introduced and propagated in a prokaryote or prokaryotic cell. In an embodiment, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In an embodiment, a prokaryote is used to amplify copies of a vector and express one or more nucleic acids, such as to provide a source of one or more proteins for delivery to a host cell or host organism.

Vectors can be designed for expression of the polynucleotides and/or polypeptides of the present invention described herein (e.g., nucleic acid transcripts, proteins, enzymes, and combinations thereof) in a suitable host cell. In an embodiment, the suitable host cell is a prokaryotic cell. Suitable host cells include, but are not limited to, bacterial cells, yeast cells, insect cells, and mammalian cells. In an embodiment, the suitable host cell is a eukaryotic cell.

In an embodiment, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In an embodiment, the host cell is a suitable insect cell. Suitable insect cells include those from Spodoptera frugiperda. Suitable strains of S. frugiperda cells include, but are not limited to, Sf9 and Sf21. In an embodiment, the host cell is a suitable yeast cell. In an embodiment, the yeast cell can be from Saccharomyces cerevisiae. In an embodiment, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In an embodiment, the vector can be a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (In Vitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In an embodiment, the vector is a baculovirus vector or expression vector and can be suitable for expression of polynucleotides and/or proteins in insect cells. In an embodiment, the suitable host cell is an insect cell. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In an embodiment, the vector is a mammalian expression vector. In an embodiment, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In an embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In an embodiment, a regulatory element can be operably linked to one or more polynucleotides of the present invention so as to drive expression of the one or more polynucleotides of the present invention described herein.

In an embodiment, the vector can be a fusion vector or fusion expression vector. In an embodiment, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In an embodiment, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In an embodiment, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In an embodiment, one or more vectors driving expression of one or more polynucleotides of the present invention described herein are introduced into a cell, such as a host cell for viral particle production and/or a target cell to which a polypeptide of the present invention is to be expressed.

### Cell-Free Vector and Polynucleotide Expression

In an embodiment, the polynucleotide encoding one or more CAA polynucleotides or polypeptides of the present invention can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In an embodiment, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In an embodiment, the polynucleotides and/or vectors thereof described herein (such as the polynucleotides of the present invention, such as a viral polynucleotides of the present invention) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In an embodiment, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA, Vol. 78(3), p. 1527-31, 1981).

In an embodiment, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In an embodiment, the vector can contain a minimal promoter. In an embodiment, the minimal promoter is the Mecp2 promoter, tRNA promoter, or U6. In a further embodiment, the minimal promoter is tissue specific. In an embodiment, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In an embodiment a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In an embodiment, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In an embodiment, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer114), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the CRISPR-Cas system described herein are typically placed under control of a plant promoter, i.e., a promoter operable in plant cells. The use of different types of promoters is envisaged.

A constitutive plant promoter is a promoter that can express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the polynucleotides of the present invention are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for expression of one or more polynucleotides of the present invention in plants can be found in e.g., Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more polynucleotides of the present invention described herein, a light-responsive cytochrome heterodimer (e.g., from Arabidopsis thaliana), and a transcriptional activation/repression domain. In an embodiment, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In an embodiment, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

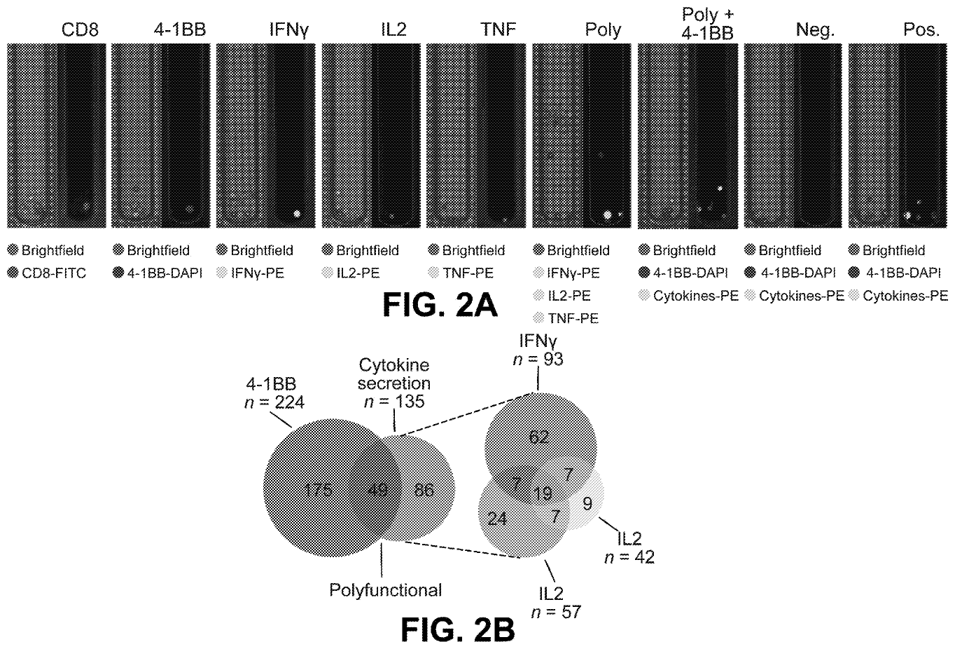

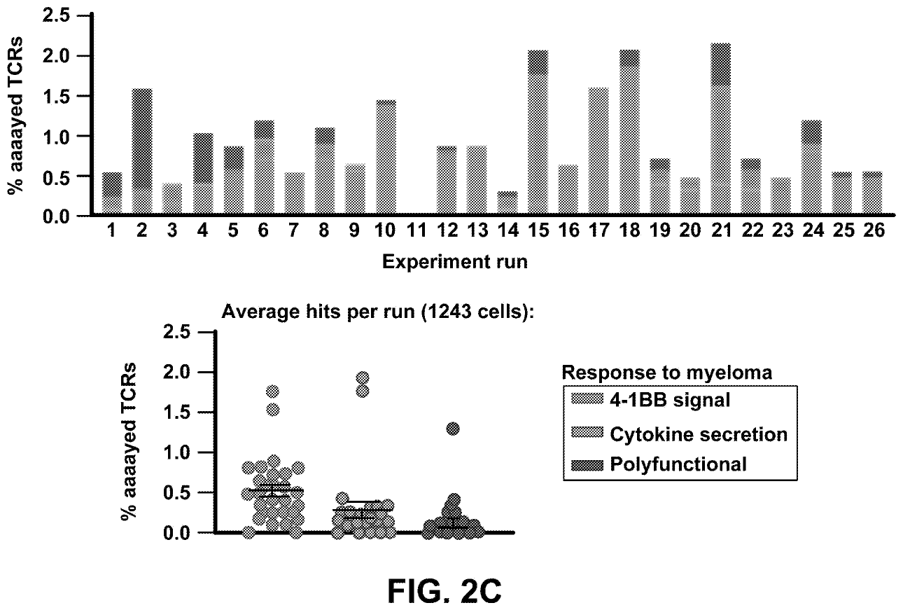

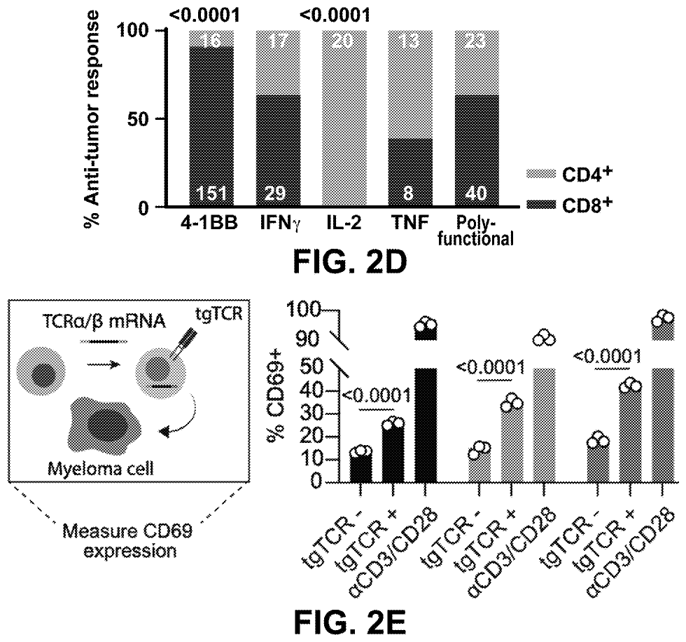

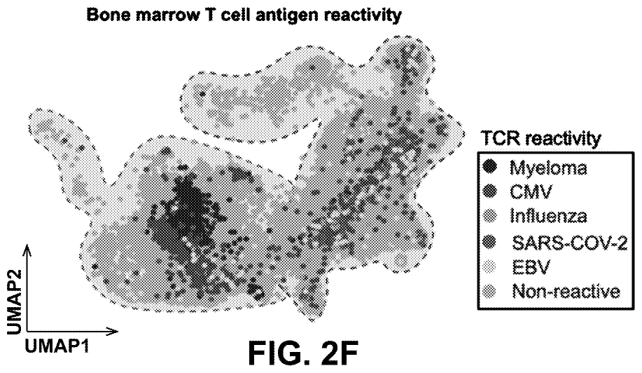

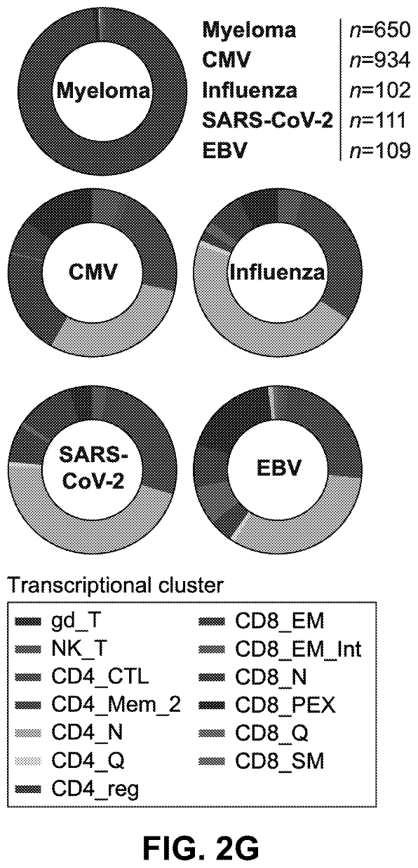

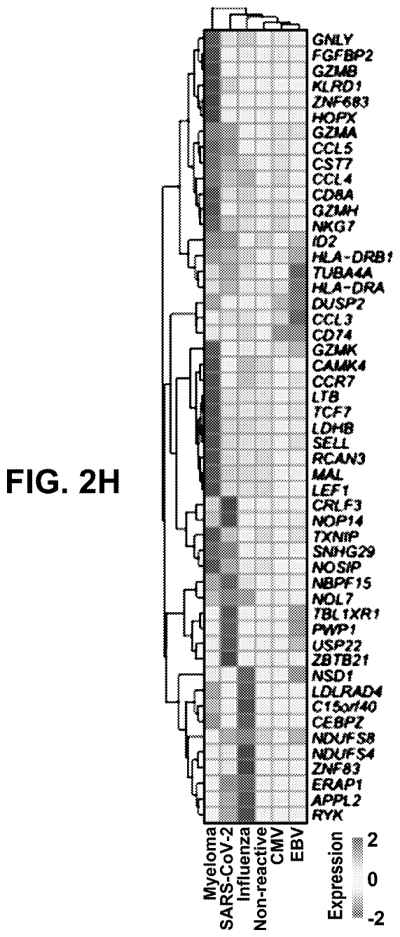

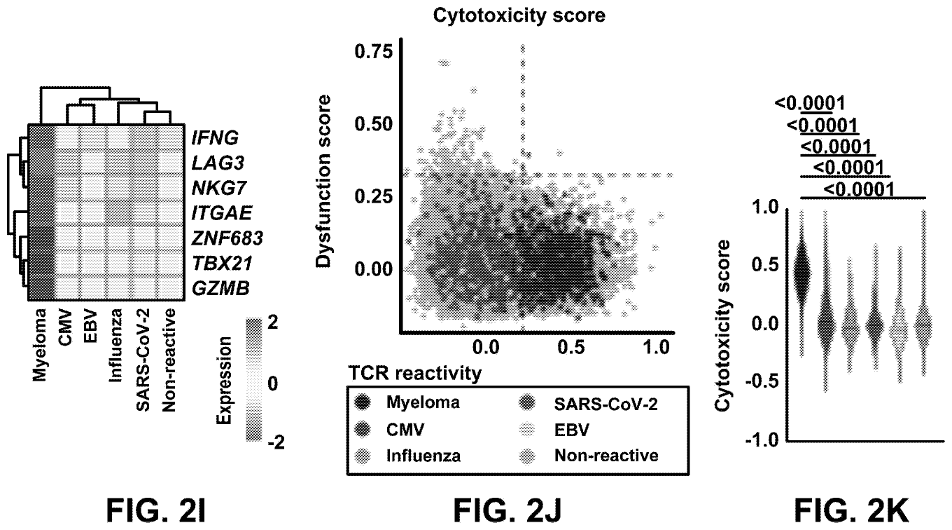

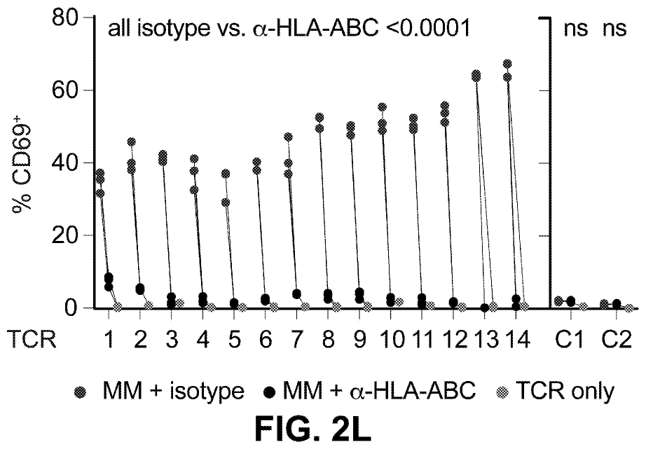

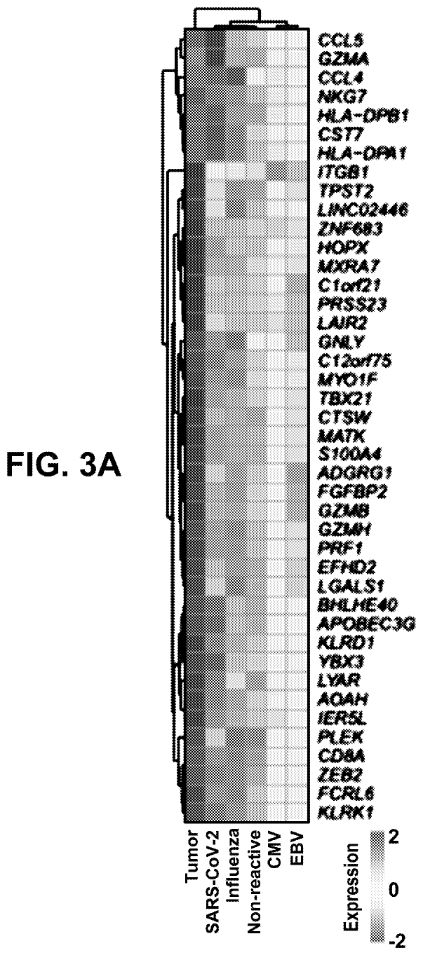

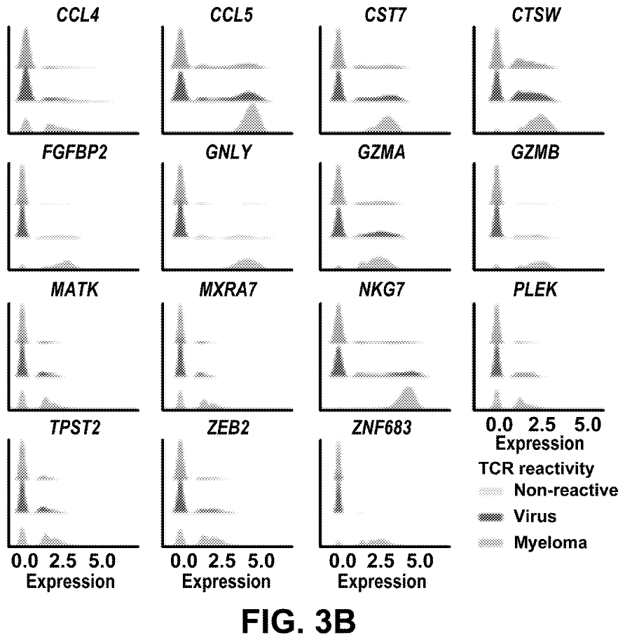

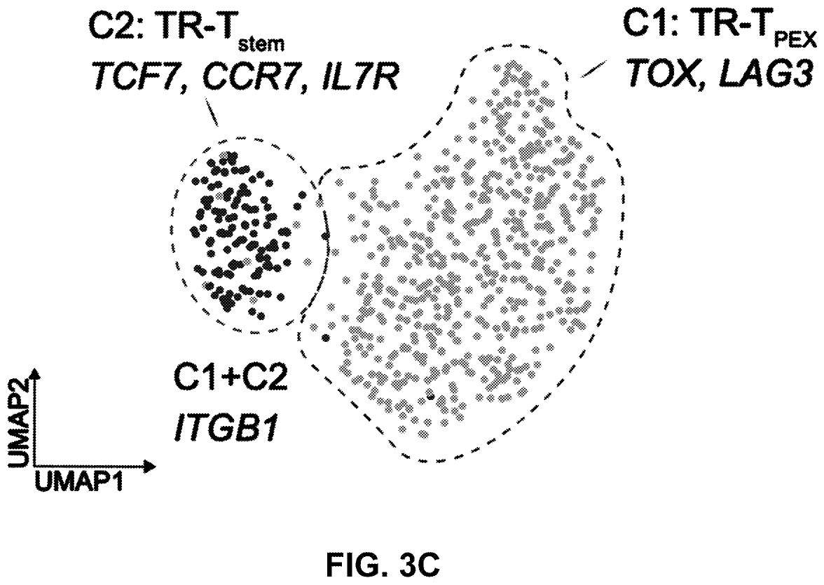

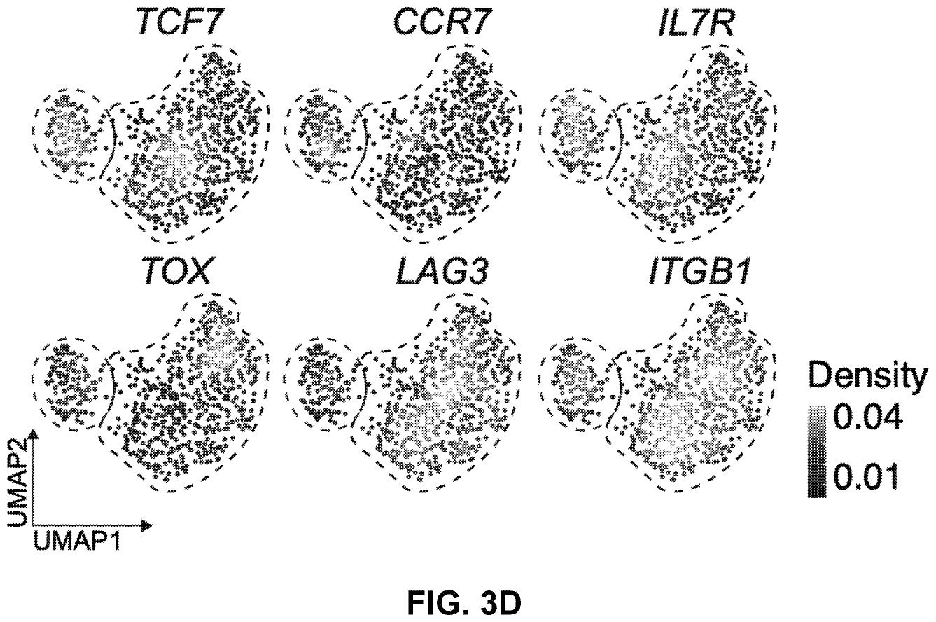

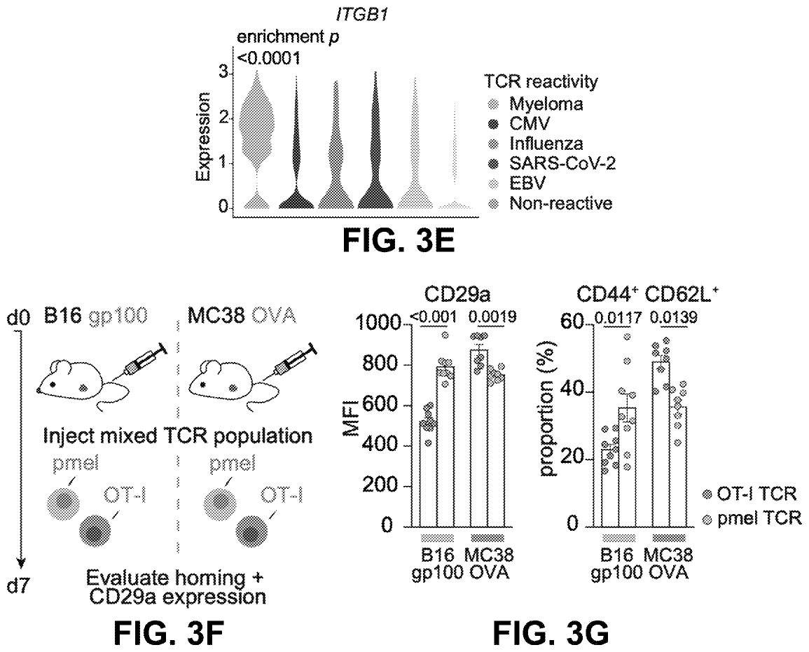

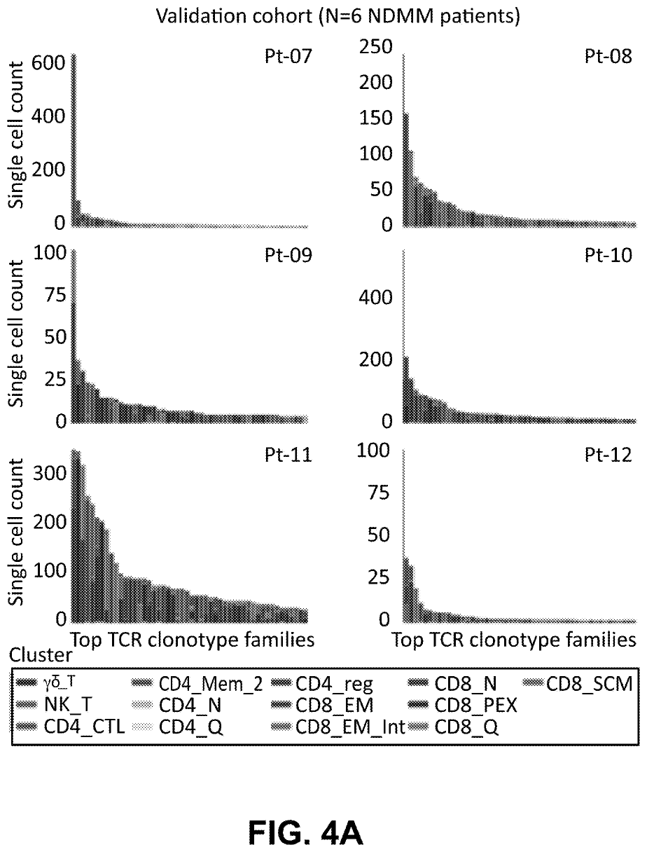

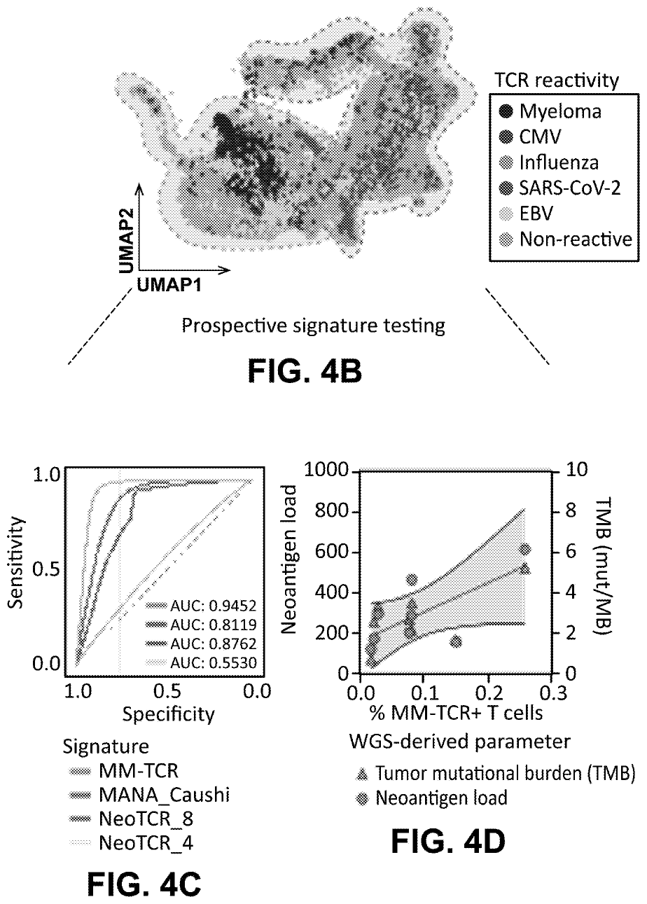

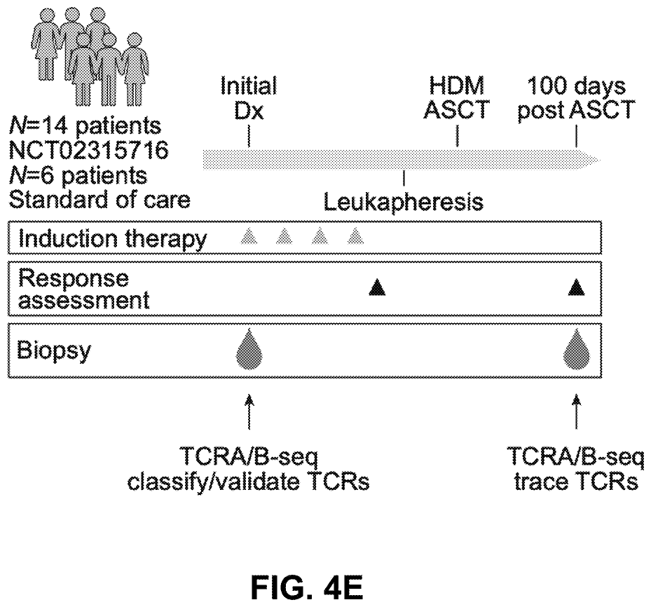

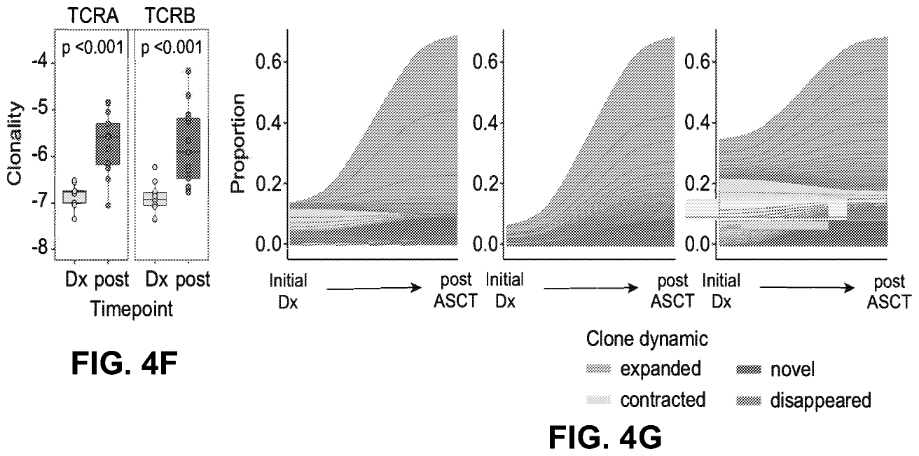

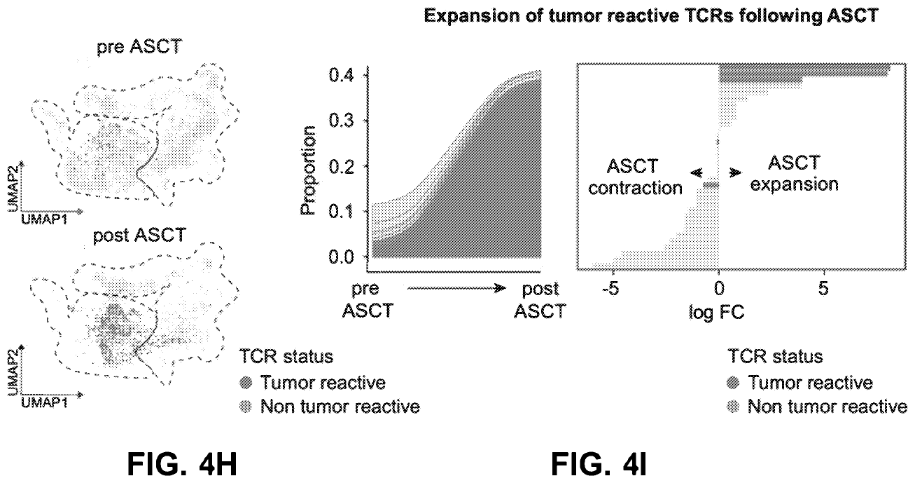

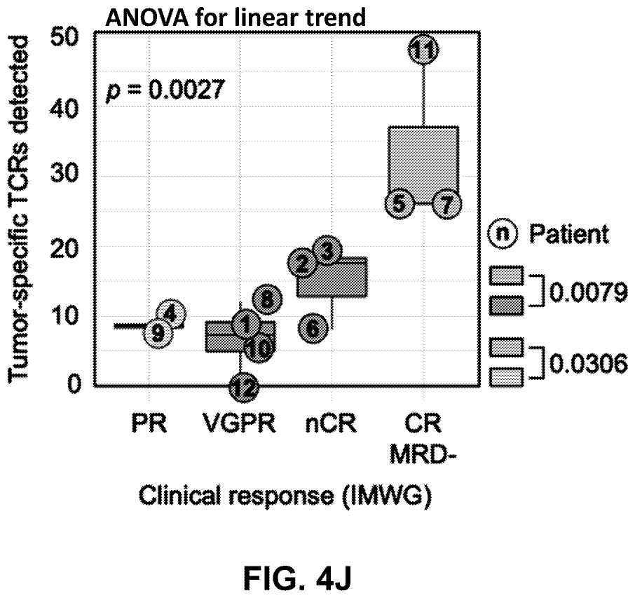

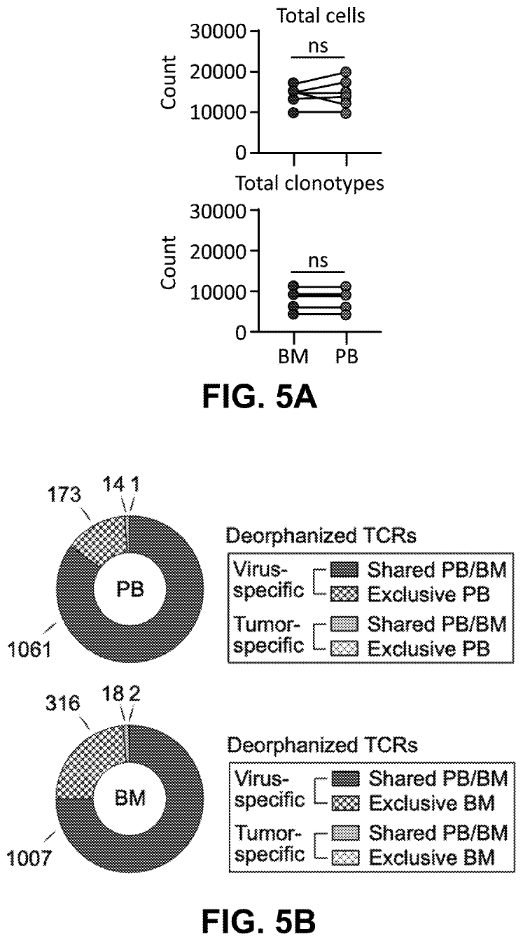

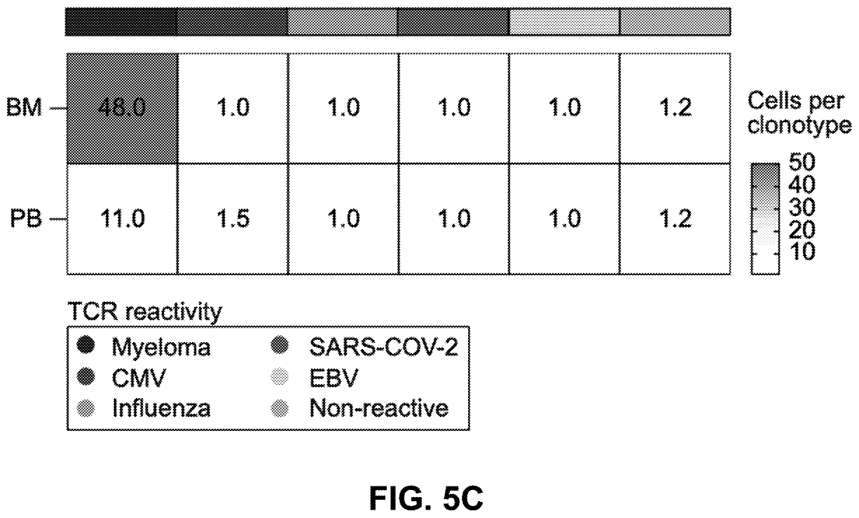

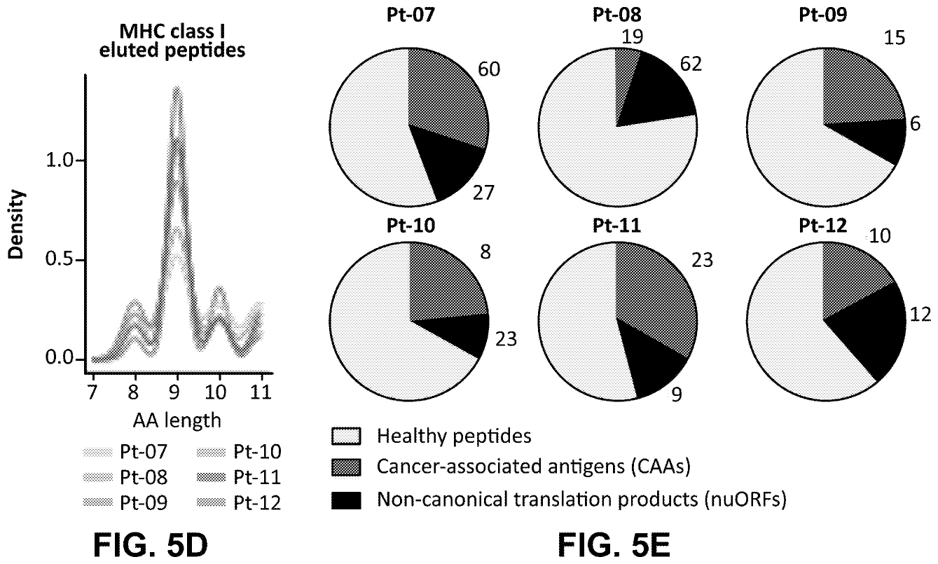

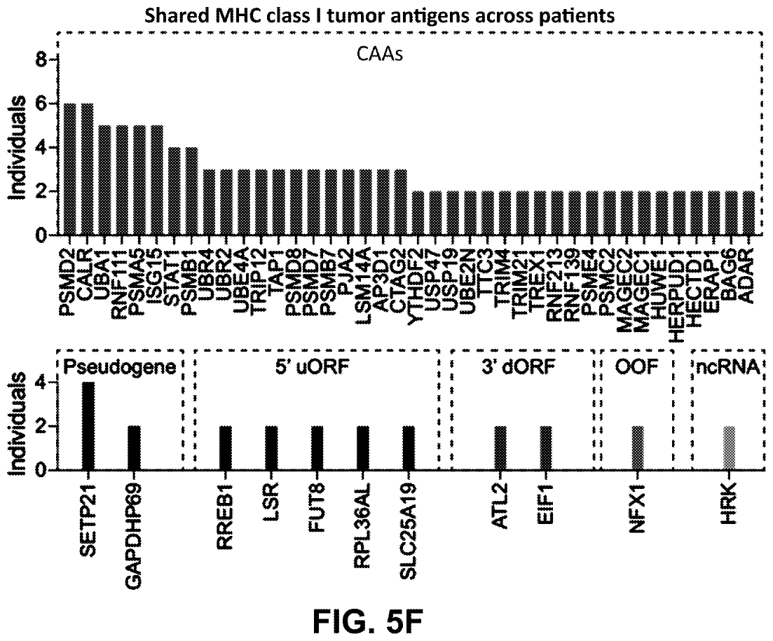

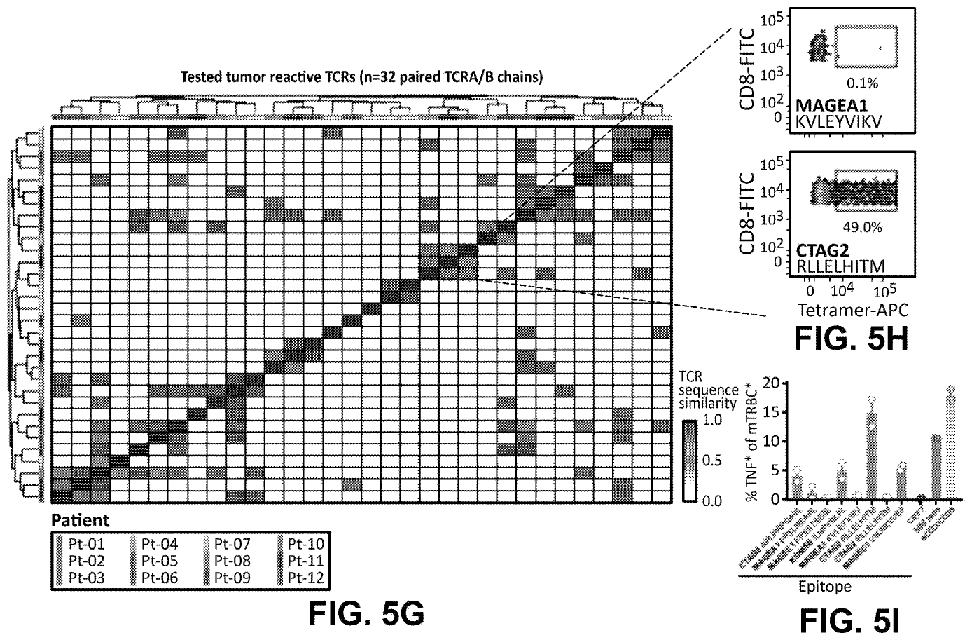

In an embodiment, the polynucleotide, vector, or system thereof can include one or more elements capable of translocating and/or expressing one or more polynucleotides of the present invention to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., genome.unmc.edu/LocSigDB/and Negi et al., 2015. Database. 2015: bav003; doi: 10.1093/database/bav003), nuclear export signals (e.g., LXXXLXXLXL and others described elsewhere herein), endoplasmic reticulum localization/retention signals (e.g., KDEL (SEQ ID NO: 306), KDXX, KKXX, KXX, and others described elsewhere herein; and see e.g. Liu et al. 2007 Mol. Biol. Cell. 18(3):1073-1082 and Gorleku et al., 2011. J. Biol. Chem. 286:39573-39584), mitochondria (see e.g., Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g. (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http: minimotifminer.org, http://mitominer.mrc-mbu.cam.ac.uk/release-4.0/embodiment.do?name=Protein % 20MTS), LocDB (see above), PTSs predictor ( ) TargetP-2.0 (http://www.cbs.dtu.dk/services/TargetP/), ChloroP (http://www.cbs.dtu.dk/services/ChloroP/); NetNES (www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the polynucleotides of the present invention can be operably linked, fused to, or otherwise modified to include a polynucleotide that encodes or is a selectable marker or tag, which can be a polynucleotide or polypeptide. In an embodiment, the polynucleotide encoding a polypeptide selectable marker can be incorporated with the polynucleotide of the present invention, such as a viral polynucleotide, such that the selectable marker polypeptide, when translated, is inserted between two amino acids between the N- and C-terminus of the polypeptide of the present invention or is present at the N- and/or C-terminus of the polypeptide of the present invention. In an embodiment, the selectable marker or tag is a polynucleotide barcode or unique molecular identifier (UMI).

It will be appreciated that the polynucleotide encoding such selectable markers or tags can be incorporated into a polynucleotide encoding one or more polypeptides of the present invention, such as a viral polypeptide, described herein in an appropriate manner to allow expression of the selectable marker or tag. Such techniques and methods are described elsewhere herein, and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more polypeptides of the present invention herein via suitable linker, such as a glycine or glycine serine linkers as short as GS or GG up to (GGGGG)3 (SEQ ID NO: 307) or (GGGGS)3 (SEQ ID NO: 308). Other suitable linkers are described elsewhere herein.

The vector or vector system can include one or more polynucleotides encoding one or more targeting moieties. In an embodiment, the targeting moiety encoding polynucleotides can be included in the vector or vector system, such as a viral vector system, such that they are expressed within and/or on the virus particle(s) produced such that the virus particles can be targeted to specific cells, tissues, organs, etc. In an embodiment, the targeting moiety encoding polynucleotides can be included in the vector or vector system such that the polynucleotide(s) and/or products expressed therefrom (e.g., polypeptides) include the targeting moiety and can be targeted to specific cells, tissues, organs, etc. In an embodiment, such as non-viral carriers, the targeting moiety can be attached to the carrier (e.g., polymer, lipid, inorganic molecule etc.) and can be capable of targeting the carrier and any attached or associated polynucleotide(s) and/or polypeptides of the present invention to specific cells, tissues, organs, etc.

### Codon Optimization of Vector Polynucleotides

As described elsewhere herein, the polynucleotide encoding one or more polypeptides of the present invention described herein can be codon optimized. In an embodiment, one or more polynucleotides contained in a vector (“vector polynucleotides”) described herein that are in addition to an optionally codon optimized polynucleotide encoding one or more polypeptides of the present invention, such as viral polypeptides, described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In an embodiment, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or B[ED1] ennetzen and Hall, J Biol Chem. 1982 Mar. 25; 257(6):3026-31. As to codon usage in plants including algae, reference is made to Campbell and Gowri, Plant Physiol. 1990 January; 92(1): 1-11; as well as Murray et al, Nucleic Acids Res. 1989 Jan. 25; 17(2):477-98; or Morton B R, J Mol Evol. 1998 April; 46(4):449-59.

The vector polynucleotide can be codon optimized for expression in a specific cell-type, tissue type, organ type, and/or subject type. In an embodiment, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In an embodiment, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In an embodiment, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In an embodiment, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

[In an embodiment, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In an embodiment, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

In an embodiment, a vector comprises one or more insertion sites, such as a restriction endonuclease recognition sequence (also referred to as a “cloning site”). In an embodiment, one or more insertion sites (e.g., about 1, about 2, about 3, about 4, about 5, about 6, about 7, about 8, about 9, about 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about 1, about 2, about 3, about 4, about 5, about 6, about 7, about 8, about 9, about 10, about 15, about 20, or more guide polynucleotides. In an embodiment, about 1, about 2, about 3, about 4, about 5, about 6, about 7, about 8, about 9, about 10, or more such guide-polynucleotide-containing vectors may be provided, and optionally delivered to a cell.

Delivery vehicles, vectors, particles, nanoparticles, formulations and components thereof for expression of one or more polynucleotides and/or polypeptides of the present invention, such as one or more viral polynucleotides and/or polypeptides, described herein are as used in the foregoing documents, such as International Patent Publication No. WO 2014/093622 (PCT/US2013/074667) and are discussed in greater detail herein.

### Viral Vectors

In an embodiment, the vector is a viral vector. The term of art “viral vector” and as used herein in this context refers to polynucleotide based vectors that contain one or more elements from or based upon one or more elements of a virus that can be capable of expressing and packaging a polynucleotide, such as a viral polynucleotide of the present invention, into a virus particle and producing said virus particle when used alone or with one or more other viral vectors (such as in a viral vector system). Viral vectors and systems thereof can be used for producing viral particles for delivery of and/or expression of one or more polynucleotides and/or polypeptides of the present invention described herein. The viral vector can be part of a viral vector system involving multiple vectors. In an embodiment, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In an embodiment, the viral vectors are configured to produce replication incompetent viral particles for improved safety of these systems.

In an embodiment, the virus structural component, which can be encoded by one or more polynucleotides in a viral vector or vector system, comprises one or more capsid proteins including an entire capsid. In an embodiment, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the CRISPR-Cas systems can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that can transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that can infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In an embodiment, an HIV-based lentiviral vector system can be used. In an embodiment, a FIV-based lentiviral vector system can be used.

In an embodiment, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8:275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for polynucleotides and/or polypeptides of the present invention described herein.

In an embodiment, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In an embodiment, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In an embodiment, the second-generation vector lacks one or more accessory virulence factors (e.g., vif, vprm, vpu, nef, and combinations thereof). Unlike the first-generation lentiviral vectors, no single second-generation lentiviral vector includes all features necessary to express and package a polynucleotide into a virus particle. In an embodiment, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In an embodiment, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In an embodiment, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g., promoter(s)) to drive expression of the features present on the packaging vector, and (iii) an “envelope vector” that contains one or more envelope protein genes and upstream promoters. In an embodiment, the third-generation lentiviral vector system can include at least two packaging vectors, with the gag-pol being present on a different vector than the rev gene.

In an embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the polypeptides and/or polynucleotides of the present invention described elsewhere herein.

In an embodiment, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In an embodiment, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Since LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see, e.g., Hanawa et al. Molec. Ther. 2002 5(3) 242-251), modified Sindbis virus envelope proteins (see, e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124:1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In an embodiment, the tropism of the resulting lentiviral particle can be tuned by incorporating cell targeting peptides into a lentiviral vector such that the cell targeting peptides are expressed on the surface of the resulting lentiviral particle. In an embodiment, a lentiviral vector can contain an envelope protein that is fused to a cell targeting protein (see, e.g., Buchholz et al. 2015. Trends Biotechnol. 33:777-790; Bender et al. 2016. PLOS Pathog. 12(e1005461); and Friedrich et al. 2013. Mol. Ther. 2013. 21:849-859.

In an embodiment, a split-intein-mediated approach to target lentiviral particles to a specific cell type can be used (see, e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In an embodiment, a covalent-bond-forming protein-peptide pair can be incorporated into one or more of the lentiviral vectors described herein to conjugate a cell targeting peptide to the virus particle (see, e.g., Kasaraneni et al. 2018. Sci. Reports (8) No. 10990). In an embodiment, a lentiviral vector can include an N-terminal PDZ domain of InaD protein (PDZ1) and its pentapeptide ligand (TEFCA (SEQ ID NO: 309)) from NorpA, which can conjugate the cell targeting peptide to the virus particle via a covalent bond (e.g., a disulfide bond). In an embodiment, the PDZ1 protein can be fused to an envelope protein, which can optionally be binding deficient and/or fusion competent virus envelope protein and included in a lentiviral vector. In an embodiment, the TEFCA (SEQ ID NO: 309) can be fused to a cell targeting peptide and the TEFCA-CPT (SEQ ID NO: 309) fusion construct can be incorporated into the same or a different lentiviral vector as the PDZ1-envelope protein construct. During virus production, specific interaction between the PDZ1 and TEFCA (SEQ ID NO: 309) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see, e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver a polynucleotide of the present invention described herein to a cell.

In an embodiment, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus, and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses. Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In an embodiment of these embodiments, the Gag, Pol, and accessory proteins are lentiviral and/or gammaretroviral. In an embodiment, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In an embodiment, the vector can be an adenoviral vector. In an embodiment, the adenoviral vector can include elements such that the virus particle produced using the vector or system thereof can be serotype 2 or serotype 5. In an embodiment, the polynucleotide to be delivered via the adenoviral particle can be up to about 8 kb. Thus, In an embodiment, an adenoviral vector can include a DNA polynucleotide to be delivered that can range in size from about 0.001 kb to about 8 kb. Adenoviral vectors have been used successfully in several contexts (see, e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In an embodiment the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In an embodiment of the helper-dependent adenoviral vector system one vector (the helper) can contain all the viral genes required for replication but contains a conditional gene defect in the packaging domain. The second vector of the system can contain only the ends of the viral genome, one or more polynucleotides of the present invention described elsewhere herein, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see, e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the polynucleotides of the present invention described herein. In an embodiment, the polynucleotide to be delivered via the viral particle produced from a helper-dependent adenoviral vector or system thereof can be up to about 37 kb. Thus, In an embodiment, an adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In an embodiment, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In an embodiment, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use for delivering the polynucleotide of the present invention described elsewhere herein. In an embodiment, a hybrid-adenoviral vector can include one or more features of a retrovirus and/or an adeno-associated virus. In an embodiment the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use for delivering one or more polynucleotides of the present invention described herein. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the CRISPR-Cas system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer than adenoviral vectors. In an embodiment the AAV can integrate into a specific site on chromosome 19 of a human cell with no observable side effects. In an embodiment, the capacity of the AAV vector, system thereof, and/or AAV particles can be up to about 4.7 kb. The AAV vector or system thereof can include one or more regulatory molecules. In an embodiment the regulatory molecules can be promoters, enhancers, repressors and the like, which are described in greater detail elsewhere herein. In an embodiment, the AAV vector or system thereof can include one or more polynucleotides that can encode one or more regulatory proteins. In an embodiment, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In an embodiment, the AAV capsid can contain 60 capsid proteins. In an embodiment, the ratio of VP1:VP2:VP3 in a capsid can be about 1:1:10.

In an embodiment, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In an embodiment, a producing host cell line expresses one or more of the adenovirus helper factors.

The AAV vector or system thereof can be configured to produce AAV particles having a specific serotype. In an embodiment, the serotype can be AAV-1, AAV-2, AAV-3, AAV-4, AAV-5, AAV-6, AAV-8, AAV-9 or any combinations thereof. In an embodiment, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, In an embodiment, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In an embodiment, an AAV vector or system thereof capable of producing AAV particles capable of targeting cardiac tissue can be configured to generate an AAV particle having an AAV-4 serotype. In an embodiment, an AAV vector or system thereof capable of producing AAV particles capable of targeting the liver can be configured to generate an AAV having an AAV-8 serotype. In an embodiment, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82:5887-5911 (2008). The AAV can be any one of the serotypes.

In an embodiment, the AAV vector or system thereof is configured as a “gutless” vector, similar to that described in connection with a retroviral vector. In an embodiment, the “gutless” AAV vector or system thereof can have the cis-acting viral DNA elements involved in genome amplification and packaging in linkage with the heterologous sequences of interest (e.g., the CRISPR-Cas system polynucleotide(s)).

In an embodiment, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In an embodiment, an AAV vector or vector system can contain or consists essentially of one or more polynucleotides encoding one or more polynucleotides of the present invention, such as one or more viral polynucleotides.

In another embodiment, the invention provides a polypeptide of the present invention operatively coupled with Adeno Associated Virus (AAV), e.g., an AAV comprising a polypeptide of the present invention as a fusion, with or without a linker, to or with an AAV capsid protein such as VP1, VP2, and/or VP3. More particularly, modifying the knowledge in the art, e.g., Rybniker et al., [ED2] J Virol. December 2012; 86(24): 13800-13804; Lux K, et al. 2005. J. Virol. 79:11776-11787; Munch R C, et al. 2013 [ED3]. Mol. Ther. 21:109-118; and Warrington K H, Jr, et al. 2004. J. Virol. 78:6595-6609, each incorporated herein by reference, one can obtain a modified AAV capsid of the invention. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a polypeptide of the present invention. Likewise, these can be fusions, with the protein, e.g., large payload protein such as a polypeptide of the present invention fused in a manner analogous to prior art fusions. See, e.g., US Patent Publication 20090215879; Nance et al., Hum Gene Ther. 26(12):786-800 (2015) and documents cited therein, incorporated herein by reference. One of ordinary skill in the art, from this disclosure, can make and use modified AAV or AAV capsid as in the herein invention, and through this disclosure one knows now that payload proteins, such as large payload proteins, can be fused to the AAV capsid. Accordingly, this approach is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus of within the family Parvoviridae or the genus Dependoparvovirus or any of the other foregoing genera within Parvoviridae is contemplated as within the invention with discussion herein as to AAV applicable to such other viruses.

In one embodiment, the invention provides a non-naturally occurring or engineered composition comprising a polypeptide of the present invention which is part of or tethered to an AAV capsid domain, i.e., VP1, VP2, or VP3 domain of Adeno-Associated Virus (AAV) capsid. In an embodiment, part of or tethered to an AAV capsid domain includes associated with associated with a AAV capsid domain. In an embodiment, the polypeptide of the present invention may be fused to the AAV capsid domain. In an embodiment, the fusion may be to the N-terminal end of the AAV capsid domain. As such, In an embodiment, the C-terminal end of the polypeptide of the present invention is fused to the N-terminal end of the AAV capsid domain. In an embodiment, an NLS and/or a linker (such as a GlySer linker) may be positioned between the C-terminal end of the polypeptide of the present invention and the N-terminal end of the AAV capsid domain. In an embodiment, the fusion may be to the C-terminal end of the AAV capsid domain. In an embodiment, this is not preferred due to the fact that the VP1, VP2 and VP3 domains of AAV are alternative splices of the same RNA and so a C-terminal fusion may affect all three domains. In an embodiment, the AAV capsid domain is truncated. In an embodiment, some or all of the AAV capsid domain is removed. In an embodiment, some of the AAV capsid domain is removed and replaced with a linker (such as a GlySer linker), typically leaving the N-terminal and C-terminal ends of the AAV capsid domain intact, such as the first 2, 5 or 10 amino acids. In this way, the internal (non-terminal) portion of the VP3 domain may be replaced with a linker. It is particularly preferred that the linker is fused to the polypeptide of the present invention. A branched linker may be used, with the polypeptide of the present invention fused to the end of one of the branches. This allows for some degree of spatial separation between the capsid and the polypeptide of the present invention. In this way, the polypeptide of the present invention is part of (or fused to) the AAV capsid domain.

In other embodiments, the polypeptide of the present invention may be fused in frame within, i.e., internal to, the AAV capsid domain. Thus, In an embodiment, the AAV capsid domain again preferably retains its N-terminal and C-terminal ends. In this case, a linker is preferred, In an embodiment, either at one or both ends of the polypeptide of the present invention. In this way, the polypeptide of the present invention is again part of (or fused to) the AAV capsid domain. In an embodiment, the positioning of the polypeptide of the present invention is such that the polypeptide of the present invention is at the external surface of the viral capsid once formed. In one embodiment, the invention provides a non-naturally occurring or engineered composition comprising a polypeptide of the present invention associated with a AAV capsid domain of Adeno-Associated Virus (AAV) capsid. As used herein, the term “associated” means fused, bound to, or tethered to. In an embodiment, the polypeptide of the present invention is tethered to the VP1, VP2, or VP3 domain. This is via a connector protein or tethering system such as the biotin-streptavidin system. In one example, a biotinylation sequence (15 amino acids) therefore is fused to the polypeptide of the present invention.

When the AAV capsid domain is fused with streptavidin, they form a highly stable association due to their very strong affinity for each other. This is especially true if the fusion is located at the N-terminus of the AVV capsid domain. Accordingly, In an embodiment, provided herein is a composition or system comprising a polypeptide of the present invention-biotin fused to a streptavidin-AAV capsid domain. The polypeptide of the present invention-biotin and streptavidin-AAV capsid domain form a single complex when the two parts are fused together. NLSs also may be incorporated between the polypeptide of the present invention and the biotin; and/or between the streptavidin and the AAV capsid domain.

In an embodiment, provided herein is a fusion of a polypeptide of the present invention with a connector protein specific for a high affinity ligand for that connector, and the AAV VP2 domain is bound to said high affinity ligand. For example, streptavidin can be the connector fused to the polypeptide of the present invention, while biotin is bound to the AAV VP2 domain. Upon co-localization, the streptavidin will bind to the biotin, thus connecting the polypeptide of the present invention to the AAV VP2 domain. The reverse arrangement also is possible. In an embodiment, a biotinylation sequence (15 amino acids) is fused to the AAV VP2 domain, in particular the N-terminus of the AAV VP2 domain. [ED4]

In an embodiment, the biotinylated AAV capsids with streptavidin-polypeptide of the present invention are assembled in vitro. This way the AAV capsids assemble in a straightforward manner and the polypeptide of the present invention-streptavidin fusion may be added after assembly of the capsid.

[In other embodiments, a biotinylation sequence (15 amino acids) is fused to the polypeptide of the present invention, which is fused with the AAV VP2 domain fused with streptavidin, wherein, in preferred embodiments, the fusion is located at the N-terminus of the AVV capsid domain.

In an embodiment, the polypeptide of the present invention and the AAV VP2 domain are fused. In an embodiment, the fusion is to the N-terminal end of the polypeptide of the present invention. In an embodiment, the AAV and polypeptide of the present invention are associated via fusion. In an embodiment, the AAV and polypeptide of the present invention are associated via fusion including a linker. Suitable linkers are discussed herein but include Gly Ser linkers. Fusion to the N-terminus of AAV VP2 domain is preferred. In an embodiment, the polypeptide of the present invention comprises at least one Nuclear Localization Signal (NLS). In a further embodiment, the present invention provides compositions comprising the polypeptide of the present invention and associated AAV VP2 domain or the polynucleotides or vectors described herein. Such compositions and formulations are discussed elsewhere herein.

Alternatively, a tether may be to fuse or otherwise associate the AAV capsid domain to an adaptor protein that binds to or recognizes a corresponding RNA sequence or motif. In an embodiment, the adaptor comprises a binding protein which recognizes and binds (or is bound by) an RNA sequence specific for said binding protein. In preferred embodiments, the MS2 binding protein recognizes and binds (or is bound by) an RNA sequence specific for the MS2 protein (see Konermann et al. December 2014, cited infra, incorporated herein by reference).

In an embodiment, the AAV capsid domain is associated with the adaptor protein, and the polypeptide of the present invention is tethered to the adaptor protein of the AAV capsid domain. In an embodiment, the polypeptide of the present invention is tethered to the adaptor protein of the AAV capsid domain via the polypeptide of the present invention being in a complex with a modified guide, see Konermann et al. In an embodiment, the modified guide is an sgRNA. In an embodiment, the modified guide comprises a distinct RNA sequence; see, e.g., International Patent Application No. PCT/US14/70175, incorporated herein by reference. In an embodiment, distinct RNA sequence is an aptamer.

In an embodiment, the positioning of the polypeptide of the present invention is such that the polypeptide of the present invention is at the internal surface of the viral capsid once formed. In one embodiment, the invention provides a non-naturally occurring or engineered composition comprising a polypeptide of the present invention associated with (i.e., fused, bound to, tethered to) an internal surface of an AAV capsid domain. In an embodiment, the polypeptide of the present invention is tethered to the VP1, VP2, or VP3 domain such that it locates to the internal surface of the viral capsid once formed, wherein the polypeptide of the present invention is tethered to the VP1, VP2, or VP3 domain via a connector protein or a tethering system such as the biotin-streptavidin system as described above and/or elsewhere herein.

### Herpes Simplex Viral Vectors

In an embodiment, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the CRISPR-Cas system of the present invention. In an embodiment where an HSV vector or system thereof is utilized, the host cell can be a complementing cell. In an embodiment, HSV vector or system thereof can be capable of producing virus particles capable of delivering a polynucleotide cargo of up to 150 kb. Thus, In an embodiment, the polynucleotide(s) of the present invention included in the HSV-based viral vector or system thereof can sum from about 0.001 to about 150 kb. HSV-based vectors and systems thereof have been successfully used in several contexts including various models of neurologic disorders. See, e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use to delivery one or more polynucleotides and/or polypeptides of the present invention of the present invention.

### Poxvirus Vectors

In an embodiment, the vector is a poxvirus vector or system thereof. In an embodiment, the poxvirus vector results in cytoplasmic expression of one or more polynucleotides and/or polypeptides of the present invention. In an embodiment the capacity of the poxvirus vector or system thereof is about 25 kb or more. In an embodiment, the poxvirus vector or system thereof includes one or more polynucleotides of the present invention described herein.

### Viral Vectors for Delivery to Plants

The systems and compositions can be delivered to plant cells using viral vehicles. In particular embodiments, the compositions and systems can be introduced in the plant cells using a plant viral vector (e.g., as described in Scholthof et al. 1996, Annu Rev Phytopathol. 1996; 34:299-323). Such viral vectors can be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector can be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses can be non-integrative vectors.

Virus Particle Production from Viral Vectors

### Retroviral Production

In an embodiment, one or more viral vectors and/or system thereof are delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In an embodiment, the suitable host cell for virus production from viral vectors and systems thereof described herein can stably express one or more genes involved in packaging (e.g., pol, gag, and/or VSV-G) and/or other supporting genes.

In an embodiment, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a viral polynucleotide of the present invention), and virus particle assembly, and secretion of mature virus particles into the culture media. Various other methods and techniques are generally known to those of ordinary skill in the art.

Mature virus particles can be collected from the culture media by a suitable method. In an embodiment, this can involve centrifugation to concentrate the virus. The titer of the composition containing the collected virus particles can be obtained using a suitable method. Such methods can include transducing a suitable cell line (e.g. NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In an embodiment, the resulting composition containing virus particles contains 1×101-1×1020 particles/mL.

Lentiviruses can be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 μL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 μm low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

### AAV Particle Production

There are two main strategies for producing AAV particles from AAV vectors and systems thereof, such as those described herein, which depend on how the adenovirus helper factors are provided (helper v. helper free). In an embodiment, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., one or more viral polynucleotide(s) of the present invention). In an embodiment, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., one or more viral polynucleotide(s) of the present invention) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Non-Viral Vectors

In an embodiment, the vector is a non-viral vector or vector system. The term of art “non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating polynucleotide(s) of the present invention and delivering said polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In an embodiment one or more CAA polynucleotides of the present invention, e.g., one or more viral polynucleotides, described elsewhere herein can be included in a naked polynucleotide. The term “naked polynucleotide,” as used herein, refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the polynucleotides of the present invention described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In an embodiment, the naked polynucleotide contains only the polynucleotide(s) of the present invention. In an embodiment, the naked polynucleotide can contain other nucleic acids and/or polynucleotides in addition to the polynucleotide(s) of the present invention. The naked polynucleotides can include one or more elements of a transposon system. Transposons and system thereof are described in greater detail elsewhere herein.

### Non-Viral Polynucleotide Vectors

In an embodiment, one or more of the CAA polynucleotides of the present invention, such as a viral polynucleotide of the present invention described elsewhere herein, can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See, e.g., Hardee et al. 2017. Genes. 8(2):65.

In an embodiment, the non-viral polynucleotide vector has a conditional origin of replication. In an embodiment, the non-viral polynucleotide vector is an ORT plasmid. In an embodiment, the non-viral polynucleotide vector has a minimalistic immunologically defined gene expression. In an embodiment, the non-viral polynucleotide vector has one or more post-segregationally killing system genes. In an embodiment, the non-viral polynucleotide vector is AR-free. In an embodiment, the non-viral polynucleotide vector is a minivector. In an embodiment, the non-viral polynucleotide vector includes a nuclear localization signal. In an embodiment, the non-viral polynucleotide vector includes one or more CpG motifs. In an embodiment, the non-viral polynucleotide vectors include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In an embodiment, the S/MAR sequence is located downstream of an actively transcribed polynucleotide (e.g., one or more polynucleotides of the present invention) included in the non-viral polynucleotide vector. In an embodiment, the S/MAR is a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In an embodiment, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) to transpose the polynucleotide to a new genome or polynucleotide. DNA transposons are those that do not require reverse transcription of the polynucleotide that is moved (or transposed) to transpose the polynucleotide to a new genome or polynucleotide. In an embodiment, the non-viral polynucleotide vector is a retrotransposon vector. In an embodiment, the retrotransposon vector includes long terminal repeats. In an embodiment, the retrotransposon vector does not include long terminal repeats. In an embodiment, the non-viral polynucleotide vector is a DNA transposon vector. DNA transposon vectors include a polynucleotide sequence encoding a transposase. In an embodiment, the transposon vector is configured as a non-autonomous transposon vector, meaning that the transposition does not occur spontaneously on its own. In some of these embodiments, the transposon vector lacks one or more polynucleotide sequences encoding proteins required for transposition. In an embodiment, the non-autonomous transposon vectors lack one or more Ac elements.

In an embodiment a non-viral polynucleotide transposon vector system includes a first polynucleotide vector that contains the polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase is expressed from the second vector; transpose the material between the TIRs on the first vector (e.g., the polynucleotide(s) of the present invention); and integrate it into one or more positions in the host cell's genome. In an embodiment, the transposon vector or system thereof is configured as a gene trap. In an embodiment, the TIRs are configured to flank a strong splice acceptor site followed by a reporter and/or other gene (e.g., one or more of the polynucleotide(s) of the present invention) and a strong poly A tail. When transposition occurs while using this vector or system thereof, the transposon inserts into an intron of a gene. This insertion of the reporter or other gene triggers a mis-splicing process, thereby activating the trapped gene.

Any suitable transposon system can be used. Suitable transposon and systems thereof include, but are not limited to: Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising polynucleotides of the present invention and/or polypeptides they encode).

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-220 December 2011).

In an embodiment, an LNP delivery vehicle can be used to deliver a virus particle containing a polynucleotides and/or polypeptides of the present invention. In an embodiment, the virus particle(s) can be adsorbed to the lipid particle, such as through electrostatic interactions, and/or can be attached to the liposomes via a linker.

[In an embodiment, the LNP contains a nucleic acid, wherein the charge ratio of nucleic acid backbone phosphates to cationic lipid nitrogen atoms is about 1:1.5-7 or about 1:4.

[In an embodiment, the LNP also includes a shielding compound, which is removable from the lipid composition under in vivo conditions. In an embodiment, the shielding compound is a biologically inert compound. In an embodiment, the shielding compound does not carry any charge on its surface or on the molecule as such. In an embodiment, the shielding compounds are polyethylenglycoles (PEGs), hydroxyethylglucose (HEG) based polymers, polyhydroxyethyl starch (polyHES) and polypropylene. In an embodiment, the PEG, HEG, polyHES, and a polypropylene weight between about 500 to 10,000 Da or between about 2000 to 5000 Da. In an embodiment, the shielding compound is PEG2000 or PEG5000.

In an embodiment, the LNP can include one or more helper lipids. In an embodiment, the helper lipid can be a phosphor lipid or a steroid. In an embodiment, the helper lipid is between about 20 mol % to 80 mol % of the total lipid content of the composition. In an embodiment, the helper lipid component is between about 35 mol % to 65 mol % of the total lipid content of the LNP. In an embodiment, the LNP includes lipids at 50 mol % and the helper lipid at 50 mol % of the total lipid content of the LNP.

Other non-limiting, exemplary LNP delivery vehicles are described in U.S. Patent Publication Nos. US20160174546, US20140301951, US20150105538, US20150250725, Wang et al., J. Control Release, 2017 Jan. 31. pii: S0168-3659(17)30038-X. doi: 10.1016/j.jconrel.2017.01.037. [Epub ahead of print]; Altιnoğlu et al., Biomater Sci., 4(12):1773-80, Nov. 15, 2016; Wang et al., PNAS, 113(11): 2868-73 Mar. 15, 2016; Wang et al., PloS One, 10(11): e0141860. doi: 10.1371/journal.pone.0141860. eCollection 2015 Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi: 10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50 (1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi: 10.1038/mtna.2011.3; WO2012135025; US20140348900; US20140328759; US 20140308304; WO 2005/105152; WO 2006/069782; WO 2007/121947; US 2015/082080; US 20120251618; U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316;

### Liposomes

In an embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In an embodiment, liposomes are biocompatible, nontoxic, deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In an embodiment, a liposome delivery vehicle can be used to deliver a virus particle containing a polynucleotides and/or polypeptides of the present invention described elsewhere herein. In an embodiment, the virus particle(s) are adsorbed to the liposome, such as through electrostatic interactions, and/or is attached to the liposomes via a linker.

In an embodiment, the liposome is a Trojan Horse liposome (also known in the art as Molecular Trojan Horses), see e.g. http://cshprotocols.cshlp.org/content/2010/4/pdb.prot5407.long, the teachings of which can be applied and/or adapted to generated and/or deliver the polynucleotides and/or polypeptides of the present invention described elsewhere herein.

Other non-limiting, exemplary liposomes include those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi: 10.1155/2011/469679; WO 2008/042973; U.S. Pat. No. 8,071,082; WO 2014/186366; 20160257951; US20160129120; US20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In an embodiment, the lipid particles are stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol) 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the polynucleotides and/or polypeptides of the present invention described elsewhere herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375:1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In an embodiment, the delivery vehicle comprises a lipidoid, such as any of those set forth in, for example, US20110293703.

In an embodiment, the delivery vehicle can be or include an amino lipid, such as any of those set forth in, for example, Jayaraman, Angew. Chem. Int. Ed. 2012, 51, 8529-8533.

In an embodiment, the delivery vehicle comprises a lipid envelope, such as any of those set forth in, for example, Korman et al., 2011. Nat. Biotech. 29:154-157.

### Lipoplexes/Polyplexes

In an embodiment, the delivery vehicles comprise lipoplexes and/or polyplexes. Lipoplexes may bind to negatively charged cell membrane and induce endocytosis into the cells. Lipoplexes may be complexes comprising lipid(s) and non-lipid components. Exemplary lipoplexes and polyplexes include FuGENE-6 reagent, a non-liposomal solution containing lipids and other components, zwitterionic amino lipids (ZALs), Ca2p (e.g., forming DNA/Ca2+microcomplexes), polyethenimine (PEI) (e.g., branched PEI), and poly(L-lysine) (PLL).

### Sugar-Based Particles

In an embodiment, the delivery vehicle is a sugar-based particle. In an embodiment, the sugar-based particles comprise GalNAc, such as any of those described in WO2014118272; US20020150626; Nair, J K et al., 2014, Journal of the American Chemical Society 136 (49), 16958-16961; Østergaard et al., Bioconjugate Chem., 2015, 26 (8), pp 1451-1455;

### Cell Penetrating Peptides

In an embodiment, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In an embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have palindromic sequences to be partially complementary to one or more of the polynucleotides of the present invention described elsewhere herein. A DNA nanoclew may be coated, e.g., coated with PEI to induce endosomal escape.

### Metal Nanoparticles

In an embodiment, the delivery vehicles comprise gold nanoparticles (also referred to AuNPs or colloidal gold). Gold nanoparticles may form complex with cargos, e.g., polynucleotides and/or polypeptides of the present invention described elsewhere herein. Gold nanoparticles may be coated, e.g., coated in a silicate and an endosomal disruptive polymer, PAsp (DET). Exemplary gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include, but are not limited to, tungsten, palladium, rhodium, platinum, and iridium particles. Other non-limiting, exemplary metal nanoparticles are described in US20100129793.

iTOP

In an embodiment, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In an embodiment, the delivery vehicles comprise polymer-based particles (e.g., nanoparticles). In an embodiment, the polymer-based particles mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In an embodiment, the polymer-based particles comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the polynucleotides and/or polypeptides of the present invention described elsewhere herein include those described in Bawage S S et al., bioRxiv 370460, Lagauzère, Sandra. (2017). Viromer® RED, a powerful tool for transfection of keratinocytes. 10.13140/RG.2.2.16993.61281, Lagauzère, Sandra. (2017). Viromer® Transfection-Factbook 2018: technology, product overview, users' data. 10.13140/RG.2.2.23912.16642. Other exemplary and non-limiting polymeric particles are described in US20170079916, US20160367686, US 20110212179, US20130302401, U.S. Pat. Nos. 6,007,845, 5,855,913, 5,985,309, 5,543,158, WO2012135025, US20130252281, US20130245107, US20130244279; US20050019923, and/or 20080267903.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO are provided in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND is a tetra-lamellar MEND (T-MEND), which may target the cellular nucleus and mitochondria. In certain examples, a MEND is a PEG-peptide-DOPE-conjugated MEND (PPD-MEND), which may target bladder cancer cells. Exemplary MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In an embodiment, pore sizes, pore chemistry, and overall particle sizes are modified for loading different types of cargos. The lipid coating of the particle also may be modified to maximize cargo loading, increase circulation times, and provide precise targeting and cargo release. Exemplary lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Exemplary inorganic nanoparticles include carbon nanotubes (CNTs) (e.g., as described in Bates K and Kostarelos K. (2013). Adv Drug Deliv Rev 65:2023-33), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome forms a complex (e.g., by binding directly or indirectly) to one or more components of the cargo. In certain examples, a molecule of an exosome is fused with first adapter protein and a component of the cargo is fused with a second adapter protein. The first and the second adapter protein may specifically bind each other, thus associating the cargo with the exosome. Exemplary exosomes include those described in Ye Y, et al., Biomater Sci. 2020 Apr. 28.

Other non-limiting, exemplary exosomes include any of those set forth in Alvarez-Erviti et al. 2011, Nat Biotechnol 29:341; El-Andaloussi et al. (Nature Protocols 7:2112-2126 (2012); and Wahlgren et al. (Nucleic Acids Research, 2012, Vol. 40, No. 17 e130).

### Spherical Nucleic Acids (SNAs)

In an embodiment, the delivery vehicle can be a SNA. SNAs are three dimensional nanostructures that comprise densely functionalized and highly oriented nucleic acids that are covalently attached to the surface of spherical nanoparticle cores. The core of the spherical nucleic acid imparts the conjugate with specific chemical and physical properties and acts as a scaffold for assembling and orienting the oligonucleotides into a dense spherical arrangement that gives rise to many of their functional properties, distinguishing them from other forms of matter. In an embodiment, the core is a crosslinked polymer. Non-limiting, exemplary SNAs include any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495: S14-S16, Choi et al., Proc. Natl. Acad. Sci. USA. 2013 110 (19): 7625-7630, Jensen et al., Sci. Transl. Med. 5, 209ra152 (2013) and Mirkin, et al., and Small, 10:186-192.

### Self-Assembling Nanoparticles

In an embodiment, the delivery vehicle is a self-assembling nanoparticle. In an embodiment, the self-assembling nanoparticles contain one or more polymers. In an embodiment, the self-assembling nanoparticles are PEGylated. Self-assembling nanoparticles are known in the art. Non-limiting, exemplary self-assembling nanoparticles include any of those set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In an embodiment, the delivery vehicle is a supercharged protein. As used herein “supercharged proteins” are a class of engineered or naturally occurring proteins with unusually high positive or negative net theoretical charge. Non-limiting, exemplary supercharged proteins include any of those set forth in Lawrence et al., 2007, Journal of the American Chemical Society 129, 10110-10112.

### Targeted Delivery

In an embodiment, the delivery vehicle allows for targeted delivery to a specific cell, tissue, organ, or system. In such embodiments, the delivery vehicle includes one or more targeting moieties that directs targeted delivery of the cargo(s). In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, mention is made of Deshpande et al, Nanomedicine (Lond). 8(9), (2013), and the documents it cites, all of which are incorporated herein by reference and the teachings of which can be applied and/or adapted for targeted delivery of one or more polynucleotides and/or polypeptides of the present invention described elsewhere herein. Mention is also made of International Patent Publication No. WO 2016/027264, and the documents it cites, all of which are incorporated herein by reference, the teachings of which can be applied and/or adapted for targeted delivery of one or more polynucleotides and/or polypeptides of the present invention described elsewhere herein. Mention is made of Lorenzer et al, “Going beyond the liver: Progress and challenges of targeted delivery of siRNA therapeutics,” Journal of Controlled Release, 203:1-15 (2015), and the documents it cites, all of which are incorporated herein by reference, the teachings of which can be applied and/or adapted for targeted delivery of one or more polynucleotides and/or polypeptides of the present invention described elsewhere herein.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis, and atherosclerosis; accordingly, folate-mediated targeting of the invention can also be used for studying, addressing or treating inflammatory disorders, as well as cancers. Folate-linked lipid particles or nanoparticles or liposomes or lipid by layers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck, and lung cells, such as head, neck and non-small-cell lung cancer cells, cells of the mouth such as oral tumor cells.

Also, as to active targeting, a lipid entity of the invention can be multifunctional, i.e., employ more than one targeting moiety such as CPP, along with Tf; a bifunctional system, e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm.

With respect to receptor-mediated targeting, one of ordinary skill in the art takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, one of ordinary skill in the art should take into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG (SEQ ID NO: 310) such as APRPG-PEG-modified (SEQ ID NO: 310). VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention, e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen, and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix.

Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA (SEQ ID NO: 311), cholesteryl-GALA (SEQ ID NO: 311) and PEG-GALA (SEQ ID NO: 311) may show a highly efficient endosomal release; a pore-forming protein listeriolysin O may provide an endosomal escape mechanism; and histidine-rich peptides have the ability to fuse with the endosomal membrane, resulting in pore formation, and can buffer the proton pump causing membrane lysis.

The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein-discussed, there is an embodiment of the invention wherein the delivery system comprises such a targeting or active targeting moiety. Likewise, Table A provides exemplary targeting moieties that can be used in the practice of the invention an as to each an embodiment of the invention provides a delivery system that comprises such a targeting moiety.

### Vaccine

In another aspect, embodiments disclosed herein are directed to the immunogenic compositions disclosed herein formulated as vaccines. A vaccine is a biological preparation that provides active acquired immunity to a particular infectious or malignant disease. Tumor specific antigens may be produced in vitro as peptides or polypeptides, which may then be formulated into a vaccine or immunogenic composition and administered to a subject. Such in vitro production may occur by a variety of methods known to one of ordinary skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide.

The present invention also contemplates the use of nucleic acid molecules as vehicles for delivering antigenic peptides/polypeptides to the subject in need thereof, in vivo, in the form of, e.g., DNA/RNA vaccines (see, e.g., WO2012/159643, and WO2012/159754, hereby incorporated by reference in their entirety).

In one embodiment, antigenic peptides may be administered to a patient in need thereof by use of an mRNA vaccine (see, e.g., Sahin, U, Kariko, K and Tureci, O (2014). mRNA-based therapeutics-developing a new class of drugs. Nat Rev Drug Discov 13:759-780; Weissman D, Karikó K. mRNA: Fulfilling the Promise of Gene Therapy. Mol Ther. 2015; 23(9):1416-1417. doi: 10.1038/mt.2015.138; Kowalski P S, Rudra A, Miao L, Anderson D G. Delivering the Messenger: Advances in Technologies for Therapeutic mRNA Delivery. Mol Ther. 2019; 27(4):710-728. doi: 10.1016/j.ymthe.2019.02.012; Magadum A, Kaur K, Zangi L. mRNA-Based Protein Replacement Therapy for the Heart. Mol Ther. 2019; 27(4):785-793. doi: 10.1016/j.ymthe.2018.11.018; Reichmuth A M, Oberli M A, Jaklenec A, Langer R, Blankschtein D. mRNA vaccine delivery using lipid nanoparticles Ther Deliv. 2016; 7(5):319-334. doi: 10.4155/tde-2016-0006; and Khalil A S, Yu X, Umhoefer J M, et al. Single-dose mRNA therapy via biomaterial-mediated sequestration of overexpressed proteins. Sci Adv. 2020; 6 (27):eaba2422). In an exemplary embodiment, mRNA encoding for an antigenic peptide is delivered using lipid nanoparticles (see, e.g., Reichmuth, et al., 2016) and administered directly to tumor tissue. In an exemplary embodiment, mRNA encoding for an antigenic peptide is delivered using biomaterial-mediated sequestration (see, e.g., Khalil, et al., 2020) and administered directly to tumor tissue.

In one embodiment, antigens are administered to a patient in need thereof by use of a plasmid. These are plasmids that usually consist of a strong viral promoter to drive the in vivo transcription and translation of the gene (or complementary DNA) of interest (Mor, et al., (1995), The Journal of Immunology 155 (4): 2039-2046). Intron A may sometimes be included to improve mRNA stability and hence increase protein expression (Leitner et al. (1997), The Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999), Adv. Parasitol. Advances in Parasitology 42:343-410; Robinson et al., (2000). Adv. Virus Res. Advances in Virus Research 55:1-74; Bohm et al., (1996). Journal of Immunological Methods 193 (1): 29-40). Multi cistronic vectors are sometimes constructed to express more than one immunogen, or to express an immunogen and an immunostimulatory protein (Lewis et al., (1999). Advances in Virus Research (Academic Press) 54:129-88).

Since the plasmid is the “vehicle” from which the immunogen is expressed, optimizing vector design for maximal protein expression is essential (Lewis et al., (1999). Advances in Virus Research (Academic Press) 54:129-88). One way of enhancing protein expression is by optimizing the codon usage of pathogenic mRNAs for eukaryotic cells. Another consideration is the choice of promoter. Such promoters may be the SV40 promoter or Rous Sarcoma Virus (RSV). Plasmids may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (EVI) in skeletal muscle, or intradermally (ID), with DNA being delivered to the extracellular spaces. This can be assisted by electroporation by temporarily damaging muscle fibres with myotoxins such as bupivacaine; or by using hypertonic solutions of saline or sucrose (Alarcon et al., (1999). Adv. Parasitol. Advances in Parasitology 42:343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42:343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42:343-410; Lewis et al., (1999). Advances in Virus Research (Academic Press) 54:129-88).

Alternative delivery methods may include aerosol instillation of naked DNA on mucosal surfaces, such as the nasal and lung mucosa, (Lewis et al., (1999). Advances in Virus Research (Academic Press) 54:129-88) and topical administration of pDNA to the eye and vaginal mucosa (Lewis et al., (1999) Advances in Virus Research (Academic Press) 54:129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Ex Vivo Cytosolic Delivery of Functional Macromolecules to Immune Cells, Sharei et al, PLOS ONE|DOI: 10.1371/journal.pone.O1 18803 Apr. 13, 2015).

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshia et al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In one embodiment, a neoplasia vaccine or immunogenic composition may include separate DNA plasmids encoding, for example, one or more antigenic peptides/polypeptides as identified in according to the invention. As discussed herein, the exact choice of expression vectors can depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

One or more antigenic peptides of the invention may be encoded and expressed in vivo using a viral based system (e.g., an adenovirus system, an adeno associated virus (AAV) vector, a poxvirus, or a lentivirus). In one embodiment, the neoplasia vaccine or immunogenic composition includes a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. patent application No. 20080254008, hereby incorporated by reference). The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

Among vectors that may be used in the practice of the invention, integration in the host genome of a cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the invention is a minimal non-primate lentiviral vector, such as a lentiviral vector based on the equine infectious anemia virus (EIAV) (see, e.g., Balagaan, (2006) J Gene Med; 8:275-285, Published online 21 Nov. 2005 in Wiley InterScience (www.interscience.wiley.com)). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the Brain, see, e.g., US Patent Publication Nos. US20110293571; US20040013648, US20070025970, US20090111106 and U.S. Pat. No. 7,259,015. In another embodiment lentiviral vectors are used to deliver vectors to the brain of those being treated for a disease.

As to lentivirus vector systems useful in the practice of the invention, mention is made of U.S. Pat. Nos. 6,428,953, 6,165,782, 6,013,516, 5,994,136, 6,312,682, and 7,198,784, and documents cited therein.

In an embodiment herein the delivery is via an lentivirus. Zou et al. administered about 10 μl of a recombinant lentivirus having a titer of 1×109 transducing units (TU)/ml by an intrathecal catheter. These sort of dosages can be adapted or extrapolated to use of a retroviral or lentiviral vector in the present invention. For transduction in tissues such as the brain, it is necessary to use very small volumes, so the viral preparation is concentrated by ultracentrifugation. The resulting preparation should have at least 108 TU/ml, preferably from 108 to 109 TU/ml, more preferably at least 109 TU/ml. Other methods of concentration such as ultrafiltration or binding to and elution from a matrix may be used.

In other embodiments the amount of lentivirus administered may be 1×105 or about 1×105 plaque forming units (PFU), 5×105 or about 5×105 PFU, 1×106 or about 1×106 PFU, 5×106 or about 5×106 PFU, 1×107 or about 1×107PFU, 5×107 or about 5×107 PFU, 1×108 or about 1×108 PFU, 5×108 or about 5×108 PFU, 1×109 or about 1×109 PFU, 5×109 or about 5×109 PFU, 1×1010 or about 1×1010 PFU or 5×1010 or about 5×1010 PFU as total single dosage for an average human of 75 kg or adjusted for the weight and size and species of the subject. One of ordinary skill in the art will be able to determine suitable dosage. Suitable dosages for a virus can be determined empirically. Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the invention, mention is made of U.S. Pat. No. 6,955,808. The adenovirus vector used can be selected from the group consisting of the Ad5, Ad35, Adl 1, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Adl 1 vectors are described in U.S. Pat. No. 6,913,922. C6 adenovirus vectors are described in U.S. Pat. Nos. 6,780,407; 6,537,594; 6,309,647; 6,265, 189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present invention. Furthermore, “gutless” adenovirus vectors, in which all viral genes are deleted, can also be used in accordance with the present invention. Such vectors require a helper virus for their replication and require a special human 293 cell line expressing both E1 a and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present invention, and can even be used for co-delivery of a large number of heterologous inserts/genes.

In an embodiment herein the delivery is via an adenovirus, which may be at a single booster dose containing at least 1×105 particles (also referred to as particle units, pu) of adenoviral vector. In an embodiment herein, the dose preferably is at least about 1×106 particles (for example, about 1×106-1×1012 particles), more preferably at least about 1×107 particles, more preferably at least about 1×108 particles (e.g., about 1×108-1×1011 particles or about 1×108-1×1012 particles), and most preferably at least about 1×109 particles (e.g., about 1×109-1×1010 particles or about 1×109-1×1012 particles), or even at least about 1×1010 particles (e.g., about 1×1010-1×1012 particles) of the adenoviral vector. Alternatively, the dose comprises no more than about 1×1014 particles, preferably no more than about 1×1013 particles, even more preferably no more than about 1×1012 particles, even more preferably no more than about 1×1011 particles, and most preferably no more than about 1×1010 particles (e.g., no more than about 1×109 articles). Thus, the dose may contain a single dose of adenoviral vector with, for example, about 1×106 particle units (pu), about 2×106 pu, about 4×106 pu, about 1×107 pu, about 2×10 pu, about 4×10 pu, about 1×10 pu, about 2×10 pu, about 4×10 pu, about 1×109 pu, about 2×109 pu, about 4×109 pu, about 1×1010 pu, about 2×1010 pu, about 4×1010 pu, about 1×1011 pu, about 2×1011 pu, about 4×1011 pu, about 1×1012 pu, about 2×1012 pu, or about 4×1012 pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: Synapsinl for all neurons, CaMKIIalpha for excitatory neurons, GAD67 or GAD65 or VGAT for GABAergic neurons, etc. Promoters used to drive RNA synthesis can include: Pol III promoters such as U6 or HI. The use of a Pol II promoter and intronic cassettes can be used to express guide RNA (gRNA).

With regard to AAV vectors useful in the practice of the invention, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein.

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

[In an embodiment herein, the delivery is via an AAV. A therapeutically effective dosage for in vivo delivery of the AAV to a human is believed to be in the range of from about 20 to about 50 ml of saline solution containing from about 1×1010 to about 1×1050 functional AAV/ml solution. The dosage may be adjusted to balance the therapeutic benefit against any side effects. In an embodiment herein, the AAV dose is generally in the range of concentrations from about 1×10 to 1×10 genomes AAV, from about 1×10 to 1×10 genomes AAV, from about 1×1010 to about 1×1016 genomes, or about 1×1011 to about 1×1016 genomes AAV. A human dosage may be about 1×1013 genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2×1013 viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In another embodiment effectively activating a cellular immune response for a neoplasia vaccine or immunogenic composition can be achieved by expressing the relevant antigens in a vaccine or immunogenic composition in a non-pathogenic microorganism. Well-known examples of such microorganisms are Mycobacterium bovis BCG, Salmonella and Pseudomona (See, U.S. Pat. No. 6,991,797, hereby incorporated by reference in its entirety). In another embodiment a Poxvirus is used in the neoplasia vaccine or immunogenic composition. These include orthopoxvirus, avipox, vaccinia, MVA, NYVAC, canarypox, ALVAC, fowlpox, TROVAC, etc. (see e.g., Verardi et al., Hum Vaccin Immunother. 2012 July; 8(7):961-70; and Moss, Vaccine. 2013; 31(39): 4220-4222).

### Vaccine or Immunogenic Composition Adjuvant

Effective vaccine or immunogenic compositions advantageously include a strong adjuvant to initiate an immune response. As described herein, poly-ICLC, an agonist of TLR3 and the RNA helicase-domains of MDA5 and RIG3, has shown several desirable properties for a vaccine or immunogenic composition adjuvant. These properties include the induction of local and systemic activation of immune cells in vivo, production of stimulatory chemokines and cytokines, and stimulation of antigen-presentation by DCs. Furthermore, poly-ICLC can induce durable CD4+ and CD8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NY-ESO-1 peptide vaccine (in addition to Montanide) showed induction of CD4+ and CD8+ T cell, as well as antibody responses to the peptide in a recent phase 1 study. At the same time, poly-ICLC has been extensively tested in more than 25 clinical trials to date and exhibited a relatively benign toxicity profile. In addition to a powerful and specific immunogen the antigen peptides may be combined with an adjuvant (e.g., poly-ICLC) or another anti-neoplastic agent. Without being bound by theory, these antigens are expected to bypass central thymic tolerance (thus allowing stronger anti-tumor T cell response), while reducing the potential for autoimmunity (e.g., by avoiding targeting of normal self-antigens). An effective immune response advantageously includes a strong adjuvant to activate the immune system (Speiser and Romero, Seminars in Immunol 22:144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, Cancer J. 16:382-391 (2010)). Among the TLR agonists, poly-ICLC (a synthetic double-stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, J Exp Med 208:2357 (2011)). In a preferred embodiment, Hiltonol®, a GMP preparation of poly-ICLC prepared by Oncovir, Inc, is utilized as the adjuvant. In other embodiments, other adjuvants described herein are envisioned. For instance, oil-in-water, water-in-oil or multiphasic W/O/W; see, e.g., U.S. Pat. No. 7,608,279 and Aucouturier et al, Vaccine 19 (2001), 2666-2672, and documents cited therein.

TABLE A

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon adenocarcinoma CC531 |
| anti-HER2 antibody | HER2 | HER2 -overexpressing tumors |
| anti-GD2 | GD2 | neuroblastoma, melanoma |
| anti-EGFR | EGFR | tumor cells overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell marker) | leukemia, lymphoma |
| cell-penetrating peptide | avβ3 | blood-brain barrier |
| cyclic arginine-glycine- | | glioblastoma cells, human umbilical |
| aspartic acid-tyrosine- | | vein endothelial cells, tumor |
| cysteine peptide | | |
| (c(RGDyC)-LP | | angiogenesis |
| (SEQ ID NO: 312)) | | |
| ASSHN (SEQ ID NO: | | endothelial progenitor cells; |
| 313) peptide | | anti-cancer |
| PR_b peptide | a5β1 integrin | cancer cells |
| AG86 peptide | a6β4 integrin | cancer cells |
| KCCYSL (SEQ ID NO: | HER-2 receptor | cancer cells |
| 314) (P6.1 peptide) | | |
| affinity peptide LN | Aminopeptidase N | APN-positive tumor |
| (YEVGHRC (SEQ ID | (APN/CD13) | |
| NO: 315)) | | |
| synthetic somatostatin | Somatostatin receptor 2 | breast cancer |
| analogue | (SSTR2) | |
| anti-CD20 monoclonal | B-lymphocytes | B cell lymphoma |
| antibody | | |
| Multiple myeloma cell | CCR10, CD53, CD10, | Multiple myeloma and resistant |
| surface marker | EVI2B, CD33, CD229, | Multiple myeloma (see e.g., Ferguson |
| | a CCA of the present | et al. Nat Commun. 2022; 13: 412) and |
| | disclosure | Atanackovic et al., Haematologica. |
| | | 2011 October; 96(10): 1512-1520. |

Other exemplary targeting moieties are described elsewhere herein, such as epitope tags and the like.

### Responsive Delivery

In an embodiment, the delivery vehicle can allow for responsive delivery of the cargo(s), e.g., one or more polynucleotides and/or polypeptides of the present invention described elsewhere herein. Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g. chemical composition, etc.), and a biologic or physiologic stimuli (e.g. environmental pH, osmolarity, salinity, biologic molecule, etc.). In an embodiment, the targeting moiety can be responsive to an external stimuli and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

The invention also comprehends redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria, and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes also can be used as a trigger to release payload. Enzymes, including MMPs (e.g., MMP2), phospholipase A2, alkaline phosphatase, transglutaminase, or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO: 316)) can be incorporated into a linker, and can have antibody targeting, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

### Cells

In an embodiment the present disclosure provides cells and organisms comprising the compositions, such as the CAA polynucleotides, polypeptides, vectors, delivery vehicles, etc. described herein. In an embodiment, the cells are producer cells and are capable of generating virus particles or other delivery vehicles (e.g., exosomes) containing the one or more polynucleotides and/or polypeptides of the present invention. The cells may be in tissue, organ, or isolated cells. Such cells may be of a unique type of cells or a group of different types of cells such as cultured cell lines, primary cells and proliferative cells. The cells may be prokaryotic cells, lower eukaryotic cells such as yeast, and other eukaryotic cells such as insect cells, plant, and mammalian (e.g., human or non-human) cells as well as cells capable of producing the vector of the invention (e.g., 293, HER96, PERC.6 cells, Vero, HeLa, CEF, duck cell lines, etc.). The cells may include cells which can be or has been the recipient of the vector described herein as well as progeny of such cells. Host cells can be cultured in conventional fermentation bioreactors, flasks, and petri plates. Culturing can be carried out at a temperature, pH, and oxygen content appropriate for a given cell. No attempts will be made here to describe in detail the various prokaryote and eukaryotic host cells and methods known for the production of the polypeptides and vectors herein.

In an embodiment, the cells, e.g., engineered cells, are eukaryotic cells, such as mammalian cells, e.g., human cells. In an embodiment, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In an embodiment, the cells are human cells. The cells typically are primary cells, such as those isolated directly from a subject and/or isolated from a subject and frozen. In an embodiment, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

### Methods of Treating Cancer Using Antigenic Compositions

In one aspect, embodiments disclosed herein are directed to methods of treating cancer by administering to a subject in need thereof the immunogenic compositions and vaccine compositions disclosed herein. In one embodiment, the immunogenic compositions and vaccine may comprise the cancer-associated antigens described herein. In one embodiment, the method comprises administering a vaccine comprising a disease-associated antigen selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC. In one embodiment, the subject is suffering from a hematological malignancy. In one embodiment, the hematological malignancy is selected from multiple myeloma, acute myeloid leukemia, or chronic lymphocytic leukemia.

In one embodiment, the vaccine is polypeptide-based and comprises the polypeptide selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

In one embodiment, the vaccine is DNA-based and comprises a DNA polynucleotide sequence encoding a polypeptide from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

In one embodiment, the vaccine is RNA-based and comprises an RNA polynucleotide sequence encoding a polypeptide from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

Generally, the methods may comprise administering a pharmaceutically effective (e.g., therapeutically effective amount or prophylactically effective amount)) amount of an immunogenic composition or pharmaceutical formulation thereof (including but not limited to a peptide, DNA, or mRNA vaccine) herein to a subject, e.g., a subject in need thereof. In some cases, the method comprises administering the composition(s), the polynucleotide(s), and/or the vector(s) herein to a subject. A pharmaceutically effective amount refers to an amount that can elicit a biological, medicinal, or immunological response in a tissue, system, or subject (e.g., animal or human) that can prevent or alleviate one or more of the local or systemic symptoms or features of a disease or condition being treated.

Described in certain example embodiments herein are methods of inducing a B-cell response and/or T-cell response to a virus in a subject in need thereof, comprising administering, to the subject, the immunogenic composition or the therapeutic composition, or a pharmaceutical formulation thereof of the present invention described elsewhere herein. In certain example embodiments, the B cell response comprises antibody production. Described in certain example embodiments herein are methods of treating a viral infection in a subject in need thereof comprising administering, to the subject in need thereof, the immunogenic composition or the therapeutic composition, or a pharmaceutical formulation thereof of the present invention as described elsewhere herein in combination with an antiviral therapeutic. Described in certain example embodiments herein are methods an infection status of a subject comprising contacting immune cells derived from a subject with the immunogenic composition or a pharmaceutical formulation thereof of the present invention as described elsewhere herein; and detecting cross-reactivity of the immune cells to the immunogenic composition.

### Multiple Myeloma-Reactive T Cell Gene Expression Signatures

As used herein a “signature” may encompass any gene or genes, protein or proteins, or epigenetic element(s) whose expression profile or whose occurrence is characteristic of multiple myeloma reactive T cells. As used herein, the terms “signature”, “expression profile”, or “expression program” may be used interchangeably.

In an embodiment of the present disclosure, multiple myeloma reactive T cells may be identified and isolated based on the detection of a multiple myeloma-reactive T cell molecular signature as disclosed in Table 6. For example, the signature profile may be used in microfluidics-based forward screening of single T cells against autologous tumor cells to identify and facilitate the isolation and expansion of MM reactive T cells in bone marrow or peripheral blood samples from therapy-naïve multiple myeloma patients.

In an embodiment, a gene expression signature of multiple myeloma reactive T cells comprises the genes GNLY, ZNF683, GZMH, FGFBP2, GZMB, NKG7, CCL5, HOPX, KLRD1, EFHD2, CD8A, CTSW, CST7, ITGB1, and BHLHE40 (sigMM).

In an embodiment, a gene expression signature of multiple myeloma reactive T cells comprises the genes GNLY, ZNF683, GZMH, FGFBP2, and GZMB (sigMM_2).

In an embodiment, a gene expression signature of multiple myeloma reactive T cells comprises the genes GNLY, ZNF683, GZMH, FGFBP2, GZMB, NKG7, CCL5, HOPX, KLRD1, and EFHD2 (sigMM_3).

In an embodiment, a gene expression signature of multiple myeloma reactive T cells comprises the genes GNLY, ZNF683, GZMH, FGFBP2, GZMB, NKG7, CCL5, HOPX, KLRD1, EFHD2, CD8A, CTSW, CST7, ITGB1, BHLHE40, LYAR, S100A4, GZMA, MXRA7, and KLRK1 (sigMM_4).

In an embodiment, a gene expression signature of multiple myeloma reactive T cells comprises the genes GNLY, ZNF683, GZMH, FGFBP2, GZMB, NKG7, CCL5, HOPX, KLRD1, EFHD2, CD8A, CTSW, CST7, ITGB1, BHLHE40, LYAR, S100A4, GZMA, MXRA7, KLRK1, SH3BGRL3, ITGA4, FCRL6, TGFB1, CCL4, ZEB2, AOAH, AHNAK, S100A10, and LGALS1 (sigMM_5).

In an embodiment, a gene expression signature of multiple myeloma reactive T cells comprises the genes GNLY, ZNF683, GZMH, FGFBP2, GZMB, NKG7, CCL5, HOPX, KLRD1, EFHD2, CD8A, CTSW, CST7, ITGB1, BHLHE40, LYAR, S100A4, GZMA, MXRA7, KLRK1, SH3BGRL3, ITGA4, FCRL6, TGFB1, CCL4, ZEB2, AOAH, AHNAK, S100A10, LGALS1, PRF1, ITGB2, CD52, TPST2, PRSS23, ANXA1, CYBA, C12orf75, LAIR2, and MATK, (sigMM_6).

In an embodiment, a gene expression signature of multiple myeloma reactive T cells comprises the genes GNLY, ZNF683, GZMH, FGFBP2, GZMB, NKG7, CCL5, HOPX, KLRD1, EFHD2, CD8A, CTSW, CST7, ITGB1, BHLHE40, LYAR, S100A4, GZMA, MXRA7, KLRK1, SH3BGRL3, ITGA4, FCRL6, TGFB1, CCL4, ZEB2, AOAH, AHNAK, S100A10, LGALS1, PRF1, ITGB2, CD52, TPST2, PRSS23, ANXA1, CYBA, C12orf75, LAIR2, MATK, S100A6, TNFAIP3, CLIC1, KLF6, Clorf21, SYNE2, HLA-DPB1, HLA-DPA1, DSTN, and CD99, (sigMM_7).

In an embodiment, a gene expression signature of multiple myeloma reactive T cells comprises the genes EFHD2, SH3BGRL3, CD52, ZNF683, S100A10, S100A6, S100A4, FCRL6, TAGLN2, Clorf21, PLEK, GNLY, CD8A, ZEB2, ITGA4, BHLHE40, LYAR, FGFBP2, HOPX, GZMA, CLIC1, HLA-DPA1, HLA-DPB1, TNFAIP3, AOAH, ANXA1, KLF6, ITGB1, PRF1, AHNAK, CTSW, PRSS23, KLRD1, KLRK1, LINC02446, RPS26, C12orf75, RGCC, GZMH, GZMB, NFKBIA, SYNE2, FOS, PPP2R5C, CRIP1, AKAP13, CYBA, CCL5, CCL4, MXRA7, GADD45B, MATK, ZFP36, TGFB1, NKG7, LAIR2, DSTN, CST7, ITGB2, TPST2, LGALS1, CD99, and FLNA (sigMM_8).

In an embodiment, a gene expression signature of multiple myeloma reactive T cells comprises one or more genes chosen from GNLY, ZNF683, GZMH, FGFBP2, GZMB, NKG7, CCL5, HOPX, KLRD1, EFHD2, CD8A, CTSW, CST7, ITGB1, BHLHE40, LYAR, S100A4, GZMA, MXRA7, KLRK1, SH3BGRL3, ITGA4, FCRL6, TGFB1, CCL4, ZEB2, AOAH, AHNAK, S100A10, LGALS1, PRF1, ITGB2, CD52, TPST2, PRSS23, ANXA1, CYBA, C12orf75, LAIR2, MATK, S100A6, TNFAIP3, CLIC1, KLF6, Clorf21, SYNE2, HLA-DPB1, HLA-DPA1, DSTN, and CD99, (sigMM_7).

In an embodiment, a gene expression signature of multiple myeloma reactive T cells comprises two or more genes chosen from GNLY, ZNF683, GZMH, FGFBP2, GZMB, NKG7, CCL5, HOPX, KLRD1, EFHD2, CD8A, CTSW, CST7, ITGB1, BHLHE40, LYAR, S100A4, GZMA, MXRA7, KLRK1, SH3BGRL3, ITGA4, FCRL6, TGFB1, CCL4, ZEB2, AOAH, AHNAK, S100A10, LGALS1, PRF1, ITGB2, CD52, TPST2, PRSS23, ANXA1, CYBA, C12orf75, LAIR2, MATK, S100A6, TNFAIP3, CLIC1, KLF6, Clorf21, SYNE2, HLA-DPB1, HLA-DPA1, DSTN, and CD99, (sigMM_7).

In an embodiment, a gene expression signature of multiple myeloma reactive T cells comprises three or more genes chosen from GNLY, ZNF683, GZMH, FGFBP2, GZMB, NKG7, CCL5, HOPX, KLRD1, EFHD2, CD8A, CTSW, CST7, ITGB1, BHLHE40, LYAR, S100A4, GZMA, MXRA7, KLRK1, SH3BGRL3, ITGA4, FCRL6, TGFB1, CCL4, ZEB2, AOAH, AHNAK, S100A10, LGALS1, PRF1, ITGB2, CD52, TPST2, PRSS23, ANXA1, CYBA, C12orf75, LAIR2, MATK, S100A6, TNFAIP3, CLIC1, KLF6, Clorf21, SYNE2, HLA-DPB1, HLA-DPA1, DSTN, and CD99, (sigMM_7).

In an embodiment, a gene expression signature of multiple myeloma reactive T cells comprises one or more genes chosen from EFHD2, SH3BGRL3, CD52, ZNF683, S100A10, S100A6, S100A4, FCRL6, TAGLN2, Clorf21, PLEK, GNLY, CD8A, ZEB2, ITGA4, BHLHE40, LYAR, FGFBP2, HOPX, GZMA, CLIC1, HLA-DPA1, HLA-DPB1, TNFAIP3, AOAH, ANXA1, KLF6, ITGB1, PRF1, AHNAK, CTSW, PRSS23, KLRD1, KLRK1, LINC02446, RPS26, C12orf75, RGCC, GZMH, GZMB, NFKBIA, SYNE2, FOS, PPP2R5C, CRIP1, AKAP13, CYBA, CCL5, CCL4, MXRA7, GADD45B, MATK, ZFP36, TGFB1, NKG7, LAIR2, DSTN, CST7, ITGB2, TPST2, LGALS1, CD99, and FLNA (sigMM_8).

In an embodiment, a gene expression signature of multiple myeloma reactive T cells comprises two or more genes chosen from EFHD2, SH3BGRL3, CD52, ZNF683, S100A10, S100A6, S100A4, FCRL6, TAGLN2, Clorf21, PLEK, GNLY, CD8A, ZEB2, ITGA4, BHLHE40, LYAR, FGFBP2, HOPX, GZMA, CLIC1, HLA-DPA1, HLA-DPB1, TNFAIP3, AOAH, ANXA1, KLF6, ITGB1, PRF1, AHNAK, CTSW, PRSS23, KLRD1, KLRK1, LINC02446, RPS26, C12orf75, RGCC, GZMH, GZMB, NFKBIA, SYNE2, FOS, PPP2R5C, CRIP1, AKAP13, CYBA, CCL5, CCL4, MXRA7, GADD45B, MATK, ZFP36, TGFB1, NKG7, LAIR2, DSTN, CST7, ITGB2, TPST2, LGALS1, CD99, and FLNA (sigMM_8).

In an embodiment, a gene expression signature of multiple myeloma reactive T cells comprises three or more genes chosen from EFHD2, SH3BGRL3, CD52, ZNF683, S100A10, S100A6, S100A4, FCRL6, TAGLN2, Clorf21, PLEK, GNLY, CD8A, ZEB2, ITGA4, BHLHE40, LYAR, FGFBP2, HOPX, GZMA, CLIC1, HLA-DPA1, HLA-DPB1, TNFAIP3, AOAH, ANXA1, KLF6, ITGB1, PRF1, AHNAK, CTSW, PRSS23, KLRD1, KLRK1, LINC02446, RPS26, C12orf75, RGCC, GZMH, GZMB, NFKBIA, SYNE2, FOS, PPP2R5C, CRIP1, AKAP13, CYBA, CCL5, CCL4, MXRA7, GADD45B, MATK, ZFP36, TGFB1, NKG7, LAIR2, DSTN, CST7, ITGB2, TPST2, LGALS1, CD99, and FLNA (sigMM_8).

In an embodiment, the detection of the disclosed gene signatures characteristic of multiple myeloma reactive T cells in a patient's bone marrow or peripheral blood is predictive of a better treatment outcome, for example, with adjuvant chemotherapy, bi-specific antibodies, checkpoint inhibitors.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use.

A “pharmaceutically acceptable carrier or excipient” as used in the specification and claims includes both one and more than one such carrier or excipient. When present, the compound can optionally be present in the pharmaceutical formulation as a pharmaceutically acceptable salt. In an embodiment, the pharmaceutical formulation can include, such as an active ingredient, a polynucleotide, polypeptide, vector, delivery vehicle, and/or cell of the present invention described in greater detail elsewhere herein.

In an embodiment, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium, and ammonium. Those of ordinary skill in the art will recognize from this disclosure and the knowledge in the art that further pharmaceutically acceptable salts for the pooled tumor specific antigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The pharmaceutical formulations described herein can be administered to a subject in need thereof via any suitable method or route, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In an embodiment, the subject in need thereof has or is suspected of having a viral infection or a symptom thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In an embodiment, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatoir anti-histamines, anti-infectives, chemotherapeutics, and combinations thereof. In an embodiment, the pharmaceutical formulation comprises an effective amount of one or more chemotherapeutics, immunomodulators, or both. Suitable immunomodulators include, but are not limited to, prednisone, azathioprine, 6-MP, cyclosporine, tacrolimus, methotrexate, interleukins (e.g., IL-2, IL-7, and IL-12), cytokines (e.g. interferons (e.g. IFN-a, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers). In an embodiment, the immunomodulator is a checkpoint blockade modulator. In an embodiment, the immunomodulator is a checkpoint blockade inhibitor.

Exemplary chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

### Effective Amounts

In an embodiment, the amount of the primary active agent and/or optional secondary agent is an effective amount, least effective amount, and/or therapeutically effective amount. As used herein, “effective amount,” “effective concentration,” and/or the like refers to the amount, concentration, etc. of the primary and/or optional secondary agent included in the pharmaceutical formulation that achieve one or more therapeutic effects or desired effect. As used herein, “least effective,” “least effective concentration,” and/or the like amount refers to the lowest amount, concentration, etc. of the primary and/or optional secondary agent that achieves the one or more therapeutic or other desired effects. As used herein, “therapeutically effective amount,” “therapeutically effective concentration,” and/or the like refers to the amount, concentration, etc. of the primary and/or optional secondary agent included in the pharmaceutical formulation that achieves one or more therapeutic effects. In an embodiment, the one or more therapeutic effects are inducing an immune response in a subject to which they are delivered, inducing a B- and/or T-cell response in a subject to which it is delivered, treating or preventing a viral infection in a subject to which it is delivered.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg, ng, μg, mg, or g or be any numerical value or subrange within any of these ranges.

In an embodiment, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In an embodiment, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In an embodiment, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In an embodiment, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In an embodiment, the effective titer can be about 1×10¹ transforming units per pL, nL, μL, mL, or L to 1×1020/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×1020 transforming units per pL, nL, μL, mL, or L or any numerical value or subrange within these ranges. In an embodiment, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In an embodiment, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In an embodiment, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In an embodiment, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In an embodiment, the given site is proximal to the administration site. In an embodiment, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Such dosage forms can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In an embodiment, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In an embodiment, compounds, molecules, compositions, vectors, vector systems, cells, or a combination thereof described herein can be the ingredient whose release is delayed. In an embodiment the primary active agent is the ingredient whose release is delayed. In an embodiment, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In an embodiment for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In an embodiment, a primary active ingredient, optional secondary active ingredient, and/or pharmaceutically acceptable salt thereof where appropriate can be in a dosage form adapted for inhalation is in a particle-size-reduced form that is obtained or obtainable by micronization. In an embodiment, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In an embodiment, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose, multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In an embodiment, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared In an embodiment, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In an embodiment, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In an embodiment, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, and combinations thereof. In an embodiment, the co-therapy and/or combination therapy comprises an effective amount of one or more chemotherapeutics, immunomodulators, or both. Exemplary chemotherapeutics and immunomodulators for Co- and Combination therapies are previously discussed in connection with additional active agents.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In an embodiment, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In an embodiment, the first one or a few initial amount(s) administered can be a higher dose than subsequent doses. This is typically referred to in the art as a loading dose or doses and a maintenance dose, respectively. In an embodiment, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

mRNA Vaccines

In an embodiment, the pharmaceutical formulations and/or immunogenic composition described herein are mRNA vaccines. In an embodiment, one or more CAA (including but not limited to conserved cancer antigen) polynucleotides or polynucleotides encoding the one or more CAA (including but not limited to conserved cancer antigen) polypeptides of the present invention described herein are included in an mRNA vaccine composition. In an embodiment, the polypeptides are immunogenic polypeptides. The mRNA vaccine composition can be administered to a subject in need thereof. In an embodiment, the vaccine is administered to a subject in an effective amount to induce an immune response in the subject.

Described herein are pharmaceutical compositions that include one or more isolated messenger ribonucleic (mRNA) polynucleotides encoding at least one CAA polypeptide or an immunogenic fragment thereof (e.g., an immunogenic fragment capable of inducing an immune response to the antigenic polypeptide), such as any of those polynucleotides described in greater detail elsewhere herein, where the isolated mRNA is formulated in a lipid nanoparticle. As used herein “antigenic polypeptide” encompasses immunogenic fragments of the antigenic polypeptide (an immunogenic fragment that is induces (or is capable of inducing) an immune response to a cancer. In an embodiment, the cancer is a blood cancer. In an embodiment, the cancer is a white blood cell cancer. In an embodiment, the cancer is multiple myeloma. In an embodiment, the mRNA encoding at least one CAA polypeptide or immunogenic fragment thereof can include an open reading frame that encodes the at least one CAA antigenic polypeptide or immunogenic fragment thereof. In an embodiment, the mRNA encoding at least one CAA antigenic polypeptide or immunogenic fragment thereof can include a non-canconical open reading frame that encodes the at least one CAA polypeptide or immunogenic fragment thereof. In an embodiment, the open reading frame encodes at least two, at least five, or at least ten CAA polypeptides and/or immunogenic fragments thereof. In an embodiment, the open reading frame encodes at least 100 antigenic polypeptides. In an embodiment, the open reading frame encodes 2-100 CAA polypeptides and/or immunogenic fragments thereof.

In an embodiment, the pharmaceutical composition comprises a plurality of lipid nanoparticles comprising a cationic lipid, a neutral lipid, a cholesterol, and a PEG lipid, wherein the plurality of lipid nanoparticles optionally has a mean particle size of between 80 nm and 160 nm; and wherein the lipid nanoparticles comprise one or more polynucleotides encoding at least one viral antigenic polypeptide or an immunogenic fragment thereof.

In an embodiment, the mRNA vaccine is multivalent. In an embodiment, the mRNA of the mRNA vaccine is codon-optimized. In an embodiment, an RNA (e.g., mRNA) vaccine further includes an adjuvant.

In an embodiment, the isolated mRNA is not self-replicating.

In some embodiment, the isolated mRNA comprises and/or encodes one or more 5′terminal cap (or cap structure), 3′terminal cap, 5′untranslated region, 3′untranslated region, a tailing region, or any combination thereof.

In an embodiment, the capping region of the isolated mRNA region may be from 1 to 10, e.g., 2-9, 3-8, 4-7, 1-5, 5-10, or at least 2, or 10 or fewer nucleotides in length. In an embodiment, the cap is absent.

In an embodiment, a 5′-cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine.

In an embodiment, the 5′terminal cap is 7 mG(5′)ppp(5′)NlmpNp, m7GpppG cap, N7-methylguanine. In an embodiment, the 3′terminal cap is a 3′-O-methyl-m7GpppG.

In an embodiment, the 3′-UTR is an alpha-globin 3′-UTR. In an embodiment, the 5′-UTR comprises a Kozak sequence.

In an embodiment, the tailing sequence may range from absent to 500 nucleotides in length (e.g., at least 60, 70, 80, 90, 120, 140, 160, 180, 200, 250, 300, 350, 400, 450, or 500 nucleotides). In an embodiment, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In an embodiment, the poly-A tail is at least 160 nucleotides in length.

In an embodiment, the at least one viral antigenic polypeptide linked to or fused to a signal peptide. In an embodiment, the isolated mRNA encoding a viral antigenic polypeptide or immunogenic fragment thereof further includes a polynucleotide sequence encoding a signal peptide. In an embodiment, the signal peptide is selected from: a HuIgGk signal peptide (METPAQLLFLLLLWLPDTTG (SEQ ID NO: 317)); IgE heavy chain epsilon-1 signal peptide (MDWTWILFLVAAATRVHS (SEQ ID NO: 318)); Japanese encephalitis PRM signal sequence (MLGSNSGQRVVFTILLLLVAPAYS (SEQ ID NO: 319)), VSVg protein signal sequence (MKCLLYLAFLFIGVNCA (SEQ ID NO: 320)) and Japanese encephalitis JEV signal sequence (MWLVSLAIVTACAGA (SEQ ID NO: 321)). In an embodiment, the signal peptide is fused to the N-terminus of at least one viral antigenic polypeptide. In an embodiment, a signal peptide is fused to the C-terminus of at least one viral antigenic polypeptide.

In an embodiment, the polynucleotides of the mRNA vaccine composition are structurally modified and/or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In an embodiment, the polynucleotide, e.g., an mRNA of an mRNA vaccine composition described herein comprises at least one chemical modification. In an embodiment, the polynucleotide, e.g., an mRNA of an mRNA vaccine composition does not comprise a chemical or structural modification.

In an embodiment, the at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In an embodiment, the chemical modification is in the 5-position of the uracil. In an embodiment, the chemical modification is a N1-methylpseudouridine. In an embodiment, the chemical modification is a N1-ethylpseudouridine.

In an embodiment, about 10%, about 15%, about 20%, about 24%, about 30%, about 35%, about 40%, about 45%, about 50%, about 55%, about 60%, about 65%, about 70%, about 75%, about 80%, about 85%, about 90%, about 95%, or about 100% of the uracil in of the CAA polypeptide or immunogenic fragment thereof encoding polynucleotide, such in the open reading frame, have a chemical modification, In an embodiment, about 100% of the uracil in the open reading frame of the viral antigenic polypeptide or immunogenic fragment thereof encoding polynucleotide have a N1-methyl pseudouridine in the 5-position of the uracil.

In an embodiment, the mRNA polynucleotide includes a stabilization element. In an embodiment, the stabilization element is a histone stem-loop. In an embodiment, the stabilization element is a nucleic acid sequence having increased GC content relative to wild type sequence.

In one embodiment, the mRNA polynucleotide may include a sequence encoding a self-cleaving peptide. The self-cleaving peptide may be, but is not limited to, a 2A peptide. As a non-limiting example, the 2A peptide has the protein sequence: GSGATNFSLLKQAGDVEENPGP (SEQ ID NO: 322), fragments or variants thereof. In one embodiment, the 2A peptide cleaves between the last glycine and last proline. As another non-limiting example, the polynucleotides of the present invention includes a polynucleotide sequence encoding the 2A peptide having the protein sequence GSGATNFSLLKQAGDVEENPGP (SEQ ID NO: 322) fragments or variants thereof.

One such polynucleotide sequence encoding the 2A peptide is GGAAGCGGAGCTACTAACTTCAGCCTGCTGAAGCAGGCTGGAGACGTGGAG GAGAACCCTGGACCT (SEQ ID NO: 323). The polynucleotide sequence of the 2A peptide may be modified or codon optimized by the methods described herein and/or are known in the art.

In one embodiment, this sequence is used to separate the coding region of two or more polypeptides of interest. As a non-limiting example, the sequence encoding the 2A peptide is between a first coding region A and a second coding region B (A-2Apep-B). The presence of the 2 A peptide results in the cleavage of one long protein into protein A, protein B and the 2A peptide. Protein A and protein B may be the same or different peptides or polypeptides of interest. In another embodiment, the 2A peptide are used in the polynucleotides of the present invention to produce two, three, four, five, six, seven, eight, nine, ten, or more proteins.

In an embodiment, the length of an mRNA included in the mRNA vaccine is greater than about 30 nucleotides in length (e.g., at least or greater than about 35, about 40, about 45, about 50, about 55, about 60, about 70, about 80, about 90, about 100, about 120, about 140, about 160, about 180, about 200, about 250, about 300, about 350, about 400, about 450, about 500, about 600, about 700, about 800, about 900, about 1,000, about 1,100, about 1,200, about 1,300, about 1,400, about 1,500, about 1,600, about 1,700, about 1,800, about 1,900, about 2,000, about 2,500, about 3,000, about 4,000, about 5,000, about 6,000, about 7,000, about 8,000, about 9,000, about 10,000, about 20,000, about 30,000, about 40,000, about 50,000, about 60,000, about 70,000, about 80,000, about 90,000 or up to and including about 100,000 nucleotides).

In an embodiment, the length of an mRNA included in the mRNA vaccine includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In an embodiment, the polynucleotides are linear. In yet another embodiment, the polynucleotides of the present invention that are circular are known as “circular polynucleotides” or “circP.” As used herein, “circular polynucleotides” or “circP” means a single stranded circular polynucleotide which acts substantially like, and has the properties of, an R A. The term “circular” is also meant to encompass any secondary or tertiary configuration of the circP.

Other RNA modifications for mRNA vaccines and production of mRNA can be as described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Publications, US20130197068, US20170043037, US20130261172, US20200030460, US20150038558, US20190274968, US20180303925, US20200276300; International Patent Application Publication Nos. WO/2018/081638A1, WO/2016/176330A1, which are incorporated herein by reference.

In an embodiment, the mRNA vaccine includes one or more additional mRNAs that encode a polypeptide adjuvant. In an embodiment, the mRNA vaccine includes one or more additional mRNAs that encode a non-viral antigen, such as an antigen to another disease causing agent.

In an embodiment, the one or more additional mRNAs that encode a polypeptide adjuvant encode a flagellin polypeptide. In an embodiment, at least one flagellin polypeptide (e.g., encoded flagellin polypeptide) is an immunogenic flagellin fragment. In an embodiment at least one flagellin polypeptide has at least 80%, at least 85%, at least 90%, or at least 95% identity to a flagellin polypeptide having a sequence identified by any one of SEQ ID NO: 54-56 of U.S. Pat. No. 10,272,150.

In an embodiment, at least one flagellin polypeptide and at least one viral and/or additional antigenic polypeptide are encoded by a single RNA (e.g., mRNA) polynucleotide. In other embodiments, at least one flagellin polypeptide and at least one viral and/or additional antigenic polypeptide are each encoded by a different RNA polynucleotide.

The isolated mRNA(s) can be made in part or using only in vitro transcription. Methods of making polynucleotides by in vitro transcription are known in the art and are described in U.S. Provisional Patent Application Nos. 61/618,862, 61/681,645, 61/737,130, 61/618,866, 61/681,647, 61/737,134, 61/618,868, 61/681,648, 61/737,135, 61/618,873, 61/681,650, 61/737,147, 61/618,878, 61/681,654, 61/737,152, 61/618,885, 61/681,658, 61/737,155, 61/618,896, 61/668,157, 61/681,661, 61/737,160, 61/618,911, 61/681,667, 61/737,168, 61/618,922, 61/681,675, 61/737,174, 61/618,935, 61/681,687, 61/737,184, 61/618,945, 61/681,696, 61/737,191, 61/618,953, 61/681,704 61/737,203; International Publication Nos WO2013151666, WO2013151668, WO2013151663, WO2013151669, WO2013151670, WO2013151664, WO2013151665, WO2013151736, WO2013151672, WO2013151671 WO2013151667, and WO/2020/205793A1; the contents of each of which are herein incorporated by reference in their entireties.

### Lipid Nanoparticles

The isolated mRNAs and other polynucleotides of the mRNa vaccine can be formulated in a lipid nanoparticle. In an embodiment, the lipid nanoparticle is a cationic lipid nanoparticle.

In an embodiment, the lipid nanoparticle comprises a molar ratio of 20-60% ionizable cationic lipid, 5-25% non-cationic lipid, 25-55% sterol, and 0.5-15% PEG-modified lipid.

In an embodiment, the cationic lipid is a biodegradable cationic lipid. In an embodiment, the biodegradable cationic lipid comprises an ester linkage. In an embodiment, the biodegradable cationic lipid comprises DLin-DMA with an internal ester, DLin-DMA with a terminal ester, DLin-MC3-DMA with an internal ester, or DLin-MC3-DMA with a terminal ester.

In an embodiment, a lipid nanoparticle comprises a cationic lipid, a PEG-modified lipid, a sterol and a non-cationic lipid. In an embodiment, a cationic lipid is an ionizable cationic lipid and the non-cationic lipid is a neutral lipid, and the sterol is a cholesterol. In an embodiment, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)-N,N-dimethyl-2-nonylhenicosa-12,15-dien-1-amine (L608), and N,N-dimethyl-1-[(1S,2R)-2-octylcyclopropyl]heptadecan-8-amine (L530). In an embodiment, the neutral lipid is 1,2-distearoyl-sn-glycero-3-phosphocholine (DSPC), the sterol is cholesterol, and the PEG-modified lipid is 1,2-dimyristoyl-racalycero-3-methoxypolyethylene glycol-2000 (PEG-DMG) or PEG-CDMA.

In an embodiment, the lipid nanoparticle is any nanoparticle described in U.S. Pat. No. 10,442,756, and/or comprises any compound described in U.S. Pat. No. 10,442,756, including but not limited to a nanoparticle according to any one of Formulas (IA) or (II) described therein.

In an embodiment, the lipid nanoparticle is any nanoparticle described in e.g., U.S. Pat. No. 10,266,485, and/or comprises any compound described in U.S. Pat. No. 10,266,485, including but not limited to a nanoparticle according to Formula (II) described therein.

In an embodiment, the lipid nanoparticle is a nanoparticle described in U.S. Pat. No. 9,868,692, and/or comprises a compound described in e.g., U.S. Pat. No. 9,868,692, including but not limited to a nanoparticle according to Formula (I), (IA), (II), (IIa), (IIb), (IIc), (IId), (IIe),

In an embodiment, a lipid nanoparticle comprises compounds of Formula (I) and/or Formula (II) as described in U.S. Pat. No. 10,272,150.

In an embodiment, the mRNA vaccine is formulated in a lipid nanoparticle that comprises a compound selected from Compounds 3, 18, 20, 25, 26, 29, 30, 60, 108-112 and 122 of U.S. Pat. No. 10,272,150.

In an embodiment, at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In an embodiment, the lipid nanoparticle has a mean diameter of 50-200 nm.

In an embodiment, a lipid nanoparticle comprises compounds of Formula (I) and/or Formula (II), as discussed below.

In an embodiment, a lipid nanoparticle comprises Compounds 3, 18, 20, 25, 26, 29, 30, 60, 108-112, or 122 as set forth in U.S. Pat. No. 10,272,150.

In an embodiment, the lipid nanoparticle has a polydispersity value of less than 0.4 (e.g., less than 0.3, 0.2 or 0.1).

In an embodiment, a plurality of lipid nanoparticles, such as when contained in a formulation, has a mean PDI of between 0.02 and 0.2.

In an embodiment, a plurality of lipid nanoparticles, such as when contained in a formulation comprising one or more polynucleotide(s), has a mean lipid to polynucleotide ratio (wt/wt) of between 10 and 20.

In an embodiment, the lipid nanoparticle has a net neutral charge at a neutral pH value.

Methods of mRNA Vaccination

The compositions described herein can be used to induce an antigen specific immune response to a virus or a viral variant. Exemplary viruses are described elsewhere herein.

In an embodiment, the methods of inducing an antigen specific immune response in a subject include administering to the subject any of the RNA (e.g., mRNA) vaccine as provided herein in an amount effective to produce an antigen-specific immune response.

In an embodiment, an antigen-specific immune response comprises a T cell response and/or a B cell response.

In an embodiment, a method of producing an antigen-specific immune response comprises administering to a subject a single dose (no booster dose) of a RNA (e.g., mRNA) vaccine of the present disclosure.

In an embodiment, the RNA (e.g., mRNA) vaccine is a combination vaccine comprising a combination of an mRNA vaccine described herein and at least one other mRNA vaccine. The at least one other mRNA vaccine can be against the same or a different virus or disease-causing agent.

In an embodiment, a method further comprises administering to the subject a second (booster) dose of an RNA (e.g., mRNA) vaccine. Additional doses of an RNA (e.g., mRNA) vaccine may be administered.

In an embodiment, the subject exhibits a seroconversion rate of at least 80% (e.g., at least 85%, at least 90%, or at least 95%) following the first dose or the second (booster) dose of the vaccine. Seroconversion is the period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In an embodiment, an RNA (e.g., mRNA) vaccine described herein is administered to a subject by intradermal, subcutaneous, or intramuscular injection. In an embodiment, the administering step comprises contacting a muscle tissue of the subject with a device suitable for injection of the composition. In an embodiment, the administering step comprises contacting a muscle tissue of the subject with a device suitable for injection of the composition in combination with electroporation.

In an embodiment, the anti-antigenic polypeptide antibody titer produced in the subject is increased by at least 1 log relative to a control. In an embodiment, the anti-antigenic polypeptide antibody titer produced in the subject is increased by 1-3 log relative to a control.

In an embodiment, the anti-antigenic polypeptide antibody titer produced in a subject is increased at least 2 times relative to a control. In an embodiment, the anti-antigenic polypeptide antibody titer produced in the subject is increased at least 5 times relative to a control. In an embodiment, the anti-antigenic polypeptide antibody titer produced in the subject is increased at least 10 times relative to a control. In an embodiment, the anti-antigenic polypeptide antibody titer produced in the subject is increased 2-10 times relative to a control.

In an embodiment, the control is an anti-antigenic polypeptide antibody titer produced in a subject who has not been administered an RNA (e.g., mRNA) vaccine of the present disclosure. In an embodiment, the control is an anti-antigenic polypeptide antibody titer produced in a subject who has been administered a live attenuated or inactivated vaccine against a virus or wherein the control is an anti-antigenic polypeptide antibody titer produced in a subject who has been administered a recombinant or purified viral protein vaccine.

In an embodiment, the control is an anti-antigenic polypeptide antibody titer produced in a subject who has been administered a virus-like particle (VLP) vaccine comprising structural proteins of the virus.

The RNA (e.g., mRNA) vaccine of the present disclosure can be administered to a subject in an effective amount (e.g., an amount effective to induce an immune response in the subject).

In an embodiment, the RNA (e.g., mRNA) vaccine is formulated in an effective amount to produce an antigen specific immune response in a subject.

In an embodiment, the effective amount is a total dose of 25 μg to 1000 μg, or 50 μg to 1000 μg. In an embodiment, the effective amount is a total dose of 100 μg. In an embodiment, the effective amount is a dose of 25 μg administered to the subject a total of two times. In an embodiment, the effective amount is a dose of 100 μg administered to the subject a total of two times. In an embodiment, the effective amount is a dose of 400 μg administered to the subject a total of two times. In an embodiment, the effective amount is a dose of 500 μg administered to the subject a total of two times.

In an embodiment, the efficacy (or effectiveness) of an RNA (e.g., mRNA) vaccine is greater than 60%.

Vaccine efficacy may be assessed using standard analyses (see, e.g., Weinberg et al., J Infect Dis. 2010 Jun. 1; 201(11):1607-10). For example, vaccine efficacy is measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas: Efficacy=(ARU−ARV)/ARU×100; and Efficacy−(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1−OR)×100.

In an embodiment, the efficacy (or effectiveness) of an RNA (e.g., mRNA) vaccine is at least 65%, at least 70%, at least 75%, at least 80%, at least 85%, or at least 90%.

In an embodiment, the vaccine immunizes the subject against one or more cancers. In an embodiment, the cancer is a blood cancer. In an embodiment, the cancer is a white blood cell cancer. In an embodiment, the cancer is multiple myeloma. In an embodiment, the cancer is acute myeloid leukemia (AML). In an embodiment, the cancer is chronic lymphocytic leukemia (CLL). Exemplary viruses and variants are described elsewhere herein.

In an embodiment, the subject to which the mRNA vaccine of the present disclosure is administered is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In an embodiment, the subject is about 12 months or younger (e.g., 12, 11, 10, 9, 8, 7, 6, 5, 4, 3, 2 months or 1 month). In an embodiment, the subject is about 6 months or younger.

In an embodiment, the subject to which the mRNA vaccine of the present disclosure is administered was born full term (e.g., about 37-42 weeks). In an embodiment, the subject was born prematurely, for example, at about 36 weeks of gestation or earlier (e.g., about 36, about 35, about 34, about 33, about 32, about 31, about 30, about 29, about 28, about 27, about 26 or about 25 weeks). For example, the subject may have been born at about 32 weeks of gestation or earlier. In an embodiment, the subject was born prematurely from about 32 weeks to about 36 weeks of gestation. In such subjects, an RNA (e.g., mRNA) vaccine may be administered later in life, for example, at the age of about 6 months to about 5 years, or older.

In an embodiment, the subject to which the mRNA vaccine of the present disclosure is administered the subject to which the mRNA vaccine of the present disclosure is administered is pregnant (e.g., in the first, second or third trimester) when administered an RNA (e.g., mRNA) vaccine.

In an embodiment, the subject to which the mRNA vaccine of the present disclosure is administered is a young adult between the ages of about 20 years and about 50 years (e.g., about 20, about 25, about 30, about 35, about 40, about 45 or about 50 years old).

In an embodiment, the subject to which the mRNA vaccine of the present disclosure is administered is an elderly subject about 60 years old, about 70 years old, or older (e.g., about 60, about 65, about 70, about 75, about 80, about 85, about 90, or about 100 or more years old).

In an embodiment, the subject to which the mRNA vaccine of the present disclosure is administered has cancer. In an embodiment, the subject to which the mRNA vaccine of the present disclosure is administered has a blood cancer. In an embodiment, the subject to which the mRNA vaccine of the present disclosure is administered has a white blood cell cancer. In an embodiment, the subject to which the mRNA vaccine of the present disclosure is administered has a multiple myeloma. In an embodiment, the subject to which the mRNA vaccine of the present disclosure is administered has acute myeloid leukemia (AML). In an embodiment, the subject to which the mRNA vaccine of the present disclosure is administered has chronic lymphocytic leukemia (CLL).

In an embodiment, the subject to which the mRNA vaccine of the present disclosure is administered is immunocompromised (has an impaired immune system, e.g., has an immune disorder or autoimmune disorder).

In an embodiment, the mRNA vaccine of the present disclosure is delivered to a subject at a dosage of between 10 μg/kg and 400 μg/kg of the nucleic acid vaccine is administered to the subject. In an embodiment the dosage of the RNA polynucleotide is 1-5 μg, 5-10 μg, 10-15 μg, 15-20μ, 10-25μ, 20-25μ, 20-50μ, 30-50μ, 40-50μ, 40-60μ, 60-80μ, 60-100μ, 50-100μ, 80-120μ, 40-120μ, 40-150μ, 50-150μ, 50-200μ, 80-200μ, 100-200μ, 120-250μ, 150-250μ, 180-280μ, 200-300μ, 50-300μ, 80-300μ, 100-300μ, 40-300μ, 50-350 μg, 100-350 μg, 200-350 μg, 300-350 μg, 320-400 μg, 40-380 μg, 40-100 μg, 100-400 μg, 200-400 μg, or 300-400 μg per dose. In an embodiment, the subject can receive 1, 2, 3, 4, 5, 6, 7, or more doses. After the initial dose (given at day zero) the subject can receive one or more (e.g., 1, 2, 3, 4, 5, 6, 7, 8, 9, 10, or more additional doses, referred to in the art as “booster” doses. The booster doses can follow the initial dose at any suitable time interval such as within days, weeks, months, or even years. In an embodiment, multiple booster doses are needed close in time after the initial dose (such as within 1, 2, 3, or 4 weeks after the initial dose) followed by a larger gap in time (e.g., months or years before subsequent booster doses are needed). In an embodiment, a first dose of the mRNA vaccine is administered to the subject on day zero. In an embodiment, a second dose of the mRNA vaccine ais administered to the subject on day 14, 21, 28, 35, 42, 49, 56, 63, 70, 77, 84 or more days after the first dose. In an embodiment, a third dose of the mRNA vaccine is administered to the subject on day 14, 21, 28, 35, 42, 49, 56, 63, 70, 77, 84 or more days after the first and/or second dose.

In an embodiment, the mRNA vaccine confers an antibody titer superior to the criterion for seroprotection for a cancer for an acceptable percentage of human subjects. In an embodiment, the cancer is a blood cancer. In an embodiment, the cancer is a white blood cell cancer. In an embodiment, the cancer is multiple myeloma. In an embodiment, the antibody titer produced by the mRNA vaccines of the invention is a neutralizing antibody titer. In an embodiment the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

In an embodiment, a unit of use vaccine comprises between 10 ug and 400 ug of one or more RNA polynucleotides encoding the CAA polypeptide(s) and/or immunogenic fragment(s) thereof and a pharmaceutically acceptable carrier or excipient, formulated for delivery to a human subject. In an embodiment, the vaccine further comprises a cationic lipid nanoparticle.

Aspects of the invention provide methods of creating, maintaining, or restoring antigenic memory to a cancer in an individual or population of individuals comprising administering to said individual or population an mRNA vaccine described herein. Aspects of the invention provide methods of creating, maintaining, or restoring antigenic memory to a blood cancer in an individual or population of individuals comprising administering to said individual or population an mRNA vaccine described herein. Aspects of the invention provide methods of creating, maintaining, or restoring antigenic memory to a white blood cell cancer in an individual or population of individuals comprising administering to said individual or population an mRNA vaccine described herein. Aspects of the invention provide methods of creating, maintaining, or restoring antigenic memory to multiple myeloma, acute myeloid leukemia (AML), and/or chronic lymphocytic leukemia (CLL) in an individual or population of individuals comprising administering to said individual or population an mRNA vaccine described herein.

In an embodiment, the methods of vaccinating a subject comprising administering to the subject a single dosage of between 25 ug/kg and 400 ug/kg of an mRNA vaccine comprising one or more RNA polynucleotides encoding a CAA polypeptide and/or an immunogenic fragment thereof in an effective amount to vaccinate the subject.

In an embodiment, the mRNA vaccines comprising one or more RNA polynucleotides encoding a CAA polypeptide and/or an immunogenic fragment thereof, wherein the RNA comprises at least one chemical modification, wherein the vaccine has at least 10-fold less RNA polynucleotide than is required for an unmodified mRNA vaccine to produce an equivalent antibody titer. In an embodiment, the RNA polynucleotide is present in a dosage of 25-100 micrograms.

In an embodiment, the mRNA vaccine comprises an LNP formulated RNA polynucleotide having an open reading frame comprising no nucleotide modifications (unmodified), the open reading frame one or more RNA polynucleotides encoding a CAA polypeptide and/or an immunogenic fragment thereof, wherein the vaccine has at least 10-fold less RNA polynucleotide than is required for an unmodified mRNA vaccine not formulated in a LNP to produce an equivalent antibody titer. In an embodiment, the RNA polynucleotide is present in a dosage of 25-100 micrograms.

In an embodiment, the mRNA vaccine comprises an LNP formulated RNA polynucleotide having an open reading frame comprising one or more modifications, the open reading frame one or more RNA polynucleotides encoding a CAA polypeptide and/or an immunogenic fragment thereof, wherein the vaccine has at least 10-fold less RNA polynucleotide than is required for an unmodified mRNA vaccine not formulated in a LNP to produce an equivalent antibody titer. In an embodiment, the RNA polynucleotide is present in a dosage of 25-100 micrograms.

In an embodiment, the method includes vaccinating a subject with a combination vaccine including at least two nucleic acid sequences encoding respiratory antigens, wherein at least one encodes a CAA or immunogenic fragment thereof wherein the dosage for the vaccine is a combined therapeutic dosage wherein the dosage of each individual nucleic acid encoding an antigen is a sub therapeutic dosage. In an embodiment, the combined dosage is 25 micrograms of the RNA polynucleotide in the nucleic acid vaccine administered to the subject. In an embodiment, the combined dosage is 100 micrograms of the RNA polynucleotide in the nucleic acid vaccine administered to the subject. In an embodiment the combined dosage is 50 micrograms of the RNA polynucleotide in the nucleic acid vaccine administered to the subject. In an embodiment, the combined dosage is 75 micrograms of the RNA polynucleotide in the nucleic acid vaccine administered to the subject. In an embodiment, the combined dosage is 150 micrograms of the RNA polynucleotide in the nucleic acid vaccine administered to the subject. In an embodiment, the combined dosage is 400 micrograms of the RNA polynucleotide in the nucleic acid vaccine administered to the subject. In an embodiment, the sub therapeutic dosage of each individual nucleic acid encoding an antigen is 1, 2, 3, 4, 5, 6, 7, 8, 9, 10, 11, 12, 13, 14, 15, 16, 17, 18, 19, or 20 micrograms.

In an embodiment, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in the subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In an embodiment, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In an embodiment, antigen-specific antibodies are measured in units of μg/ml or are measured in units of IU/L (International Units per liter) or mIU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay

Further embodiments are illustrated in the following Examples, which are given for illustrative purposes only and are not intended to limit the scope of the invention.

### EXAMPLES

### Example 1—Molecular Signatures of T Cells Targeting Multiple Myeloma

In this study, both antigen-agnostic and antigen-specific screening techniques were used to analyze bone marrow T cells (BMTCs) from patients with multiple myeloma, aiming to chart TCR reactivities and transcriptional profiles. Together with the generation of tumor MHC immunopeptidomes, the TCR landscape in diseased human bone marrow was delineated, unveiling the cellular and functional complexity of naturally occurring tumor- and virus-specific BMTCs. These findings highlight a transcriptionally unique subset of BMTCs that exhibit endogenous anti-tumor activity. It was found that conserved BMTC responded to public antigens. TCRs that target antigens derived from the corrupted immunoglobulin in multiple myeloma cells were also identified. Furthermore, the detection of tumor-reactive TCRs prior to treatment correlated with enhanced clinical responses to induction chemotherapy and bispecific antibody administration. Highlighting the therapeutic importance of autologous stem cell transplantation, it was shown an increase in tumor-reactive TCRs within stem cell grafts. These TCRs are selectively transplanted and exhibit long-term persistence upon re-infusion into patients.

Without being bound by theory, these results shed light on how BMTCs contribute to shaping anti-myeloma T cell immunity, emphasizing the importance of endogenous T cell responses against blood cancers.

### Global Transcriptomic Profiles of Bone Marrow T Cell Repertoires

To detect tumor-reactive TCRs and establish a transcriptional signature of T cells recognizing multiple myeloma, bone marrow biopsies and peripheral blood were collected at initial disease diagnosis. Samples from patients with newly diagnosed multiple myeloma (NDMM; n=20; patients 01-20) were used for TCR screening and single-cell profiling and split into a retrospectively acquired establishment cohort (Pts 01-06) and a prospectively acquired validation cohort (Pts 07-20) to allow prospective testing of any developed signature. To query the feasibility of our forward screening platform in diseases other than multiple myeloma, additional proof-of-concept experiments in patients with acute myeloid leukemia (AML; n=7) and chronic lymphocytic leukemia (CLL; n=3) were performed. To further investigate the clinical relevance of these findings, TCRs were sequenced and analyzed from two clinical cohorts: bone marrow T cells of patients undergoing autologous stem cell transplantation in NCT023157161 (ASCT; n=14; patients 21-34), as well as patients treated with the BCMAxCD3 bispecific antibody (bsAb) monotherapy in NCT03269136 (bsAb; n=18; patients 35-52), were classified for their antigen specificity and their TCRs traced over time.

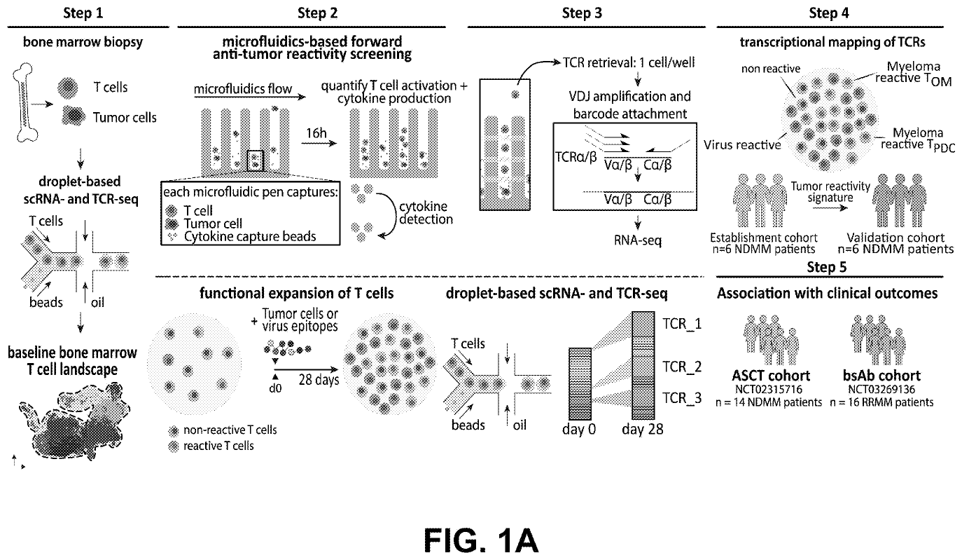

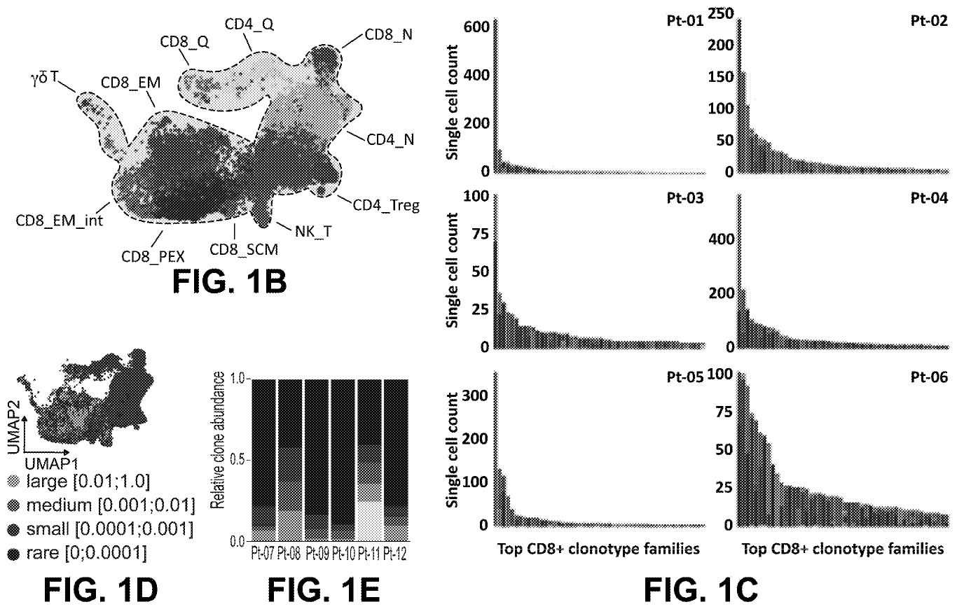

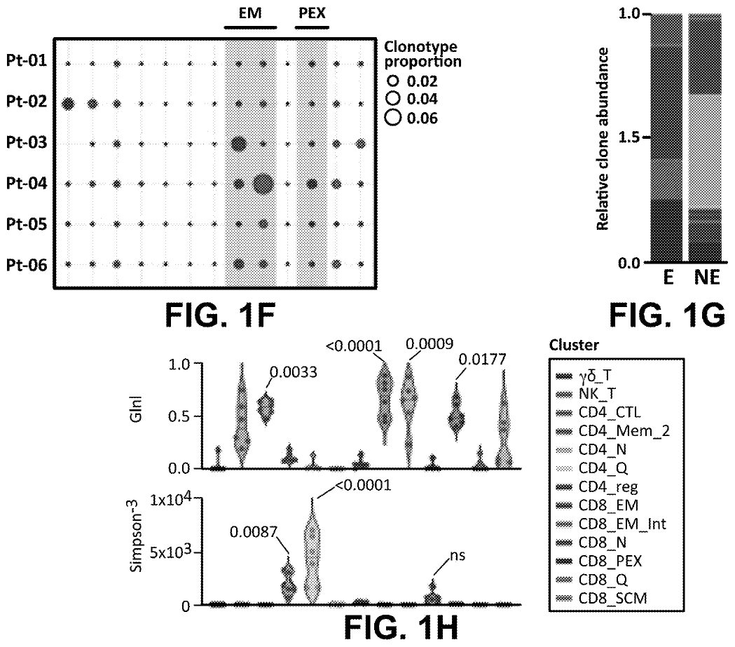

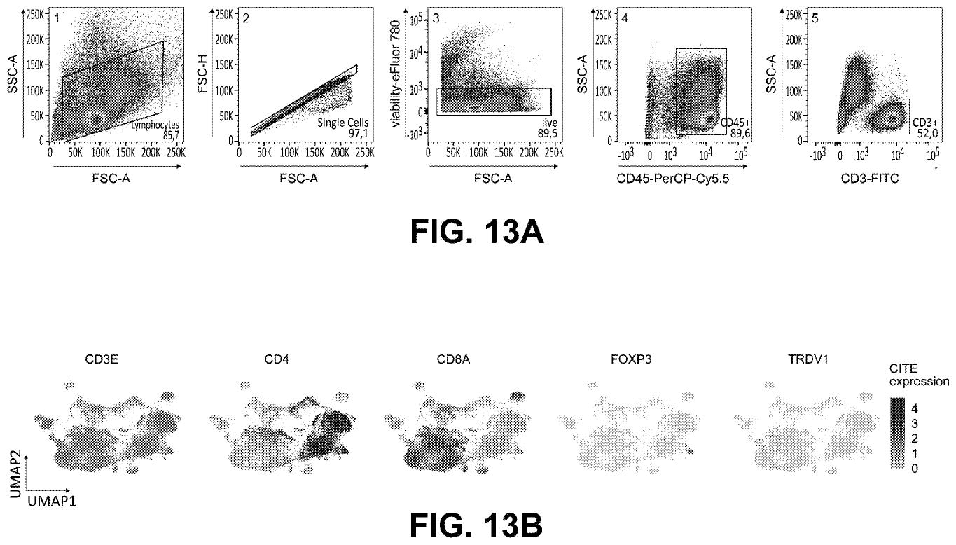

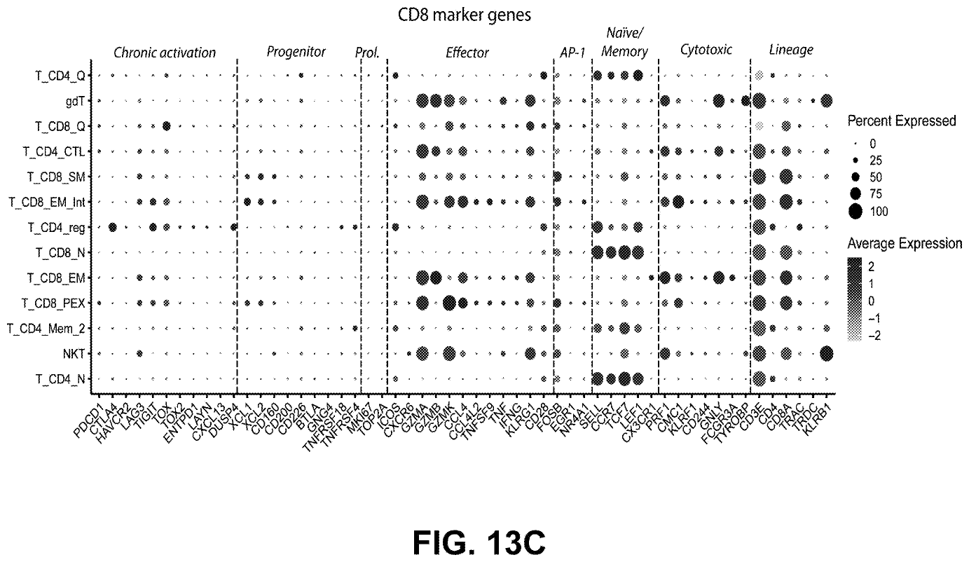

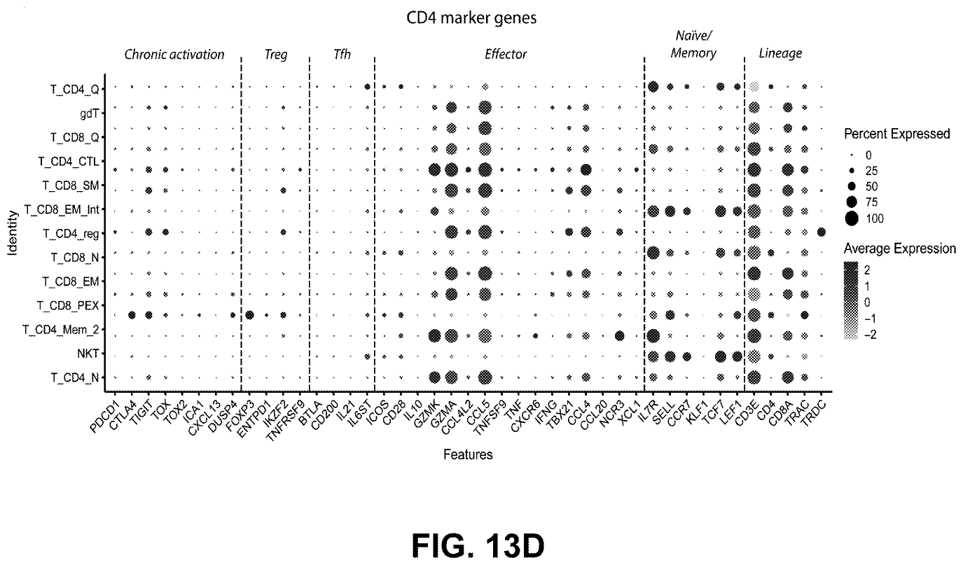

NDMM was chosen as a proof-of-principle entity due to the presence of well-established and highly expressed surface protein markers to enable sorting of malignant cells. NDMM furthermore less frequently disrupts the bone marrow microenvironment and suppresses lymphopoiesis compared to other malignant hematological diseases, which enabled testing of several thousand bone marrow-derived T cells per patient. To characterize the phenotype and clonality of bone marrow T cells (BMTC), high-throughput single-cell RNA sequencing (scRNA-seq) and single-cell V(D)J sequencing (scVDJ-seq) was used coupled with the detection of surface proteins using cellular indexing of transcriptomes and epitopes by sequencing (CITE-seq), thereby defining the baseline in vivo transcriptional state of each T cell (FIG. 1A, step 1; FIGS. 13A & 13B). Identification of tumor-reactive TCRs was performed in parallel using 1) a microfluidics-based forward screening approach of single BMTCs exposed to single autologous tumor cells (FIG. 1A, step 2) and 2) a functional expansion of tumor-reactive T cells assay⁴ on BMTCs. This antigen-dependent approach was informed by prior antigen discovery in patient tumor samples by prediction of whole genome sequencing (WG-seq) and RNA-seq-derived cancer associated antigens (CAAs) and neoepitopes as well as class I HLA immunoprecipitation (IP) followed by LC-MS/MS analysis. (FIG. 1A, step 2). Optically screened T cells were retrieved from their microfluidic reaction chambers and subjected to combined TCR alpha and beta chain sequencing (TCRA/B-seq), while functional expansion cultures were sequenced after 10 days of tumor- or virus epitope stimulation using ultradeep TCRVβ-seq (FIG. 1A, step 3). The data from both assays were then integrated and used to identify and phenotype antigen-reactive T cells by matching each TCR to its baseline transcriptional state using the CDR3 amino acid sequence as a unique endogenous barcode of a given clone (FIG. 1A, step 4). The derived transcriptional signature of tumor-reactive BMTCs was tested for its viability to predict tumor-reactive TCRs in the prospectively acquired patient cohort (FIG. 1A, step 4) and queried its association with clinical outcomes in the patient cohorts outlined above (FIG. 1A, step 5).

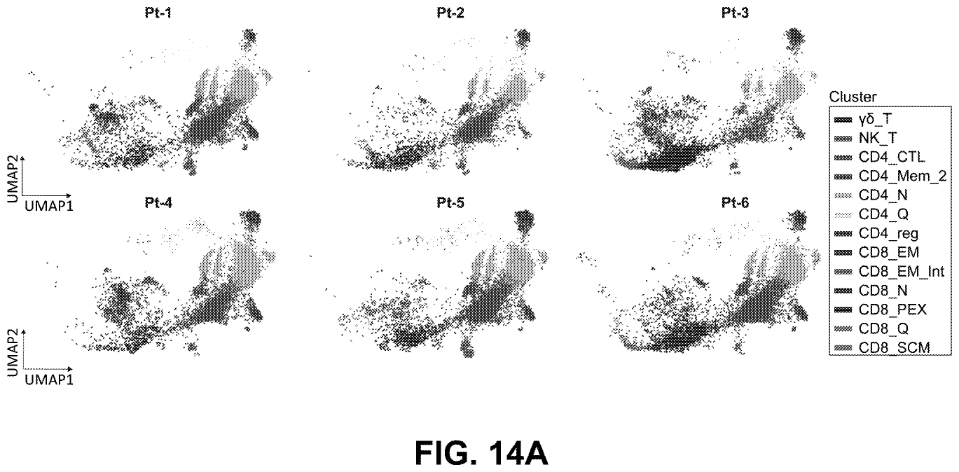

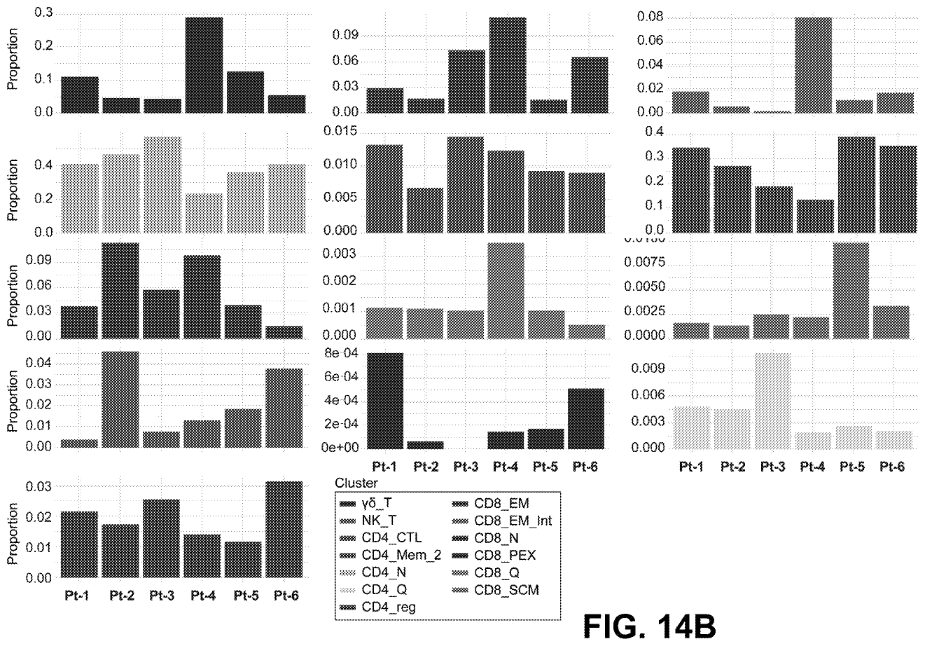

The dataset of transcriptomes from 187,015 cells representing 132,501 unique TCRs clonotypes was equally derived from all 20 patients (FIGS. 14A & 14B). BMTC of the establishment NDMM cohort clustered into 13 subsets (FIG. 1b), classified based on RNA and surface protein expression of T cell-related genes; we only considered T cells with a successfully captured TCR (FIGS. 13C & 13D). Three major states of CD8⁺ BMTC were observed: effector-memory (CD8_EM), progenitor exhausted (CD8_PEX), and stem-like memory (CD8_SM) BMTC. CD4+ BMTC were enriched for naïve and memory states, and A considerable amount of regulatory T (Treg)-like cells were observed in agreement with other studies in NDMM⁵. Seven additional minor clusters included natural killer (NK)-like and quiescent (Q) T cells (FIG. 1B).

The relationship between phenotype and TCR clonality was evaluated in 135,501 unique clonotypes identified by scVDJ-seq at initial diagnosis (FIGS. 1C-1F). In all patients, highly expanded clonotype families were distributed predominantly in cells with CD8⁺ effector-memory and exhausted phenotypes (FIG. 1C), which showed increased clonality of TCRs (FIGS. 1D, 1F, & 1F (top)). By contrast, non-expanded BMTC and correspondingly, highly diverse TCRs in the diseased bone marrow were significantly enriched for naïve and memory CD4⁺ cells (FIG. 1H (bottom), FIGS. 14C & 14D).

### Functional Phenotypes of T Cells Targeting Hematological Cancer Cells

To identify tumor-reactive TCRs, a microfluidics-based forward screen was performed, whereby single BMTC were co-cultured with autologous myeloma cells for 16 h. BMTC and myeloma cells were isolated from the same bone marrow biopsy specimen. The median purity of tumor cell preparations was >96% and a total of 27,625 single T cells for recognition of patient-autologous tumor cells were screened and functionally tested. Each microfluidic reaction chamber further contained capture beads to detect the T cell-derived cytokines interleukin-2 (IL-2), interferon-gamma (IFN-γ), and tumor necrosis factor (TNF), and was assayed for expression of surface 4-1BB (CD137) on T cells upon tumor recognition. If these signals of tumor reactivity were detected, this T cell was retrieved from its reaction chamber and subjected to TCRA/B-seq (FIG. 2A).

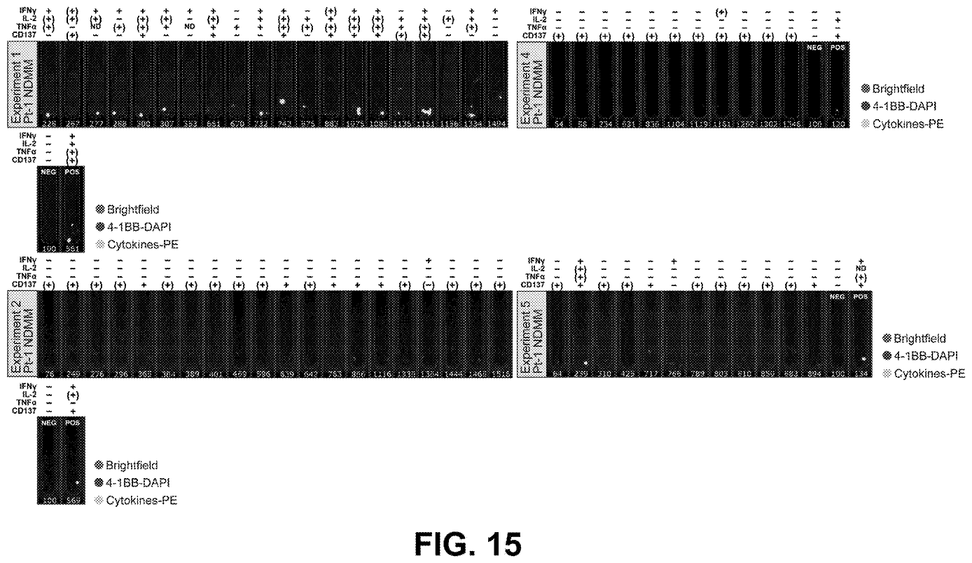

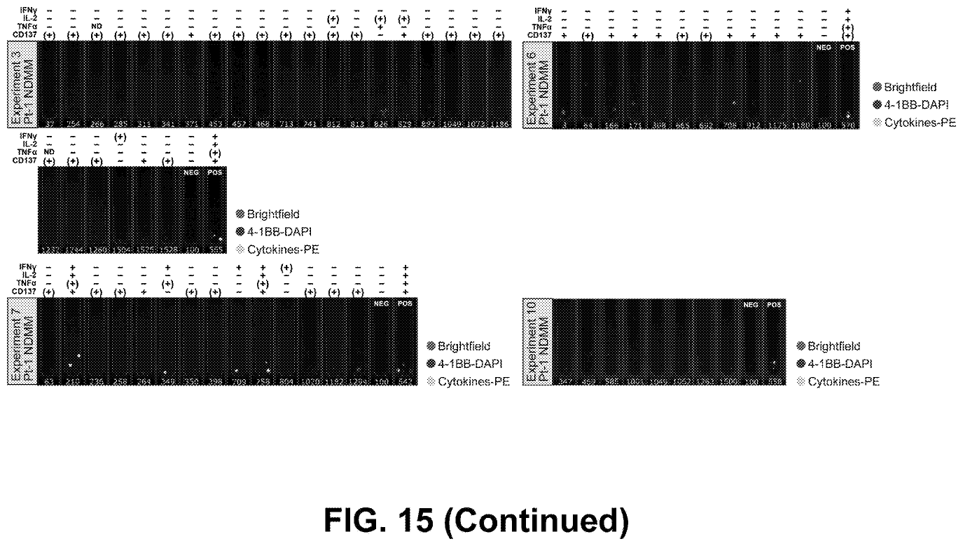

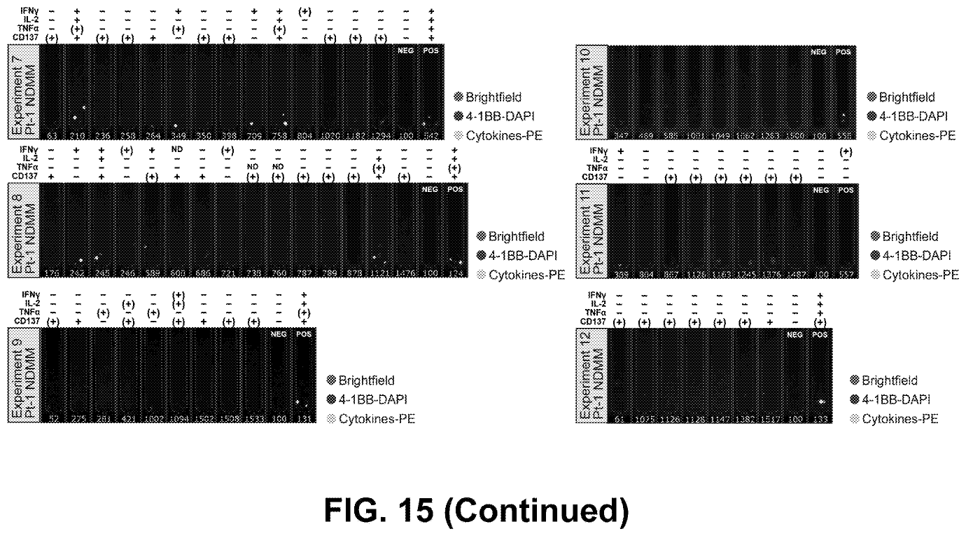

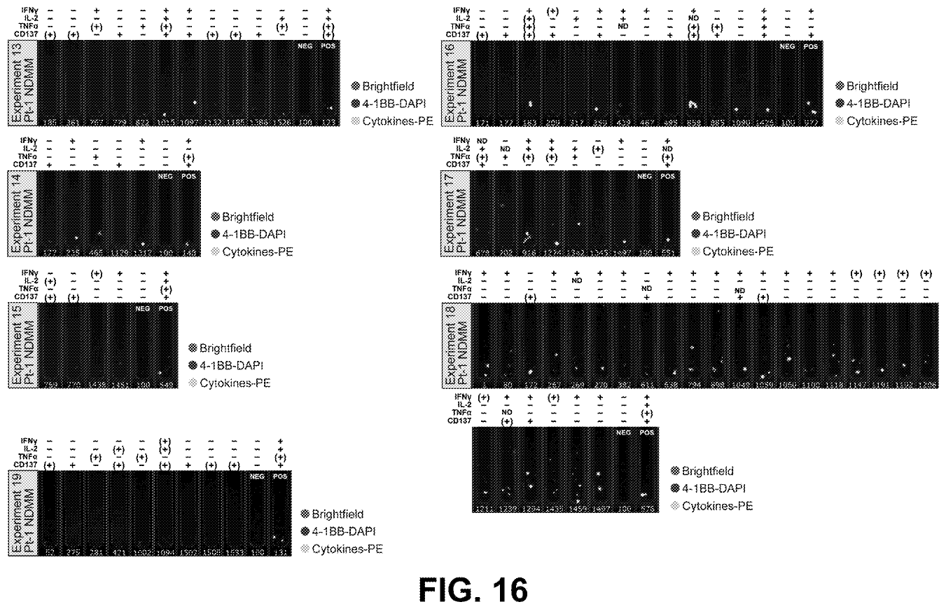

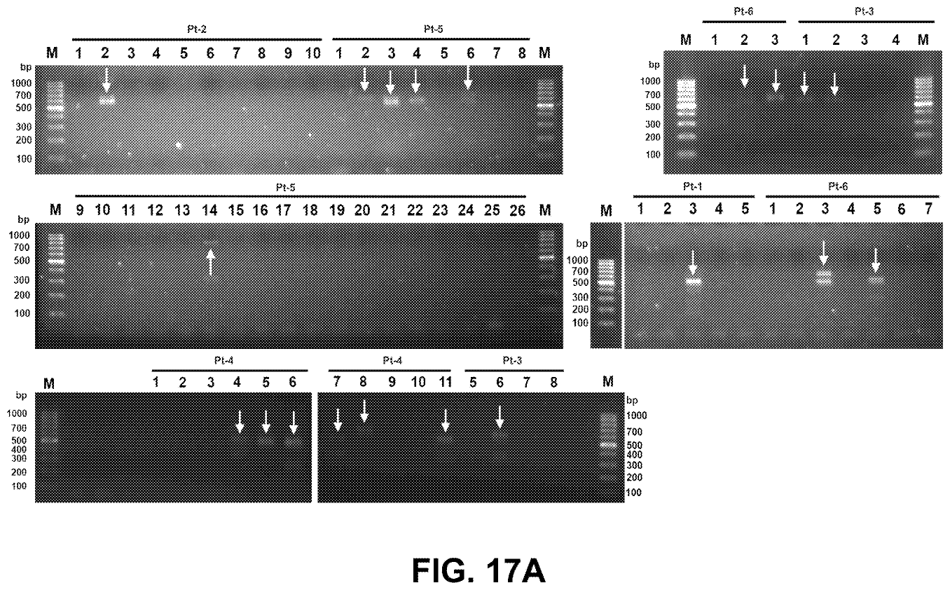

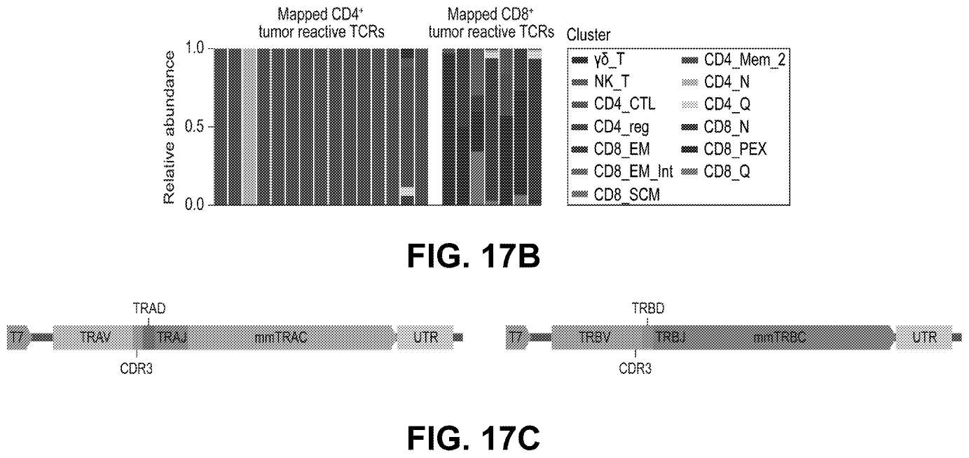

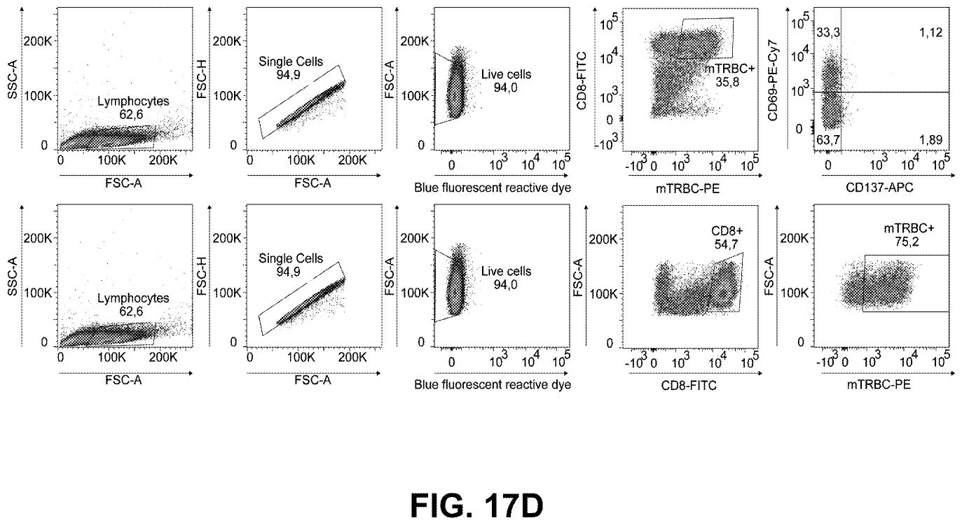

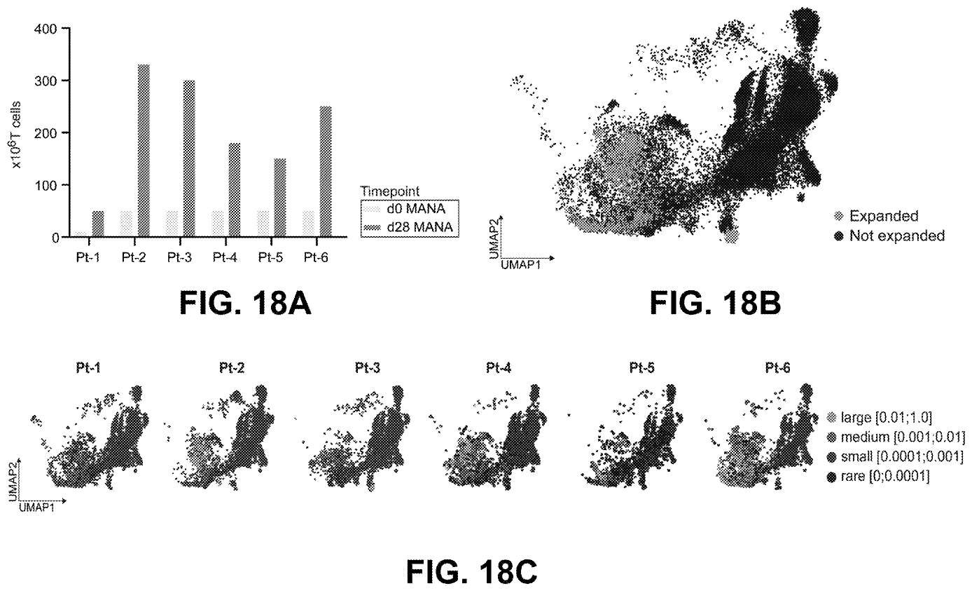

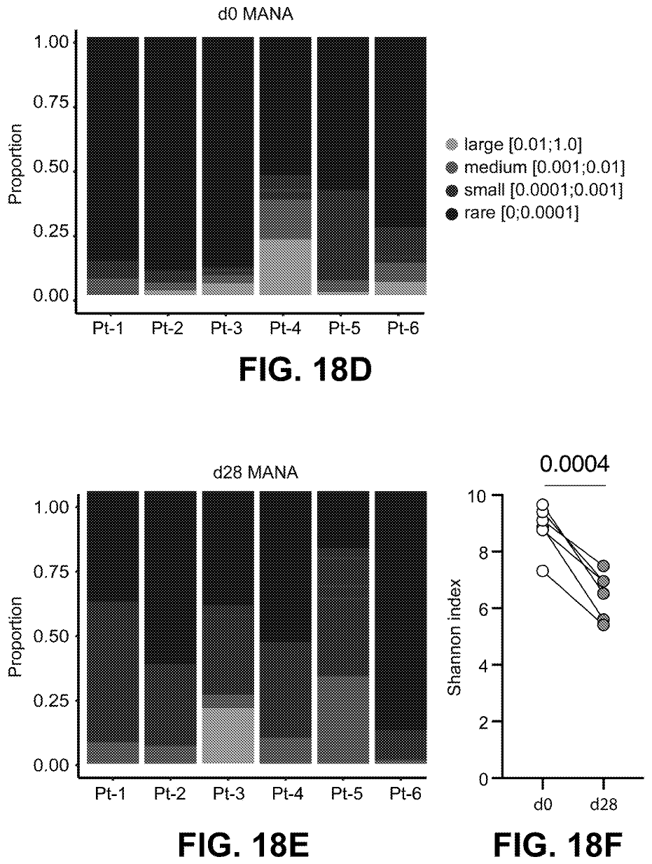

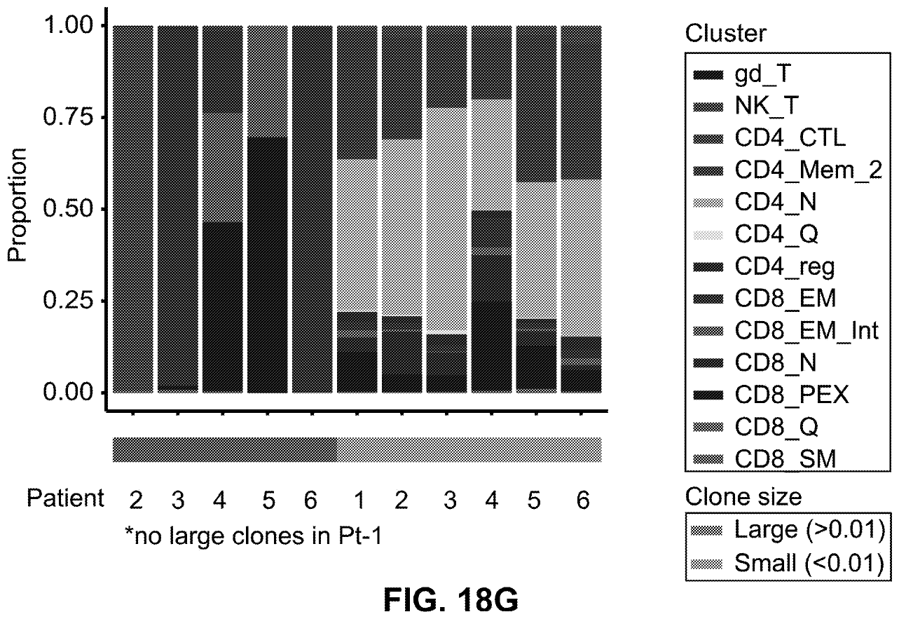

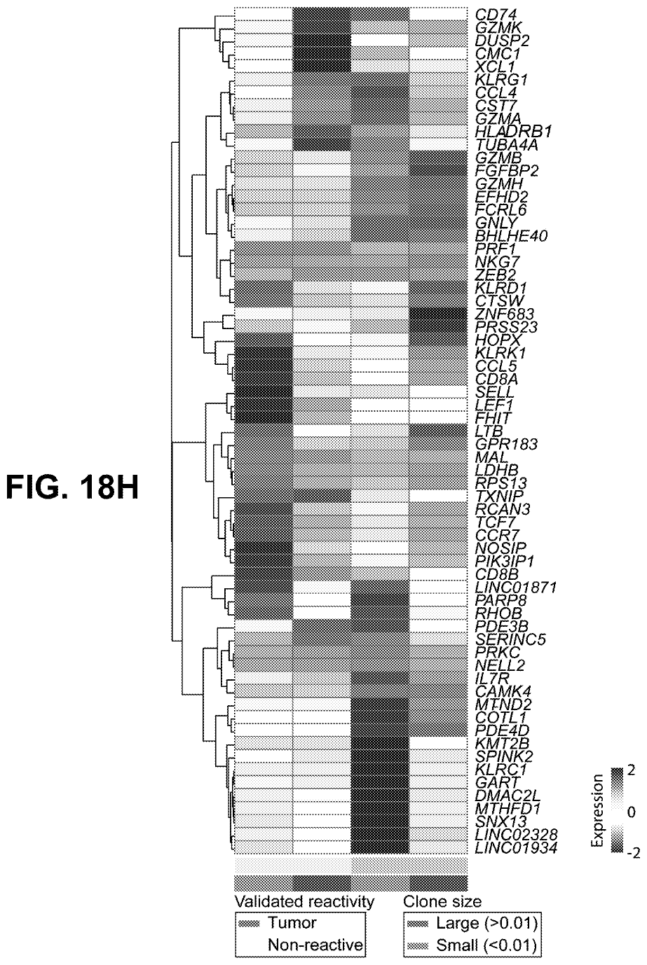

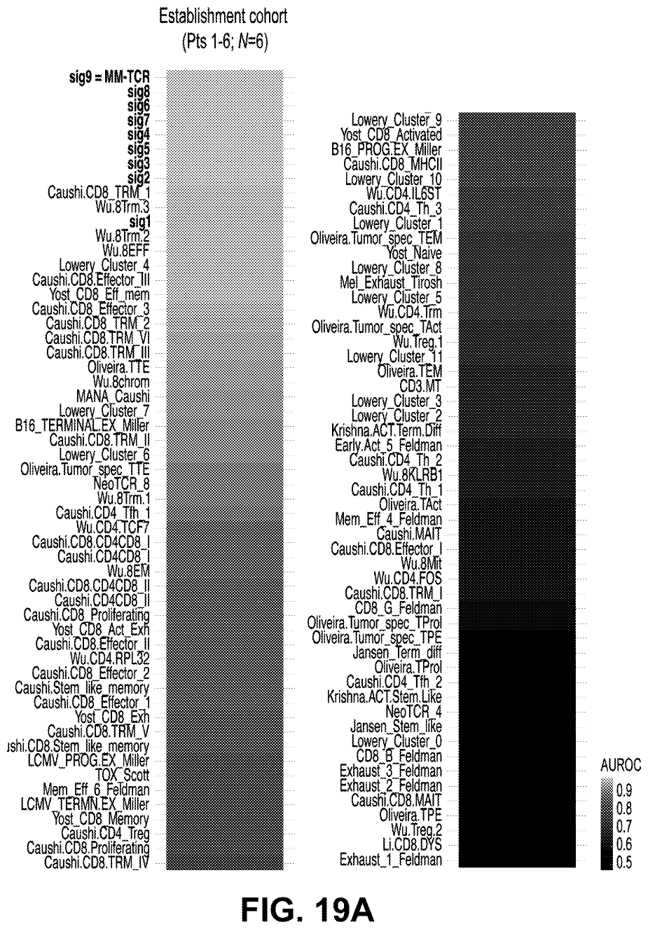

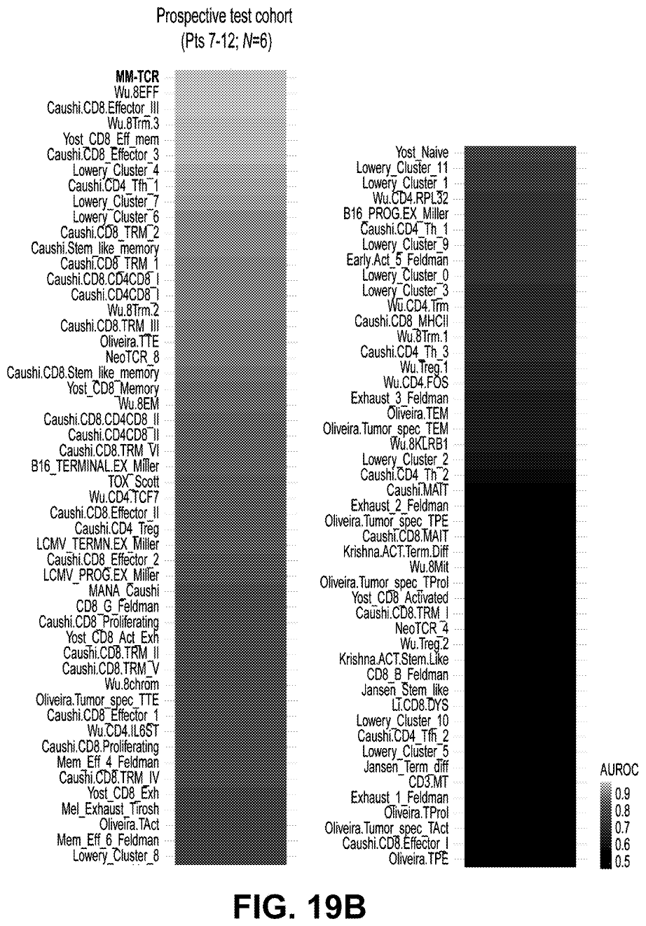

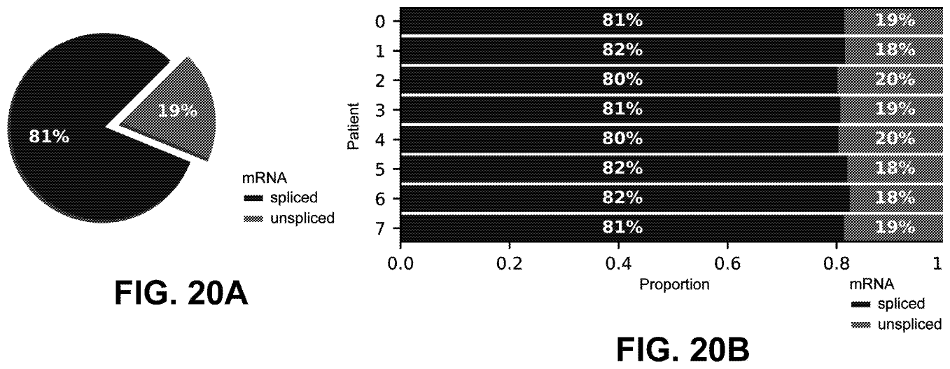

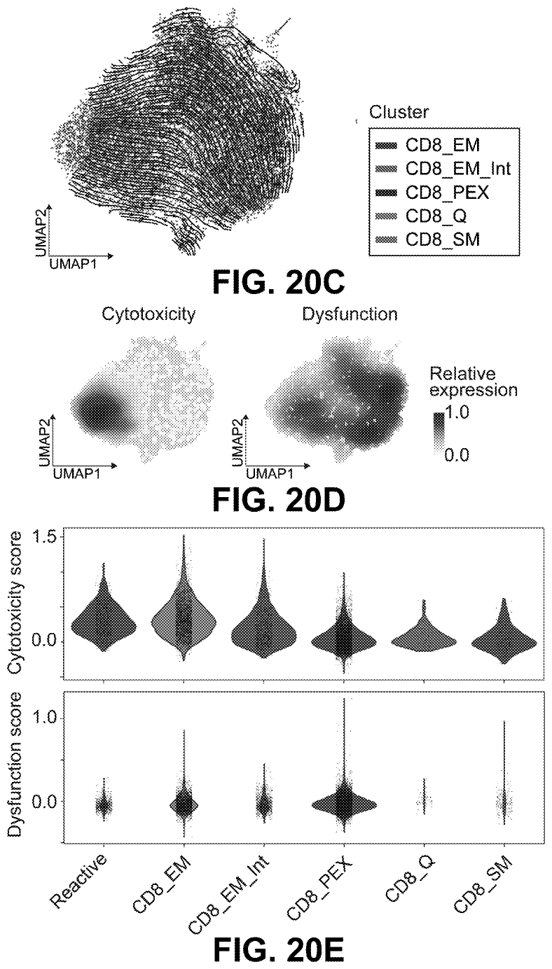

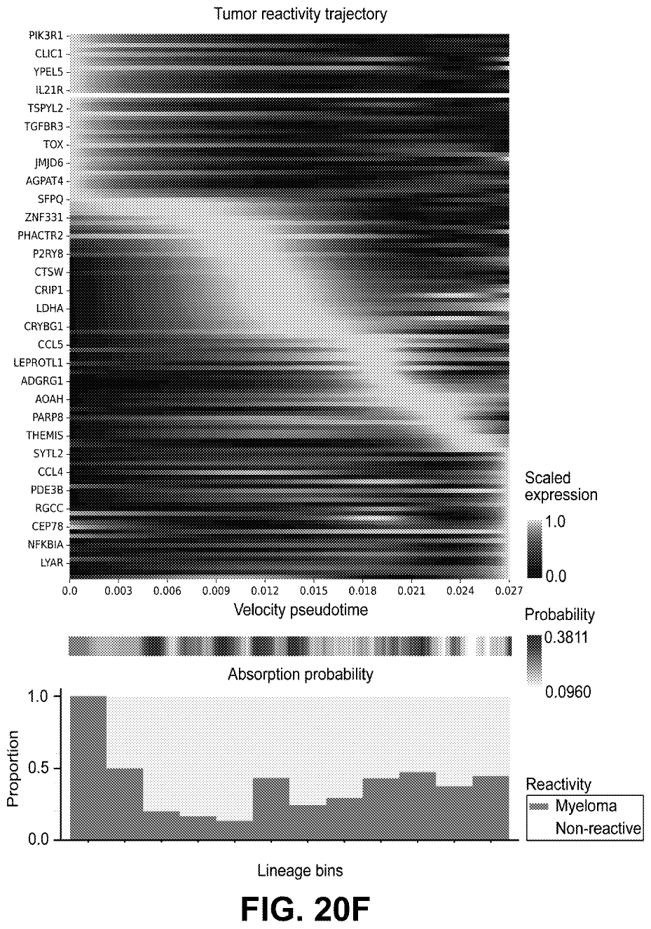

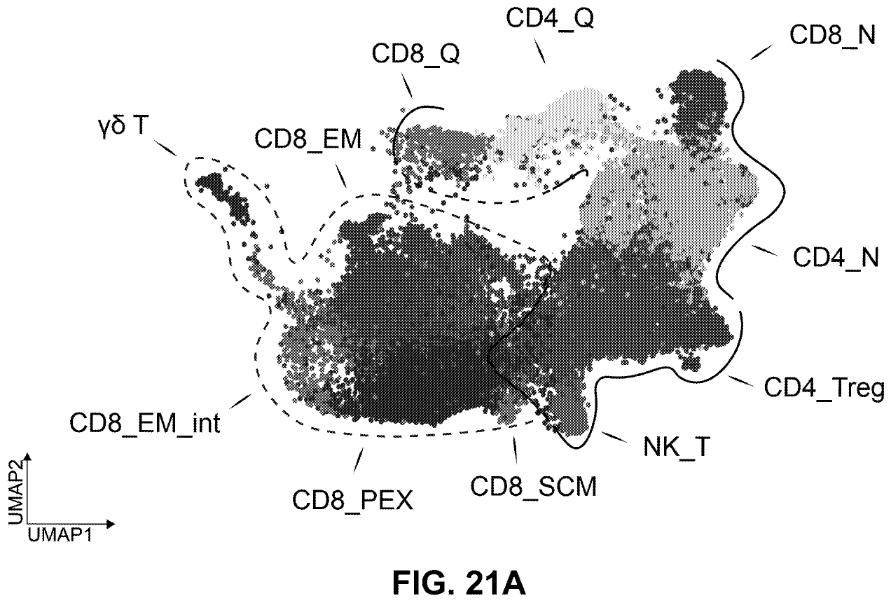

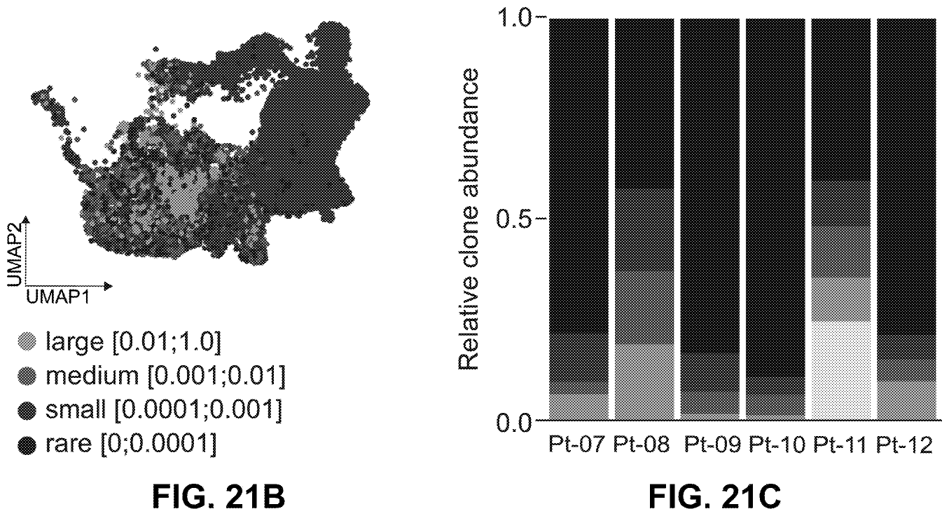

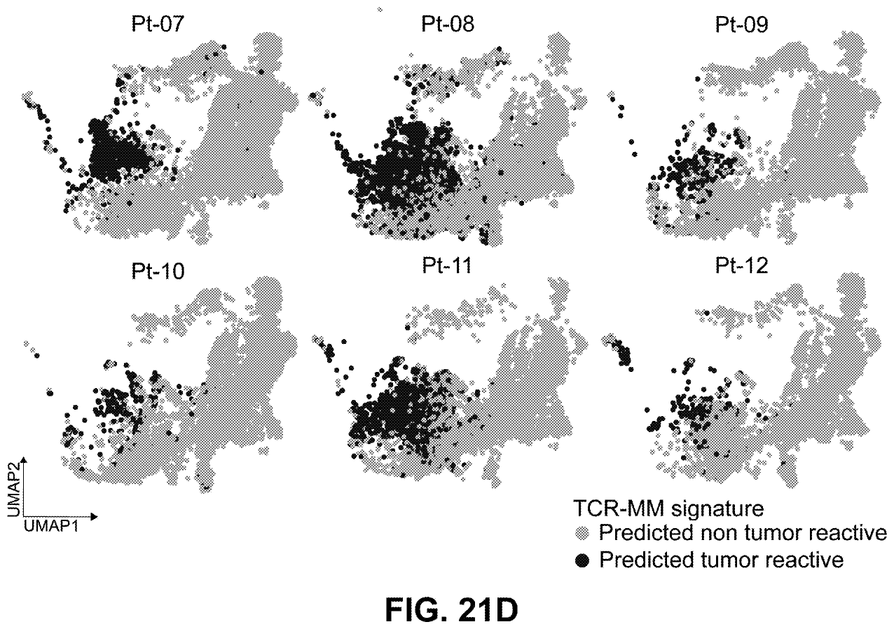

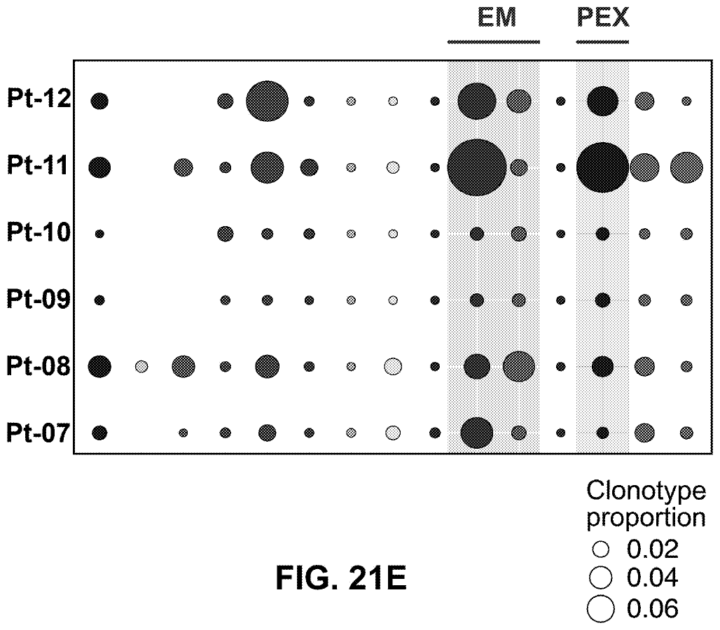

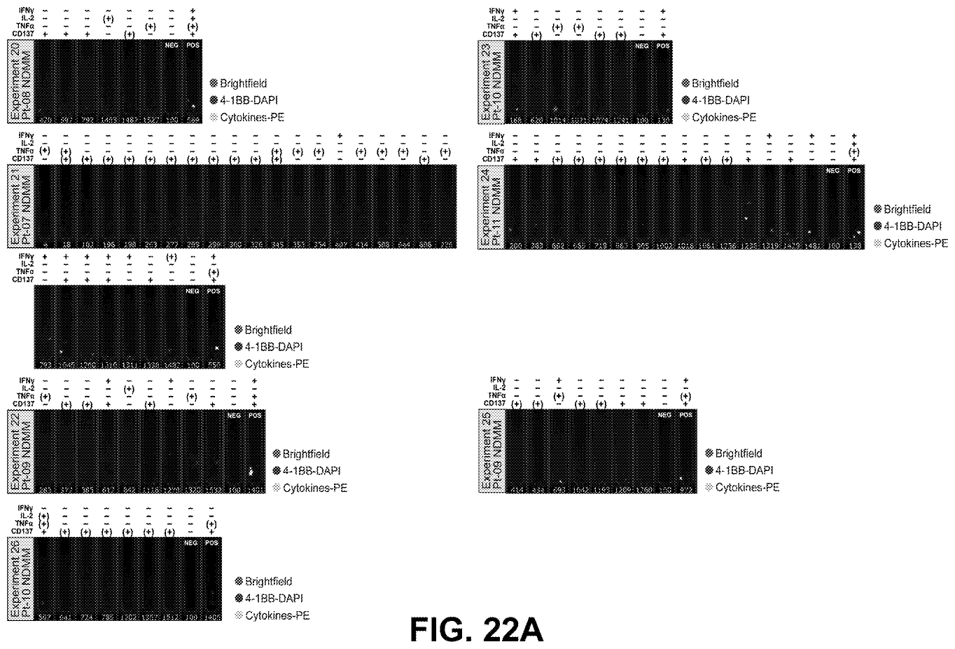

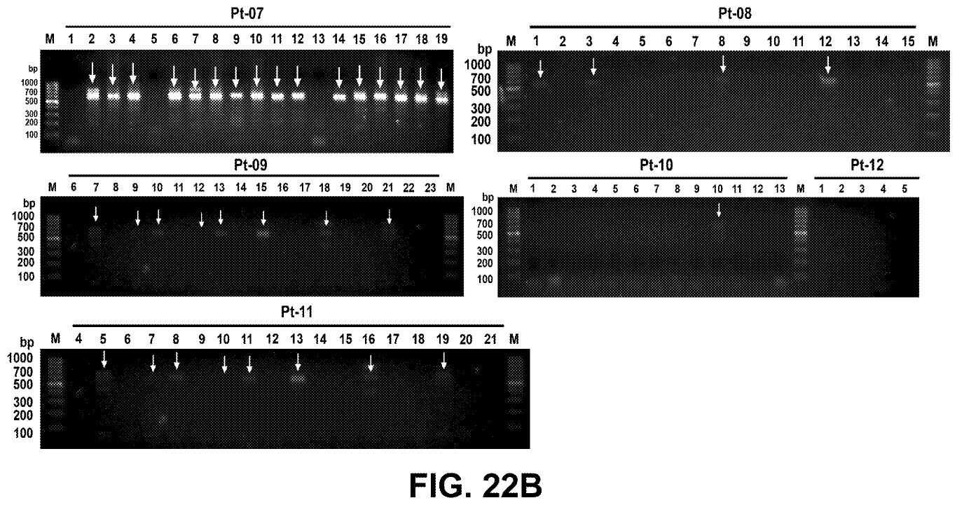

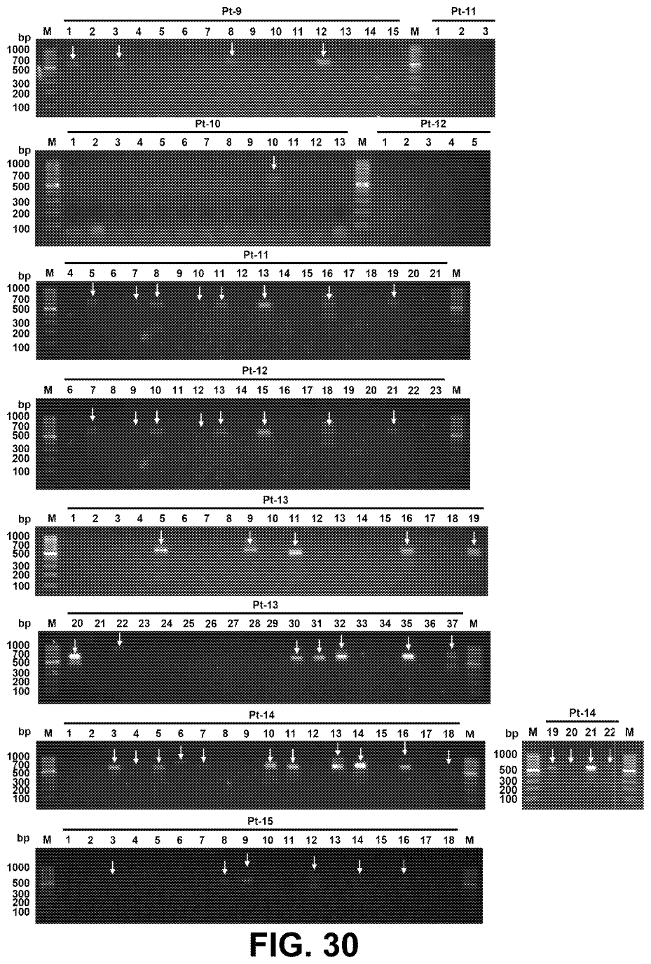

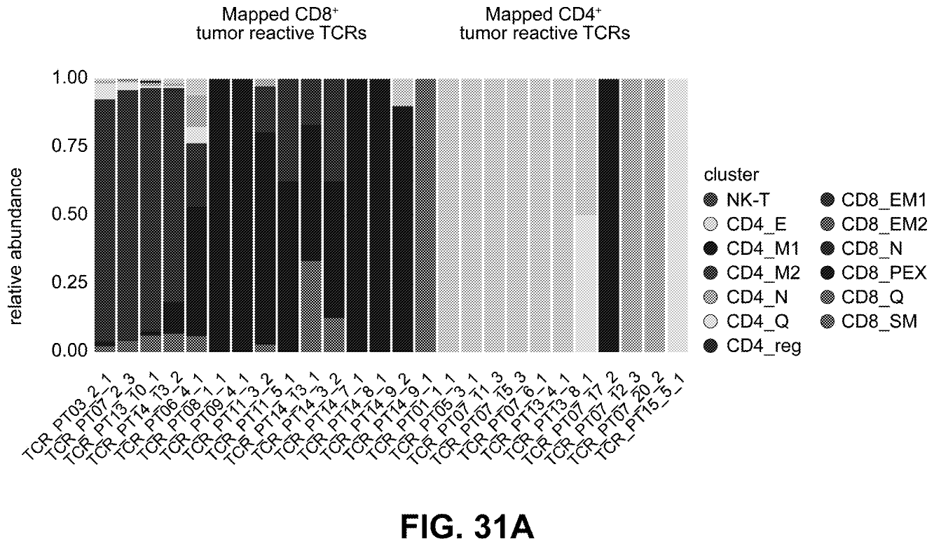

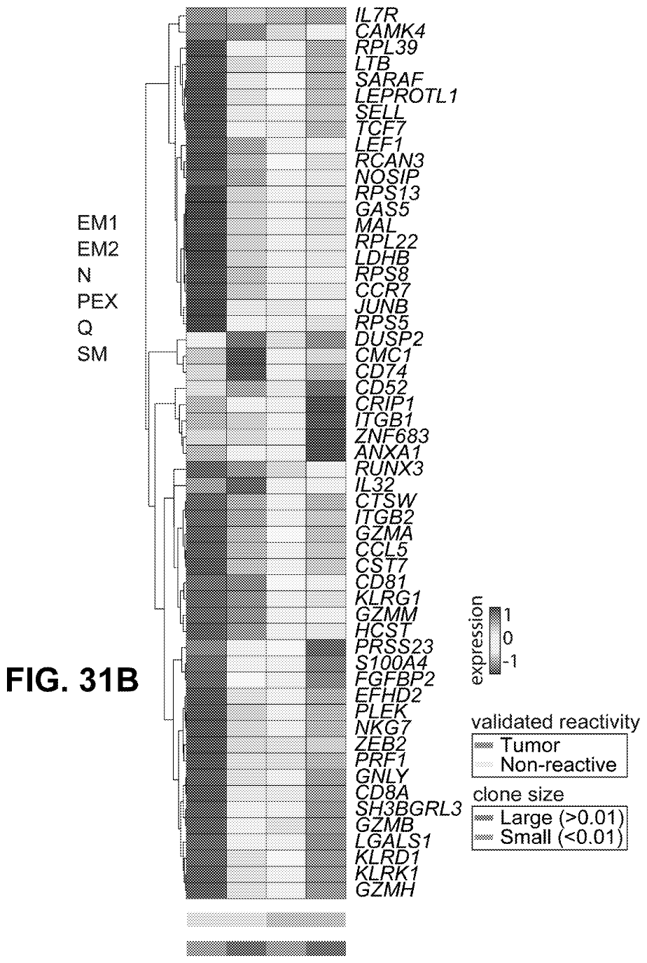

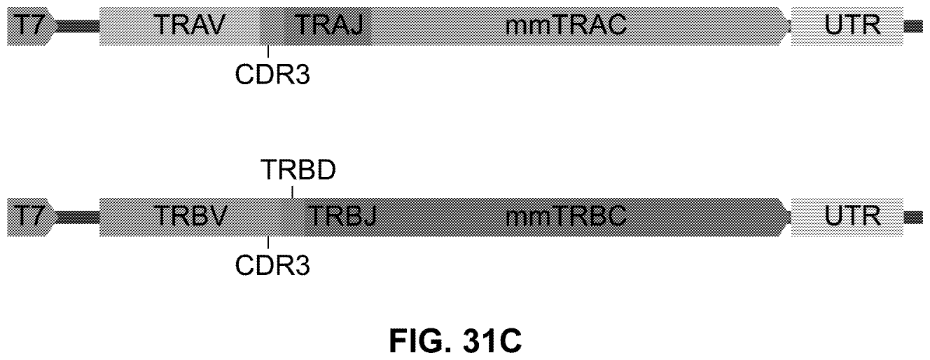

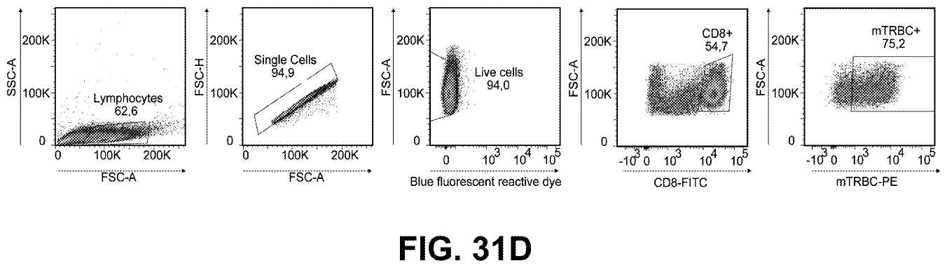

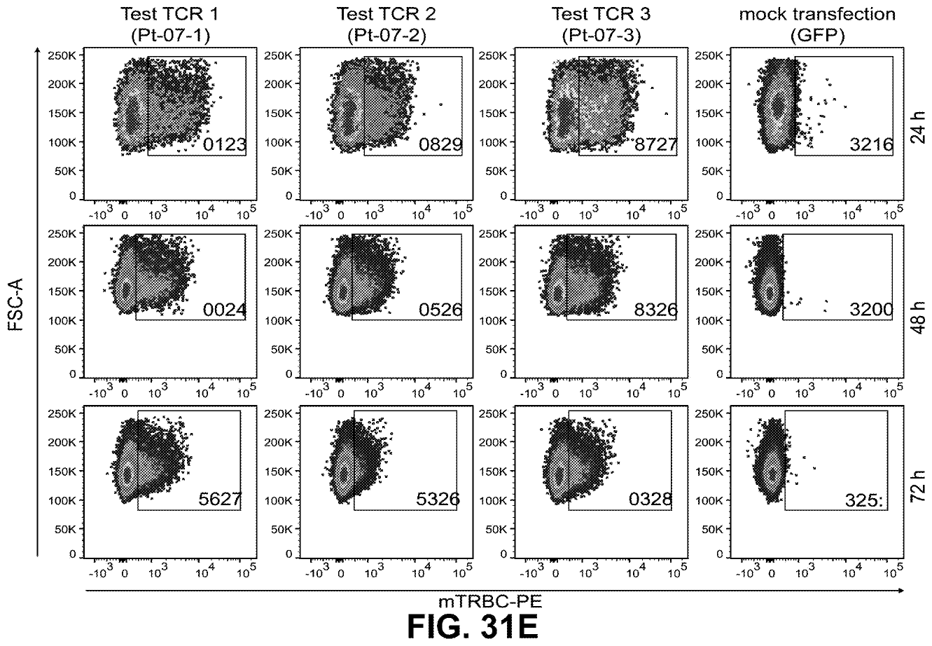

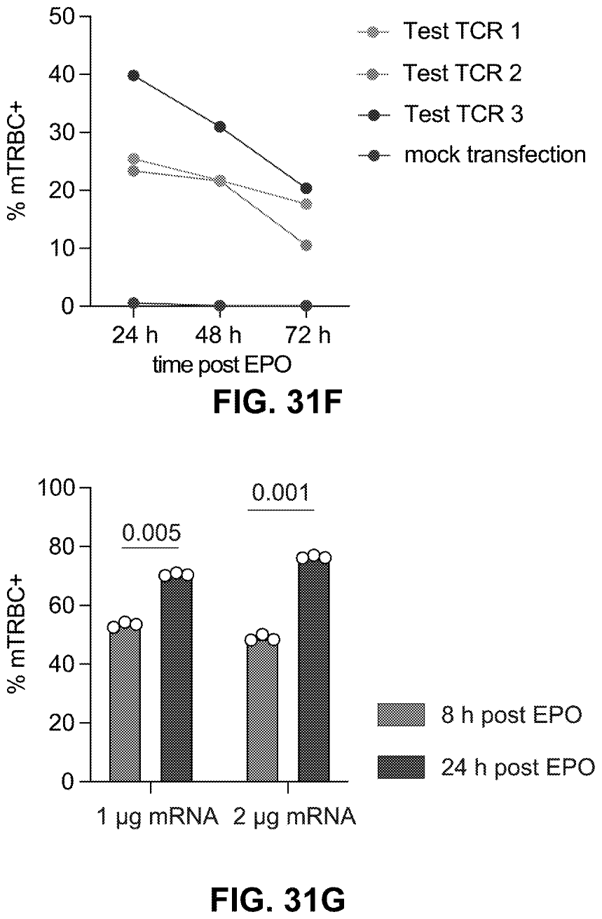

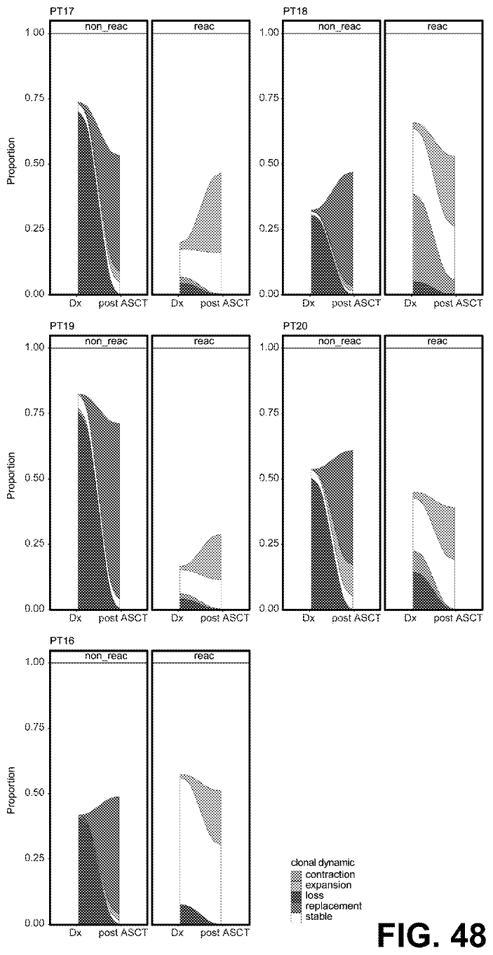

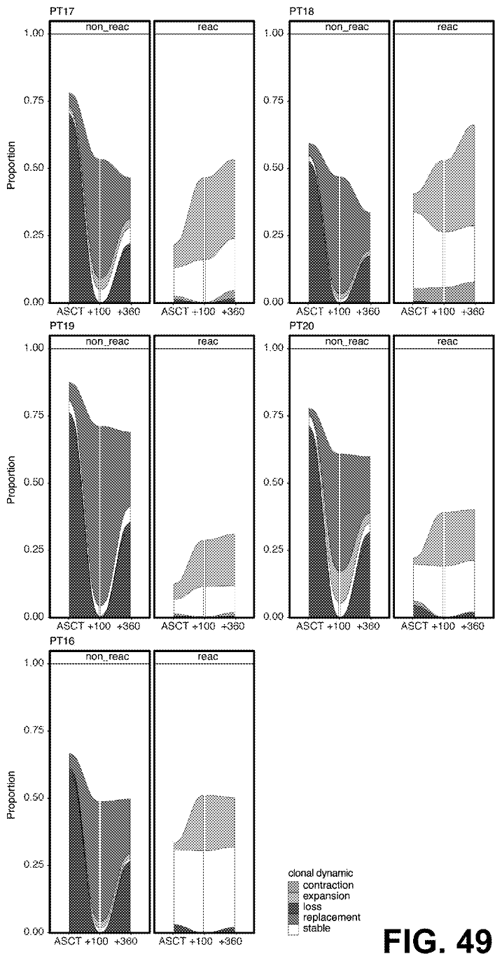

A total of n=576 BMTC exhibiting these signs of myeloma reactivity were captured in the cohort (~23 events per experiment), most of which expressed CD137 on their surface or, alternatively, secreted at least one of the cytokines in response to myeloma cell contact. A subset of polyfunctional BMTC that exhibited both expression of CD137 and cytokine secretion were further observed. IFN-γ was the most frequently secreted cytokine (93 events), followed by TNF and IL-2 (62 and 39 events, respectively) (FIG. 2B, FIGS. 15, 16, & 28). An average of 0.36% assayed BMTC were detected in all patients with at least one signal of anti-tumor reactivity, which were subsequently retrieved for TCRA/B-seq (FIGS. 2C & 2D, FIG. 30, FIG. 48). CD8⁺ T cells were preferentially enriched among detected events with CD137 surface protein expression, whereas all T cells that secreted IL-2 alone in response to tumor cell exposure were CD4⁺ (FIG. 2E, FIG. 31A). One potential limitation of any functional TCR reactivity testing is a detection bias against dysfunctional clonotypes or CD4⁺ cells with limited cytotoxicity. However, several CD4⁺ tumor-reactive TCRs with a predominantly progenitor-exhaustion phenotype were detected and mapped in the establishment patient cohort, including T cells with limited, but confidently detectable tumor-reactive cytokine secretion (FIG. 2D, FIG. 31A). Lastly, the frequency of tumor-reactive clones in the bone marrow correlated with gene expression profiles: less abundant tumor-reactive clones expressed markers such as SELL or IL7R and resembled bona fide naïve T cells, whereas more abundant clones expressed NK cell-like cytotoxic genes including GZMB, GNLY, PRF1, and NKG7 (FIG. 31B).

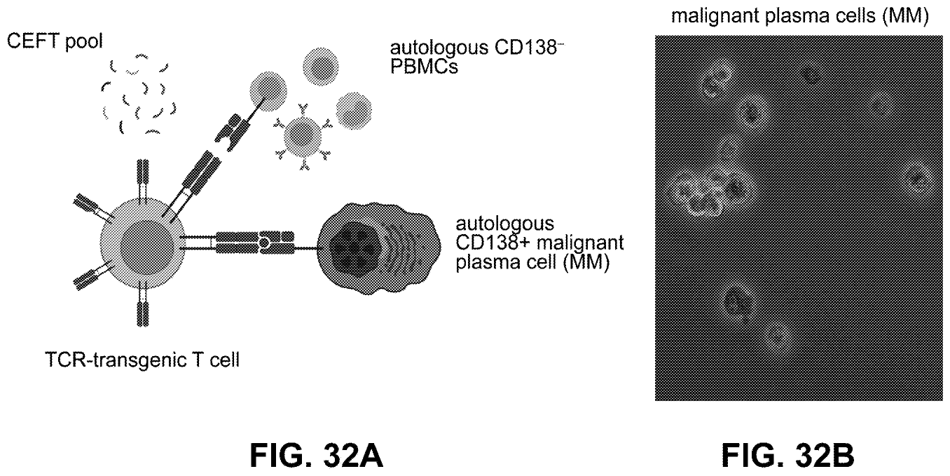

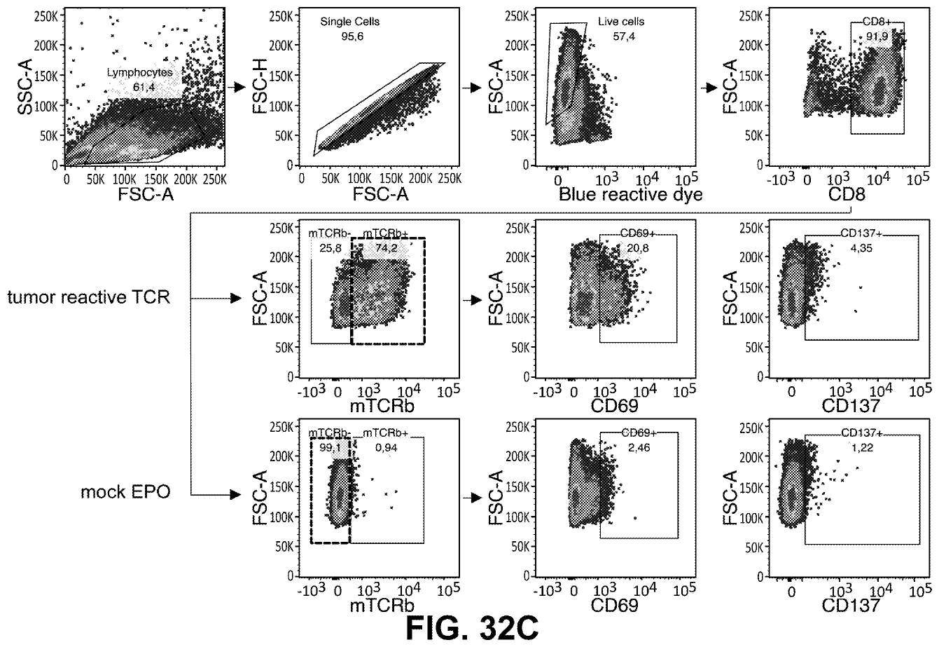

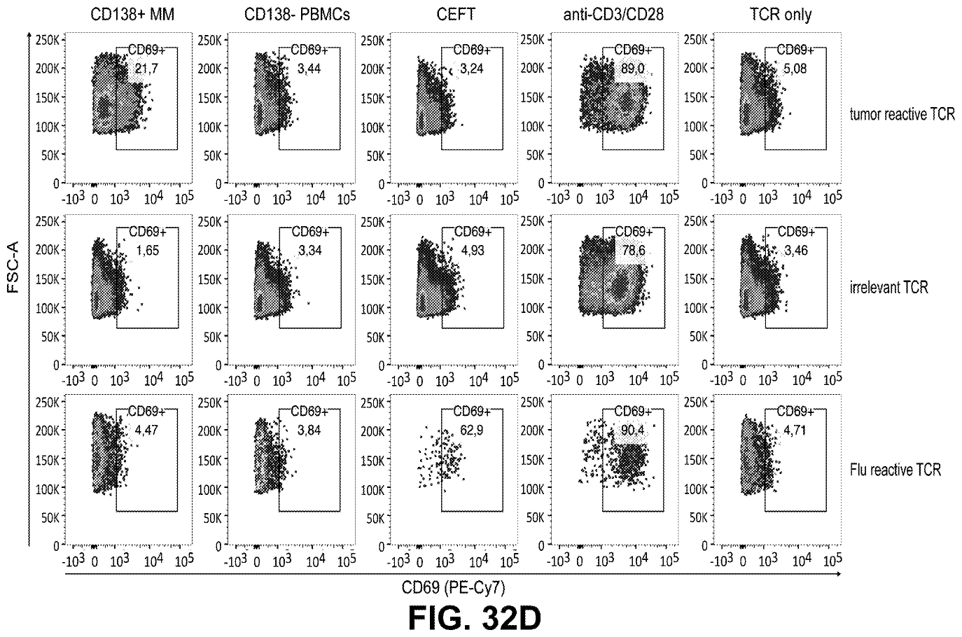

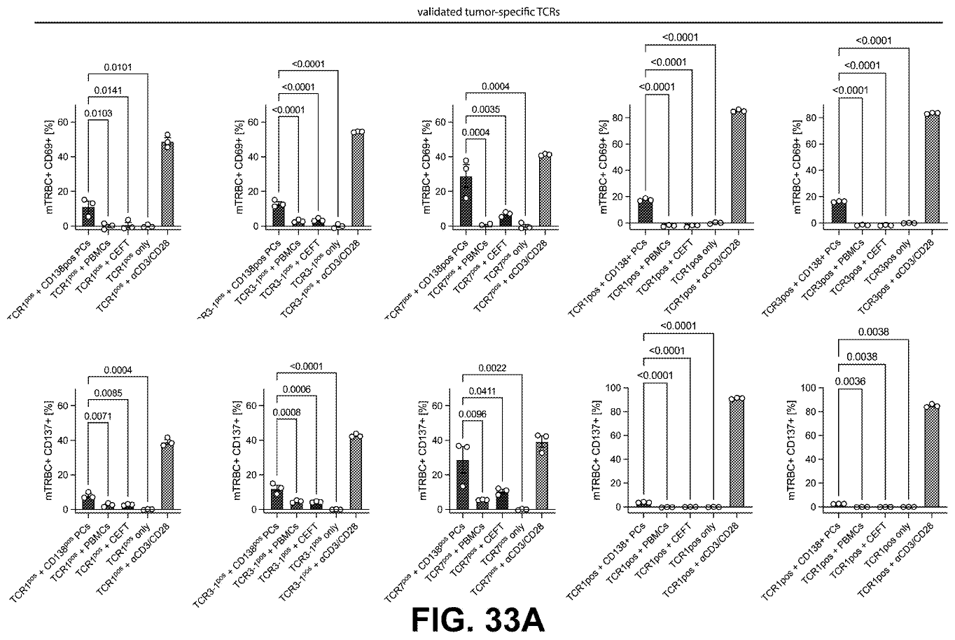

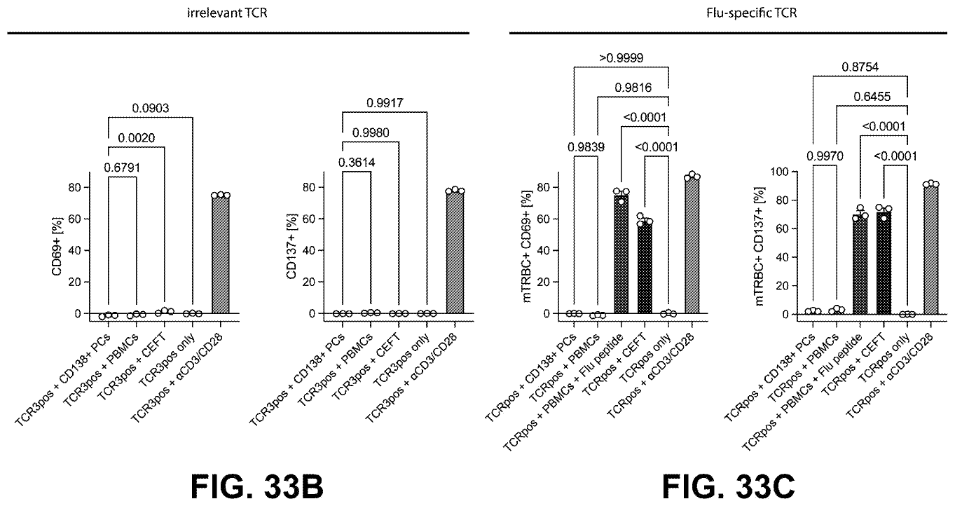

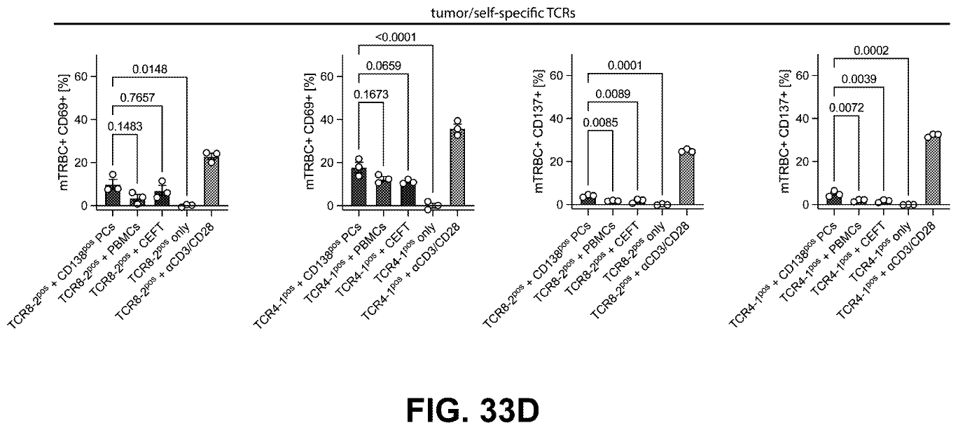

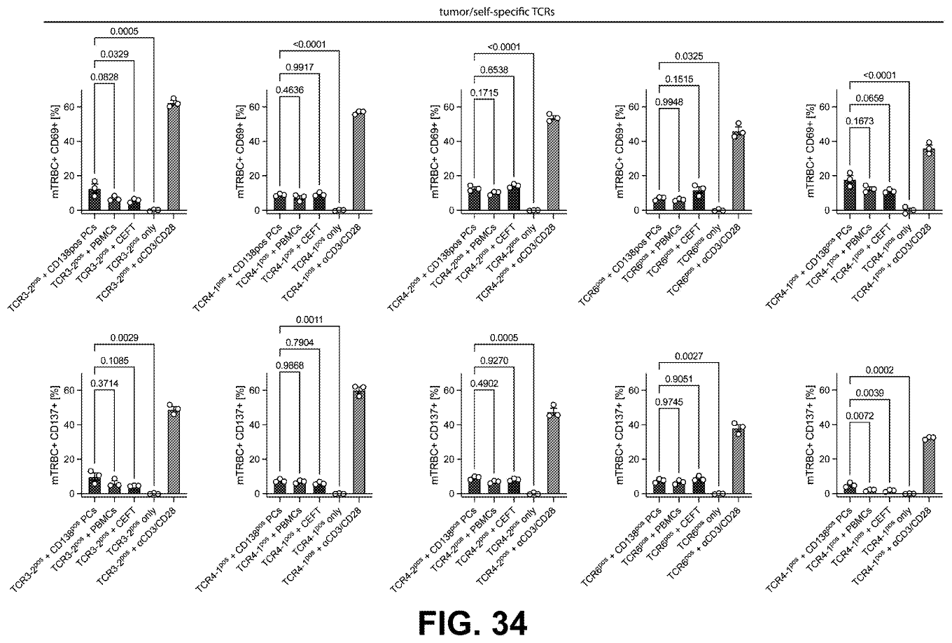

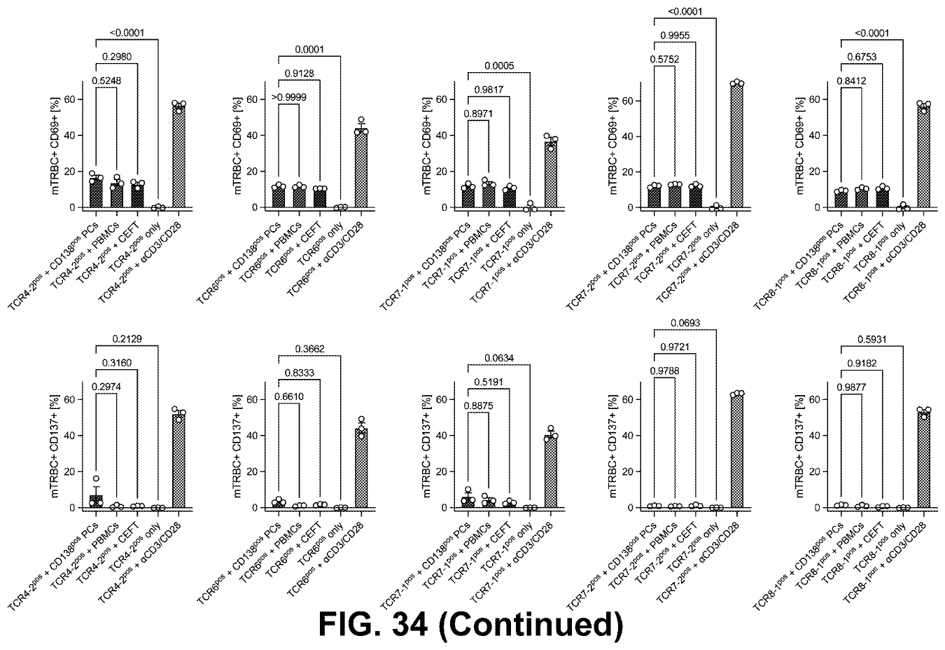

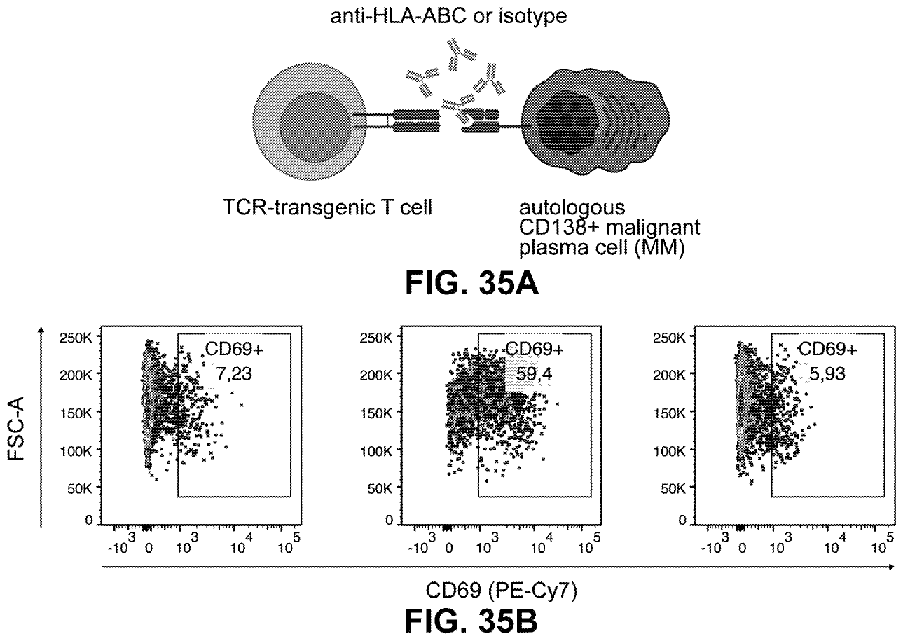

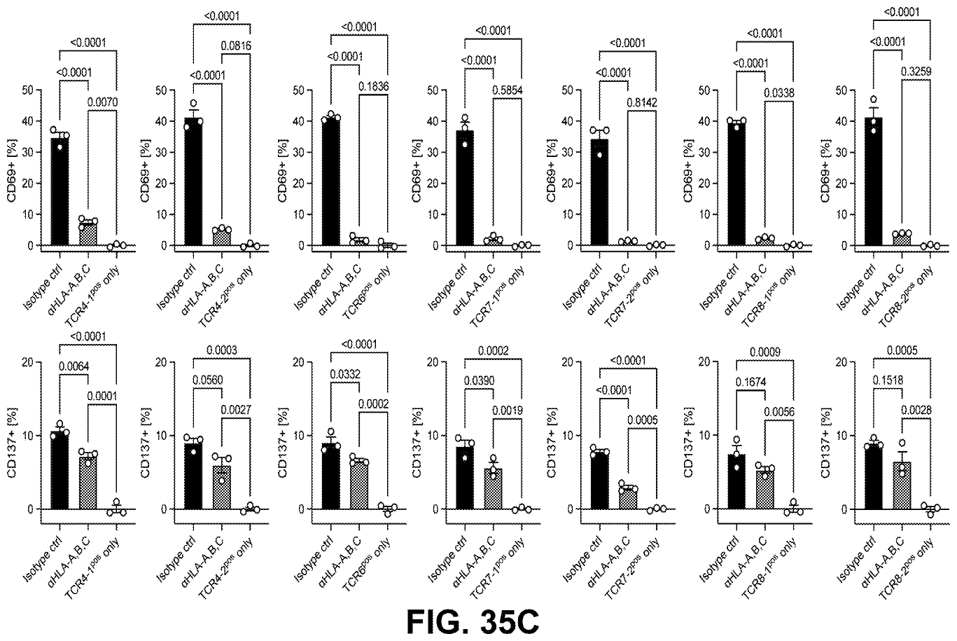

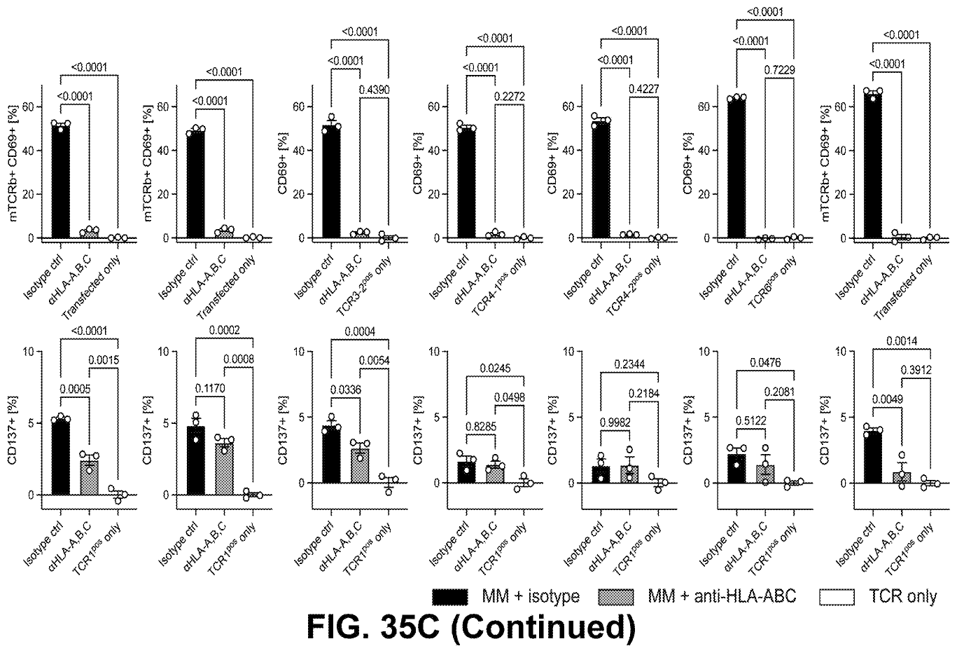

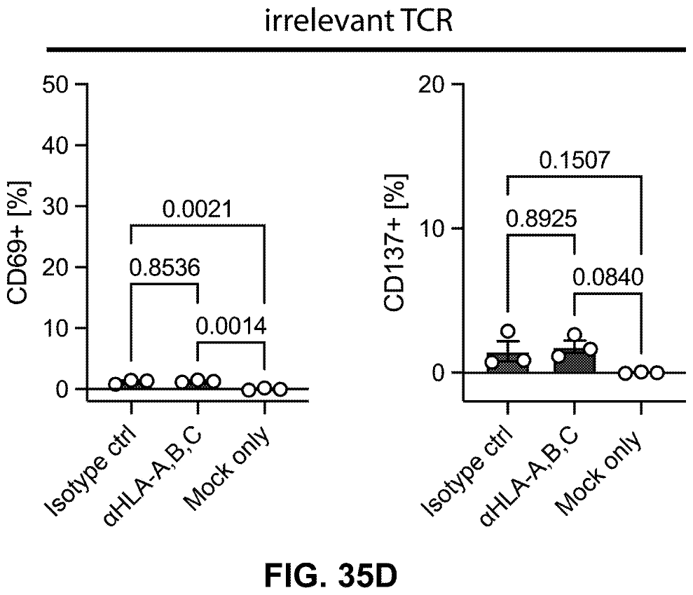

The screening approach was validated by independently assessing the tumor reactivity of identified TCRs and delineated their capacity to recognize tumor cell, viral epitope, or autoreactive targets in patients with available viable tumor cells and PBMCs (FIG. 2F). Upon reconstruction, assembly, in vitro transcription (IVT) and transgenic TCR-mRNA transfection of patient-autologous T cells from peripheral blood, individual TCRs were analyzed from the screen by multiparametric flow cytometry (FIGS. 31D-31G). Surface expression of transgenic TCRs, as well as increased transduction of the TCR signal and activation, detected as upregulation of CD69 and CD137 surface expression, were measured upon co-culture of transgenic TCR-carrying cells against patient-individual multiple myeloma cells, PBMCs and a virus epitope pool (CEFT) and compared to cells transfected with an irrelevant or no TCR (FIG. 2F, FIG. 32). From these experiments, 10 TCRs could be validated as tumor-specific, although some demonstrated tumor- and self-reactivity or tumor- and virus-reactivity (FIG. 2G, FIGS. 33 & 34). Functional HLA-ABC blocking experiments were further performed and it was found that the recognition of multiple myeloma cells in all tested TCRs was dependent on canonical MHC class I, mirroring the preferential detection of CD8⁺ T cells in our BMTC screen (FIG. 2H, FIG. 35A-35D).

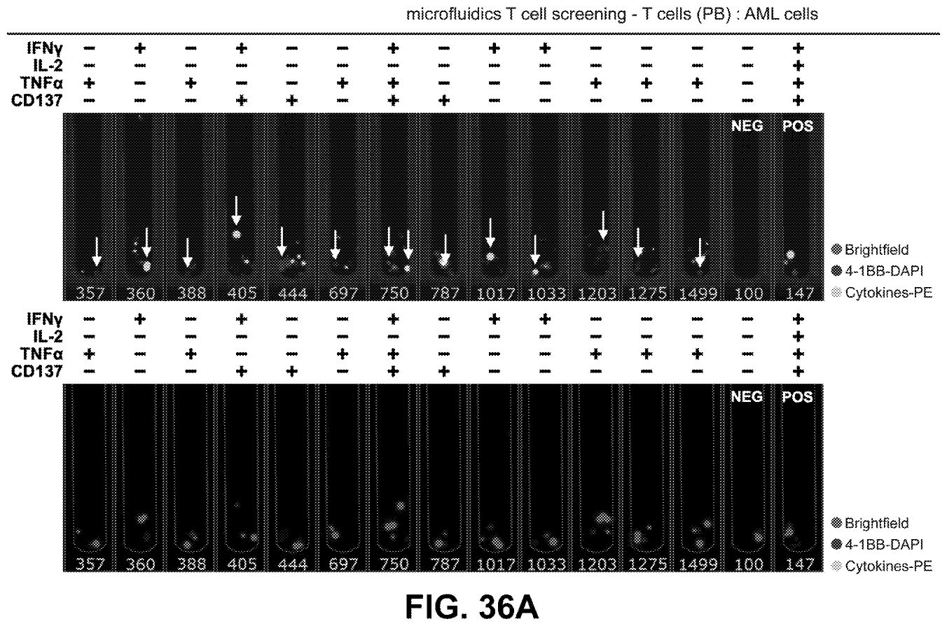

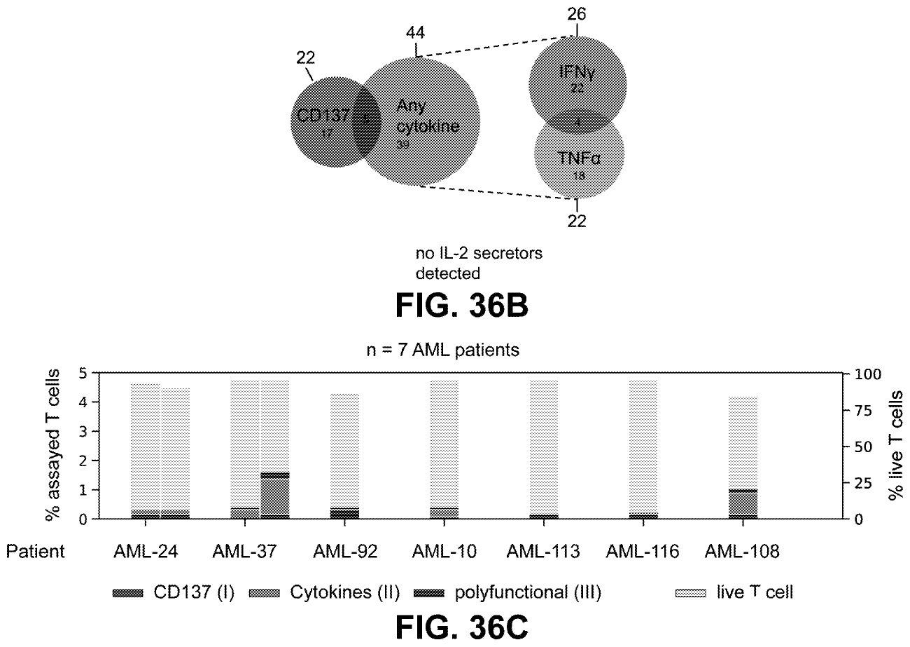

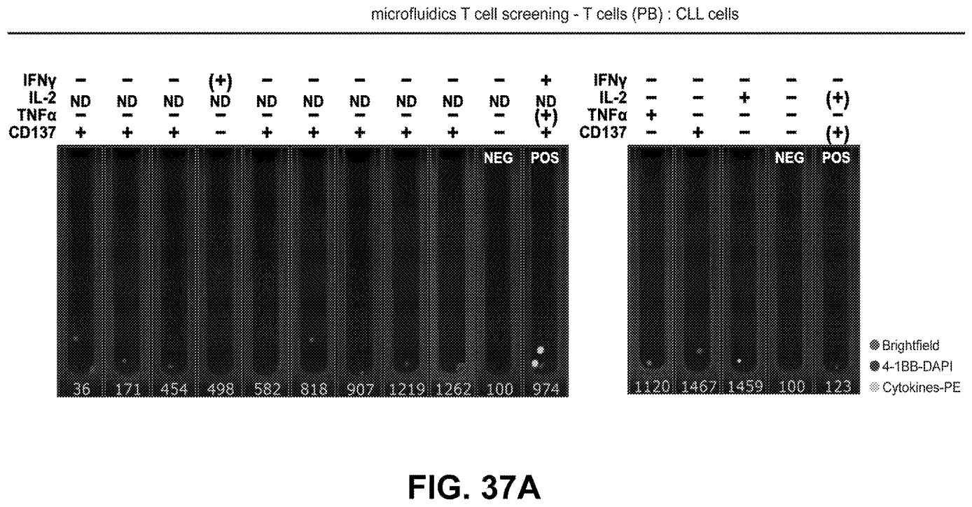

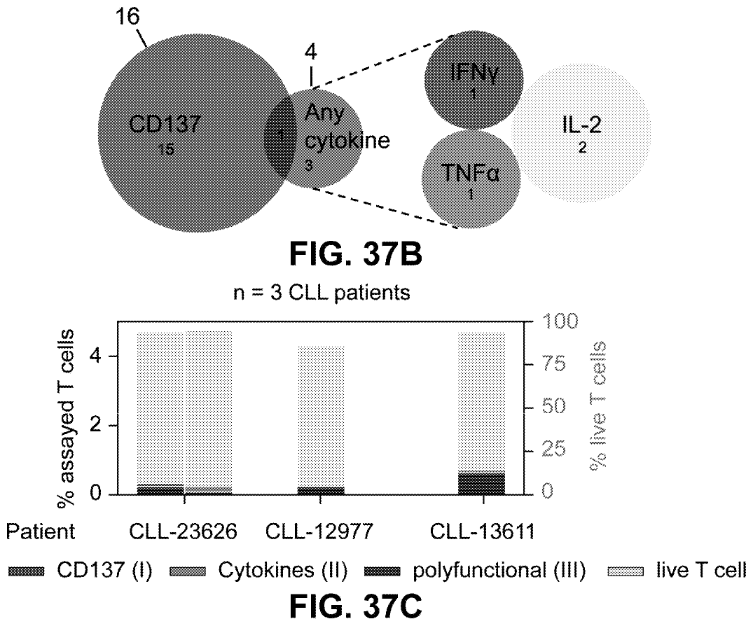

To test the ability of the forward screening approach to detect tumor-reactive T cells beyond multiple myeloma, additional experiments were performed in samples from newly diagnosed AML and CLL patients. As these diseases, by definition, are enriched for tumor cells in the peripheral blood, circulating T cells and tumor cells were isolated from cryopreserved PBMC samples (FIGS. 36 & 37). Considering only correctly loaded reaction chambers, tumor-reactive T cells were found in all 7 tested AML patient samples, albeit no IL-2 secretors were did not detected (FIGS. 36A-36C). Similarly, T cells exposed to CLL cells preferentially demonstrated CD137 upregulation and clonal proliferation relative to cytokine secretion (FIGS. 37A-37C).

As the in vitro screen inevitably perturbs the transcriptome of each assayed T cell, combined TCRA/B chain sequences were recovered and then all validated tumor-reactive TCRs were mapped to the expression states of the corresponding captured and sequenced BMTCs to delineate their phenotype in the baseline biopsy dataset of each patient using the CDR3 nucleotide sequence as clonal barcode (methods). This resulted in back projection of n=2,133 tumor-reactive T cells from n=62 TCR clonotypes to the scRNA/VDJ-seq dataset due to TCR clonotype sharing (26 unique TCRs).

### Characterization of Antigen-Specific Bone Marrow T Cells

Given the median count and distribution of unique BMTC clonotypes in the patient biopsies and considering the benchmarks of our screening platform, the minimal frequency of an individual TCR in the population to be sampled at least once with ≥95% probability is approximately 0.2% (or 42 cells per clone). Although this approach would most likely detect a substantial number of functional or clonally expanded tumor-reactive BMTCs, it ignores smaller or antigen-inexperienced T cell clones that could in principle harbor TCRs with a specificity for tumor antigens. To additionally consider these rare tumor-reactive TCRs and further experimentally identify TCRs with virus reactivity, a series of functional BMTC expansion assays was performed, analogous to other studies that discovered tumor-reactive TCRs in solid tumors⁵.

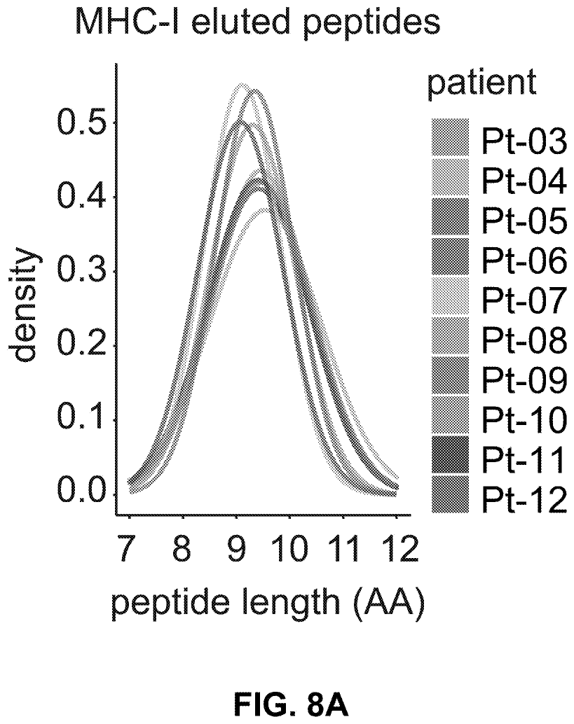

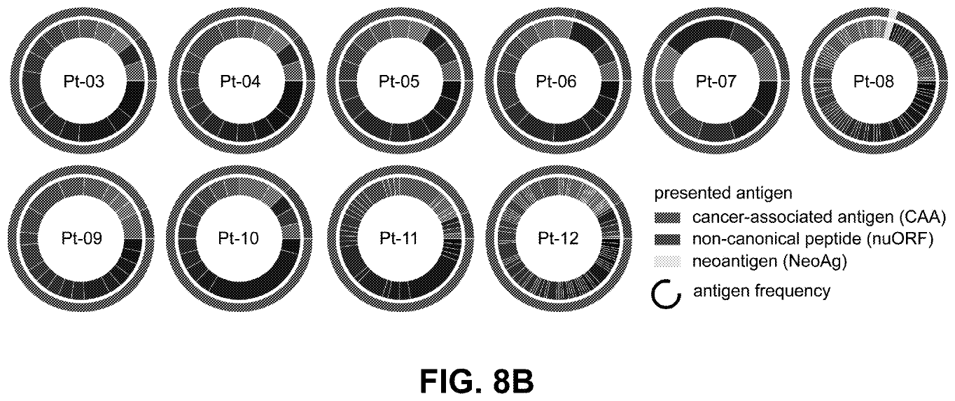

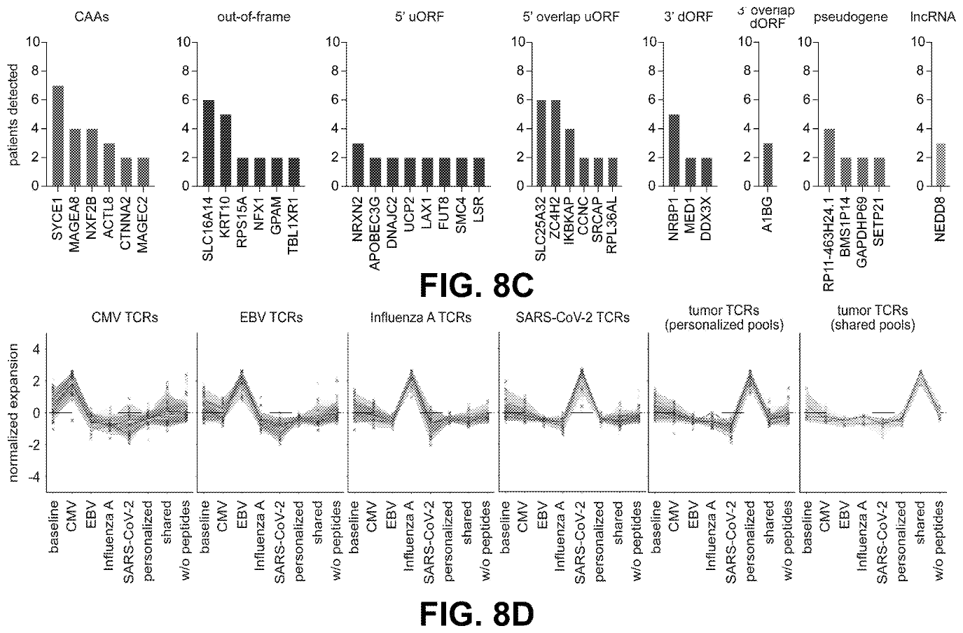

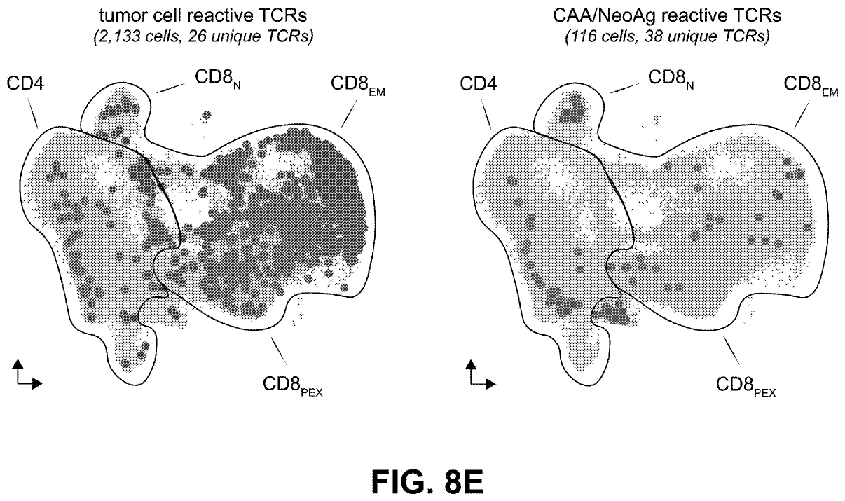

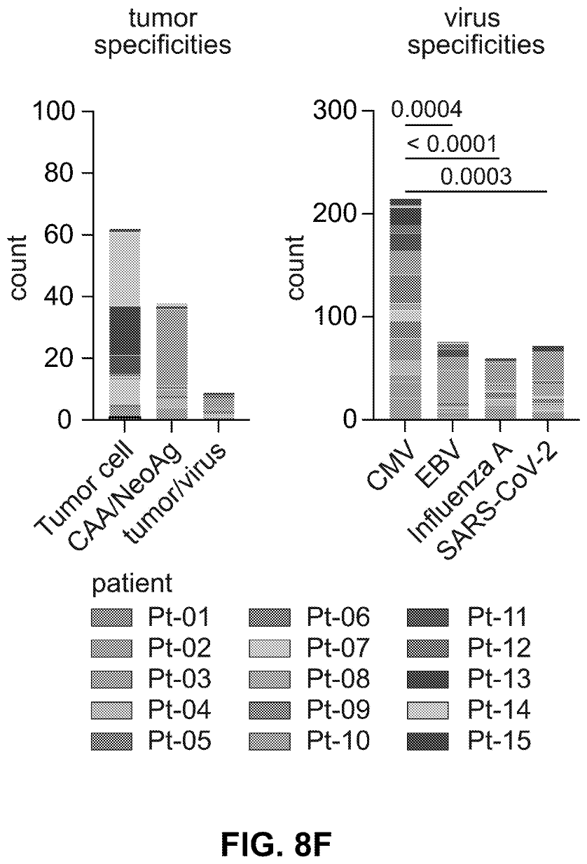

This approach, however, has the significant limitation that it cannot be performed in an antigen- or HLA-agnostic fashion. Following HLA haplotyping of tumor and germline samples in all 20 patients (Table 10), immunoprecipitation of MHC class I:peptide complexes was therefore performed using sorted malignant CD138⁺ multiple myeloma cells derived from the bone marrow of the patients and identified surface-presented tumor antigens by liquid chromatography tandem mass spectrometry (LC-MS/MS) analysis to generate full cancer class I HLA immunopeptidomes. As expected, most class I-eluted peptides were 9 amino acids (AA) in length (FIG. 8A). For each patient, “healthy” peptides were separated from “non-healthy” peptides presented on multiple myeloma cells. All healthy peptides mapping to patient germline sequences were classified as self-antigens for downstream studies. Non-healthy peptides were mostly derived from cancer-associated antigens (CAAs; FIG. 8B) or originating from non-canonical translation products (nuORFs; FIG. 8B)⁶. It has recently been shown that peptides originating from nuORFs can be displayed on MHC class I of cancer cells, acting as additional sources of tumor-specific antigens⁷. Interestingly, it was observed that some of these MHC class I-presented antigens were common among several patients. Specifically, one peptide was found originating from synaptonemal complex central element protein 1 (SYCE1) in 7/10 patient samples we tested, as well as several other CAA or nuORF-derived epitopes with complete AA sequence homology in multiple patient tumor samples (FIG. 8C).

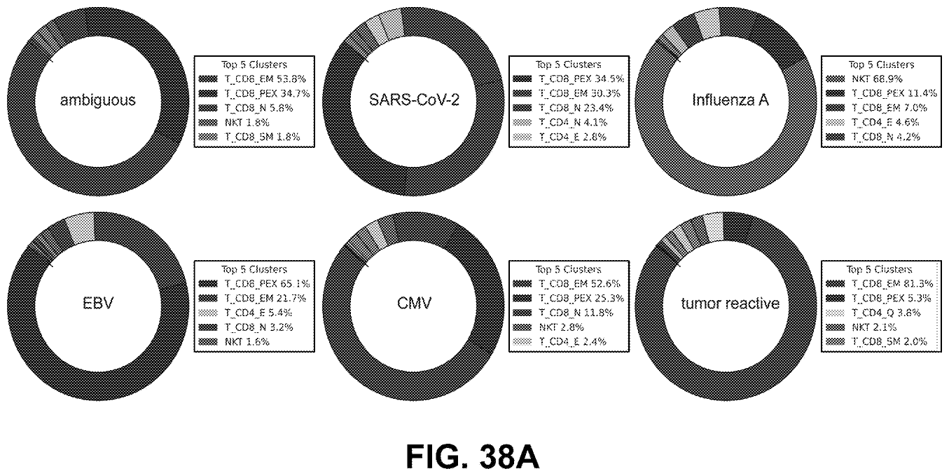

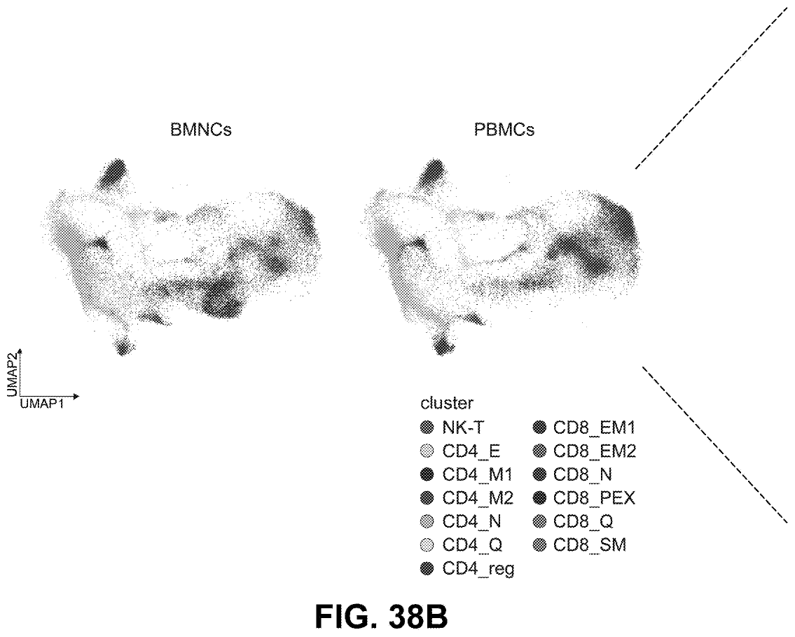

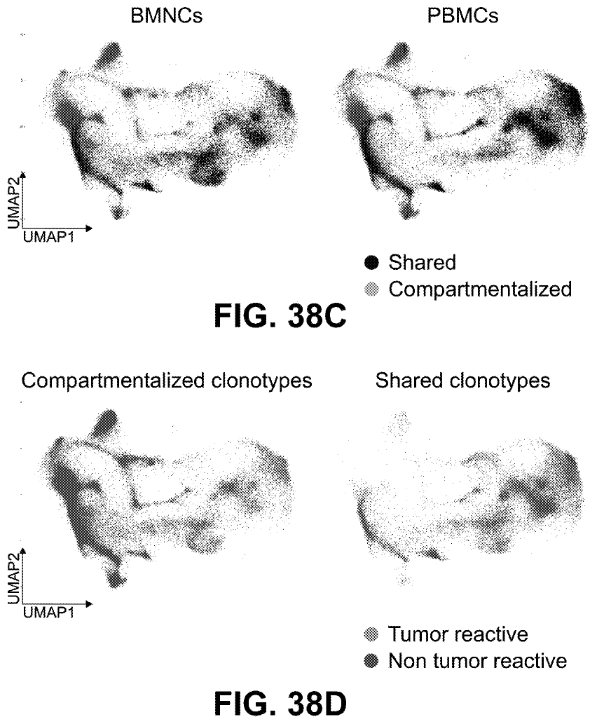

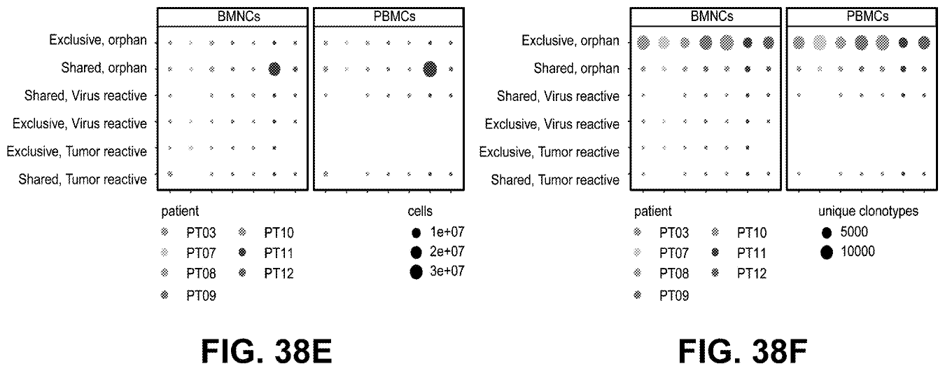

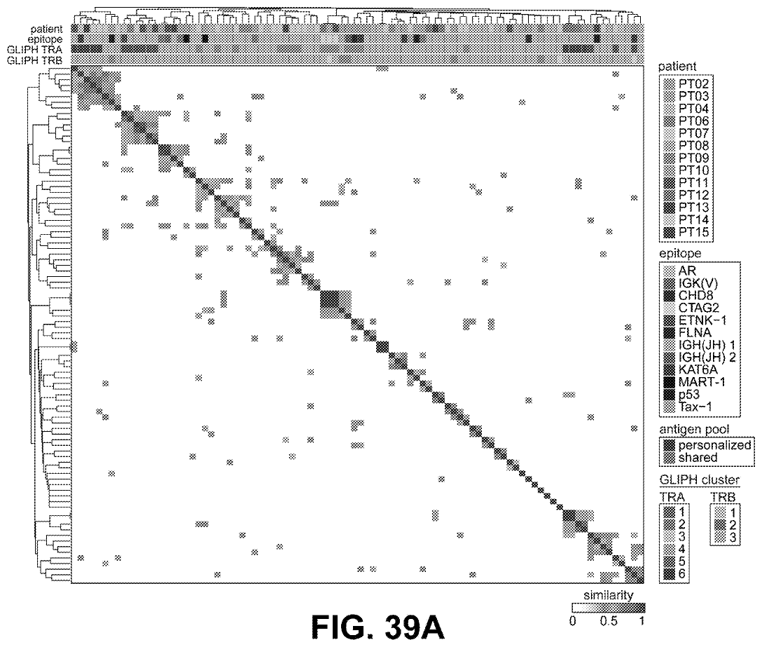

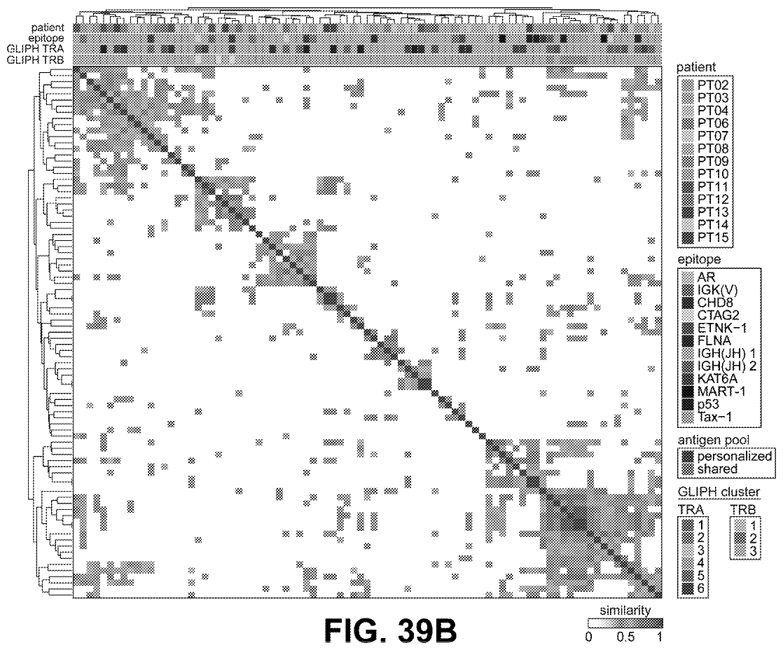

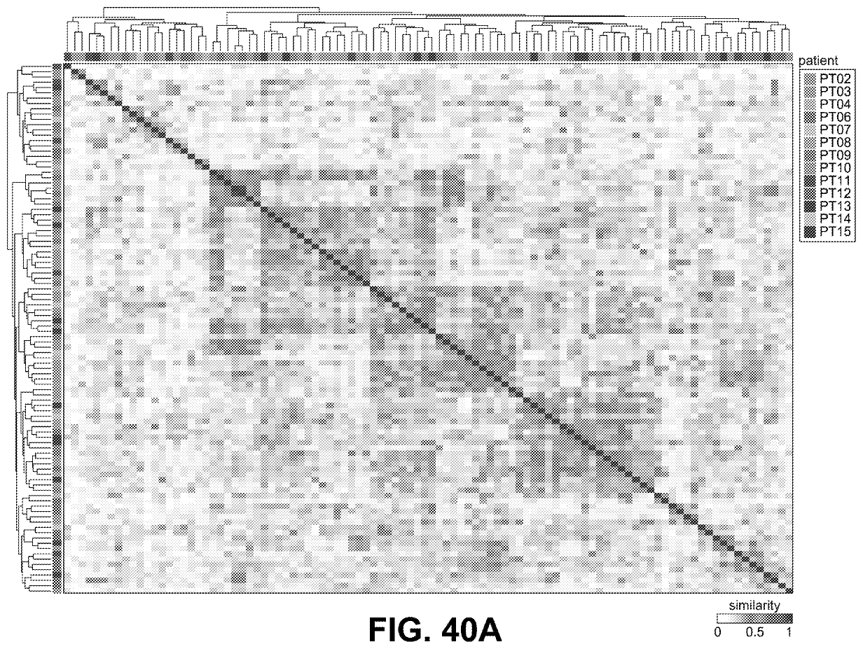

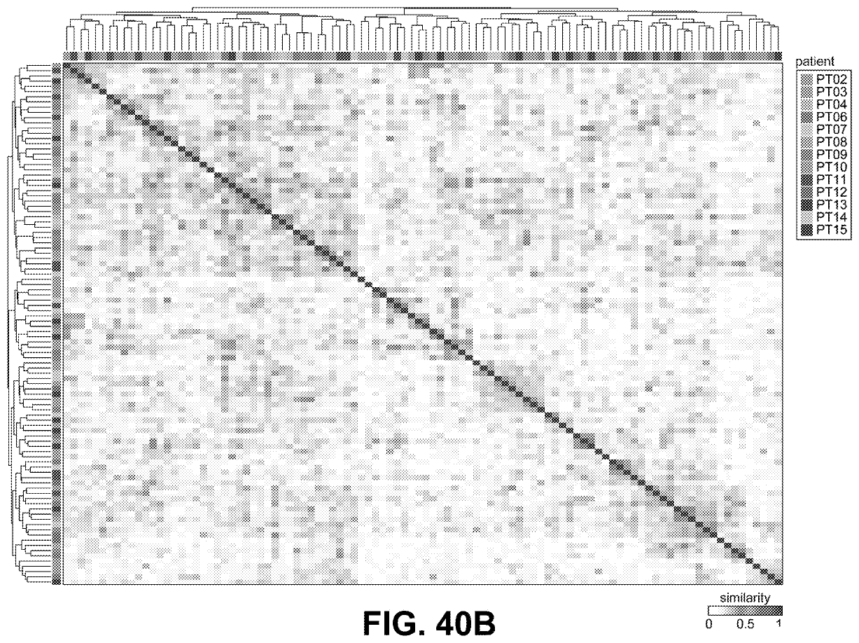

Notably, when the mass spectra for all potential WGS/RNA-seq predicted neoepitopes were queried, only one neoantigen was found in Pt-08, which was eluted from the tumor peptide:MHC class I complex (FIG. 8B). Recently, immunoglobulins or B cell receptors (BCR) of lymphoid malignancies have been suggested as alternative sources of neoantigens found only in B cell-derived cancers⁸⁻¹¹. Non-Hodgkin's lymphomas including multiple myeloma typically originate from B lymphocytes that have successfully rearranged their immunoglobulin genes through V(D)J recombination, and they may also undergo somatic hypermutation. Specifically, in follicular lymphomas and multiple myeloma, immunoglobulin gene selection bias and somatic hypermutation are frequently detected^{8,9}. The ability of HLA complexes to present fragments of these immunoglobulin idiotypes and their subsequent recognition by T cells have been observed in humans¹⁰⁻¹³. The BCR sequences of the main multiple myeloma clonotype were reconstituted in each patient of the cohort, predicted antigens with high binding affinity to the respective patient's tumoral HLA haplotypes and generated personalized BCR antigen libraries. These libraries as well as MHC immunopeptidome-derived antigen pools were used for further epitope mapping of the forward screening-derived tumor-reactive TCRs by co-culture of TCR-transgenic reporter cells and patient-derived immortalized B lymphoblastoid cell lines (B-LCLs) used as antigen-presenting cells (FIG. 38D). These cells express GFP upon TCR engagement as well as an anti-CD19 single-chain fragment variable (scFv) which labels the peptide loaded antigen presenters, together serving as two redundant readouts¹⁴ (FIG. 38D). With the exception of one TCR, all reconstituted TCRs were robustly expressed (FIG. 38F). Through this method, one BCR-derived neoantigen-specific TCR (Pt-11_TCR_5) was identified. This TCR recognized a peptide from the autologous multiple myeloma IGKV chain (CDR1 region; LYKENNKEYL (SEQ ID NO: 324)) (FIG. 39A).

Next, the MANA functional expansion of specific T cells assay (MANAFEST) was performed on 9 of the 10 individuals on whom MHC immunopeptidome profiling was conducted. The MANAFEST assay detects antigen-specific T cell responses through clonal expansion of TCR clonotypes following co-culture with presenter cells and target antigens as soluble peptide¹⁵. Two synthesized peptide pools were used to enrich putative tumor-reactive T cells. The first patient-individualized pool contained MHC class I immunopeptidomics-derived private antigens the second pool contained all antigens shared by >50% of patients in the study (Additionally, MHC class I-restricted pools of SARS-CoV-2 spike and nucleoprotein, cytomegalovirus (CMV), Epstein-Barr virus (EBV) and influenza A virus epitopes were queried for TCR reactivity in parallel. After 10 days of functional expansion, we subjected all cultures to ultradeep TCRVβ-seq and delineated statistically robust TCR clone sizes pre- and post-assay in response to each antigen pool by FEST analysis⁴. From all individuals, 123 total MANA-specific TCRs (88 TCRs reactive against private antigens, 35 TCRs reactive against shared antigens), 104 CMV-specific TCRs, 86 EBV-reactive TCRs, 88 Influenza A-specific TCRs, and 88 SARS-CoV-2-specific TCRs were identified. In addition to several repetitively detected virus-specific TCRβ chains, three TCRβ chains in cultures exposed to shared tumor antigens were identical in at least two patients, suggesting convergent TCRβ selection against shared viral, but also tumor-derived antigens. Importantly, no MANAFEST-determined or public HLA-matched TCR sequences with known viral specificities against CMV, EBV, SARS-CoV-2, or Influenza A could be matched to previously identified myeloma reactive TCRs¹⁶. Lastly, back projection of all MANAFEST-identified TCRs and public HLA-matched TCR sequences to the bone marrow repertoires of the studied multiple myeloma patients was performed to complement the forward screening-derived TCRs.

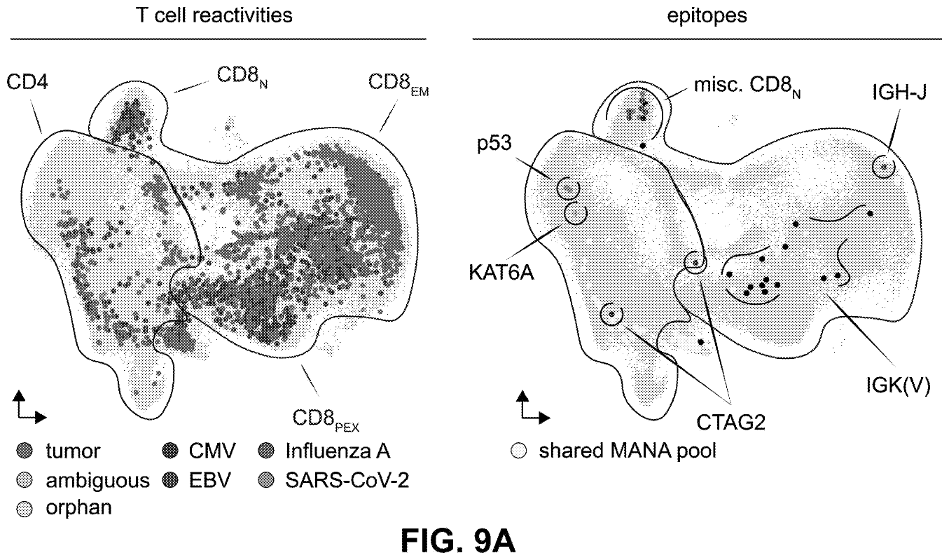

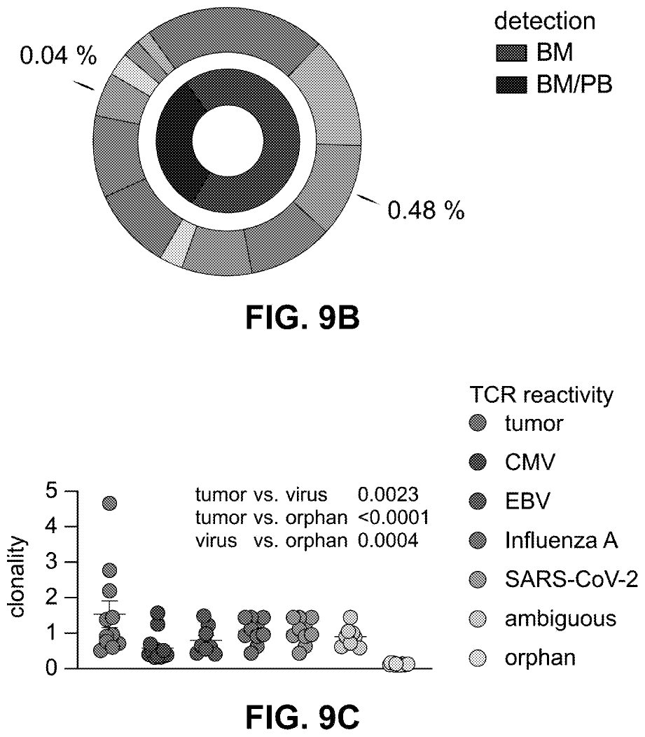

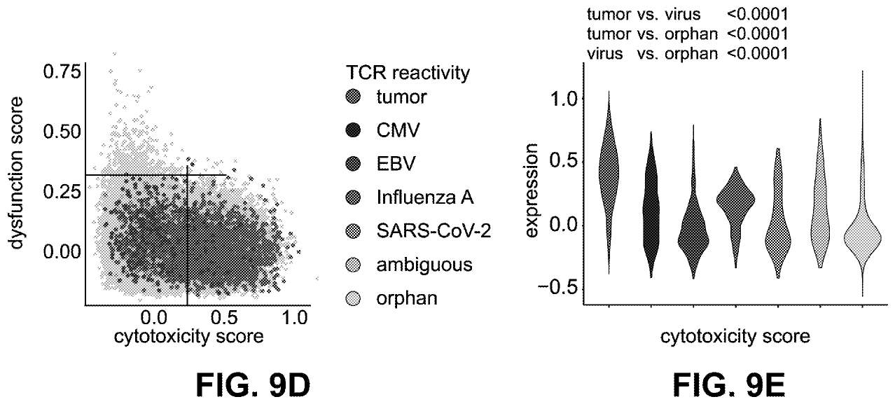

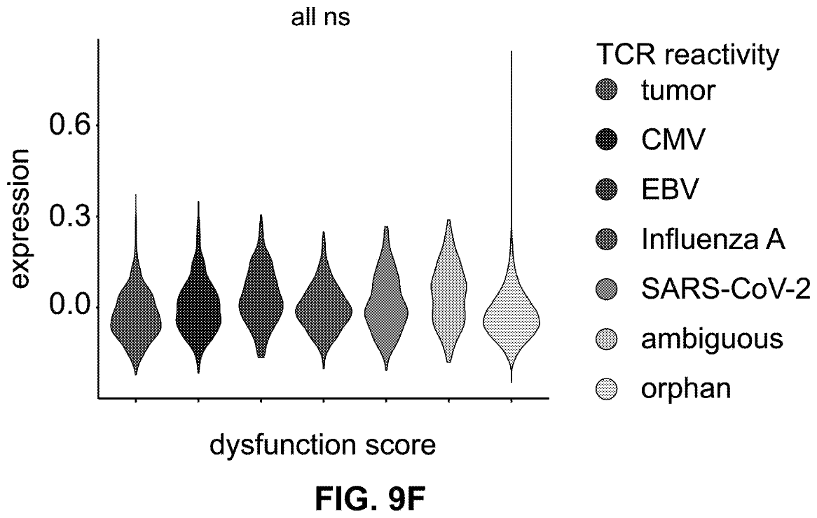

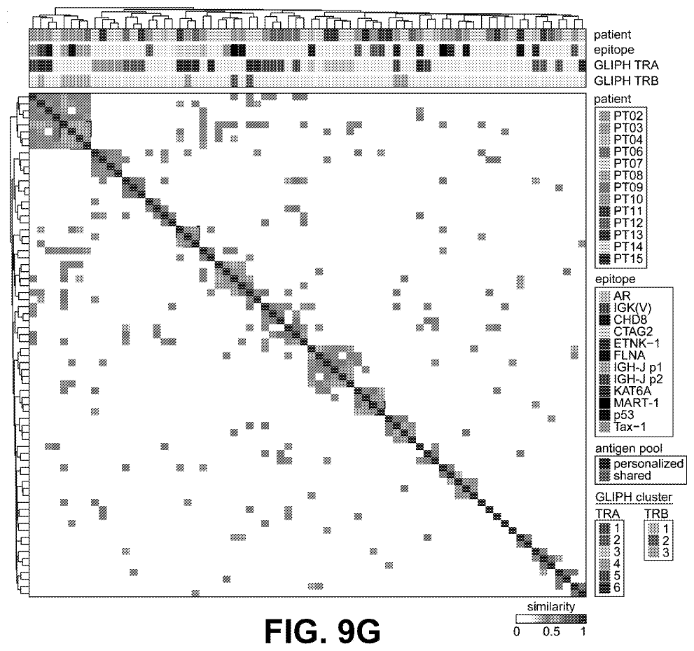

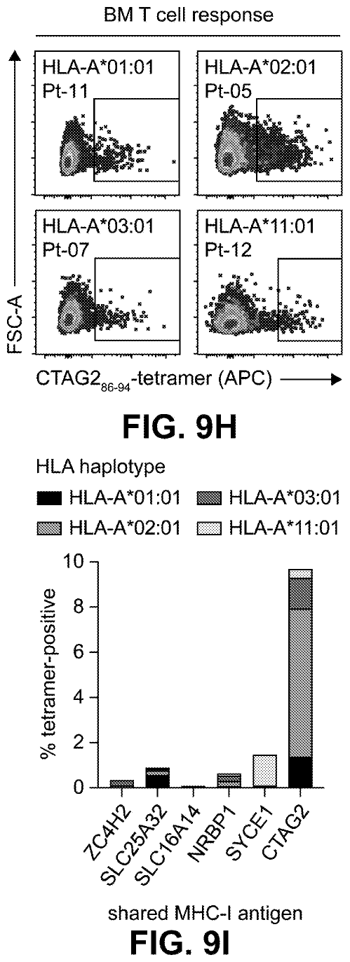

By integrating all antigen-agnostic and antigen-dependent screening data, we charted a map of antigen-specific BMTC. Twenty-six tumor-reactive TCRs were mapped from the forward screen and 38 TCRs identified as reactive against private and shared antigens (FIGS. 8E & 8F). Together, 1.29% of T cells in the diseased bone marrow were identified to be multiple myeloma reactive (FIG. 9A). Among these, TCR11729 and TCR15434 are of particular interest, with TCR11729 recognizing the CTAG2 antigen, which is expressed across multiple patients and HLA haplotypes, and TCR15434 recognizing a patient-specific mutation in the IGKV gene). In line with previous reports, most non-tumor-reactive clones were either CD4⁺ or enriched for viral specificities (FIG. 9A, FIG. 38A). Representative of healthy bone marrow immune repertoires, CMV specificities were significantly enriched among virus-specific BMTCs^{17,18} (FIG. 8F).

To investigate the compartmentalization of BMTC clones, we conducted combined scRNA/TCR sequencing of T cells obtained from matched bone marrow and peripheral blood samples. The behavior of antigen-specific clonotypes was tracked in seven patients who exhibited abundant tumor-reactive BMTC populations (FIG. 9B, FIGS. 38B-38D). Analysis revealed a higher prevalence of virus-specific TCRs relative to tumor-specific TCRs in the peripheral blood, which translated into a significantly more stable presence of circulating virus-specific TCRs compared to tumor-reactive clones. This enriched repertoire of virus-reactive specificities likely contributes to host immunosurveillance and might infiltrate the bone marrow via blood perfusion or recognition of non-tumor antigens¹¹. Vetted tumor-reactive TCR clonotypes were relatively scarce among circulating cells (0.48%, FIG. 9B, FIG. 38E). However, they exhibited higher clonality when compared to virus-specific or orphan clonotypes in the bone marrow, suggesting expansion of these clonotypes within the tumor microenvironment (FIG. 9C) 19. Here, stimulation by tumor antigens could be responsible for site-specific clonal expansion and the acquisition of the previously observed effector/memory phenotype. Comparing our data to signatures of dysfunctional and cytotoxic gene programs²⁰, it was found that TCRs with viral specificities shared the functional profile of bystander cells in the diseased bone marrow. The majority of myeloma reactive TCRs, however, expressed a cytotoxicity program at baseline in this treatment-naïve patient population (FIG. 9D-9F).

### Shared T Cell Responses Against Cancer-Associated Antigens

As was detected several public class I HLA-presented tumor antigens in patients together with singular events of TCRB chain sharing among individual MANAFEST cultures, it was asked whether chronic exposure to these shared cancer antigens could induce convergent selection of TCR motifs in the bone marrow. Reduction of BMTC to their CDR3 TCRA/B chain motifs enabled us to generate motif networks of clonally expanded TCRs with sequence similarities (FIG. 9G)²¹. Convergent TCRs were defined as T cell clones found in more than one individual that share at least one variable gene and CDR3 AA sequence with at least one other clone, but might differ at the nucleotide level (i.e., “public” rearrangements)²²⁻²⁴. Following a bootstrapping approach to determine significance thresholds of these AA similarities, it was found tumor- and virus-specific repertoires contained clustered groups of receptors coming from the bone marrow of multiple patients that share core CDR3 sequence similarities, together with a dispersed set of diverse ‘outlier’ sequences (FIGS. 39 & 40).

Within all vetted tumor-reactive TCRs in our cohort, one noticeable cluster of similarity contained a public immunoglobulin J-chain (IGHJ)-specific TCRβ CDR3 sequence that was found in three multiple myeloma patients (Pt-02, Pt-06, Pt-12). IGJ was recently identified as a target antigen in multiple myeloma. IGJ is highly expressed in the majority of patient multiple myeloma samples, whereas expression in healthy tissues of non-B-cell origin is absent²⁵. Indeed, moderately high expression of IGH-J was found in tumor samples of the multiple myeloma cohort (FIG. 38B).

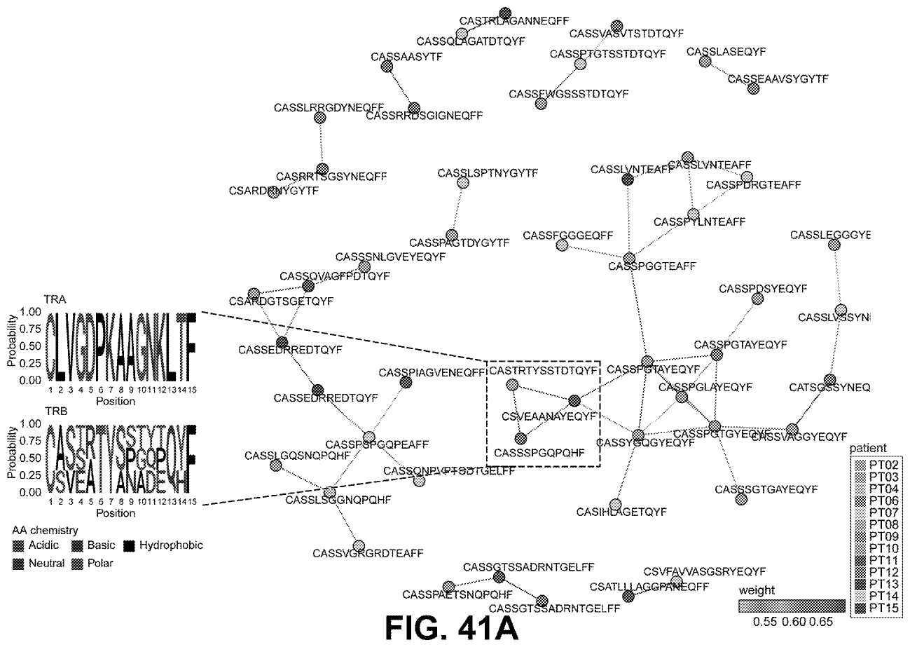

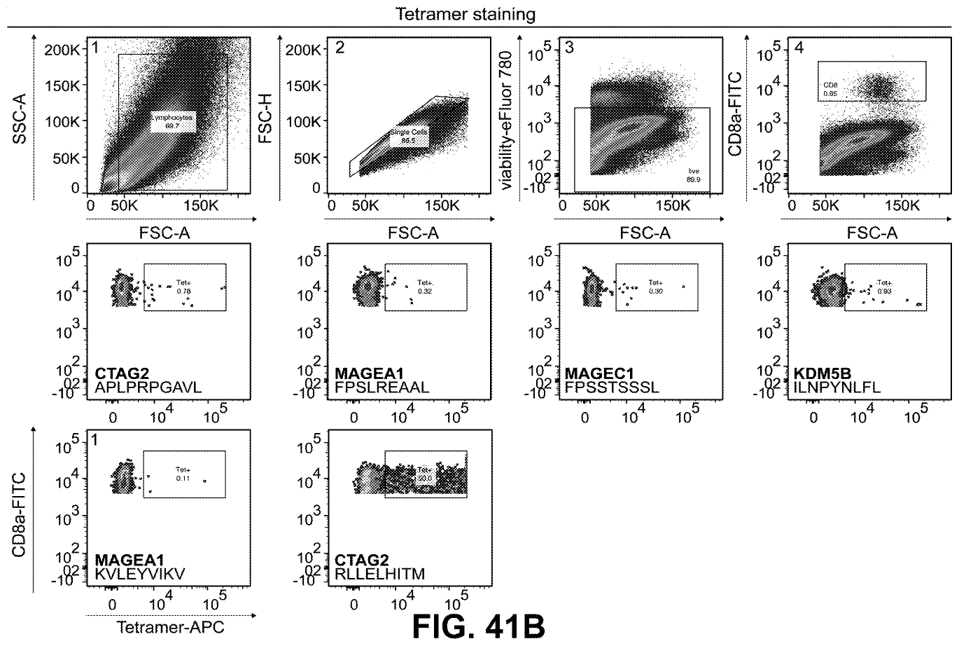

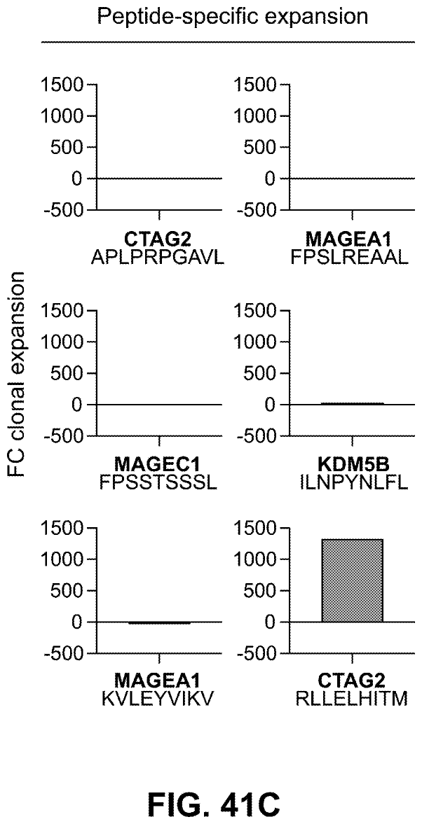

Another cluster of increased similarity contained a fully shared TCRA CDR3 sequence that was found in three multiple myeloma patients (Pt-06, Pt-08, Pt-11) together with individual, but partially overlapping TCRB CDR3 sequences (FIG. 9G, FIG. 41A). Using the available tumoral MHC class I ligandome information and cells from Pt-08, it was shown that CTAG2⁴⁶⁻⁴⁸ (RLLELHITM (SEQ ID NO: 128)) was the cognate antigen for this TCR. This epitope was identified by staining TCR-transgenic cells with MHC class I tetramers loaded with CTAG2⁴⁶⁻⁴⁸ (compared to all other non-healthy MHC class I-eluted peptides (FIG. 41B). Additionally, T cells transfected with this TCR clonally expanded in a functional expansion assay using this, but none of the other identified cancer-derived epitopes (FIG. 41C) 4. These data, together with the fact that Pt-06, Pt-08, and Pt-11 have non-overlapping HLA haplotypes, suggested that there might be recurrent BMTC responses in multiple myeloma patients to shared tumor antigens across multiple HLA haplotypes.

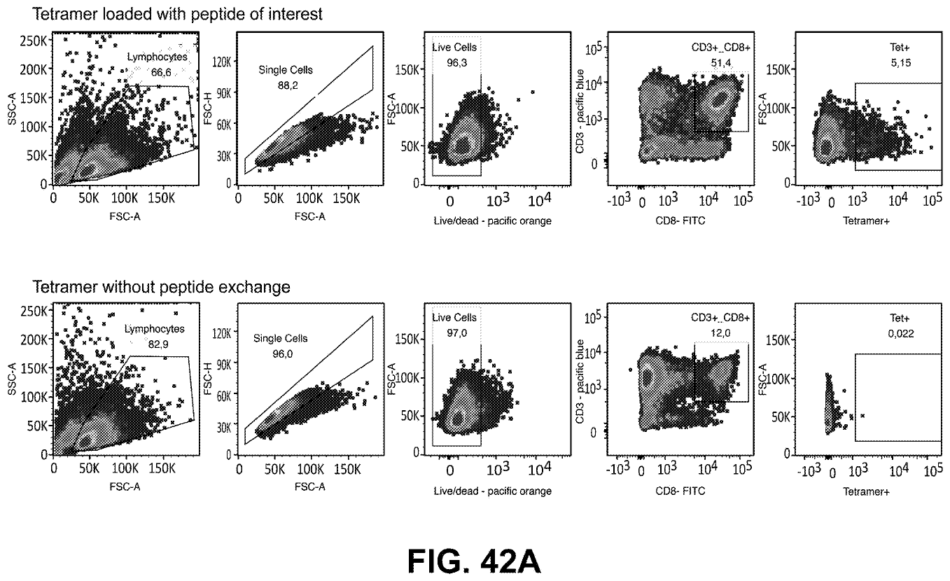

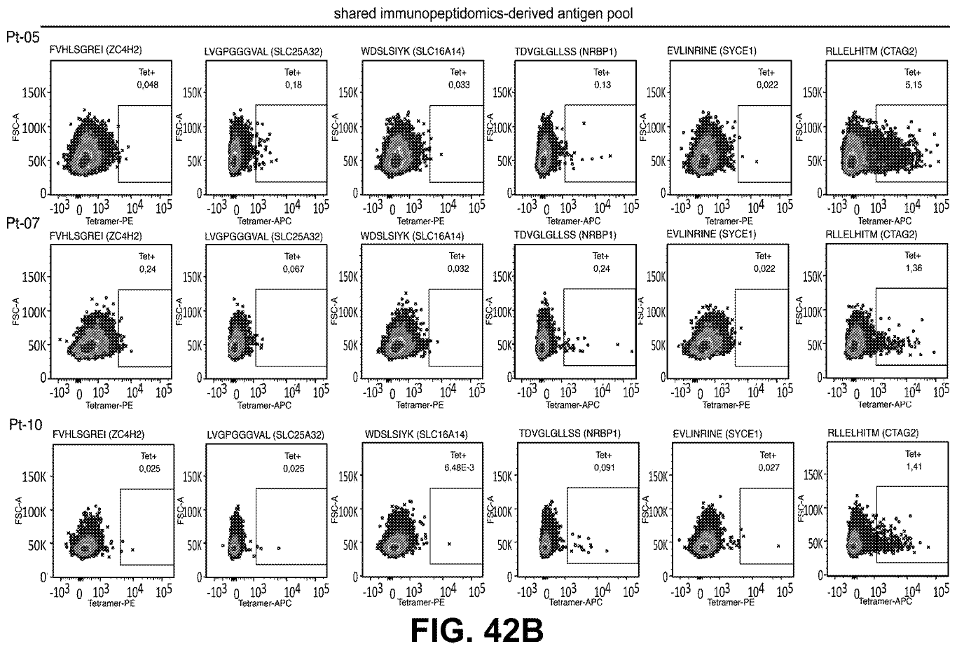

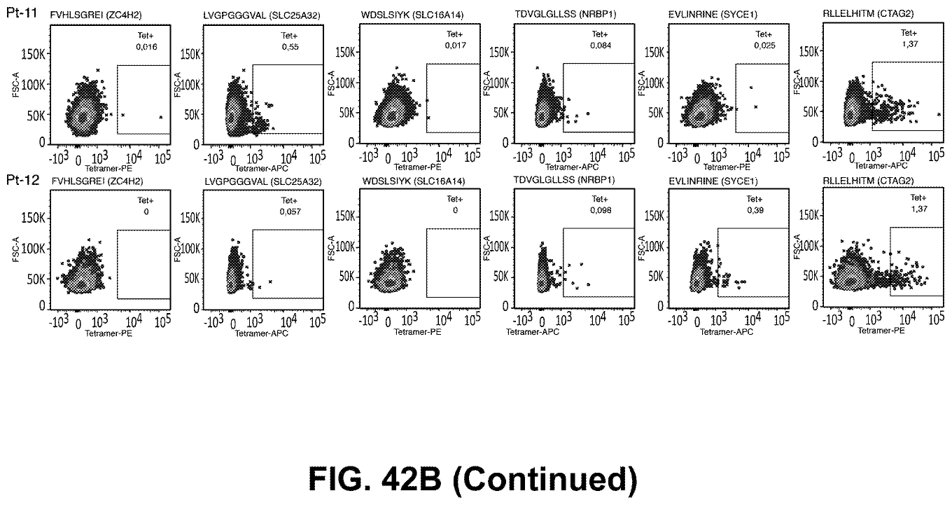

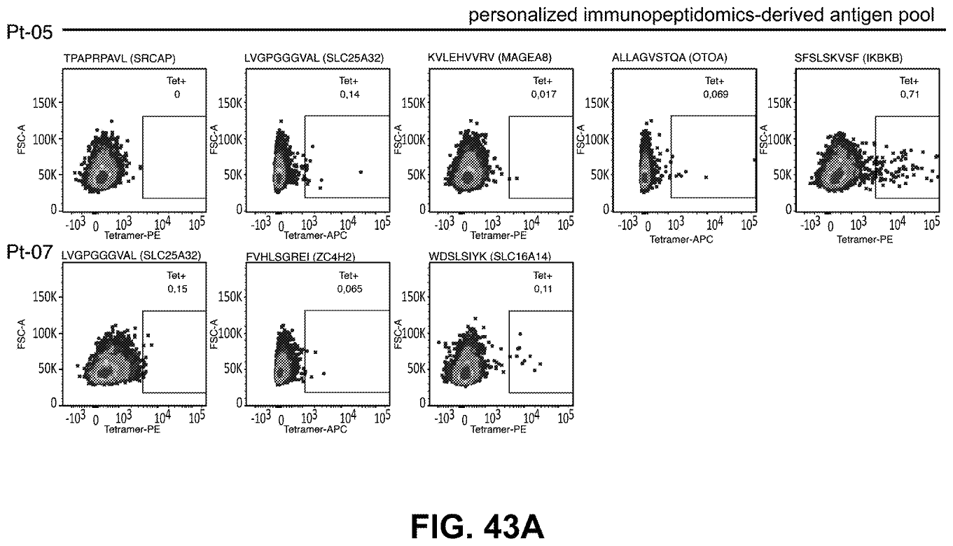

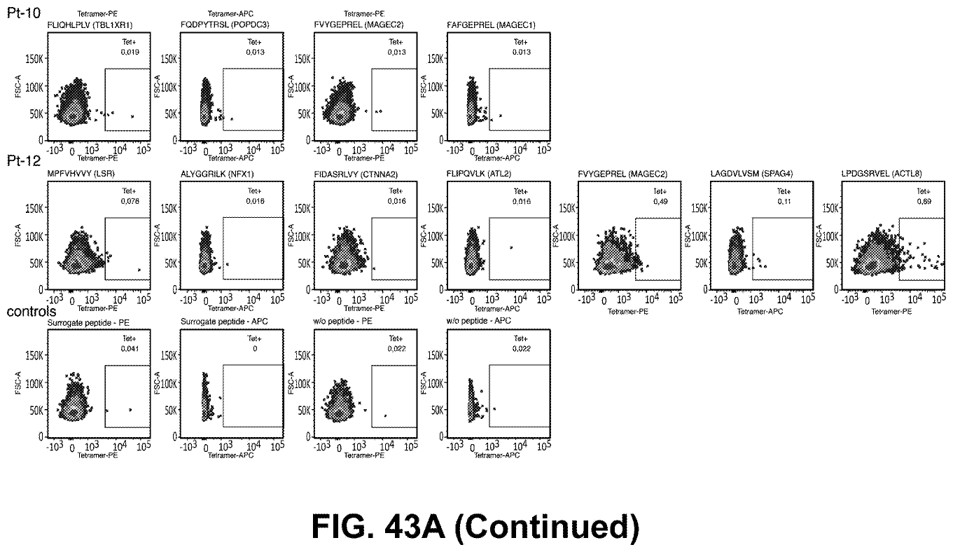

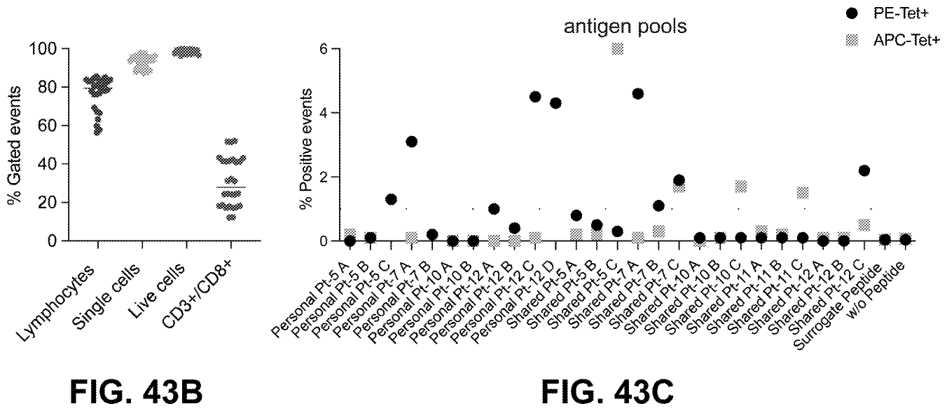

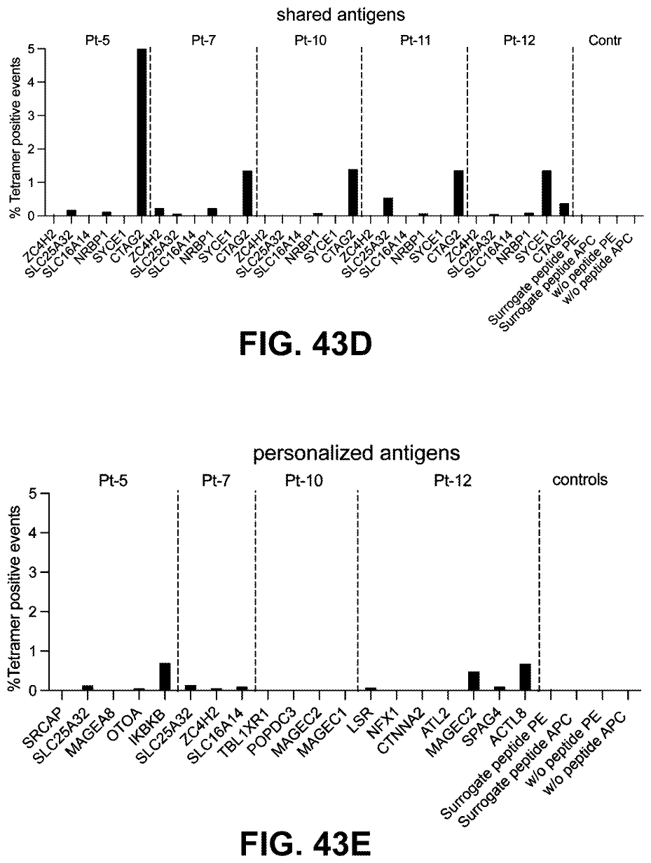

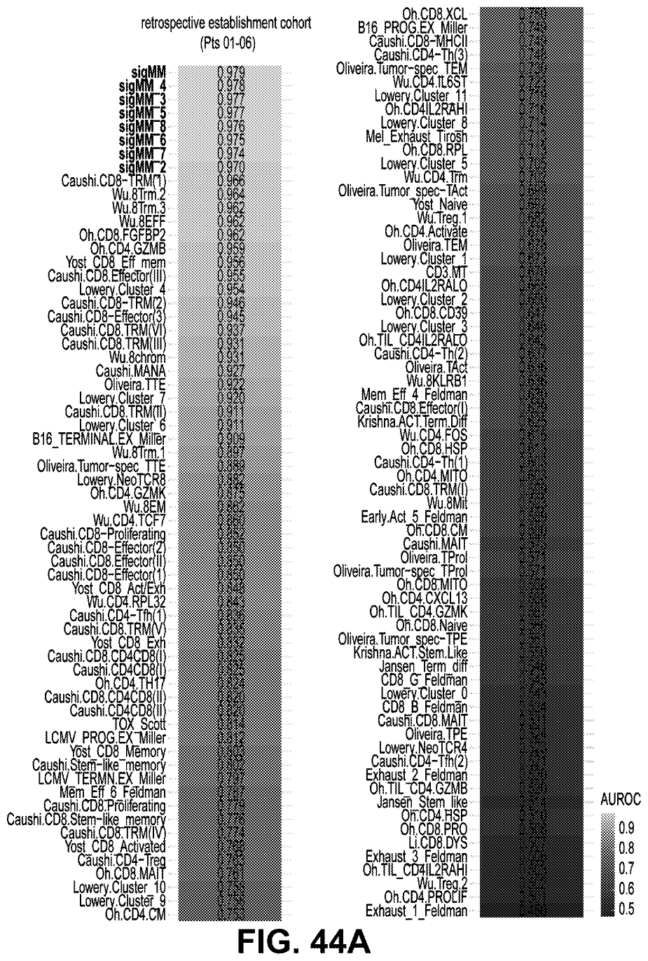

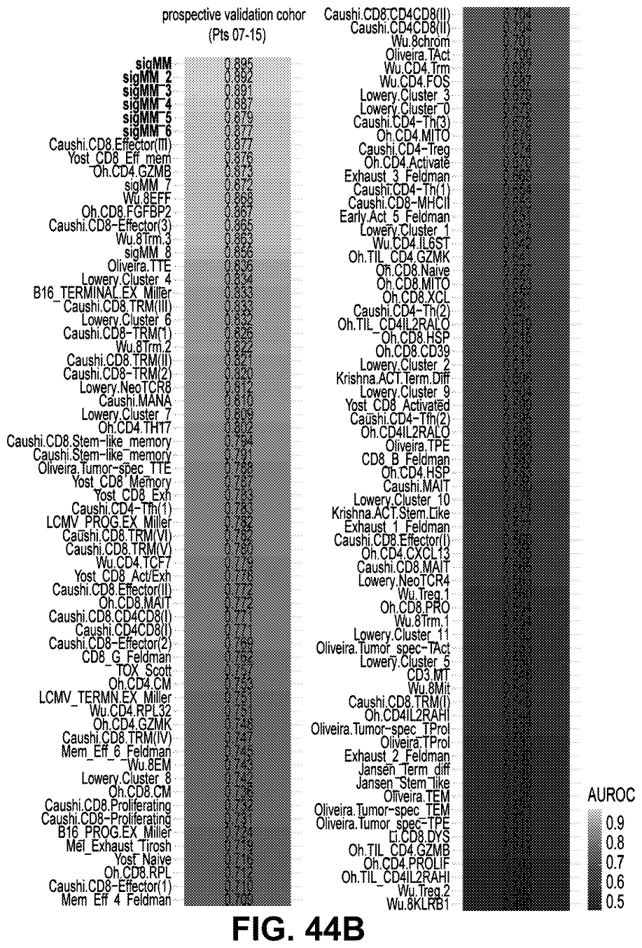

To address this question, flow cytometry was performed with MHC tetramers on bone marrow samples from patients with distinct HLA haplotypes (FIG. 41A). These tetramers were matched to each patient's HLA markup and individually loaded with the immunopeptidome-derived peptides shared by >50% of our patients (FIG. 41B) or personalized peptides (FIG. 44A). Indeed, while limited endogenous BMTC reactivity was found against personalized tumor-derived antigens, several patients demonstrated a reactivity against the same shared antigen sequences presented on MHC tetramers matched to their individual HLA (FIG. 42B-42E). The highest frequencies of peptide-specific T cells were found against tetramers loaded with CTAG2⁴⁶⁻⁴⁸ relative to the other tested shared antigens (FIG. 42D). T cells from patients of four different HLA-A supergroups were able to recognize RLLELHI™-loaded (SEQ ID NO: 128) tetramers (FIGS. 9H & 9I). Together with previous data, these findings suggest that CTAG2⁴⁶⁻⁴⁸ is an HLA-promiscuous antigen inducing frequent T cell responses and convergent TCR selection in multiple myeloma patients.

In summary, shared tumor antigen landscapes has been characterized that can be detected in several patients as early as the initial diagnosis of multiple myeloma. Shared cancer-associated antigens and convergently selected TCRs could in principle be exploited for vaccination approaches or adoptive cell therapies in this and similar malignancies with limited neoepitope loads²⁶.

### Conserved Transcriptional Signatures of Tumor-Reactive BMTCs

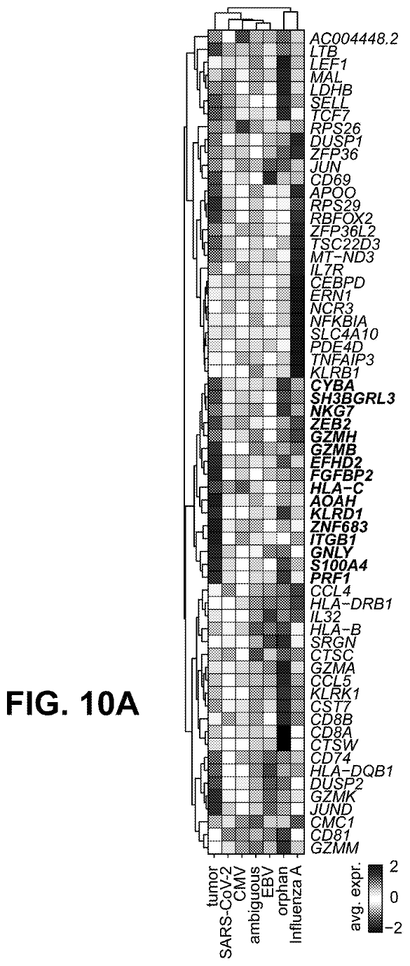

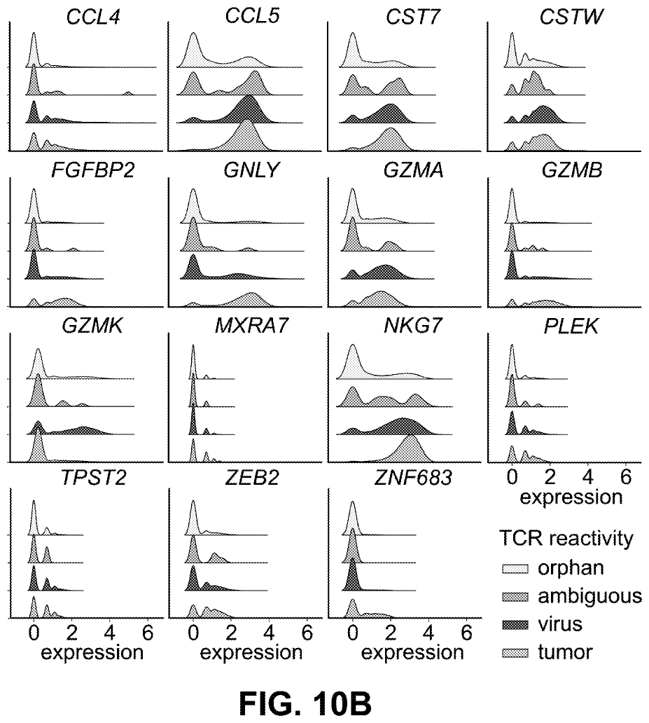

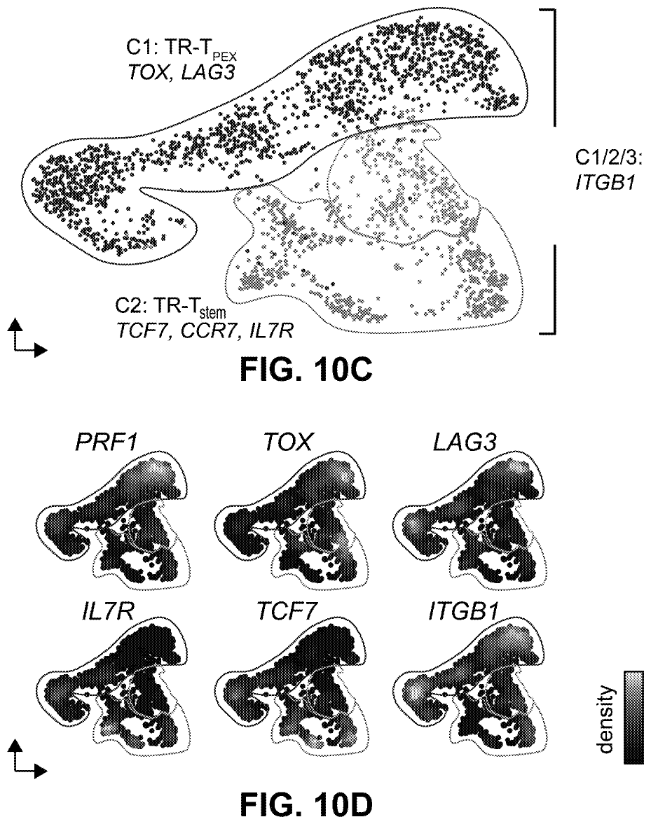

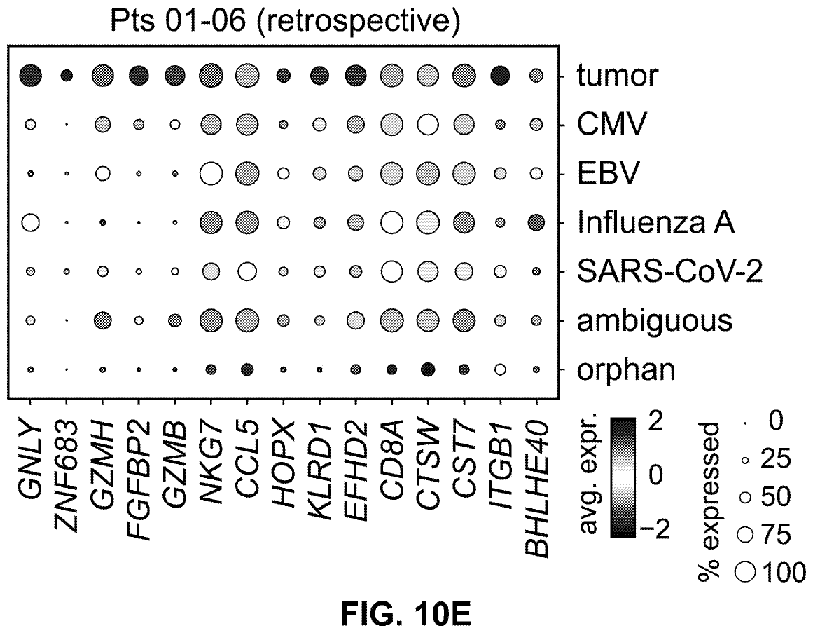

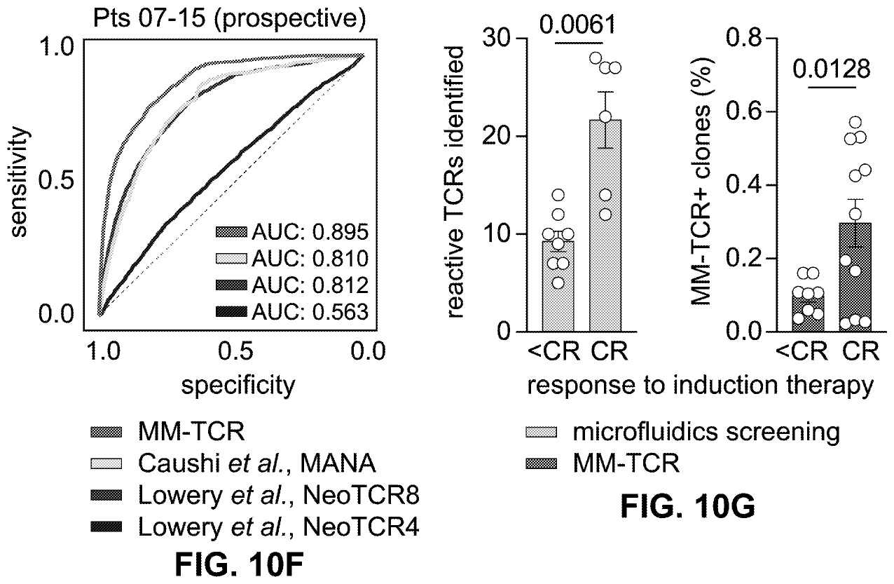

To improve understanding of the transcriptional differences among tumor-reactive BMTC, a differential gene expression analysis followed by more granular re-clustering of the 2,249 cells carrying successfully mapped tumor-reactive TCRs (FIGS. 10A & 10B) was performed. Most tumor-reactive T cells acquired an effector-memory state that was characterized by co-expression of cytotoxic molecules (GZMB, GZMH, GNLY) and markers of tissue residency (ZNF683, CCL4) (FIGS. 10A & 10B). Virus-specific TCRs demonstrated distinct expression patterns: CMV- and EBV-reactive BMTC were distinct from SARS-CoV-2 reactive TCRs and downregulated fate regulators ZBTB21²⁷ and USP22²⁸, while Influenza A-specific TCRs expressed proliferation and mitochondrial stress related genes but did not demonstrate signs of cytotoxic activity, unlike myeloma-reactive TCRs (FIG. 10A).

Through re-clustered analysis, three distinct transcriptional clusters (FIG. 10C, FIGS. 13B & 13C) were identified. These clusters were comprised of 1) tumor-reactive TR-T_{PEX} cells, which resembled CD8_PEX T cells co-expressing features of cytotoxicity (PRF1 and GZMB) with known exhaustion markers TOX and LAG3, 2) tumor-reactive TR-T_{STEM} cells, which bore similarities to memory T cells with stem-like properties with high expression of memory markers (TCF7, CCR7, IL7R) and low expression of exhaustion markers and effector cytokines and 3) an intermediate state (FIGS. 10C & 10D). Upon analyzing the cellular states in relation to TCR clonality, it was found that 61.8% of the cells derived from tumor-reactive TCR clonotypes were skewed toward tumor-reactive TR-T_{PEX} phenotypes, even though individual cells expressing a given TCR clonotype could demonstrate any phenotype (FIG. 31A). 23.5% of tumor-reactive cells adopted the tumor-reactive TR-T_{STEM} states. Thus, the activation and differentiation of anti-tumor CD8⁺ BMTC within the bone marrow microenvironment favored their accumulation as CD8_PEX cells rather than less-differentiated stem-like effectors. Consistent with this notion, it was found that clones representing >1% of TCRs in baseline biopsies were mostly found within the CD8_EM and CD8_PEX transcriptional clusters (FIG. 14D).

Based on the shared transcriptional features of all detected and successfully mapped tumor-reactive T cells in the patients, 9 gene signatures were generated using the top genes differentiating tumor—from virus-specific T cells in our cohort followed by iterative shortening followed by retrospective receiver operating characteristic (ROC) analysis on the establishment cohort's tumor-reactive TCRs. The iteration of this gene list with the highest combination of sensitivity and specificity on the training set (n=947 cells, AUC: 0.979) comprised 15 genes and was more accurate in predicting tumor-reactive TCRs in the bone marrow than other recently published MANA-derived and neoantigen-specific signatures, likely as these signatures were derived from the highly perturbed microenvironment of high TMB solid tumors that have been treated with immune checkpoint inhibitors (ICI)^{19,20,29,32,33,38,39,40} (FIG. 44A). This gene set was proceeded with and defined as a ‘MM-TCR’ signature for subsequent analyses (FIG. 10E).

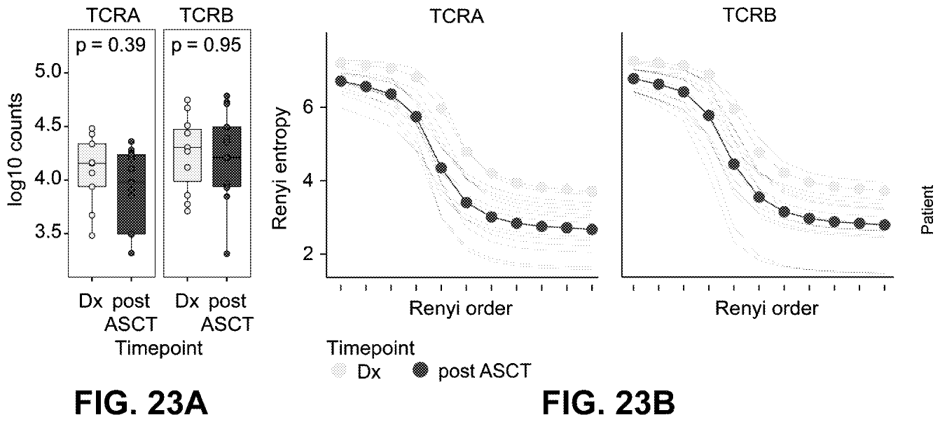

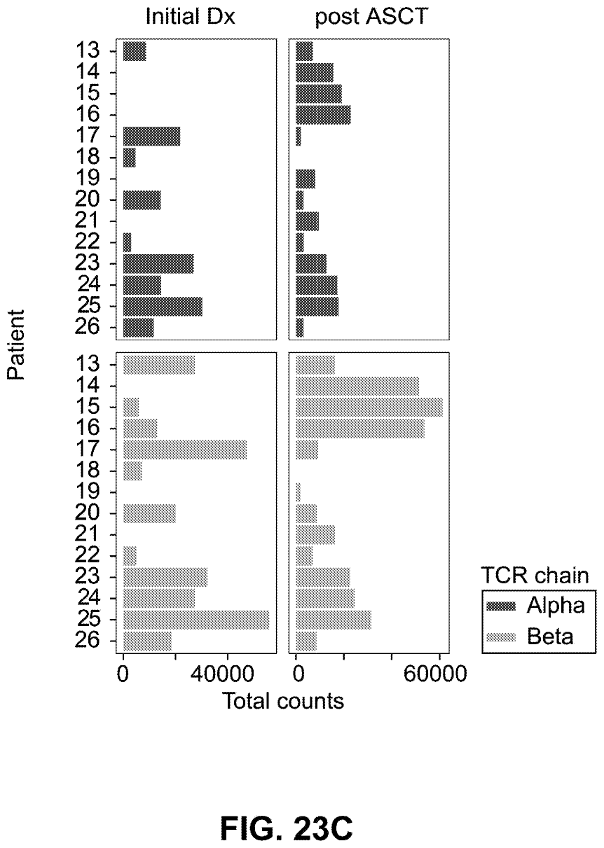

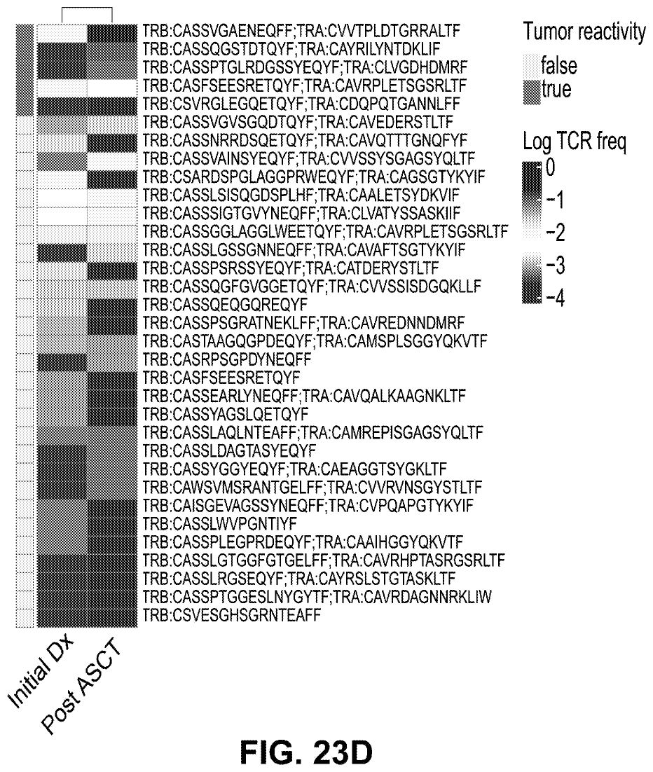

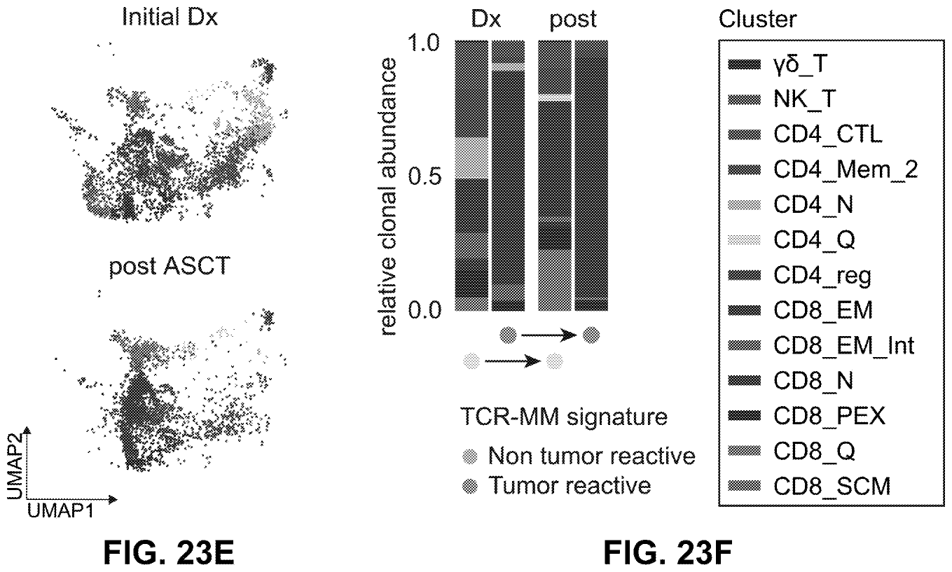

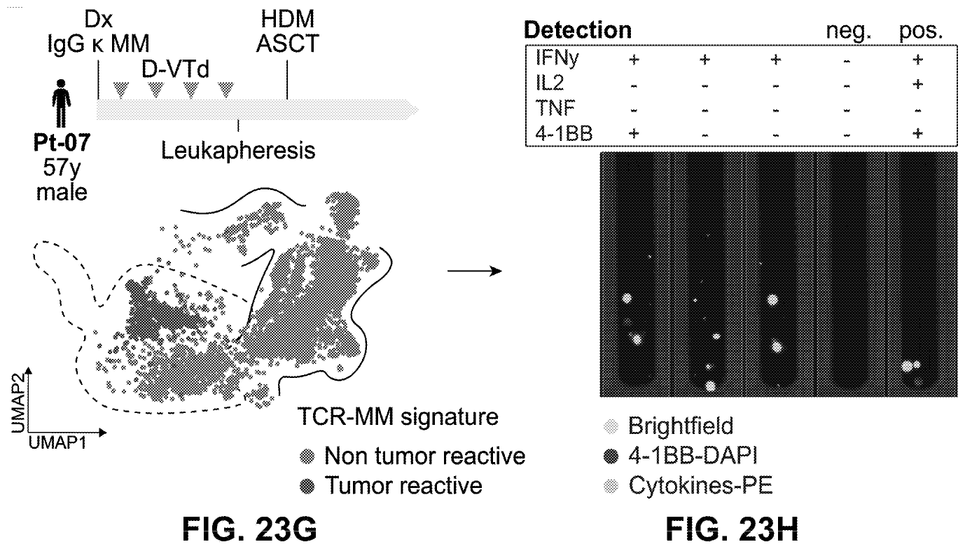

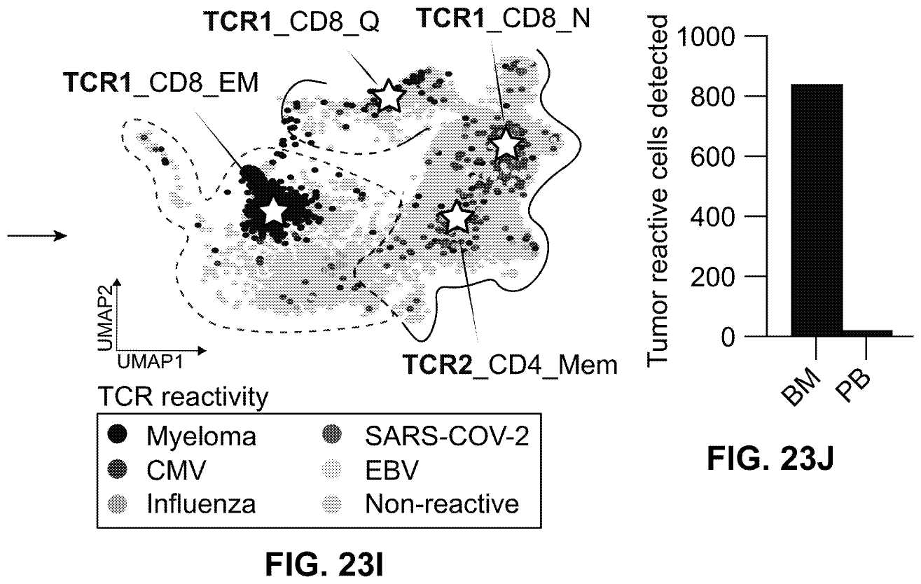

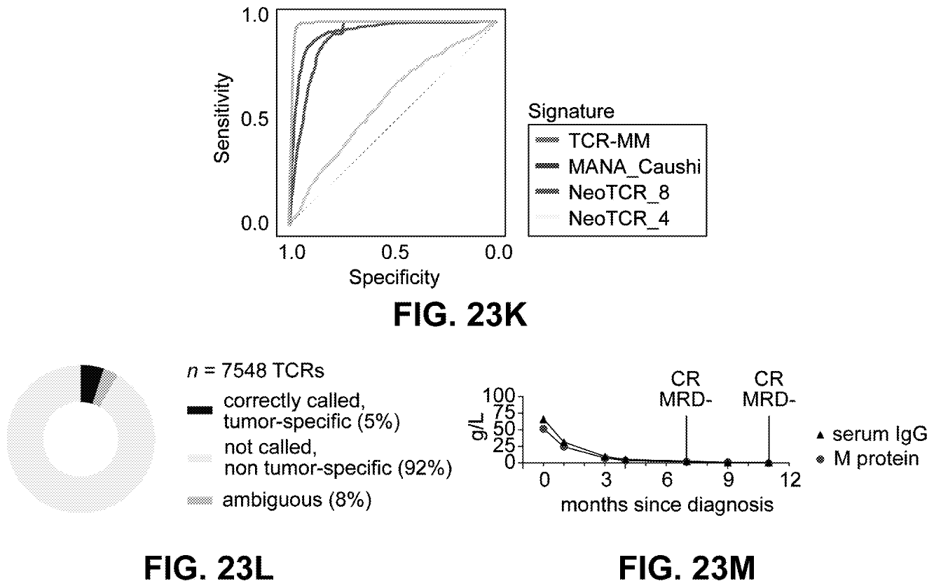

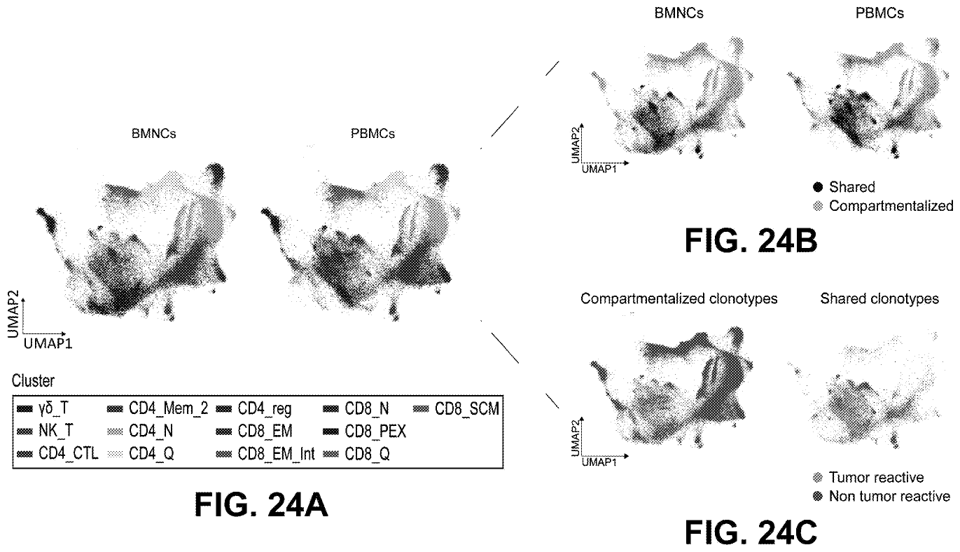

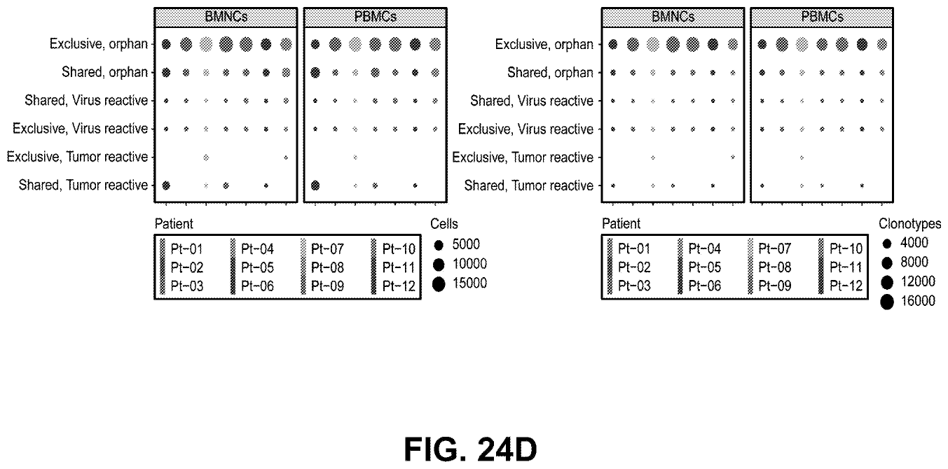

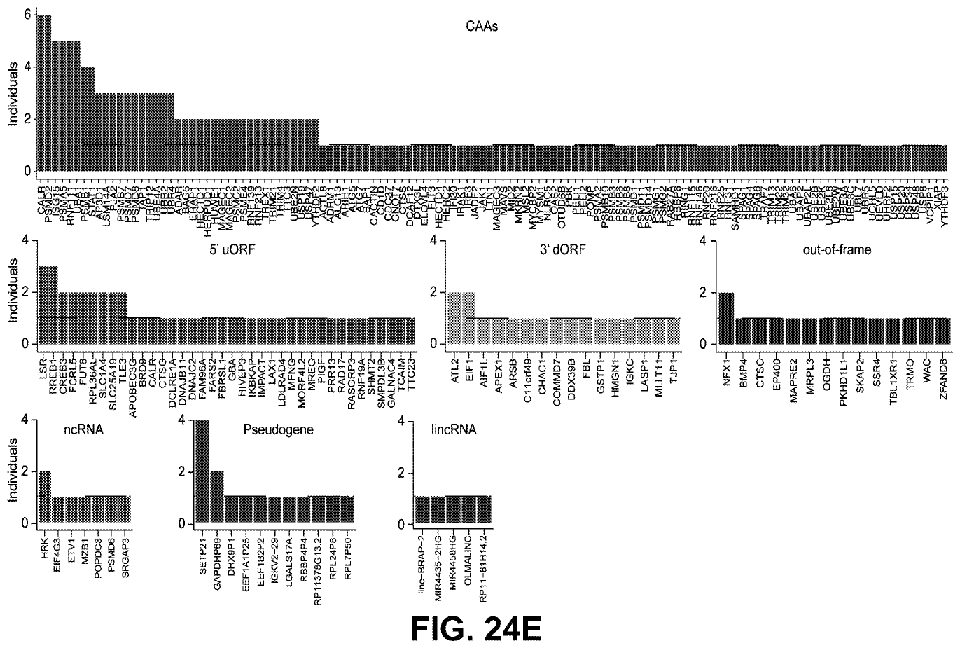

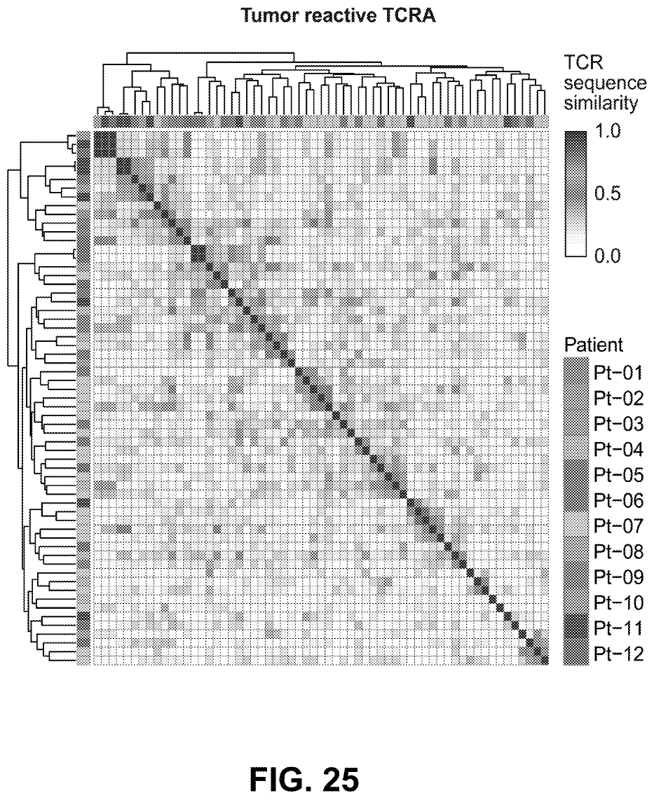

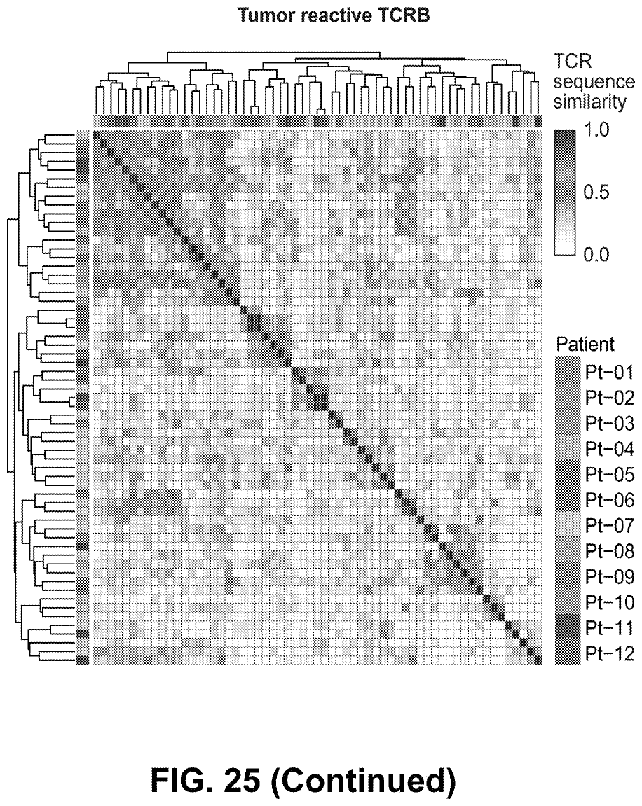

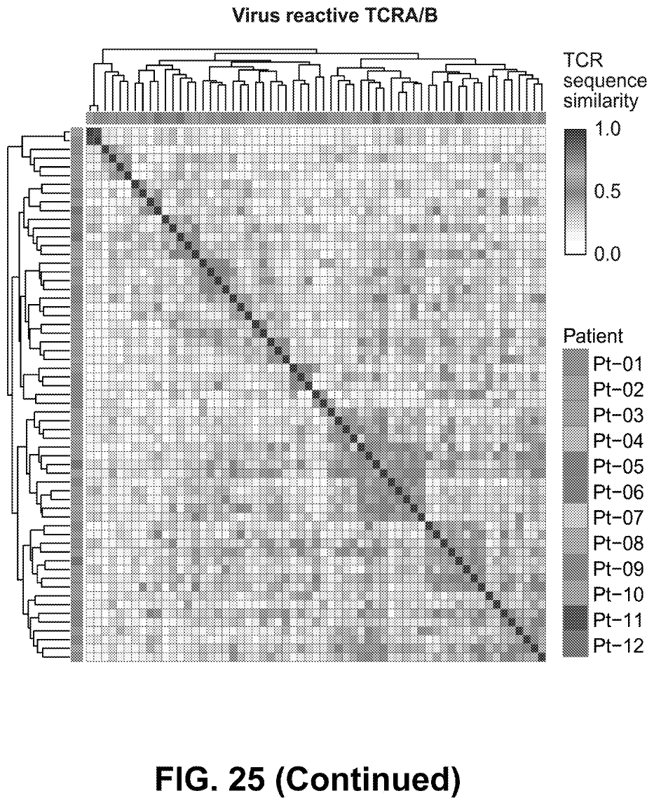

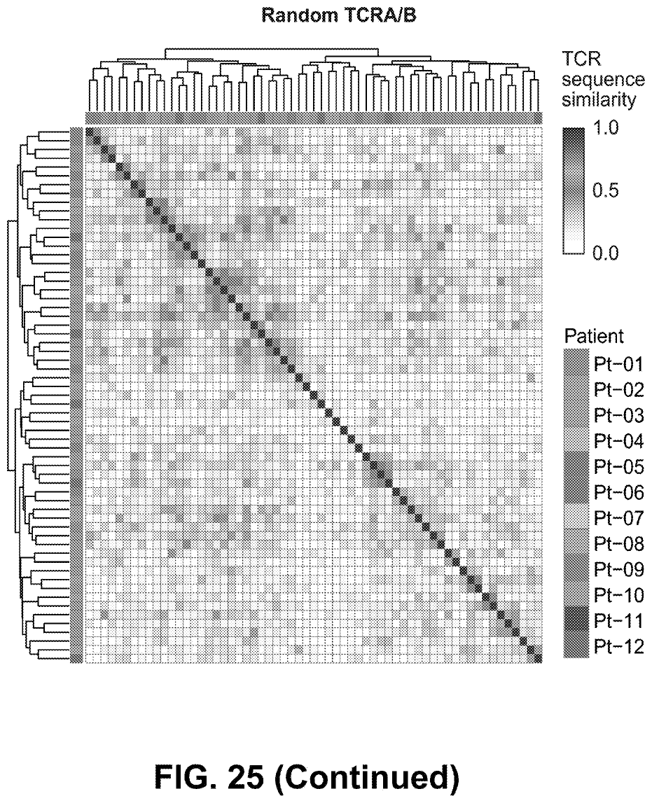

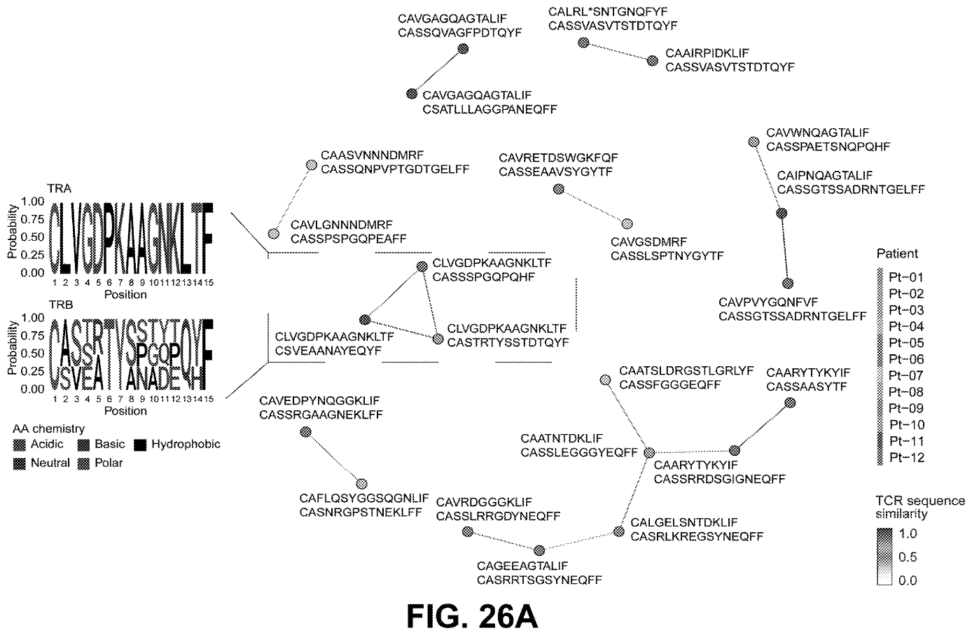

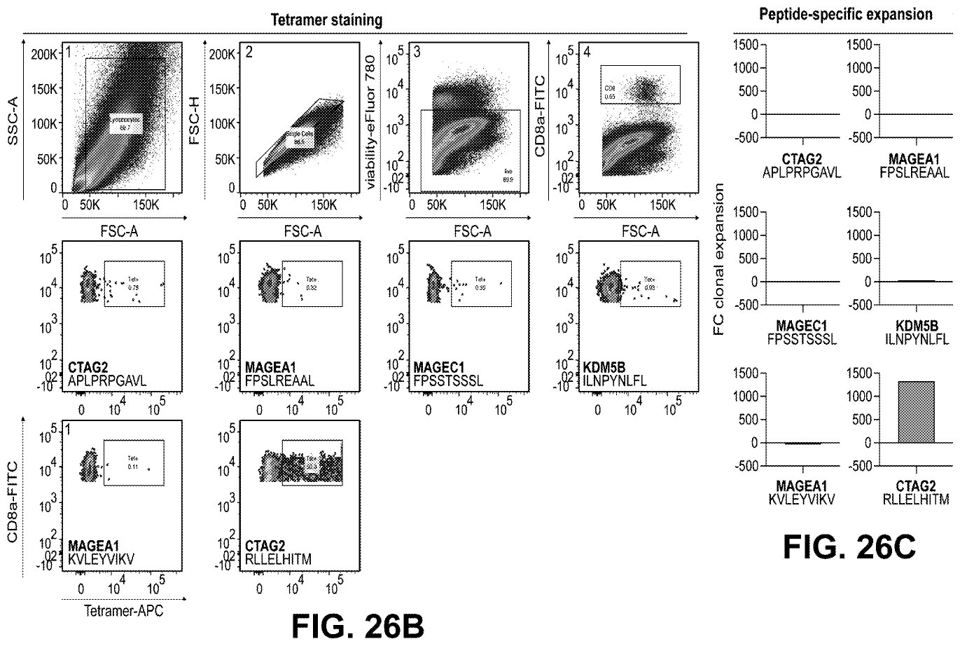

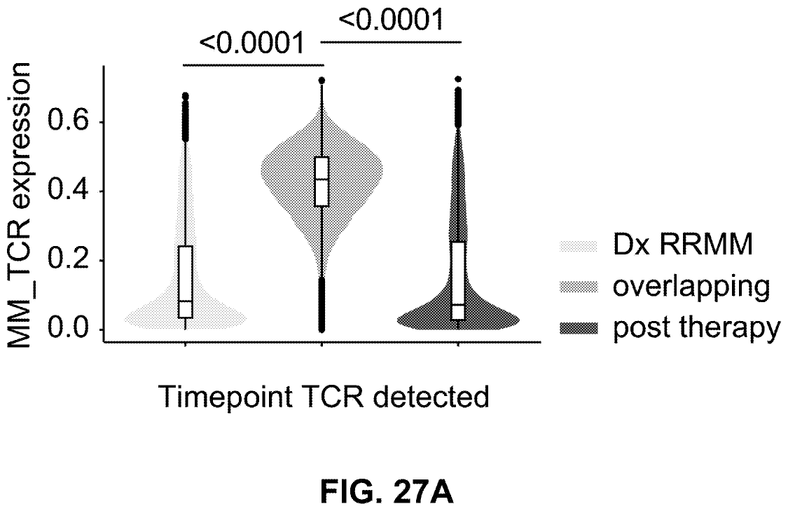

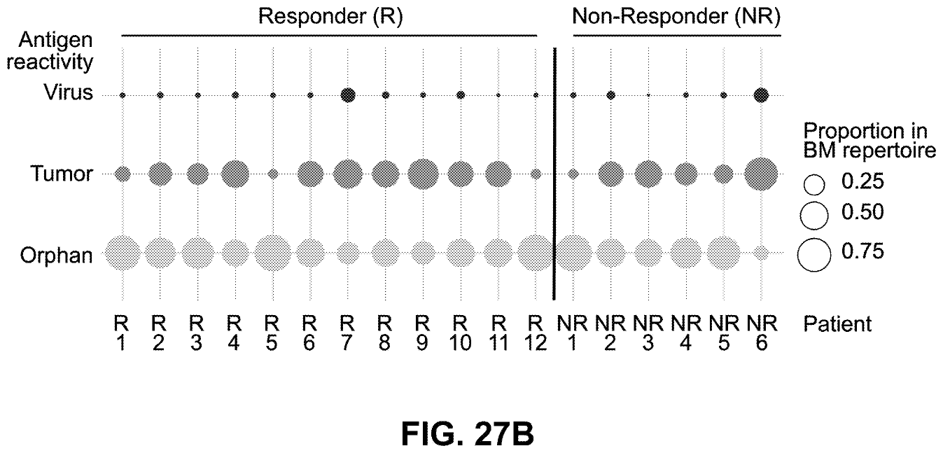

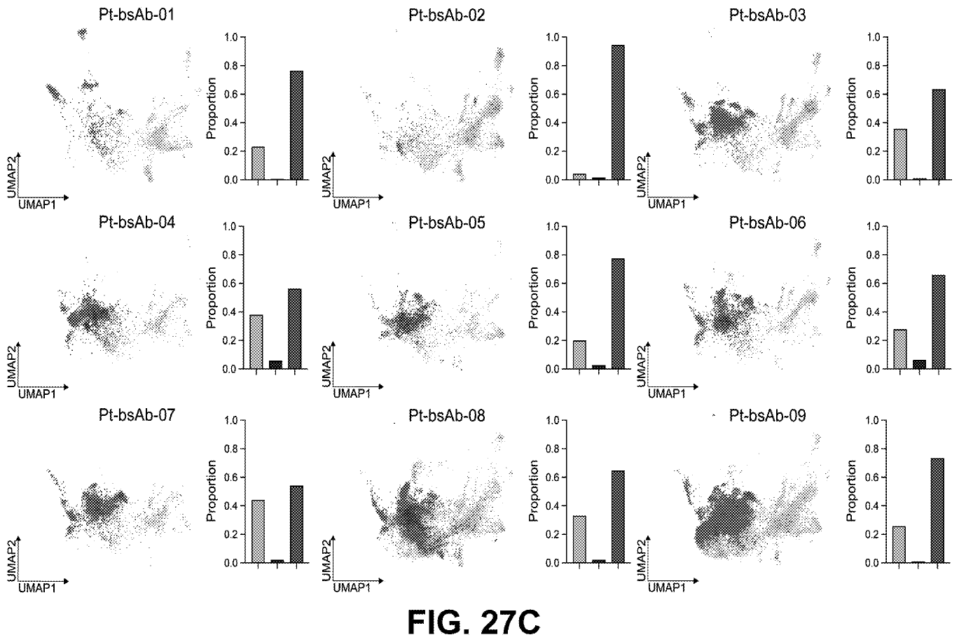

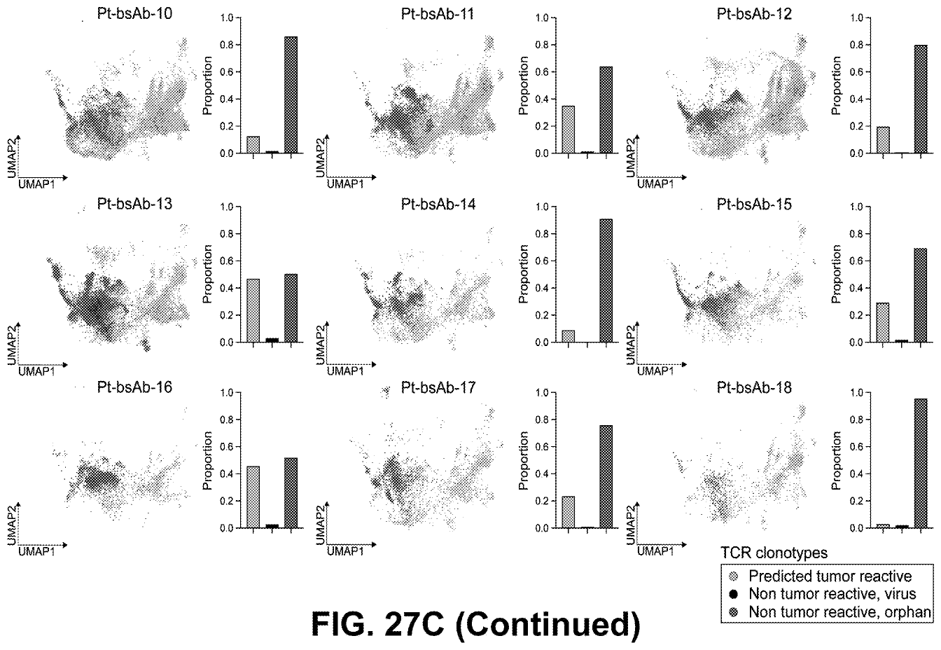

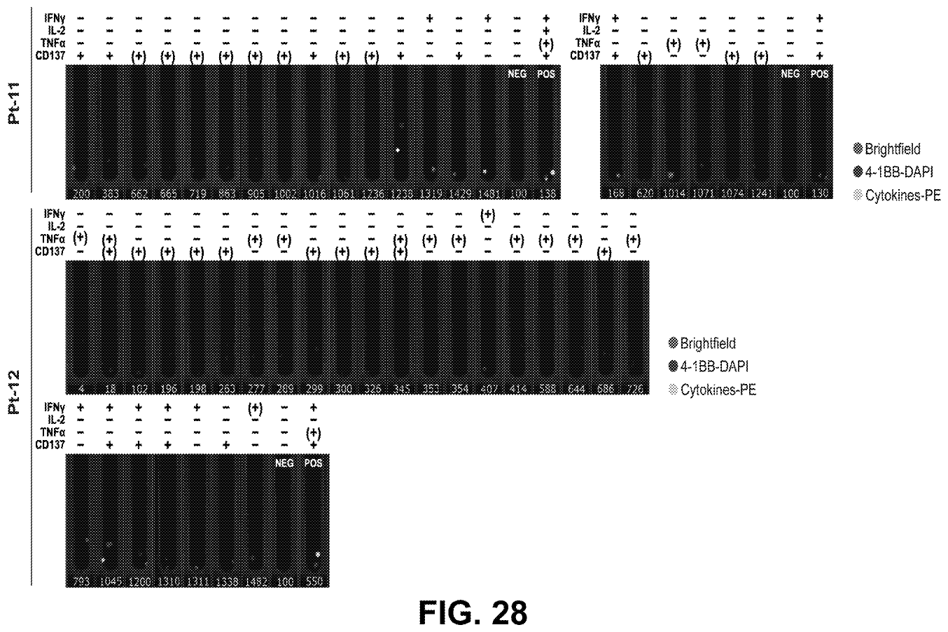

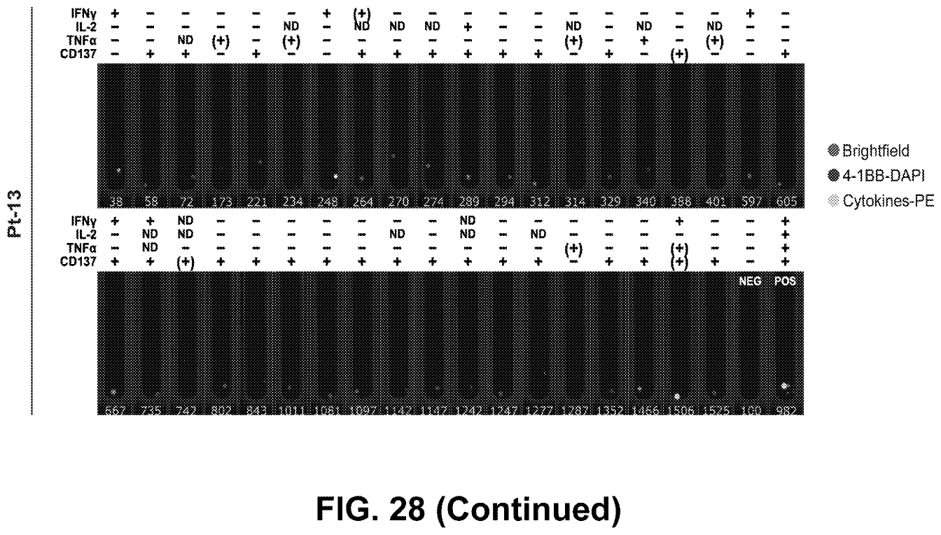

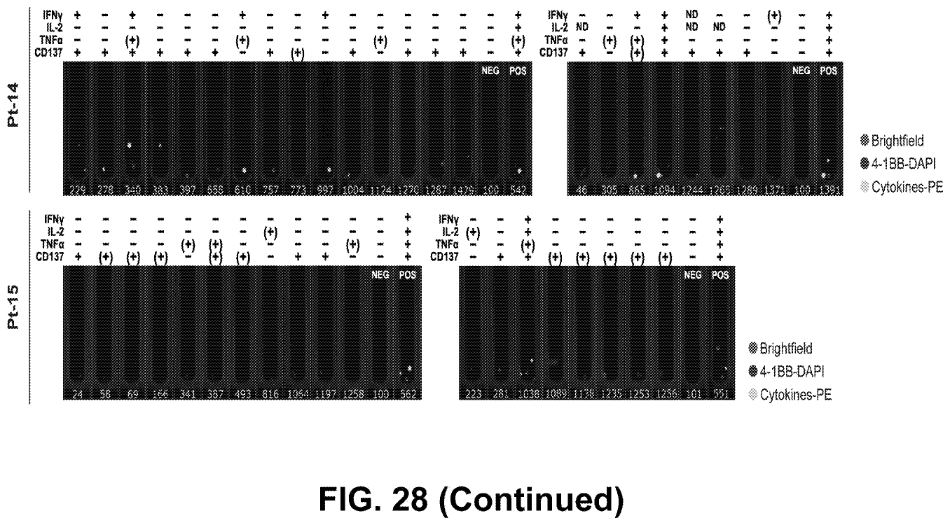

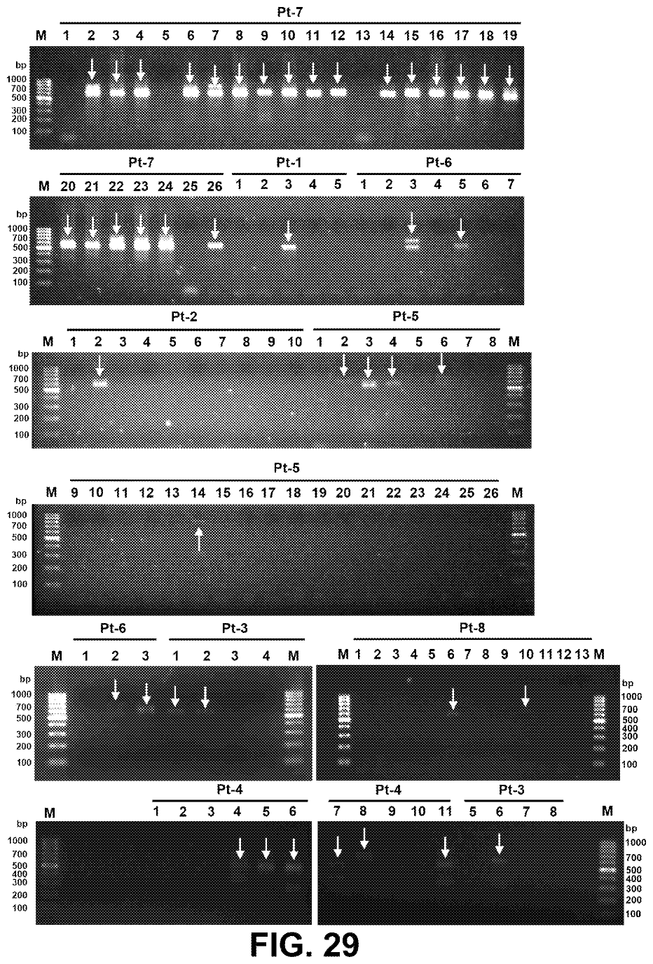

To examine whether the identified conserved transcriptional program of tumor-reactive BMTC can be used to prospectively identify TCRs targeting multiple myeloma, the predictive value of the MM-TCR signature was benchmarked to other published T cell signatures by performing a ROC analysis on individual TCRs from the independently sampled patients Pt-07 to Pt-15. These independently sampled patients were treated with current standard of care induction regimens followed by autologous stem cell transplantation (ASCT) (FIG. 9A)⁴⁹. The prospective benchmarking was performed by initial scRNA/TCR sequencing of patient bone marrow, followed by classification of all TCRs in each patient's dataset for tumor reactivity using the MM-TCR or other published TCR signatures, and finally, tumor reactivity screening of these patients' bone marrow samples to confirm whether TCRs classified as tumor-reactive were indeed activated when co-cultured with multiple myeloma cells (FIGS. 16 & 28). Of all tested signatures, the MM-TCR signature demonstrated the highest combination of sensitivity and specificity to identify tumor-reactive BMTC (n=1302 cells, AUC: 0.895) and outperformed recently published MANA-derived and neoantigen-specific signatures, such as MANA_Caushi (AUC: 0.810), NeoTCR_8 (AUC: 0.812) and NeoTCR_4 (AUC: 0.563) (FIG. 10F, FIG. 23B)^{38,39}.

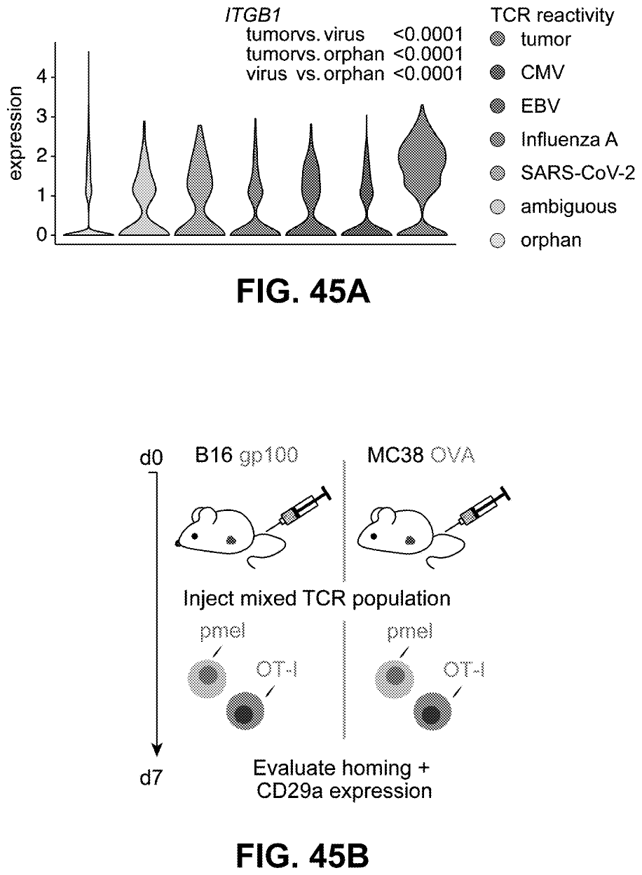

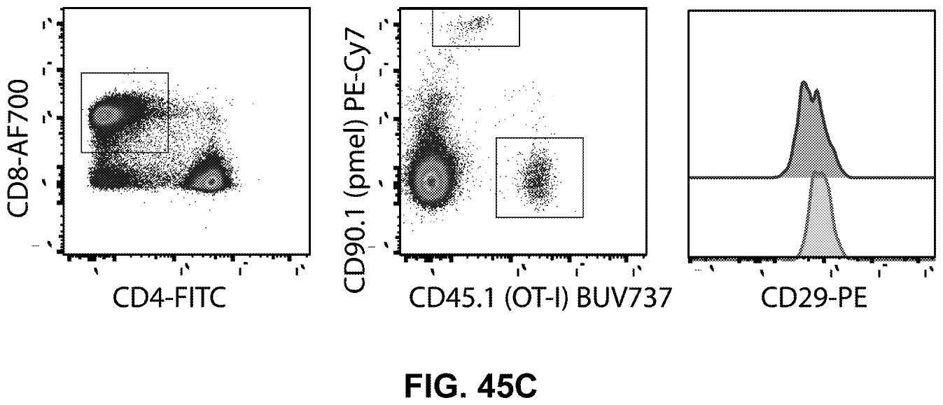

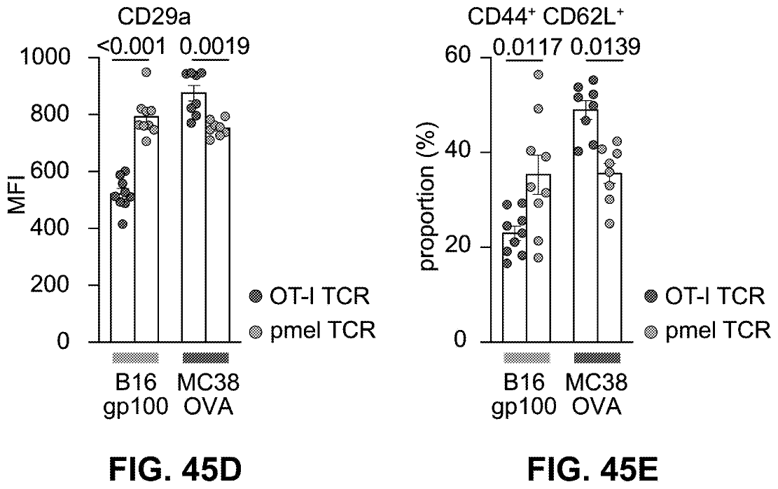

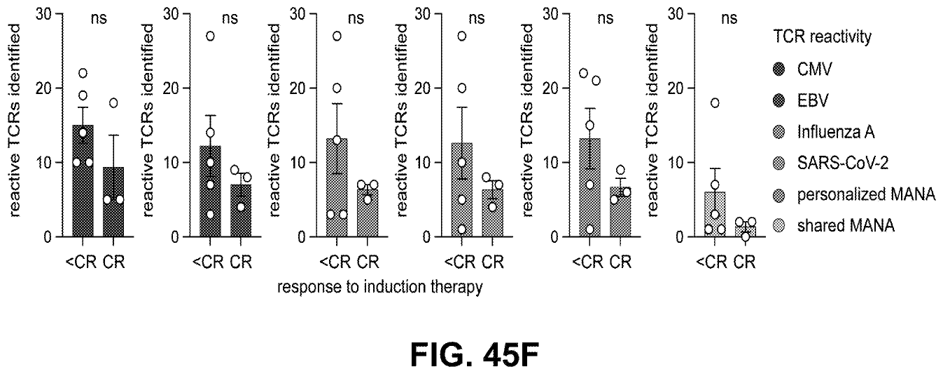

Interestingly, it was found that the ITGB1 gene, which encodes integrin beta-1, also known as CD29, was uniformly expressed by both tumor-reactive TR-T_{PEX} cells and TR-T_{STEM} cells (FIG. 16D). Previous research has indicated that CD29 surface expression is upregulated on activated T cells and involved in the formation of immunological synapses between T cells and antigen-presenting cells⁵⁰. Additionally, CD29 has been shown to be important for T cell trafficking to lymphoid organs and inflamed tissues⁵¹ and, recently, to mediate TCR activation-dependent T cell dysfunction in synergy with TOX⁵². Here, ITGB1 expression was found to be specifically restricted to tumor-reactive, compared to virus-specific BMTC (FIGS. 16A & 16E, FIG. 45A). The functional relevance of CD29 in tumor-reactive T cells was therefore investigated. To this end, naïve TCR-transgenic T cells was co-injected recognizing either relevant or irrelevant tumor antigens in tumor-bearing mice and evaluated their CD29 expression and memory differentiation (FIG. 45B). Adoptively transferred T cells were detected in blood, tumors, and tumor-draining lymph nodes (TDLN) by CD90.1 (transferred pmel T cells) or CD45.1 (transferred OT-I T cells) in wild-type C57BL/6J mice carrying subcutaneous tumors. In these two independent tumor models (B16 gp100 and MC38 OVA), it was found that CD29 protein was significantly upregulated on homed T cells carrying the tumor-reactive TCR relative to the non-reactive TCR (FIGS. 45C & 45D). This was accompanied by a significantly increased proportion of memory cells from tumor-reactive clonotypes compared to non-tumor-reactive clonotypes in TDLN in both models (FIG. 45E). Together, this data suggests that in multiple myeloma patients and immunocompetent mouse models, tumor-associated T cells express CD29 protein as a functionally relevant component of the observed tumor reactivity signature.

### Clinical Relevance of Tumor-Reactive TCRs in Multiple Myeloma

Tumor-reactive T cells were found to recognize shared or private tumor antigens expressed by multiple myeloma cells. The link between tumor-reactive T cells and clinical response to multiple myeloma would be strengthened by finding that the abundance of these cells correlates with clinical outcomes. Previously published signatures of tumor-reactive TCRs in solid tumors have not been demonstrated to be applicable to deciphering clinically relevant TCR dynamics, likely due to the impracticability of repeated tumor sampling and because prior studies have exclusively focused on tissue that has been exposed to immunotherapy^{6,32,38,39,40} Therefore the number of tumor-reactive T cells present at the time of initial diagnosis was examined and its correlation with the patients' response to induction therapy based on the IMWG Uniform Response Criteria^{53,54} was examined. In line with previous studies in solid tumors, serological and radiographic responses were not associated with the prevalence or frequency of MANA-specific T cells. In fact, the observed frequency of viral-specific T cells in bone marrow was associated with inferior clinical responses (FIG. 45F)³⁸. However, whenever microfluidic tumor reactivity screening was conducted, a significant association between initial bone marrow abundance of tumor-reactive TCRs were discovered and clinical response to induction therapy. Patients with a higher count of tumor-reactive T cells in their bone marrow upon diagnosis displayed a notably deeper therapy response. For those who achieved a complete response (CR), an average of more than 21 tumor-reactive events per screen (FIG. 10G) were observed. Similarly, a high frequency of pre-existing BMTCs expressing the MM-TCR transcriptional signature above threshold was predictive of achieving CR upon induction (immuno-) chemotherapy (FIG. 10G). Without being bound by theory, it was postulated that the clinical response in multiple myeloma might be associated with the abundance of tumor-reactive TCRs in a preserved functional state (as identified by forward screening). It was next hypothesized that this association would be the strongest with active immunotherapies such as bispecific T cell engagers (bsAb), as these depend on functional T cell responses to achieve clinical benefit⁴⁵. Prospectively inferring the target specificities of those T cells activated by bsAb could inform the development of strategies aiming to optimize long-term therapeutic efficacy while concurrently mitigating T-cell-mediated adverse effects.

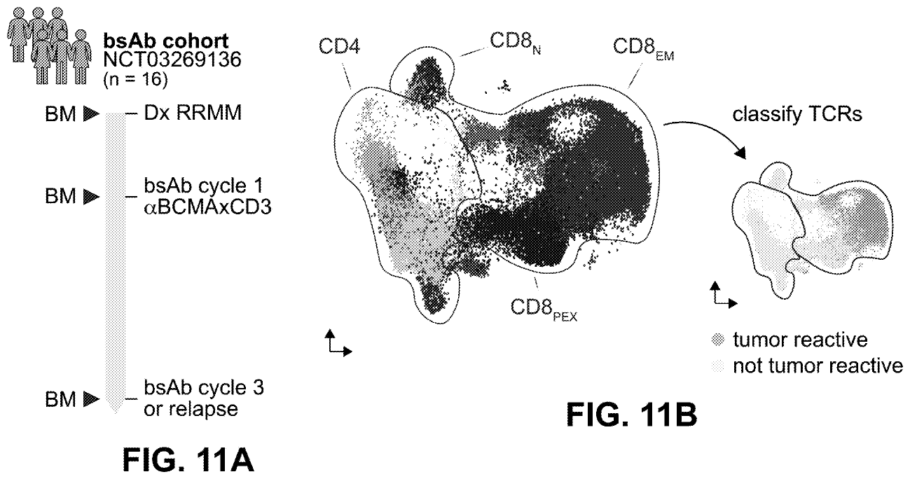

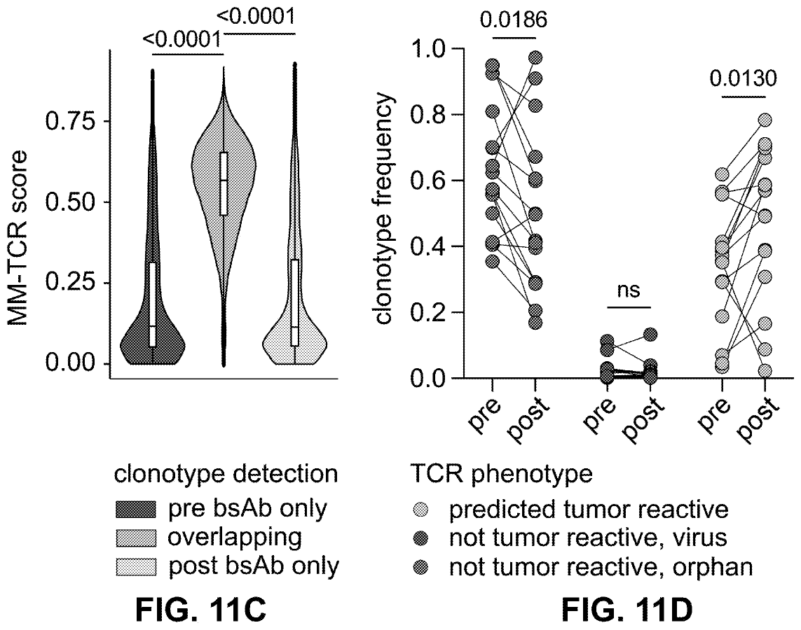

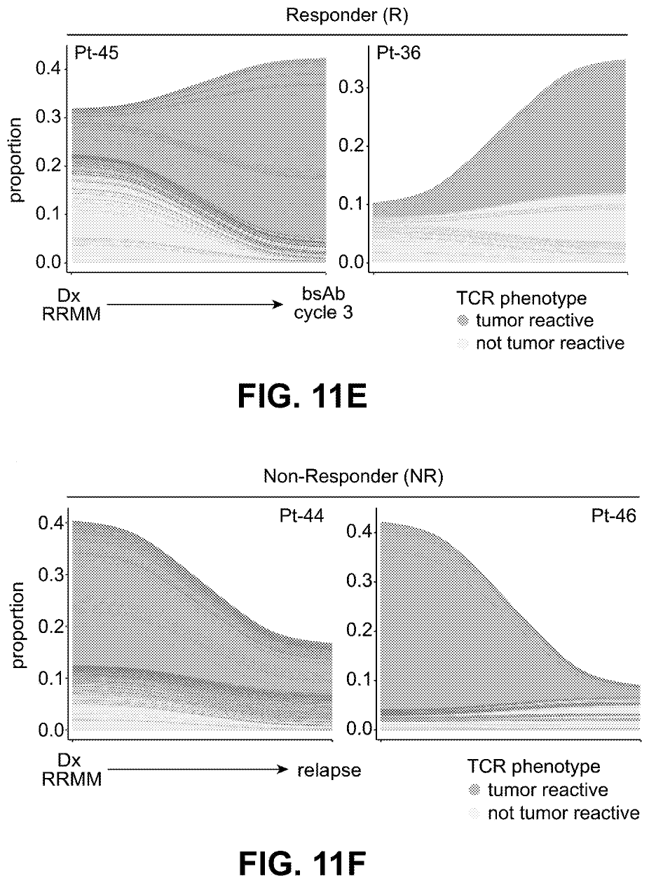

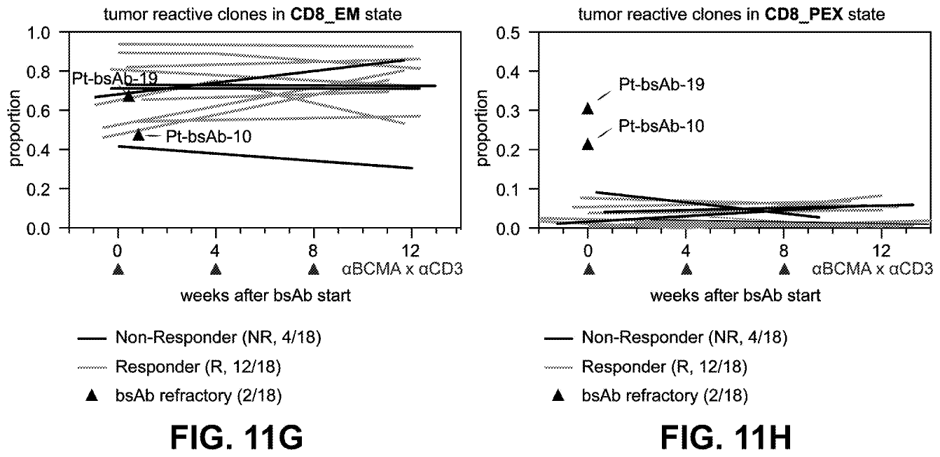

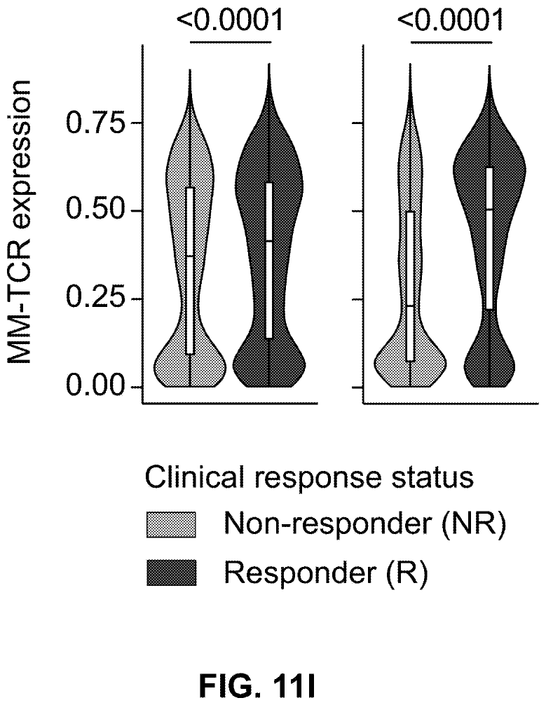

To explore the prognostic relevance of our signature and to understand bsAb-induced T cell activation in the bone marrow, serial bone marrow biopsies were performed before treatment start, post cycle 1 (approx. after 30 days) and post cycle 3 (approx. after 90 days) or at relapse in a cohort of 18 patients with relapsed/refractory multiple myeloma (RRMM) on treatment with bispecific antibodies (bsAb) and subjected the isolated T cells to combined scRNA/VDJ-seq (FIG. 11A). This clinical trial cohort was treated with BCMAxCD3 bsAb monotherapy, as previously reported (ClinicalTrials.gov identifier: NCT03269136^{55,56}). This resulted in a dataset of 143,472 BMTC across the three sampling timepoints (FIG. 11B). Frequencies of T cell clones, classified according to their antigen specificity, were measured among tissue-resident T cells through sequencing of TCR α/β chains in timepoint-matched bone marrow biopsies. Following classification using the MM-TCR signature, TCRs with a tumor reactivity phenotype in all RRMM patients were identified irrespective of other clinical parameters, though in varying frequencies, and furthermore found that clonal overlap between timepoints and clonal expansion in response to bsAb therapy were largely limited to predicted tumor-reactive, but not virus-specific or orphan TCRs (FIGS. 11C & 11D).

Finally, the relationship between the levels of bone marrow tumor-reactive T cells and clinical outcome was explored by analyzing the longitudinally available tissue in 16 patients of this clinical trial cohort. When tracing the resulting clonotypes over time, it was found that, in clinically responding patients, tumor-reactive clonotypes demonstrated clonal expansion in response to bsAb treatment (FIG. 11E). Conversely, tumor-reactive TCR clones remained stable in abundance or contracted on-therapy in clinical non-responders (FIG. 11F). Consistent with previous analyses, tumor-associated CD8⁺ TR-TEM were stable and predominant among tumor-reactive clonotypes in most of the analyzed patients (FIG. 11G). Conversely, TR-T_{PEX} were quite rare, but persisted at levels that correlated with clinical outcome; two patients who were refractory to bsAb treatment within 1 month displayed higher levels of tissue-resident TCRs in a CD8_PEX state (FIG. 11H). These dynamics mirrored the previously described proportions of exhausted-like T cells within the relapsed/refractory multiple myeloma immune repertoire, highlighting how the balance of functional tumor-reactive versus exhausted clonotypes can potentially identify patients with response versus resistance to bispecific antibody therapy⁴⁵. Finally, T cells of patients who experienced disease progression expressed lower levels of the MM-TCR signature of tumor reactivity relative to clinical responders to bsAb therapy, an effect that was pronounced post therapy (FIG. 11I).

Taken together, these data indicate that the MM-TCR signature can be used to prospectively identify tumor-reactive TCRs in multiple myeloma patients with high sensitivity and specificity. It was further observed that their abundance is associated with clinical responses to anti-myeloma treatments at therapy induction and in relapse.

### Transfer of Tumor-Reactive T Cell Clones by Autologous Stem Cell Transplantation

Current standard treatment in transplant-eligible multiple myeloma patients consists of induction (immuno-) chemotherapy followed by ASCT^{57,58}. The benefit of this approach lies in tumor reduction and regeneration of a functional immune system, thereby increasing the depth and duration of an antitumor response⁵³. Although induction therapy and ASCT frequently result in sustained clinical responses in hematological malignancies, these have not been linked to the presence of endogenous tumor-reactive T cells. The potential clinical relevance of tumor-reactive TCRs in induction therapy and ASCT was therefore explored.

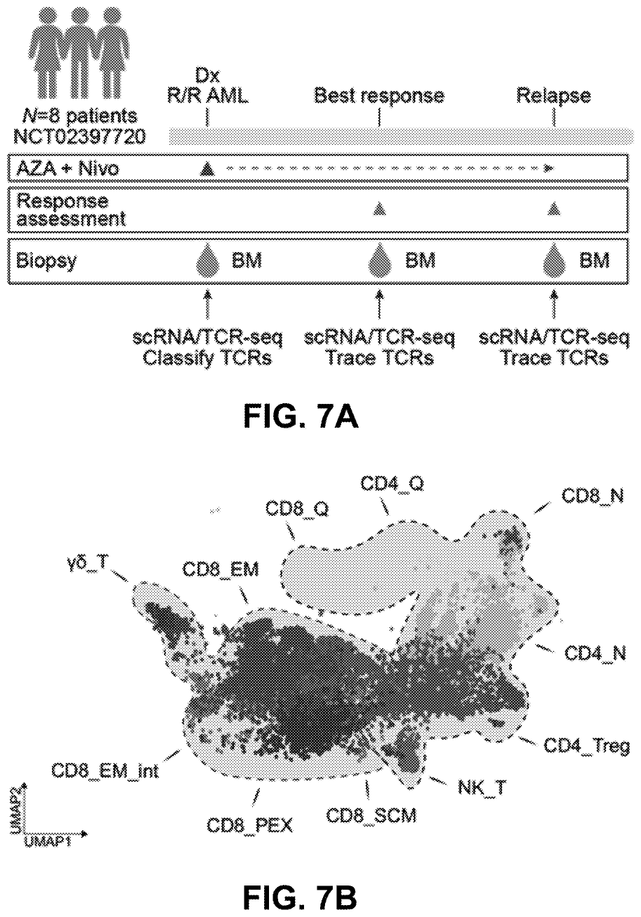

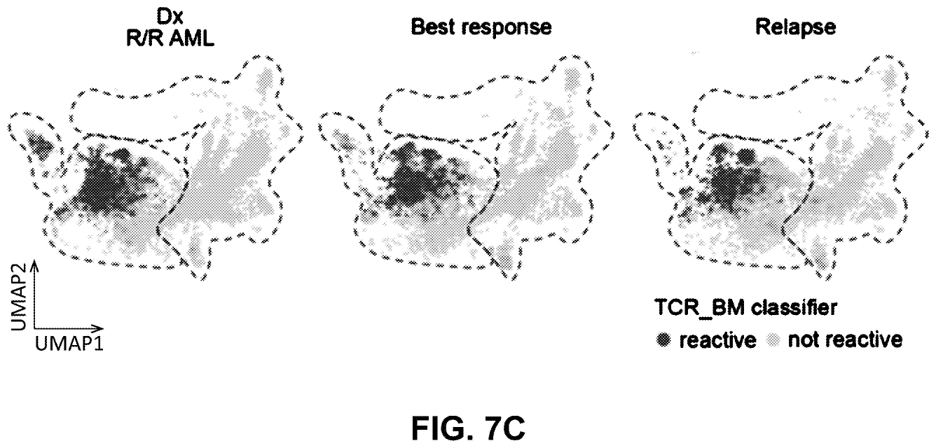

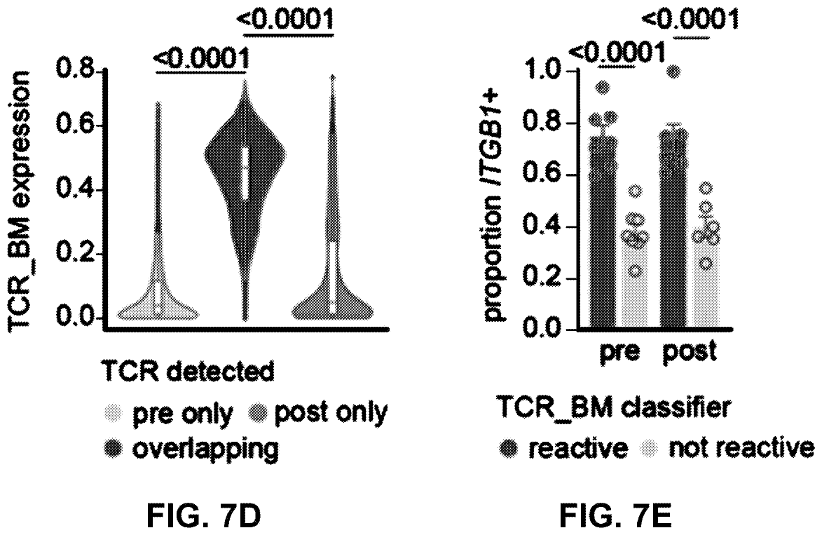

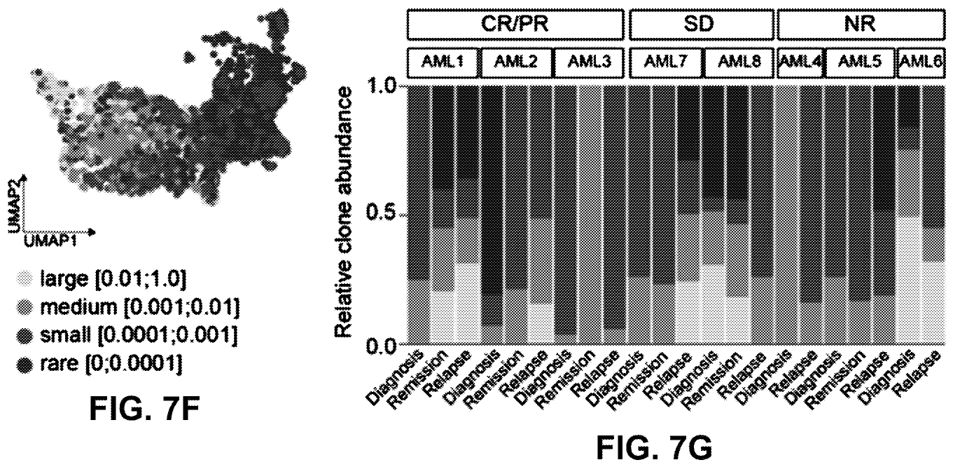

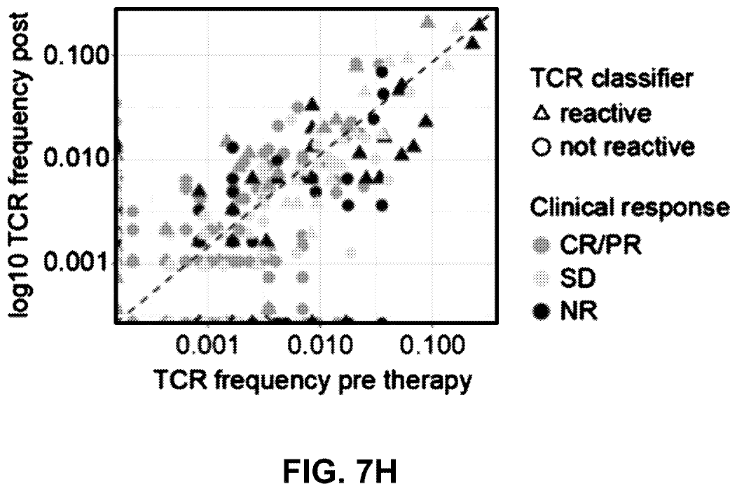

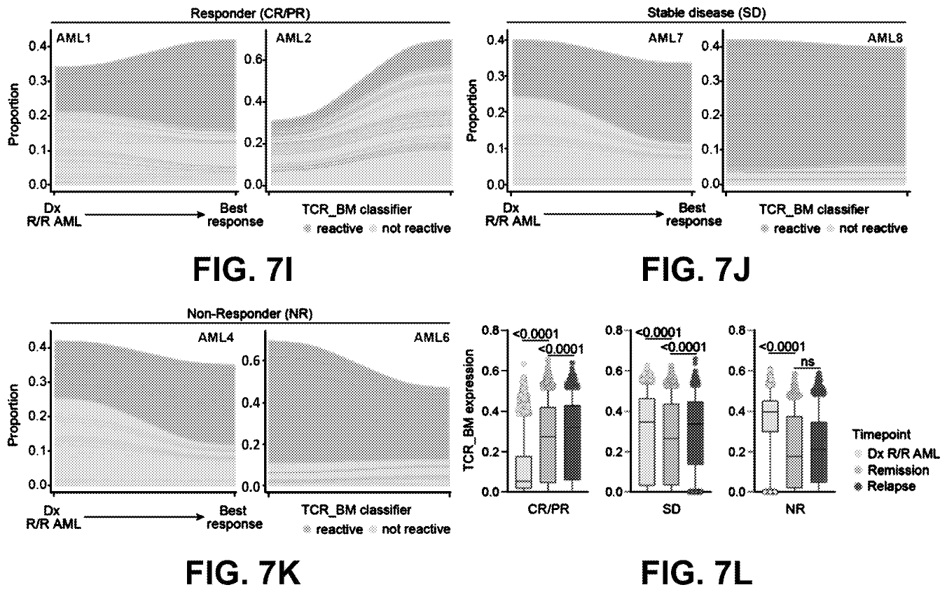

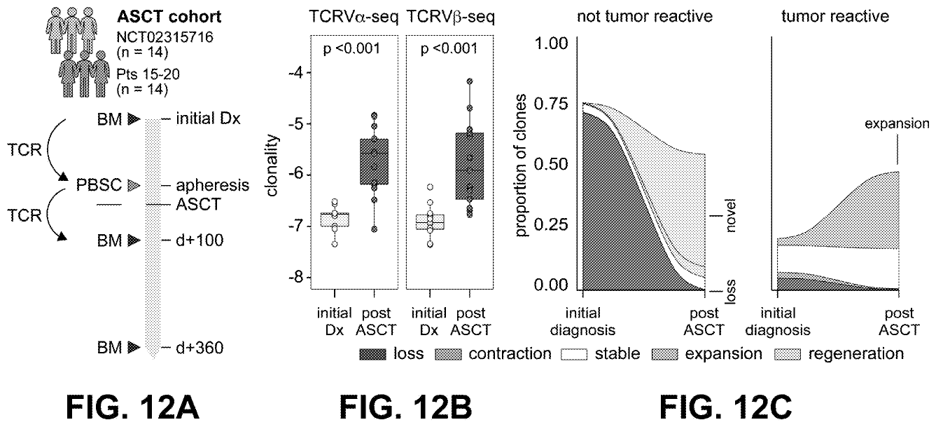

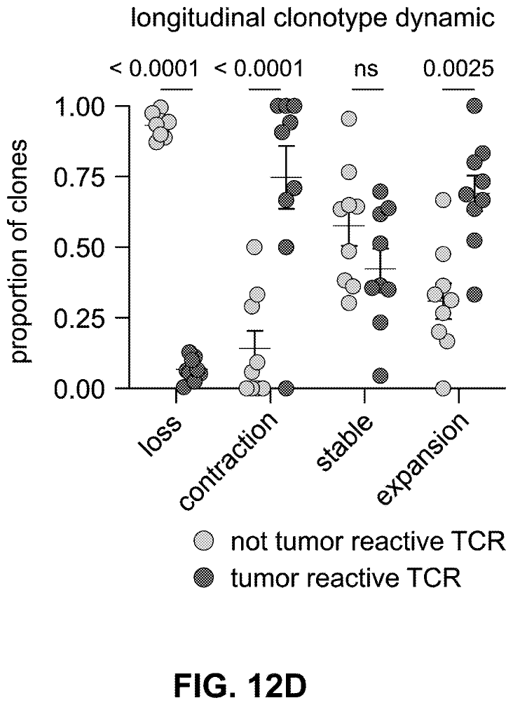

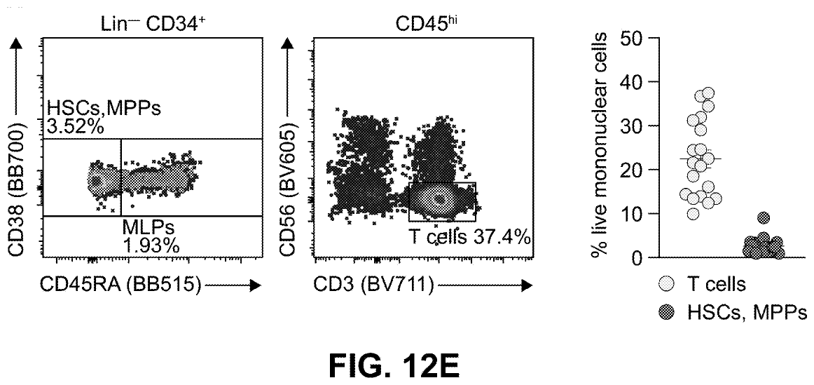

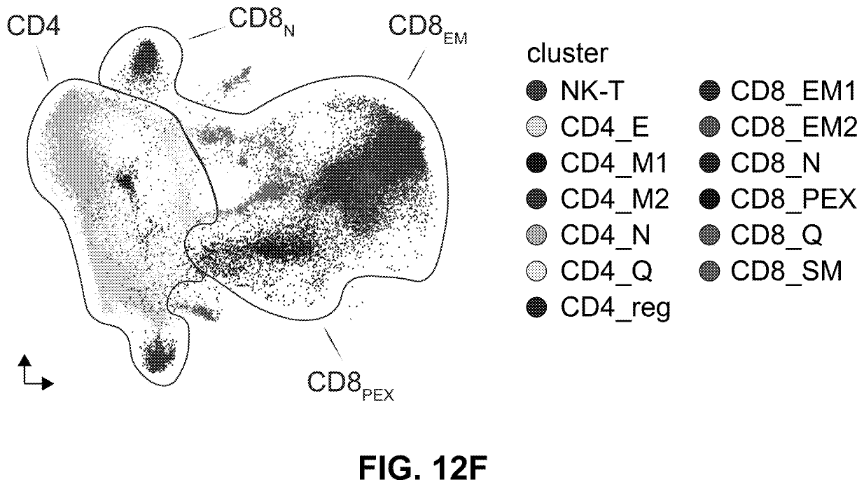

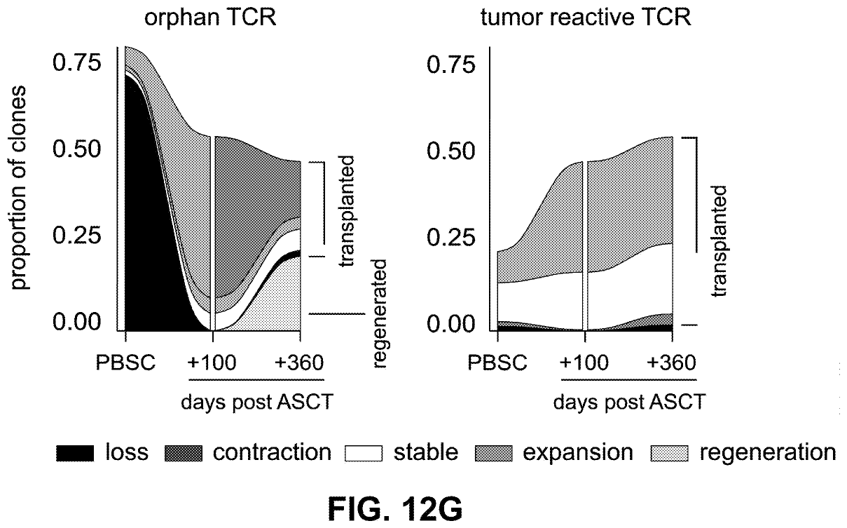

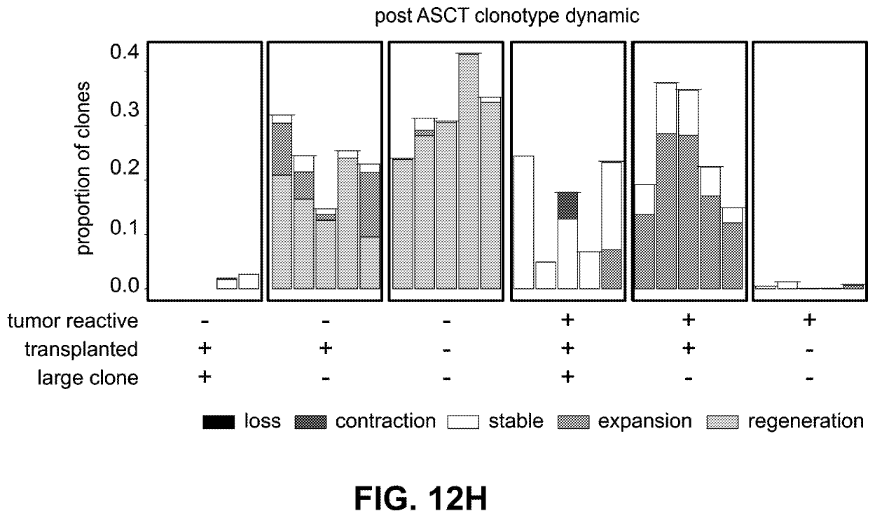

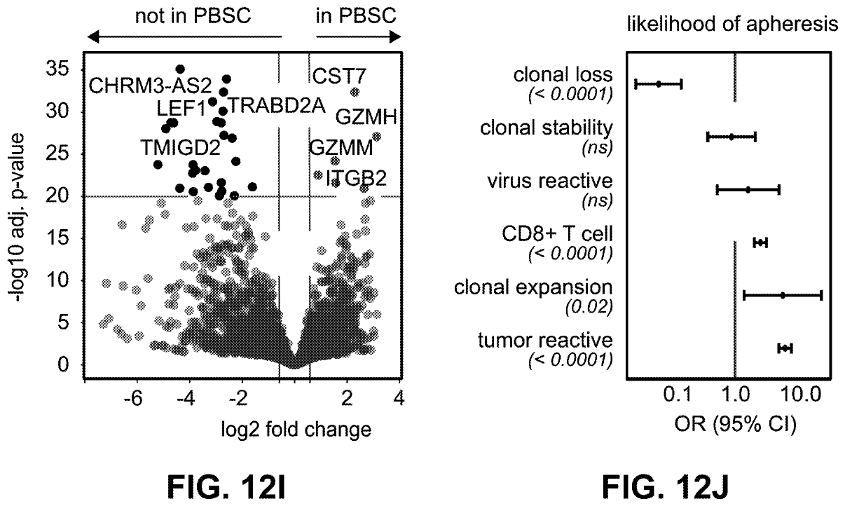

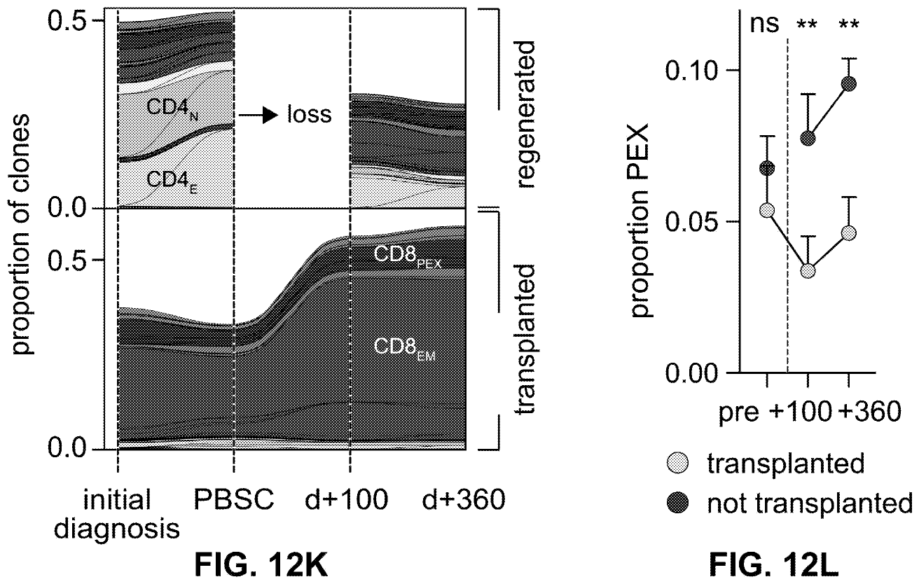

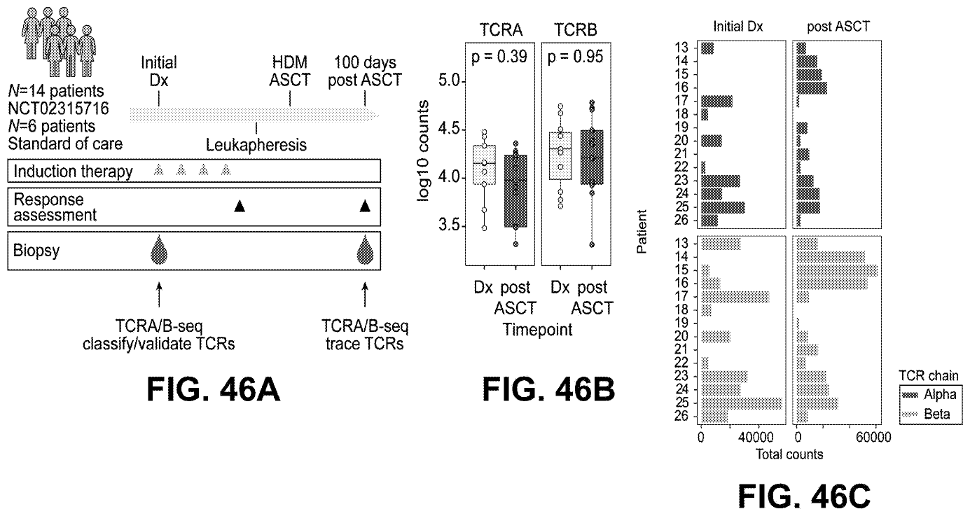

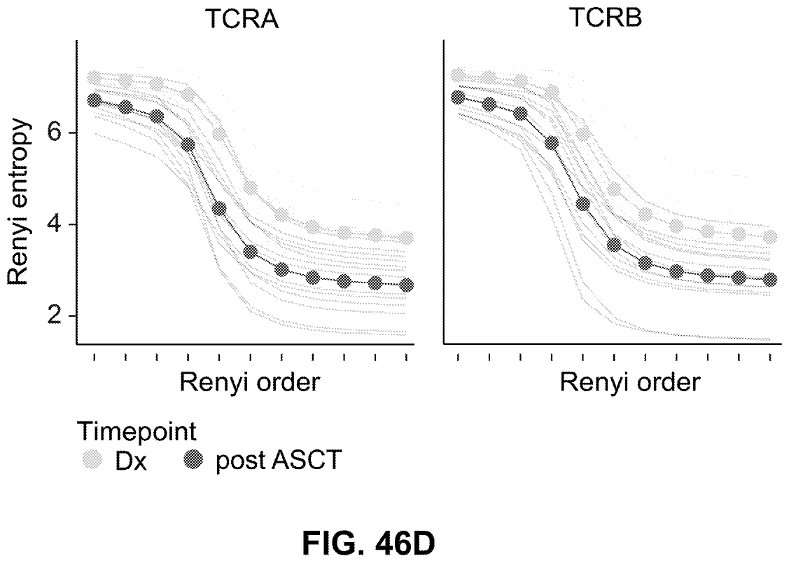

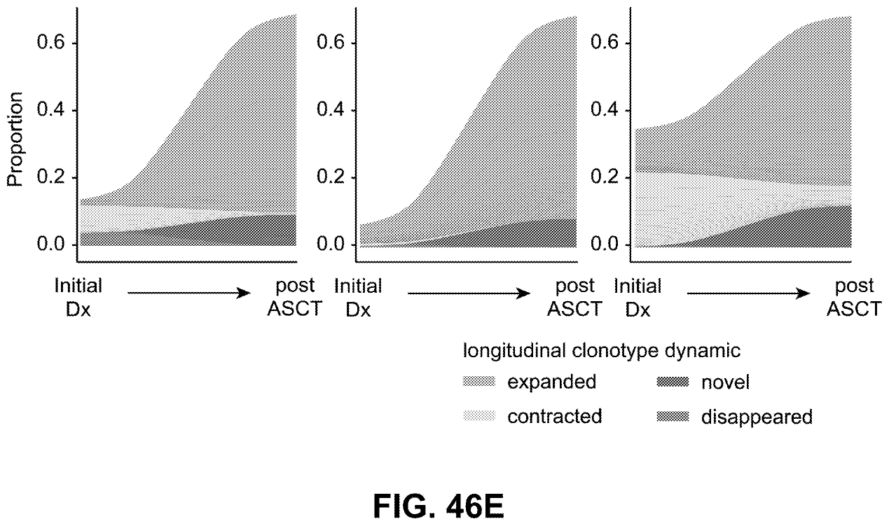

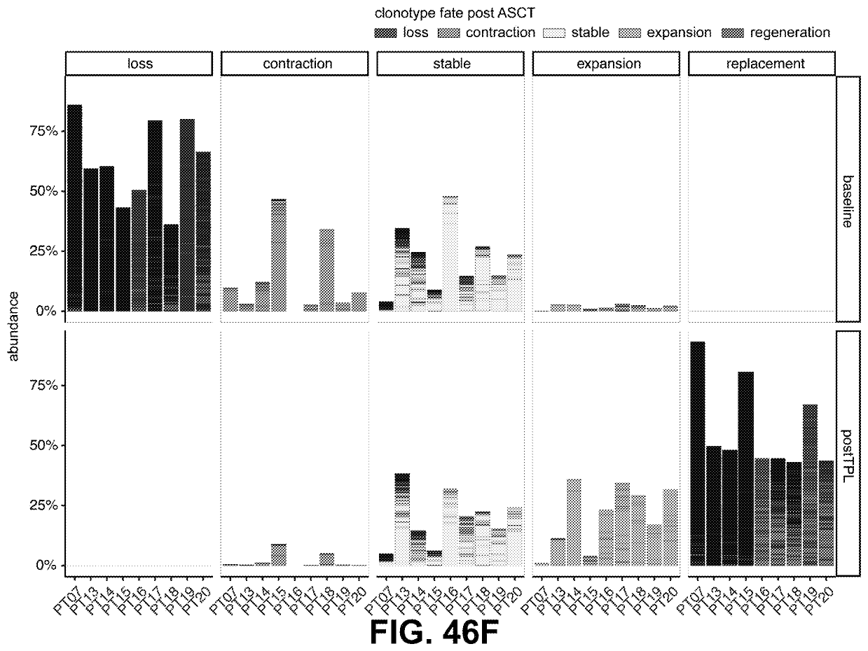

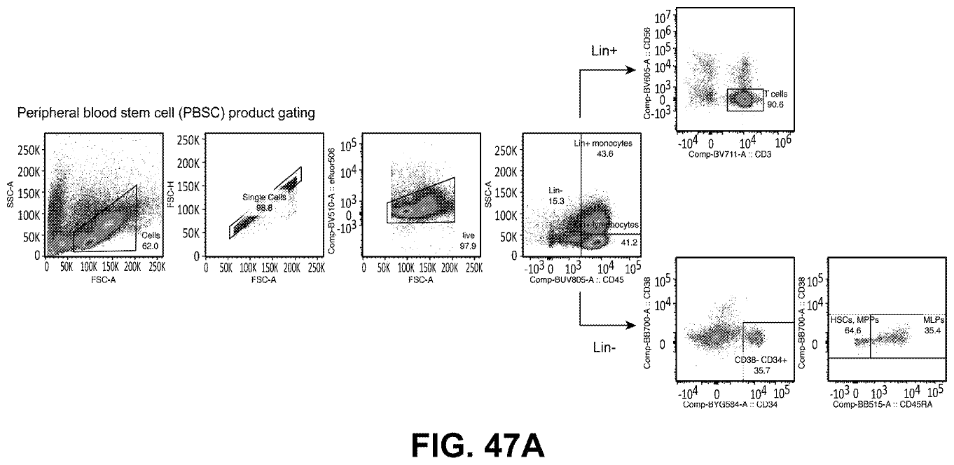

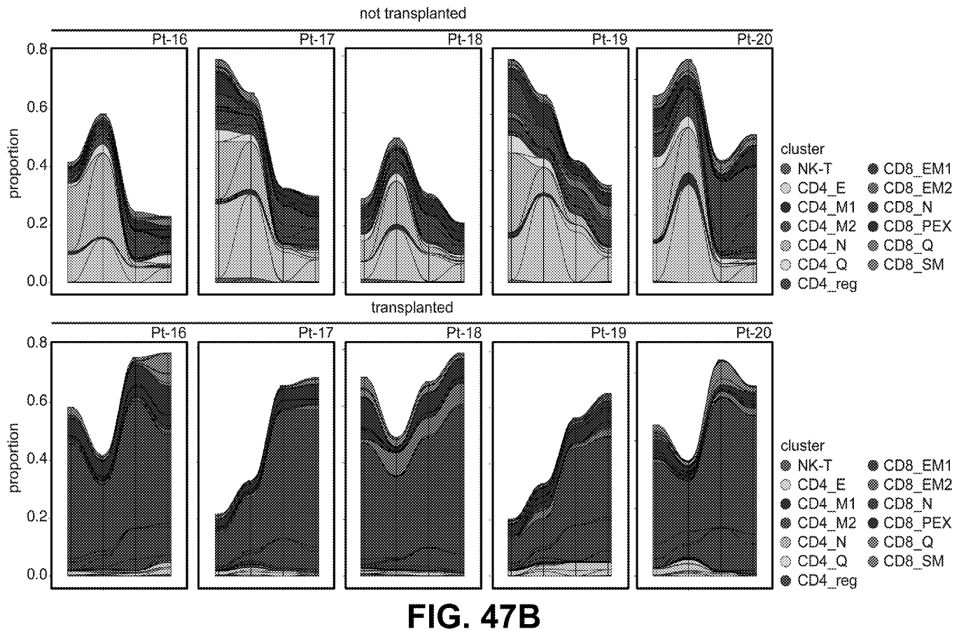

For this analysis, bone marrow biospecimens obtained from patients in the phase II Cardamon trial were utilized, in which patients received carfilzomib, cyclophosphamide, and dexamethasone (KCd) followed by ASCT (ClinicalTrials.gov identifier: NCT02315716) in addition to patients with longitudinal sampling post-ASCT (FIG. 46A)⁵⁹. To study the clonal dynamics of tumor-reactive BMTC, longitudinal TRVα/β-seq was performed along with scRNA/VDJ-seq from all patients with available vitally cryopreserved tissue before treatment initiation and following ASCT. Although overall counts of BMTC clones did not differ between initial diagnosis and the post-ASCT time point (d100 after stem cell infusion; FIGS. 46B & 46C), TCR clonality was significantly increased after ASCT (FIG. 12A, FIG. 46D). This suggests that cancer treatment and immune reconstitution in patients undergoing ASCT involves clonal T cell responses in the bone marrow niche. TCR clonotypes were classified for tumor reactivity based on their transcriptional profile at baseline using the MM-TCR signature and followed their clonal dynamics over the course of induction therapy and ASCT. Given the uncertainty in defining complete TCR losses or gains due to binomial sampling bias, overlapping clones were mapped across multiple sampling time points for downstream analyses. A bootstrapping approach was applied to filter clonotypes with statistically robust clonal dynamics. In short, there was independent sampling 10,000 times from the total TCR repertoire of each patient at paired time points pre- and post-ASCT, calculating empirical p values followed by correction for multiple hypothesis testing to identify individual non-random TCR clonotype dynamics. In general, a dichotomy between strongly expanding and contracting T cell clones was observed in response to ASCT (FIG. 46F). This was accompanied by the loss or gain of some TCRs that could only be detected before or after ASCT, respectively (FIG. 12B, FIG. 46F). Surprisingly, most T cell clones classified as tumor-reactive at baseline were still present or even demonstrated clonal expansion following ASCT. Conversely, tumor-reactive TCRs were either lost following ASCT or newly detected post ASCT, suggesting their regeneration after myeloablation (FIGS. 7B & 7C, FIG. 47).

In transplant-eligible patients with newly diagnosed multiple myeloma, peripheral blood stem cell (PBSC) collection with granulocyte-colony-stimulating factor (G-CSF), high-dose chemotherapy with melphalan (HDM) and ASCT is a standard-of-care^{57,60,61}. It is highly unlikely that almost 50% of the bone marrow TCR repertoire are regenerated with the exact V(D)J rearrangements that were present before ASCT. Without being bound by theory, it was hypothesized that any persistent TCRs are either not fully ablated by high-dose melphalan (HDM) or bypass the procedure entirely by re-infusion together with PBSC products. To address this question, aliquots of the PBSC products from 19 multiple myeloma patients, including from five products that were re-infused into Pts 15-20 of our cohort, were accessed. Analyzing these apheresis products by flow cytometry, an average of ~4% Lin−CD34⁺ stem cells (HSCs/MPPs) were found, which is in line with current stem cell mobilization apheresis protocols⁶² (FIGS. 12D & 12E, FIG. 47A). However, an average of ~23% of all viable mononuclear cells in PBSC products were T cells (FIGS. 12D & 12E, FIG. 47A). Single-cell RNA/TCR-seq to further phenotype these PBSC product-contained T cells and identify their TCR sequences was performed. A representation of all canonical CD4⁺ and CD8⁺ T cell subsets (FIG. 12F, FIG. 47B) were found. Assuming that the PBSC product is the main source of TCR clonotypes originating from the pre-transplantation repertoire, whereas post-ASCT-exclusive clones are likely regenerated as part of immune reconstitution, TRA and TRB sequences were paired to trace tumor- and non-reactive TCRs in the bone marrow for up to one year post ASCT (FIGS. 12G & 12H, FIG. 48). Most non-reactive clonotypes contained in the PBSC product were not detected in post ASCT bone marrow, indicating that they were either not successfully transferred, in circulation, or lost early after ASCT. Successfully transferred, but non-reactive clonotypes demonstrated early expansion in the bone marrow after ASCT (d+100) and were persistent one year after ASCT (d+360), while the regeneration of novel TCRs d+100 and d+360, together occupying about half of the reconstituted bone marrow TCR repertoire 1 year after ASCT, were detected. Tumor-reactive clones in the PBSC product, however, were almost entirely transplanted and long-term persistent, with some TCRs demonstrating continuous clonal expansion post-ASCT in all patients (FIG. 12G, FIGS. 48 & 49). Although highly abundant tumor-reactive TCRs at the initial diagnosis stage were detected, the observed global expansion of this compartment was largely driven by tumor-reactive clones with a baseline abundance <1% (FIG. 12H).

It was next sought to better understand the biological determinants of successful TCR apheresis and engraftment post ASCT. Differential gene expression indicated that genes associated with naïve (LEF1) or resting (TRABD2A) T cells were enriched in BMTCs that are not found in PBSC products, while genes associated with cytotoxicity (GZMH, GZMM), as well as ITGB2, an isoform of ITGB1, were preferentially expressed by T cells found in PBSC products (FIG. 12I).

Without being bound by theory, based on these data, it was hypothesized that preferential enrichment of tumor-reactive TCRs in mobilized PBSC products contributes to the observed high transplantation and persistence rates of these clones. To assess the likelihood of a TCR clonotype being in the PBSC product, odds ratios were calculated based on a logistic regression model adjusted for patient-specific random effects. It was found that being predicted as tumor-reactive substantially increases the likelihood of a T cell clone being in the PBSC product (OR=1.89, p<0.0001), as does being a CD8+ T cell (OR=0.96, p<0.0001) or being in an expansion state pre-apheresis (OR=1.80, p=0.0151), while, expectedly, clonal loss pre-apheresis significantly decreases this likelihood (OR=−2.88, p<0.0001). Conversely, virus reactivity and clonal stability do not significantly affect the likelihood of successful apheresis of a TCR (FIG. 12J). Without being bound by theory, it is therefore suggested that the significant predictors, among them tumor reactivity state, but not virus specificity, indicate their importance in the process leading to the presence of TCRs in the PBSC product.

Studies in solid tumors have consistently associated tumor recognition with dysfunctional T cell phenotypes. However, in the patients, it was found that tumor-reactive clonotypes in largely memory-like and CD8_PEX states that demonstrate further clonal expansion after ASCT. It was therefore aimed to understand whether ASCT affects the cell composition of T cell clonotypes that survive the apheresis and transplantation process. Based on longitudinal single-cell tracing of all TCR clonotypes and the phenotypes of their member cells from initial diagnosis to 1 year post ASCT, it was found that, in line with previous results, most pre-existing CD4⁺ BMTCs are not found in the PBSC product and therefore lost upon ASCT, before likely being regenerated anew from engrafted HSCs (FIG. 12K, FIG. 48). Successfully transplanted BMTCs, however, were largely composed of terminally differentiated CD8⁺ cell states already committed at initial diagnosis, with most post-ASCT proliferation detected in CD8_EM cells (FIG. 12K, FIG. 48B). Notably, although the proportion of cells in a CD8_PEX state was similar between transplanted and non-transplanted clonotypes pre-ASCT, successfully transplanted clonotypes exhibited a substantially reduced proportion of member cells in a CD8_PEX state following ASCT that was maintained for the entire observation time post-ASCT. Conversely, newly identified bone marrow TCRs post-ASCT demonstrated a higher CD8_PEX cell proportion than either non-transplanted cells at initial diagnosis or successfully transplanted cells. This suggests that apheresis, processing of PBSC products, and re-infusion of T cells results in the preferential loss of cells with limited fitness (such as CD8_PEX) within transplanted TCR clonotypes, potentially affecting the expansion and long-term persistence of these clonotypes (FIG. 12L).

In summary, the presence of tumor-reactive TCRs in PBSC were unexpectedly detected and their transfer and long-term persistence post-ASCT with profound effects on the patient immune repertoire were demonstrated. These systemic dynamics mirrored the proportions and cellular states of tumor-reactive cells in the bone marrow, highlighting how the frequency, phenotype, and successful transplantation of tumor-reactive TCRs can potentially distinguish between patients with durable control versus relapse of multiple myeloma.

### Discussion

This study demonstrates that a clinically significant and transplantable innate anti-tumor response resides within a rare subset of bone marrow T cells that target multiple myeloma.

By integrating single-cell sequencing and functional profiling of thousands of human T cells, antigen specificities, phenotypes, and clonal dynamics of bone marrow TCRs were defined in multiple myeloma as a use case for one of the most prevalent hematological malignancies with limited endogenous immunogenicity. The recurrent detection of these tumor-responsive TCRs in patients with multiple myeloma demonstrated their presence in therapy-naïve bone marrow as well as their enrichment following cancer treatment. A conserved gene signature of tumor reactivity is described in these cells that diverges from lymphocytes infiltrating solid tumors and includes ITGB1/CD29 as an indicator of antigen-specific T cell homing. These findings facilitate the identification of tumor-reactive TCRs to inform future cellular therapies in multiple myeloma and further suggest the clinical relevance of immune responses against shared tumor antigens in hematological malignancies and across stem cell transplantation⁶²⁻⁶⁵.

Antigen specificities and phenotypes of tumor-reactive TCRs in hematological cancers have not been systemically characterized due to the low immunogenicity of these diseases and the limiting detection threshold for rare tumor-reactive events outside of the solid tumor microenvironment. Here, this issue has been addressed by developing and applying a multiplexed optical barcoding workflow for TCR specificity. It allows for rapid forward screening of single blood or bone marrow T cells and mapping of TCR specificity to the original cell state in situ. The ability of the screening method to identify bona fide tumor-reactive T cells was challenged by functionally testing reconstituted TCR chains and performing proof-of-concept experiments in AML and CLL patients in addition to multiple myeloma. Importantly, all experiments were performed in patients that were treatment-naïve. Together, this emphasizes the generalizability of this approach and the derived gene signatures in contrast to previous studies that have examined TCR specificities and phenotypes exclusively in patients receiving immune checkpoint blockade^{6,32,38,39,40}.

The frequency of tumor-reactive clones in the bone marrow correlated with their gene expression profiles and clinical outcomes: smaller tumor-reactive clones, preferentially detected by antigen-dependent assays, expressed markers of and resembled genuine naïve T cells, whereas more abundant clones co-expressed programs of cytotoxicity and progenitor exhaustion. It is tempting to speculate which microenvironmental cues caused these small tumor-reactive clones to be either unable to clonally expand ab initio or prevent them from recognizing their cognate antigen. Although longitudinal bone marrow biopsies were performed in all patients, this question remains open due to potential binomial sampling error and lack of real-time resolution.

These results support prior single cell phenotyping studies of solid tumors, showing that tumor-reactive T cells are enriched within differentiated cellular states⁶⁶ with very few stemlike TILs⁶⁷. However, dysfunctional tumor-reactive T cells were less abundant in the bone marrow microenvironment of multiple myeloma than in solid tumors, which might reflect the unique set of microenvironmental cues in the BM niche^{6,40,46,47}. Accordingly, the MM-TCR signature was derived to classify patient-specific tumor-reactive TCRs this disease. The MM-TCR signature might potentially also be applicable in other hematological cancers, provided that the bone marrow microenvironment is not fully disrupted.

A substantial amount of neoepitopes were not in the patients. However, in line with recent studies in several less-immunogenic cancers that have demonstrated the relevance of immune responses against alternative cancer antigens, including CAAs and non-canonical translation products, similar non-private antigens were found to be MHC class I-presented in multiple myeloma by means of immunopeptidome analyses^{68,69}. It was observed that these non-privately expressed antigens can be shared among individuals and result in similarly convergent TCR motifs in MM patients, sometimes across multiple HLA haplotypes. However, larger studies including more diverse study populations are required to determine the detection frequencies of these events.

Finally, evidence was uncovered that tumor-reactive TCRs are selectively enriched in clinical peripheral blood stem cell products, expand, and persist long-term upon autologous stem cell transplantation, and are associated with clinical responses in newly diagnosed as well as refractory multiple myeloma. In patients with RRMM, clinical non-response was underlaid by a failure of potential tumor-reactive TCRs to expand upon immunotherapy-mediated stimulation. It has been previously shown that in patients with progressive disease, chronic bsAb stimulation and persistent tumor burden resulted in an increased fraction of BMTC locked into exhausted states⁴⁵. These data therefore emphasize the importance of disentangling TCR specificity and exhaustion state to selectively activate ‘phenotypically accessible’ and tumor-targeting TCRs to achieve a productive anti-tumor response. Indeed, several studies in solid tumors have proposed that successful anti-tumor responses induced by immunotherapy may originate from novel specificities created systemically or may be the result from reactivated intratumoral tumor-reactive T cells with stem-like properties⁴⁰. The evidence of persistent tumor-reactive clones with distinct transcriptional profiles in stem cell products might inform future personalized cell therapies. It is tempting to speculate that modifications in stem cell mobilization and hematopoietic stem cell transplantation protocols can modulate or amplify tumor reactive TCR responses.

Together, these data suggest that tumor recognition results in a conserved transcriptional state of bone marrow T cells with implications for transplantation immunology and the treatment of multiple myeloma. The tools and rationale are provided in this study for identifying and monitoring T cell responses to enhance endogenous immunity against hematological malignancies.

### Methods

### Human Subjects

Patients with Newly Diagnosed Multiple Myeloma

Bone marrow aspirates and peripheral blood were obtained from 20 donors at diagnosis (pre-treatment/baseline) and, when available, 100 days after autologous stem cell transplantation (ASCT) and 360 days after ASCT. Quality control aliquots from transfused HSC-mobilized PBSC products were obtained from 5 of these patients. Written informed consent was obtained by all patients and donors prior to this study conformed to the principles set out in the WMA Declaration of Helsinki and in the Department of Health and Human Services Belmont Report. Ethical approval for the isolation and functional testing and sequencing of bone marrow aspirates and peripheral blood was obtained from the Heidelberg Medical Faculty Ethics Committee (reference number S-096/2017).

Patients with Newly Diagnosed Multiple Myeloma in Clinical Trial NCT02315716

Bone marrow aspirates were obtained from 14 donors at diagnosis (pre-treatment, baseline) and 100 days after autologous stem cell transplantation (ASCT). Written informed consent was obtained by all patients and donors prior to this study conformed to the principles set out in the WMA Declaration of Helsinki and in the Department of Health and Human Services Belmont Report. Ethical approval for the isolation and functional testing of bone marrow aspirates and peripheral blood was obtained (research ethics committee reference: 07/Q0502/17).

Patients with Relapsed/Refractory Multiple Myeloma in Clinical Trial NCT03269136

Bone marrow aspirates were obtained from 18 donors at diagnosis (pre-treatment, baseline), after one cycle of treatment (post bsAb cycle 1) and after three cycles of treatment (post bsAb cycle 3) or at relapse, whichever occurred first. Written informed consent was obtained by all patients and donors prior to this study conformed to the principles set out in the WMA Declaration of Helsinki and in the Department of Health and Human Services Belmont Report. This study was approved by the University of Calgary Institutional review board and all patients provided written informed consent for tissue sequencing and review of patient medical records for detailed demographic, pathologic, and treatment information (Ethics ID: HREBA.CC-21-0248).

Patients with Acute Myeloid Leukemia (AML)

Peripheral blood was obtained from 5 donors at diagnosis (pre-treatment/baseline. Written informed consent was obtained by all patients and donors prior to this study conformed to the principles set out in the WMA Declaration of Helsinki and in the Department of Health and Human Services Belmont Report. Ethical approval for the isolation and functional testing of peripheral blood was obtained from the Heidelberg Medical Faculty Ethics Committee (reference number S-169/2017).

Patients with Chronic Lymphocytic Leukemia (CLL)

Peripheral blood was obtained from 5 donors at diagnosis (pre-treatment/baseline. Written informed consent was obtained by all patients and donors prior to this study conformed to the principles set out in the WMA Declaration of Helsinki and in the Department of Health and Human Services Belmont Report. Ethical approval for the isolation and functional testing of peripheral blood was obtained from the Heidelberg Medical Faculty Ethics Committee (reference number S-686/2018) and the Broad Institute's biosafety committee (IBC-2017-00146).

### Processing of Human Bone Marrow Samples

Bone marrow aspirates were 1:1 diluted in preparation buffer (PBS with 0.1% BSA and 2 mM EDTA), and mononuclear cell separation was performed by density centrifugation (Bicoll separating solution, Biochrom) with diluted bone marrow cells (centrifugation 20 min, 1300g). Cells were carefully aspirated and washed with preparation buffer (centrifugation 5 min at 470g). Red blood cells were lysed using RCL buffer (155 mM NH4Cl, 10 mM KHCO3, 0.1 mM EDTA) for 10 min at room temperature and bone marrow cells were washed (centrifugation 5 min, 470g) and resuspended in preparation buffer. Malignant plasma cells were freshly isolated using CD138 MicroBeads, human (Miltenyi Biotec) according to the manufacturer's instructions and frozen in 90% FCS (Sigma-Aldrich) supplemented with 10% DMSO and stored in liquid nitrogen until further use. Non-plasma bone marrow mononuclear cells were frozen after cell counting at 1×10⁷ cells per aliquot in 90% FCS (Sigma-Aldrich) supplemented with 10% DMSO and stored in liquid nitrogen until further use.

### Processing of Human Peripheral Blood Samples

Peripheral blood samples were 1:1 diluted in preparation buffer (PBS with 0.1% BSA and 2 mM EDTA), and mononuclear cell separation was performed by density centrifugation (Bicoll separating solution, Biochrom) with diluted peripheral blood cells (centrifugation 20 min, 1300g). Cells were carefully aspirated and washed with preparation buffer (centrifugation 5 min at 470g). Red blood cells were lysed using RCL buffer (155 mM NH4Cl, 10 mM KHCO3, 0.1 mM EDTA) for 1 min at room temperature and cells were washed (centrifugation 5 min, 470g) and resuspended in preparation buffer. After cell counting, 1×10⁷ cells were frozen per aliquot in 90% FCS (Sigma-Aldrich) supplemented with 10% DMSO and stored in liquid nitrogen until further use.

### Cell Lines

For the generation of MC38-OVA cells, the full chicken ovalbumin coding sequence was cloned into pLenti CMV Puro DEST using the Gateway cloning system. Virus was produced in HEK293T cells using PMD.2 and PsPax2 helper plasmids. MC38 cells were transduced and cultured in DMEM supplemented with 10% fetal bovine serum (FBS, Sigma-Aldrich), 100 U/ml Penicillin and 100 μg/ml Streptomycin (Invitrogen) and 1 μg/ml puromycin (Invitrogen).

B16F10 cells were a kind gift of Günther J. Hämmerling (Division of Molecular Immunology, DKFZ Heidelberg). Cells were cultured in DMEM (Sigma-Aldrich) supplemented with 10% FBS, 100 U/ml Penicillin and 100 μg/ml Streptomycin (Invitrogen). All cells were cultured at 37° C. temperature and at 5% CO₂, CD8+ Jurkat reporter cell line was cultured in GlutaMAX-containing RPMI supplemented with 10% heat-inactivated FBS and 1% penicillin-streptomycin. B lymphoblastoid cell lines (B-LCL) were grown in GlutaMAX-containing RMPI 1640 Medium supplemented with 10% FBS 1% penicillin-streptomycin, 50 mM β-mercaptoethanol, 1 mM sodium pyruvate and 5 mL MEM Non-Essential Amino Acids, further referred to as B cell media. HEK293T cells were cultured in DMEM supplemented with 10% FBS and 1% penicillin-streptomycin.

### Generation of TCR-Transgenic Jurkat Cell Lines

Fully humanized TCRs were orders as custom synthesis from Twist Bioscience in a third-generation pLVX-EF1α-IRES-Puro lentiviral expression vector (631253, Takara). HEK293T were cotransfected with a VSV-G envelope expression plasmid (pMD2.G) and a packaging plasmid (psPAX2) following the TransIT-Virusgen Transfection (MIR6700, Mirus) guidelines. Viral supernatants were harvested 48 hours post transfection and filtered supernatants were added onto Jurkat reporter cells (cite T FINDER). The following day viral supernatant was replenished with fresh T cell media and selection of TCR-transgenic Jurkat cells with puromycin (1 μg/ml) was started 96 hours after transduction.

### Antigen-Agnostic Screening of Tumor-Reactive T Cells

BMNCs were isolated as described above. Dead cells were removed using a dead cell removal kit (Miltenyi Biotec). Untouched CD8+ T cells were isolated using a CD8+ T Cell isolation kit, human (Miltenyi Biotec) according to the manufacturer's instructions. Patient matched CD138+ plasma cells were isolated from thawed bone marrow mononuclear cells using CD138 MicroBeads, human (Miltenyi Biotec). Tumor-reactive T cells were identified in single cell interaction assays on the Lightning™ optofluidic system (PhenomeX). A full clean workflow was carried out on the Lightning system using BLI cleaning solution (PhenomeX) and double distilled water (ddH2O). An OptoSelect™ chip (PhenomeX) was wetted with wetting solution (PhenomeX) according to the manufacturer's instructions. Human IFNγ, IL-2, and TNFα capture beads (BioLegend) were loaded into the NanoPen™ chambers of the OptoSelect™ chip using opto-electropositioning technology. Subsequently, single T cells and patient matched myeloma cells were transferred into each NanoPen™ of the OptoSelect™ chip. As a negative control, one of 12 field of views (FOVs) of the OptoSelect™ chip was loaded with T cells and cytokine capture beads only. As a positive control, a different FOV was loaded with T cells and human T-Activator beads (ThermoFisher Scientific). Cells were co-cultured on the OptoSelect™ chip with cell culture media at 36° C. and 5% CO₂ for 12 h. The OptoSelect™ chip was perfused with a detection antibody mix (72 μL LEGENDplex human Th panel detection antibodies, 4 μL Brilliant Violet 421-anti-hCD137 antibody and 4 μl FITC anti-human CD8 antibody, all from BioLegend) for 30 min. Cells and cytokine capture beads were washed with cell culture medium for 45 min. Subsequently, the OptoSelect™ chip was perfused with a streptavidin-PE solution (8 μL LEGENDplex Streptavidin-PE (BioLegend) in 72 μL PBS) for 30 min. Cells and cytokine capture beads were washed with cell culture medium for 45 min. Fluorescence and brightfield images (DAPI, FITC, CY5, PE, OEP filter) of the cells and cytokine capture beads were acquired at 10× magnification. Fluorescence images were analyzed with Image Analyzer 2.1.9.21 (PhenomeX).

### Export and TCR Sequencing of Tumor-Reactive T Cells

Reactive T cells were unloaded from the respective NanoPens™ of the OptoSelect™ chip using OEP™ and exported into 10 μL of TCL (2×) buffer (Qiagen) in a 96-well PCR plate (Eppendorf). 30 μL of mineral oil (Sigma-Aldrich) was layered on top of each T cell export and cells were centrifuged at 1000 g for 5 min at RT. RNA was isolated using 20 μL of RNAClean XP magnetic beads (Beckman Coulter). Magnetic beads were washed twice with 80% ethanol (Sigma-Aldrich), dried at RT for 2 min and resuspended in 4 μL of ice-cold RT mix 1 (1.0 μl/well TCRseq primer 1, 1.0 μL/well dNTP mix, 0.2 μL/well RNase inhibitor, 1.8 μL/well ddH₂O, all from PhenomeX). The export plate was incubated at 72° C. for 3 min, followed by a gradual cool to 4° C. 3 μL of ice-cold RT mix 2 (0.5 μl/well TCRseq primer 2, 1.3 μL/well RT buffer, 1.0 μL/well RT additive, 0.1 μL/well RNase inhibitor, 0.1 μL/well RT enzyme, all from PhenomeX) was added to each well of the export plate. First-strand cDNA was synthesized using a thermal cycler (50 min at 42° C., 10 cycles of 2 min at 50° C. and 2 min at 42° C., followed by 5 min at 85° C.) and purified using 20 μL of MagBind Total Pure NGS magnetic beads (Omega Bio-tek). Magnetic beads were washed twice with 50 μL of 80% ethanol, dried at RT for 2 min and cDNA was eluted by adding 30 μL of nuclease-free water. Agarose gel electrophoresis and DNA quantification (Qubit 4 fluorometer, ThermoFisher Scientific) were performed to verify TCR recovery of each exported T cell. A new PCR plate was prepared on ice (5.0 μL/well PCR enzyme K, 0.4 μL/well TCR amp primer mix (PhenomeX), 3.6 μL/well ddH₂O, 1.0 μl/well purified cDNA) and V(D)J regions of the TCR alpha and beta chains were amplified using a thermal cycler (3 min at 96° C., 23 cycles of 20 s at 98° C., 30 s at 70° C., 20 s at 72° C., followed by 5 min at 72° C.). A reaction mixture, containing unique index pairs for each TCR amplicons, was set up in a new 96-well plate on ice (5.0 μl/well PCR Enzyme K, 0.5 μL/well TCR Index (N7XX and S5XX, Illumina), 4.0 μL/well ddH₂O, and 0.5 μL/well amplified TCR). Different TCR amplicons were barcoded in an indexing PCR (3 min at 96° C., 10 cycles of 20 s at 98° C., 30 s at 70° C., 20 s at 72° C., followed by 5 min at 72° C.). 2 μl of each PCR reaction mixture was pooled and the indexed library was purified using 0.8 volumes of MagBind Total Pure NGS magnetic beads. DNA was washed twice with 80% ethanol; magnetic beads were dried at RT for 3 min and the indexed library was eluted from the beads. The indexed TCR amplicon multiplex was sequenced at a concentration of 10 nM on an Illumina MiSeq system (paired-end 300 bp). TCR V(D)J and CDR3 sequences were identified using MiXCR. TCR sequences with more than 100 reads and productive CDR3 region (no stop codon and in-frame junction) were analyzed. Uncovered sequence regions of full-length TCRα and TCRβ sequences were reconstructed with published TCR sequences exhibiting the highest sequence homology aligned with IMGT/V-QUEST.

### Antigen-Dependent Screening of Tumor and Virus-Specific T Cells

A modified version of the MANAFEST assay was used to evaluate T cell responsiveness to tumor and viral antigens¹⁵. 350,000 CD138-depleted BMNCs containing T cells from patients Pt-04 to Pt-12 were stimulated in vitro with MHC class I-restricted CMV, EBV, Influenza A, SarsCoV and HHV6 peptide pools (jpt Peptide Technologies) as well as with personalized tumor antigen pools (MHC immunopeptidome-derived; synthesized at >90% purity by GenScript), a shared tumor antigen pool (MHC immunopeptidome-derived; synthesized at >90% purity by GenScript) and without peptide for 10 days. CD138-depleted BMNCs were cultivated in culture medium (X-VIVO 20 (Lonza) containing 5% of human serum) with 1 μg/ml of each tumor antigen, viral antigen or without peptide (as a reference for non-specific or background clonotypic expansion). On day 3, half of the medium was replaced with fresh medium containing cytokines at a final concentration of 50 IU/ml recombinant human IL-2 (Novartis), 25 ng/ml recombinant human IL-7 and 25 ng/ml recombinant human IL-15 (both from Biolegend). On day 7, half of the medium was replaced with fresh medium containing cytokines at a final concentration of 100 IU/ml IL-2, 25 ng/ml IL-7 and 25 ng/ml IL-15. On day 10, cells were harvested, washed twice with PBS and the cell pellets were snap-frozen. gDNA isolation and UltraDeep TCRβ sequencing using the Immunosequencing hsTCRβ_v4b workflow were performed by Adaptive Biotechnologies.

### Validation of Tumor Specificity of Tumor-Reactive TCRs by Autologous Co-Cultures

IVT and mRNA Production of V(D)J Coding Gene Fragments

V(D)J-coding gene fragments were synthesized by Twist Biosciences. 25 ng gene fragment DNA coding for the V(D)J regions of TCR alpha and beta chains flanked by BsaI restriction sites was mixed with 50 ng of plasmid T7-alpha-TRAC, 50 ng of plasmid T7-beta-TRBC and 10 μl BsaI (New England Biolabs) and Golden Gate Reaction Mix was incubated in a thermocycler (30 cycles of 1 min at 42° C., 1 min at 16° C., followed by 5 min at 60° C.). 0.5 μl of the reaction mixture was incubated with 1 μl of 1:10 diluted forward primer, 1 μl of 1:10 diluted reverse primer, 5 μl of Clone Amp™ HiFi PCR Premix (ThermoFisher Scientific) and 3 μl of ddH2O in a thermocycler (30 s at 98° C., 30 cycles of 5 s 98° C., 5 s 62° C., 10 s at 72° C., followed by 30 s at 72° C.). The ligated DNA fragment was purified using 0.8 volumes of MagBind Total Pure NGS magnetic beads (Omega Bio-tek) and eluted in 10 μl of ddH2O. The ligation of the DNA fragment was verified by Sanger sequencing (Eurofins Genomics) and 1 μg of DNA was in vitro transcribed using the T7 mScript™ standard mRNA production system (Cellscript). 5′-capped, 3′-polyadenylated mRNA was purified using 0.8 volumes of RNAClean XP magnetic beads (Beckman Coulter) and eluted in 40 μl ddH₂O.

### Rapid T Cell Expansion for Tumor Specificity Validation

10×10⁶ PBMCs isolated from healthy HLA-matched donors were thawed and 0.5×10⁶ CD8+ T cells were sorted using a CD8+ T cell isolation kit, human (Miltenyi Biotec). CD8+ T cells were co-cultured with feeder cells (10×10⁶ PBMCs irradiated at 40 Gy with Gammacell 1000 Elite irradiator) in X-VIVO™ 15 hematopoietic cell media supplemented with 2% human AB serum, 300 U/ml recombinant human IL-2 (Novartis Pharmaceuticals) and an initial concentration of 30 ng/ml CD3 antibody (OKT3, ThermoFisher Scientific) in T25 cell culture flasks (Cellstars). Half of the cell culture media was replenished with fresh media 3 times a week. At day 10 post start of co-culture, T cells were used for electroporation with IVT-transcribed mRNA of respective TCRA and TCRB chains to be tested.

mRNA Transfection and Functional Testing of Transgenic TCR-Expressing T Cells

2×10⁶ expanded T cells in 20 μL nucleofector P3 solution (Lonza) were transfected with 1 μg of TCR coding mRNA in each well of a 96-well unit using an 4D-Nucleofector™ electroporator (EO-115 program, Lonza). Cells were rested for 10 min at RT, 180 μl of per-warmed TexMACS™ Medium (Miltenyi Biotec) was added and electroporated cells were incubated for 24 h in a 48-well plate. 50,000 electroporated autologous T cells and 50,000 CD138+ malignant plasma cells were co-cultured in 200 μl cell culture media in a 96-well U-bottom plate for 16 h at 37° C. As controls, TCR-transgenic T cells were cultured with autologous CD8-depleted PBMCs, 1 μg/ml CEFT peptide pool (JPT Peptide Technologies), or alone. As positive control, T cells were stimulated with human T cell TransAct™ (Miltenyi Biotech) according to the manufacturer's instructions. In addition, all former conditions were tested for mock electroporated autologous T cells (without TCR encoding mRNA). Flu-specific TCR mRNA-transfected autologous CD8+ T cells were co-cultured with CD138+ plasma cells and the aforementioned controls. Cells were washed twice with staining buffer (DPBS, 3% FBS, 2 mM EDTA), incubated with human Fc Block™ (BD Biosciences) for 10 min at RT and stained with CD8-FITC, mTRBC-PE, CD69-PE-Cy7 and CD137-APC antibodies (all from BioLegend) for 20 min on ice in the dark. A 1:1000 diluted live/dead blue viability dye (Life Technologies) was used. Compensation was performed using OneComp eBeads™ (ThermoFisher Scientific). Fixed cells were acquired on the BD Canto II (BD Biosciences) running BD FACSDiva Software. Data were analyzed using FlowJo (v.10.8.1; Tree Star).

### Mice and Treatment

C57BL/6J wild-type mice were purchased from Janvier. All mice were 7-8 weeks of age at use. Mice were kept under specific pathogen-free conditions and 12 h day/night cycle at the animal facility of the DKFZ Heidelberg. All animal procedures followed the institutional laboratory animal research guidelines and were approved by the governmental authorities (Regional Administrative Authority Karlsruhe, Germany. Animal approval protocol G179/19).

For subcutaneous tumor injection, 2×10⁵ MC38-OVA cells and 5×10⁴ B16F10 cells in 100 μl PBS were mixed 1:1 with Matrigel™ (Corning) and injected into the shaved right flank. After nine (MC38-OVA) or six (B16) days of tumor growth, 2×10⁶ pmel T cells and 2×10⁶ OT-I T cells were mixed in a 50:50 ratio and injected intravenously in tumor-bearing mice. 3 days after transfer, blood, spleen, tumor-draining lymph nodes and tumor were extracted. Tumors were manually cut into pieces, digested with 50 μg/ml Liberase™ (Sigma) for 30 minutes and subsequently mashed through 100 μm and 70 μm cell strainers to obtain a single cell suspension. Spleen and lymph nodes were mashed through 70 μm strainers only.

### Flow Cytometry of Murine Tissue Samples

50 μl of whole blood or dissociated and digested tissues were stained with 50 μl antibody cocktail and fixed using fix/lyse solution according to manufacturers' protocol (Thermo Fisher). Cells were blocked with rat anti-mouse CD16/32 antibody (0.5 mg per well, eBioscience) and stained with respective flow cytometry antibodies. eFluor 780 fixable viability dye (eBioscience) was used according to manufacturer's protocol to exclude dead cells. Samples were acquired immediately on a ZES Cell Analyzer (Biorad) equipped with the following lasers: 355 nm, 405 nm; 488 nm; 561 nm; 640 nm.

### Single-Cell RNA Sequencing and Data Preprocessing

For scRNA/TCR/CITE-seq, BMNC were counted, split in 8 aliquots per patient sample and separately stained with TotalSeq™ hashtag antibodies (BioLegend) and fluorescently labeled antibodies before pooling for flow cytometry. Viable, CD45+ CD3+ cells were isolated by fluorescence-activated cell sorting on a BD Aria Fusion™ device (FIG. 13A). Single-cell capture, reverse transcription, and library preparation were carried out on the Chromium platform (10× Genomics) with the Single Cell 5′ reagent v2 kit (10× Genomics) according to the manufacturer's protocol using 40,000 cells as input per channel. Each pool of cells was tested for library quality and library concentration was assessed. Each of the final libraries were paired-end sequenced (26 and 92 bp) on one Illumina NovaSeq 6000 S2 lane. Raw sequencing data containing single-cell RNA, VDJ and cell hashing information were processed using CellRanger (version 7.1) with default processing parameters and the ‘multi’ command. The GRCh38 reference genome (version 2020-A) and GRCh38 V(D)J reference (version 7.1.0) were used accordingly.

### Single-Cell Transcriptomic Analyses

### Quality Control and Normalization

The single-cell RNA sequencing data were demultiplexed, and cells were classified into three categories: Hashtag-oligo singlets, doublets, or unassigned, using the HTODemux command in Seurat v5.0.164 with default settings. For each patient sample, additional heterotypic doublets were identified using the scDblFinder package. Both hashtag information and heterotypic doublet identifications were utilized to remove doublets from the datasets for all downstream analyses. Before proceeding further, we selected only those cells that exhibited a productive ab-TCR. All datasets underwent normalization using SCTransform70 and were compared to a reference dataset of pre-annotated bone marrow single-cell RNA-seq from patients newly diagnosed with myeloma. This comparison enabled us to eliminate all CD3-cells, which were artifacts of sorting impurities from our single-cell RNA-seq data. SCTransform-based reference mapping was employed, as implemented in the MapQuery function in Seurat⁷¹, following the guidelines provided in the vignette (satijalab.org/seurat/articles/sctransform_v2_vignette.html). Cells that exhibited more than 200 detected RNA features, contained less than 10% mitochondrial RNA, and were identified as non-doublets and pure CD3+ T-cells were designated as ‘HighQuality’.

### Clustering of Tumor-Reactive Cells

To cluster the validated MM-reactive T cells separately, we first isolated cells that were annotated with tumor-reactive TCRs. These cells were then integrated afresh using Harmony version 0.1.1⁷² and Seurat⁷¹, with the integration performed on a per-patient basis using default settings. The top 15 PCA dimensions, calculated based on the 2,500 most variable features (with TCR variable genes excluded), were used for this integration. Clusters were determined based on the top 15 Harmony dimensions at a resolution setting of 0.5, merged and annotated manually based on marker gene expression. The expression of features within these clusters was depicted using density plots, facilitated by the Nebulosa package version 1.8.0⁷³.

scTCR Analysis

Throughout this study, clonotypes and clonotype overlaps were identified based on the alpha and beta CDR3 amino acid sequence as called by the Cellranger Algorithm and added to the scRNA data. When multiple alpha or beta chains were annotated for a cell, the top expressed of each chain was filtered. Unique TCRs throughout the paper were called per patient (Pat-TCR). Diversity indices were calculated in R using the vegan v.2.6-4 package (cran.r-project.org/web/packages/vegan/index.html) of TCRs and respective counts within the cluster per patient. Clonotype proportion was calculated among the TCRs within the post QC dataset per patient/dataset.

### Back Projection of Antigen-Specific TCRs to Single-Cell Transcriptomes

For mapping of tumor-reactive TCRs identified by forward microfluidics-based screening, CDR3 sequences from both TRA and TRB chains were back-projected onto the patient-specific single-cell RNA/TCR sequencing dataset. Clonotypes were annotated using CellRanger, providing corresponding CDR3 sequences for either TRB or both TRA and TRB. Pairwise alignment was employed to compare the nucleotide and amino acid sequence similarities between the extracted and annotated sequences. The CDR3 amino acid sequences were evaluated for similarities and overlaps across all patients in both cohorts. When a perfect alignment was found for either both TRA and TRB or just the TRB sequences of a single-cell TCR (scTCR) clonotype with a known tumor-reactive TCR, that scTCR clonotype was classified as tumor-reactive. This method identified 62 unique single-cell defined clones that matched with a tumor-reactive CDR3 sequence, representing a total of 2133 single cells across all sampled patients.

For mapping of MANA epitope-specific TCRs, baseline and post MANA culture samples underwent Adaptive ImmunoSeq ultradeep TCRVb-seq sequencing. Post-expansion samples from patients (PT4-PT12) were grouped by expansion type (shared peptides, personalized peptides, CMV, InfluenzaA, EBV, and SARS-CoV-2) and then demultiplexed back to individual patients for analysis, using only TCRs present in the respective baseline sample. Statistical significance of expansion by group was assessed using the FEST tool (www.stat-apps.one.jhmi.edu/FEST/) with an odds ratio threshold of 2 and an estimated number of cells per well at 250,000. TCRs that were prevalent before expansion (baseline percent >1) were not included in further analysis. Significantly expanded TCRs were then matched to the single-cell dataset for each patient based on exact CDR3 beta amino acid sequence matching.

Mapping of virus-reactive TCRs found in public databases was performed by exact matching of the CDR3 beta amino acid sequences of TCRs with those in the VDJdb database¹⁶ as of Jan. 25, 2024. The particular focus was on TCRbeta chains annotated for “CMV,” “EBV,” “InfluenzaA,” and “SARS-CoV-2.” Public tumor-reactive TCRs were mapped by exact matching of the CDR3 beta amino acid sequences of TCRs with the McPas database⁷⁴ as of Feb. 1, 2024, and also included TCRs validated for reactivity against TP53 and RAS by prior studies, including Levin et al. (2021)⁷⁵, Kim et al. (2022)⁷⁶, and Malekzadeh et al. (2019)⁷⁷. For each patient, only TCRs from these databases that were annotated with matching HLA haplotypes (haplotype group: e.g., HLA-A*02) were considered. In the mapping process to single-cell datasets, MHC class I-restricted TCRs were mapped exclusively to CD8/NKT annotated single cells, and MHC class II-restricted TCRs were mapped only to CD4 annotated single cells. TCRs identified as reactive to multiple viruses or both viruses and tumors were marked as ambiguous unless specified otherwise.

### Differential Gene Expression (DGE) and Establishment of Gene Signatures

DGE was carried out using presto v. 1.0.0 (github.com/immunogenomics/presto) wilcoxauc(assay=“data”), TCR variable genes were filtered from DGE. Results were visualized with the pheatmap v. 1.0.12 using the scaled values of the average gene expression (generated using AverageExpression( ) per respective ident or using the Seurat⁷¹ package functions.

For signature generation of Myeloma-reactive T cells the dataset was split into establishment (PT1-6) and validation cohort (PT7-15). MM signature (sigMM) was generated based on DGE of the establishment_cohort by downsampling the each reactivity group to max. 2000 cells, further filtering the top markers (p_val_adj<0.01, log FC>0.5) of Myeloma-reactive cells (as derived from forward microfluidics-based screening, database/literature matching and MANA assay) in comparison to respective Virus-Epitope reactive (CMV, EBV, InfluenzaA, Sars_CoV_2, ambiguous) and Bystander T cells.

For DGE of epitope reactivity in the complete cohort (Pat 1-15) the same procedure as above was used but with downsampling of 5000 cells per reactivity group. Nine different iterations of MM-TCR were tested by slicing top features ordered by DGE-auc value. Final signature (MM-TCR) included the top 15 genes ordered by auc ((p_val_adj<0.01, log FC>0.5).

Predictional value of the here established MM-signature and further literature derived signatures³⁹ were tested for gene-set enrichment (GSEA) in MM reactive T cells using the AUCell v.1.24.078 package as demonstrated in Lowery et al (2022)³⁹ based on counts data. AUROC curves and AUC values were generated using the pROC v.1.18.5 package⁷⁹, here tumor-reactive cells in the establishment_cohort and validation cohort were termed 1 and all non-tumor-reactive cells 0 (closed world assumption) and used as the response-argument, the AUCell score (enrichment score) was then used as predictor for the ROC analyses using the roc( ) function. AUROC curves were plotted by the ggroc( ) function. The coords( ) function was used to determine the AUCell-score (enrichment score) threshold of 0.48 exhibiting sensitivity and specificity >0.8 in the validation cohort (AUC: 0.8953, threshold: 0.48, specificity: 0.801, sensitivity: 0.807). (establishment cohort: AUC: 0.9795; threshold: 0.48, specificity: 0.938, sensitivity: 0.952).

### Module Scores of T Cell Dysfunction and Cytotoxicity²⁰

### T Cell Dysfunction Score:

“LAG3”, “HAVCR2”, “PDCD1”, “PTMS”, “FAM3C”, “IFNG”, “AKAP5”, “CD7”, “PH LDA1”, “ENTPD1”, “SNAP47”, “TNS3”, “CXCL13”, “RDH10”, “DGKH”, “KIR2DL4”, “LYST”, “MIR155”, “RAB27A”, “CSF1”, “CTLA4”, “TNFRSF9”, “CD27”, “CCL3”, “ITGAE”, “PAG1”, “TN FRSF1B”, “GALNT1”, “GBP2”, “MYO7A”

### T Cell Cytotoxicity Score:

“FGFBP2”, “CX3CR1”, “FCGR3A”, “S1PR5”, “PLAC8”, “FGR”, “Clorf21”, “SPON2”, “CD300A”, “TGFBR3”, “PLEK”, “S1PR1”, “EFHD2”, “KLRF1”, “FAM65B”, “C1orf162”, “STK38”, “SORL1”, “FCRL6”, “TRDC”, “EMP3”, “CCND3”, “KLRB1”, “SAMD3”, “ARL4C”, “IL7R”, “GNLY”

Analysis of Paired BMNC and PBMC scRNA/TCR Datasets

Paired PBMC and BMNC samples from the same patients were processed in the same way as described above resulting in paired data for (N=7) patients. PBMC scRNA/TCR data was annotated using the Seurat R package, employing the previously described reference mapping workflow as for the BMNCs. Reactivity of scRNA/TCR clonotypes to tumors and viruses was determined by mapping to either the single-TCR-sequencing dataset or the VDJdb database as described above. TCR clonotypes were classified as shared between tissues if there was a matching CDR3 amino acid sequence across samples, identified using custom analyses and the scRepertoire R package. Overlap, frequency, and distribution of these clonotypes across tissues were analyzed using the dplyr and ggplot R packages.

### Individual Clonotype Dynamics Across Autologous Transplantation

Paired scRNA and scVDJ-seq data from patients before and after stem cell transplantation (n=9) was used to link clonotypes between timepoints and to annotate clonotype abundance. For this, a match of the TRA and TRB sequences were considered a clonal overlap. Relative clonotype abundance for each timepoint in the study was assessed. To assess if clonotype changes are statistically robust, a bootstrapping approach as defined in Friedrich et al. (2023)³⁴ was applied. In short, by independently sampling 10000 times from the total TCR-repertoire found at the paired timepoints pre and post transplantation for each patient, individual empirical cumulative distribution functions (edcf) were calculated for each patient. This was done in R using the package suite tidymodels and specifically rsample and stats. These edcf-functions were used to calculate empirical p values if an individual TCR-clonotype shows a statistically significant increase or decrease between the timepoints. To control for multiple hypothesis testing, we corrected all calculated p-values using the Benjamini-Hochberg procedure in R using p.adjust. On the level of individual TCR clonotypes the dynamic change is then characterized into five different categories, either expansion (significant increase), contraction (significant reduction), stable (no significant change), complete loss (not found after transplantation) or replacement (only found after transplantation).

### Clone Based Tumor Reactivity Prediction

To annotate complete clones as tumor-reactive based on the previously established reactivity signature sigMM, a classification based on aggregated expression per clone was applied. Clone-level scRNA-seq counts were aggregated using the AggregateExpression function in Seurat v5. On the clone-aggregated expression the signature was scored using AUCell. Each clone with a AUCell score above 0.48 for the signature sigMM was called reactive.

### CDR3 Similarity and Network Analysis

After the identification of tumor-reactive and the annotation of virus-reactive TCRs as described above, the sequences from all different classes of TCRs were extracted from TCR and scRNA/TCR sequencing data were used for similarity analyses. All analyses were implemented in custom R functions. TCR sequences of non-annotated clonotypes from scRNA/TCR were used as bystander-TCRs. CDR3 TRA and TRB sequence similarities were evaluated using local alignment with BLOSUM45 and a gap opening cost of 10 as described previously⁸⁰⁻⁸² utilizing the msa package in R. To normalize local alignment scores, they were divided by the self-alignment score of the query-sequence. The separated scaled similarity was aggregated by calculating a geometric mean for scaled TRA and TRB similarity for each pairwise comparison. A bootstrapping methodology was employed to establish a similarity score significance threshold. In this method all pairwise comparisons of non-annotated TRA-TRB pairs were calculated using the same approach outlined above. This resulted in a background distribution from over 100.000 TCR sequences from TCR-clones found in the bone marrow. Two thresholds at either a 95% or 99% percentile of these background distributions were calculated. The normalized similarities of virus-reactive TCRs were analyzed with the same approach. Across patients, similarities of tumor-reactive TCR TRA-TRB were computed. Additionally, gliph as implemented in the R package turbogliph (github.com/HetzDra/turboGliph) was used to identify clusters of TRA or TRB sequences. Gliph-based clusters were calculated using the turbogliph function independently for TRA and TRB chains of the included clonotypes. Visualization was rendered through heatmaps filtering values above the 95% bootstrapping limit using the ComplexHeatmap R package. To show that gliph was able to identify similarities in either TRA or TRB, each TRA or TRB identified gliph-cluster is shown together with the pairwise similarity analysis. A graphical representation of similarities filtered with the 99% threshold was created with the igraph and ggraph R packages. Distinct clusters of TRA-TRB similarity had their sequence motifs for TRA and TRB illustrated using the msa and ggseqlogo R packages.

scRNA/VDJ-Seq Analysis of PBSC Products

Longitudinal assessment of the bone marrow of multiple myeloma at baseline, after stem cell transplantation and after one year of maintenance was done for (N=5) patients using scRNA and scVDJ sequencing. Additionally, the PBSC product of those five patients was analyzed by scRNA and scVDJ sequencing. Data from pooled scRNA-seq was processed with the pipeline Cellranger (7.1.0) and subsequently using a custom pipeline combining hashtag and SNP-based demultiplexing as implemented in vireo⁸³, souporcell⁸⁴ and Seurat v5⁷¹. After filtering all cells without a productive TCR, cells from 4 paired timepoints were used for downstream analysis. Datasets were mapped to the same reference of pre-annotated bone marrow T cell single-cell RNA-seq from newly diagnosed myeloma patients as above. The fine grained annotation of T-cell subtypes could then be used for further analysis. GSEA of the MM reactive signature was carried out as described above using the package AUCell⁷⁸ based on counts data of T-cells. Using paired TRA and TRB sequences, clonotypes were identified as shared or distinct across timepoints. Clonotype dynamics were assessed as described and implemented above.

### Analysis of Likelihood of Apheresis

To assess the likelihood of a TCR-clonotype being in the PBSC product, we calculated odds ratios based on a logistic regression model. On a single TCR-clonotype level, the previously annotated predicted tumor-reactivity, virus-reactivity, CD4/CD8 lineage status and the clonal dynamic were used as predictors for the model. A linear mixed-effects model (using the lme4 package in R) was implemented to assess the influence of the different predictors on the individual clone being in the PBSC product. It was adjusted for patient-specific random effects to account for intra-patient variability. The logistic regression model predicted odds ratios based on the predicted reactivity, virus reactivity, T cell subtype (CD4 or CD8), and clonal dynamics between diagnosis and after the transplantation as implemented above.

### Integration of Clinical Trial Cohort Sequencing Data

Additional datasets were mapped to the same reference of pre-annotated and unsorted bone marrow single-cell RNA-seq from newly diagnosed myeloma patients as above. This enabled us to identify and filter impurities and to map all samples to a fine-grained annotation of T cells in the diseased bone marrow. GSEA of the BM reactivity signature was carried out as described above using AUCell v.1.20.2⁷⁸ based on counts data. Virus reactivity was also determined based on exact CDR3 AA TRB matches with VDJdb¹⁶ as described above.

Analysis of Longitudinal RRMM Bone Marrow Samples with Bispecific Antibody Exposure

bsAb datasets³⁴ were mapped to the same reference of pre-annotated bone marrow T cell single-cell RNA-seq from newly diagnosed myeloma patients as above. TCR info was filtered and calculated as described above and only cells with productive TCR were analyzed. GSEA of the sigMM signature was carried out as described above using AUCell⁷⁸ v.1.24.0 based on counts data with the same thresholds as established before. Virus reactivity was also determined based on exact CDR3 aa beta match with VDJdb¹⁶ as described above, without HLA-filtering. Overlapping TCRs were identified per patient using the full clonotype CDR3 amino acid sequence. Overlapping clonotypes with a ratio >0.4 of cells above threshold across timepoints were termed reactive for the alluvial analysis. Plots of overlapping TCRs were generated using the ggalluvial (v.0.12.5) package.

Analysis of Paired BMNC and PBMC scRNA/TCR Datasets

Paired peripheral blood mononuclear cell (PBMC) and bone marrow mononuclear cell (BMNC) samples from each patient were processed using the CellRanger pipeline (10× Genomics). PBMC scRNA/TCR data was annotated using the Seurat R package, employing the above-described reference mapping workflow as for the BMNCs. Clonotype specificity of scRNA/TCR clonotypes was determined by mapping to the patient-individual scTCRseq dataset annotated by experimentally tested tumor-reactive clones and the VDJdb¹⁶ database as described above. TCR clonotypes were classified as shared between tissues if there was a matching CDR3 amino acid sequence across samples, identified using the scRepertoire⁸⁵ package in R. Overlap, frequency, and distribution of these clonotypes across tissues were analyzed using the dplyr and ggplot R packages.

### Deep TCRB Sequencing of Clinical Trial Samples

Genomic DNA was isolated from peripheral blood samples using QIAamp DNA isolation kit (QIAGEN) as per the manufacturer's instructions. TCR beta chain (TCRB) deep sequencing was performed on purified DNA from isolated bone marrow or blood mononuclear cells to detect rearranged TCRβ gene sequences using hsTCRB Kit (Adaptive Biotechnologies) according to the manufacturer's protocol. The prepared library was sequenced on an Illumina MiSeq. TCR identification, error correction and CDR3 was performed using the Decombinator pipeline⁸⁰, available at github.com/innate2adaptive/Decombinator. Samples with <2000 total TCR counts were removed. TCR α and β-chains were aggregated to clonotypes for each donor by amino acid CDR3 sequence. Clonality was defined as the inverse of diversity calculated using the Shannon diversity index and Rényi entropy as implemented by the vegan package (cran.r-project.org/web/packages/vegan/index.html). To account for differences in sample size, a weighted subsampled of 2000 counts was taken 100 times for each sample as the mean diversity calculated.

### Whole-Genome Sequencing of Tumor Cells and Germline Controls

WGS was performed on an Illumina NovaSeq 6000 instrument with S4 flow cells in paired-end mode (2×151 bp). Previously described protocols were used for the extraction of nucleic acids, library preparation, and computational processing⁸⁶.

### Neoepitope Prediction

To predict specific neoepitopes, we established an analysis workflow pipeline. It starts with somatic variant calling from CD138+ MM cells to identify possible sites of neoepitope formation, followed by HLA-haplotyping with OptiType 1.3.1 and binding prediction using netMHCpanV.4.1^{87,88}. To narrow an initial large set of possible neoepitopes down to those most likely to be actionable, variant allele frequencies were analyzed. For these analyses, tumor cell data were compared with germline data from PBMCs, thereby eliminating non-somatic neoantigens. Moreover, non-coding variants were excluded from further analysis. The remaining potential sites of neoepitope formation underwent HLA-binding prediction to further reduce the set of potentially actionable neoepitopes. Since HLA class I alleles predominantly bind to 8-9 mer peptide fragments, we chose to focus on the identification of 8-9 mer neoepitopes derived from non-silent SNVs or indels which were then filtered based on their eluted ligand %-rank meeting the cut-off of 2%. To minimize the risk of predicting neoepitopes that are expressed in healthy tissue, potential self-antigens were removed by filtering the predicted neoepitopes against the UniProtKB protein database using the tool PeptideMatch V.1.089. The remaining neoepitopes were then ranked by the allele frequency of the observed coding variant to offset issues arising from tumor heterogeneity.

### MHC Class I Immunopeptidomics on CD138+ Multiple Myeloma Cells^{6,46-48,90}

### Low-Input Immunoprecipitation of HLA-I: Peptide Complexes

Immunoprecipitation of multiple myeloma cells occurred in two batches. Pt-01 through Pt-08 constituted the first batch, while Pt-09 through Pt-12 constituted the second batch. CD138+ cells isolated from multiple myeloma patient bone marrow aspirates were thawed on ice, lysed in 0.2 mL of lysis buffer at 4° C. (20 mM Tris-HCl pH 7.5 (Invitrogen, Waltham, Massachusetts, USA, 15567027), 1 mM Ethylenediaminetetraacetic acid (EDTA) (Invitrogen, Waltham, Massachusetts, USA, 15575-038), 100 mM NaCl (Sigma-Aldrich, St. Louis, Missouri, USA, 71386-1L), 6 mM MgCl₂ (Sigma-Aldrich, St. Louis, Missouri, USA, 63069-100ML), 60 mM Octyl β-d-glucopyranoside (Sigma-Aldrich, St. Louis, Missouri, USA, 08001-25G), 1 mM phenylmethylsulfonyl fluoride (PMSF) (Sigma-Aldrich, St. Louis, Missouri, USA, 93482-250ML-F), 0.2 mM Iodoacetamide (Thermo Fisher Scientific, Waltham, Massachusetts, USA, A39271), 1.50% Triton X-100 (Sigma-Aldrich, St. Louis, Missouri, USA, T9284-500ML), 1× C0mplete protease inhibitor tablet-EDTA free (Sigma-Aldrich, St. Louis, Missouri, USA, 11873580001), 10 mM NaF (Sigma-Aldrich, St. Louis, Missouri, USA, S7920), 1:100 dilution Phosphatase Inhibitor Cocktail II (Sigma-Aldrich, St. Louis, Missouri, USA, P0044), 1:100 dilution Phosphatase Inhibitor Cocktail III (Sigma-Aldrich, St. Louis, Missouri, USA, P5726), 110 mM Sodium Butyrate (Sigma-Aldrich, St. Louis, Missouri, USA, B5887), 2 μM suberoylanilide hydroxamic acid (SAHA) (Sigma-Aldrich, St. Louis, Missouri, USA, SML0061), 10 mM nicotinamide (Sigma-Aldrich, St. Louis, Missouri, USA, N3376), and 50 μM PR-619 (Lifesensors, Malvern, PA, USA, SI9619: PR-619) in pre-conditioned 0.6 mL Eppendorf tubes, and incubated for 30 min with 0.5 μL benzonase (Sigma-Aldrich, St. Louis, Missouri, USA, E1014-25KU). Eppendorf tubes were pre-conditioned by rinsing twice with 500 μL high-performance liquid chromatography (HPLC) water, incubating overnight with 500 μL HPLC water, and then rinsing twice again with 500 μL HPLC water. After incubating in lysis buffer, the lysates were centrifuged at 15,000 rcf for 20 min at 4° C.

To pre-clear the samples, the supernatants were transferred to another set of pre-conditioned 0.6 mL Eppendorf tubes containing ~10 μL phosphate buffered saline (PBS)-washed Gammabind Plus Sepharose beads (Sigma-Aldrich, St. Louis, Missouri, USA, GE17-0886-01) and incubated by end-over-end rotation at 4° C. for 1 hr. Gammabind Plus Sepharose beads were pelleted at 1500 rcf for 1 min at 4° C. The pre-cleared supernatants were transferred to pre-conditioned 0.6 mL Eppendorf tubes containing PBS-washed Gammabind Plus Sepharose beads (Sigma-Aldrich, St. Louis, Missouri, USA, GE17-0886-01) and 5 μg anti-HLA antibody (W6/32) (Bio X Cell, Lebanon, New Hampshire, USA, BP0079). HLA-I peptide complexes were captured on beads by end-over-end incubation at 4° C. for 3 hr. HLA-IP beads were pelleted at 1500 rcf for 1 min at 4° C.

HLA-IP beads were washed on top of a 10 μm PE fritted filter plate (Agilent, Santa Clara, California, USA, S7898A) that was activated with 1 mL acetonitrile (ACN) and equilibrated with 3×1 mL PBS on a positive pressure manifold (Waters Corporation, Milford, Massachusetts, USA, 186006961). HLA-IP beads were transferred to the activated filter plate with 4° C. 1 mL PBS. 500 μL PBS was added to the HLA-IP tube to pick up any remaining beads and transfer them to the filter plate. Beads were washed 2× with 4° C. 1 mL wash buffer (20 mM Tris pH 7.5 (Invitrogen, Waltham, Massachusetts, USA, 15567027), 1 mM EDTA (Invitrogen, Waltham, Massachusetts, USA, 15575-038), 100 mM NaCl (Sigma-Aldrich, St. Louis, Missouri, USA, 71386-1L), 60 mM Octyl β-d-glucopyranoside (Sigma-Aldrich, St. Louis, Missouri, USA, 08001-25G), and 0.2 mM Iodoacetamide (Thermo Fisher Scientific, Waltham, Massachusetts, USA A39271)) and 2× with 4° C. 1 mL 10 mM Tris-HCl pH 7.5.

### Low-Input HLA-I Peptide Elution and Desalting

All HLA IP samples were acid eluted and desalted in two desalting steps (primary and secondary). For the acid elution and primary desalt of the first batch of samples, Sep-Pak tC18 96-well plate with 40 mg sorbent per well (Waters, Milford, Massachusetts, USA, 186002320) was equilibrated with 2×1000 μL methanol, 500 μL 99% ACN/0.1% FA, and 4×1000 μL 1% FA on a positive pressure manifold (Waters Corporation, Milford, Massachusetts, USA, 186006961). Beads were resuspended in 400 μL 3% ACN/5% FA in the filter plate and the filter plate was stacked on top of the equilibrated Sep-Pak tC18 plate. At this step, 50 fmol of Retention Time Standards (iRTs) (JPT Peptide Technologies, Berlin, Germany, RTK-1-10 pmol) were spiked into each sample. The HLA-IP beads were washed 2×200 μL 1% FA. HLA-I peptides were acid eluted from the beads by three, five-minute incubations with 500 μL of 10% acetic acid. The peptides were desalted by 4×1000 μL 1% FA (after the first FA wash, the filter plate was removed) and eluted from the plate with 250 μL 15% ACN/1% FA and 2×250 μL 50% ACN/1% FA, frozen at −80° C., and lyophilized.

For the primary desalt of the second batch of samples, a μElution Sep-Pak tC18 96-well plate with 5 mg sorbent per well (Waters Corporation, Milford, Massachusetts, USA, 186002318) was equilibrated with 2×900 μL methanol, 500 μL 99% ACN/0.1% FA, and 4×900 μL 1% FA. HLA-IP beads were washed in 200 μL 3% ACN/5% FA in the filter plate which was placed on top of the equilibrated Sep-Pak tC18 plate. At this step, 50 fmol of Retention Time Standards (iRTs) (JPT Peptide Technologies, Berlin, Germany, RTK-1-10 pmol) were spiked into each sample. The HLA-IP beads were washed 2×200 μL 1% FA. HLA-I peptides were acid eluted from the HLA-IP beads by three, five-minute incubations with 500 μL of 10% acetic acid. The peptides were desalted by 4×900 μL 1% FA (after the first FA wash, the filter plate was removed) and eluted from the plate with 250 μL 15% ACN/1% FA and 2×250 μL 50% ACN/1% FA, frozen at −80° C., and lyophilized.

The dried HLA-I peptides were stored at −80° C. until reduction and alkylation were performed on the second batch of samples. HLA-I peptides were reconstituted in 100 μL 10 mM Tris-HCl pH 7.5 (Invitrogen, Waltham, Massachusetts, USA, 15567027). 2 μL of 250 mM dithiothreitol (DTT) (Thermo Fisher Scientific, Waltham, Massachusetts, USA, A39255) was added to the sample for a final concentration of 5 mM and incubated for 20 min at 50° C. while shaking at 1000 rpm. 6 μL of 250 mM iodoacetamide (IAA) (Thermo Fisher Scientific, Waltham, Massachusetts, USA, A39271) was added to the sample for a final concentration of 15 mM and incubated for 30 min at room temperature while shaking at 1000 rpm in the dark. The reaction was quenched with 2 μL of 250 mM DTT, for a final concentration of 5 mM, and incubated for 15 min at room temperature while shaking at 1000 rpm. The sample was brought to 3% ACN/5% FA by adding 90 μL of 6.67% ACN/11% FA.

For both sets of samples, a secondary desalt was performed on the sample by mirco-scaled basic reversed phase separation on SDB-XC stage tips. Stage tips were set up with two punches of SDB-XC material (CDS analytical, previously Empore 3M, Oxford, Pennsylvania, USA, 98-0604-0223-1). Stage tips were equilibrated with 2×100 μL methanol, 100 μL 50% ACN/1% FA, and 3×100 μL 1% FA. The lyophilized peptides were resuspended in 200 μL 3% ACN/5% FA, loaded onto the equilibrated stage tip, and ran through twice to maximize peptide retrieval. HLA-peptides were desalted with 3×100 μL 1% FA. HLA-peptides were eluted from the SDB-XC stage tips with 60 μL 5% ACN/1% FA, 60 μL 10% ACN/1% FA, and 60 μL 50% ACN/1% FA. Combined elutions were frozen at −80° C., lyophilized, and stored at −80° C.^{85,86}

### LC-MS/MS Analysis

HLA-I immunopeptidome data collection by LC-MS/MS was performed as described previously⁹⁰. The first batch of HLA-I peptides were resuspended in 5 μL 3% ACN/5% FA and 4 μL were injected onto a timsTOF SCP (Bruker, Billerica, Massachusetts, USA). HLA-I peptides were loaded onto a 30 cm analytical column with 1.9 μm ReproSil-Pur C18 silica beads (Dr Maisch HPLC GmbH, Ammerbuch, Germany), packed in-house into a PicoFrit 75 μm diameter fused silica column with a 10 μm emitter (New Objective, Littleton, Massachusetts, PF360-75-10-N-5). HLA-I peptides were eluted using a stepped gradient on a nanoElute LC (Bruker, Billerica, Massachusetts, USA) ranging from 2-15% Solvent B (0.1% FA in ACN) over 60 min, 15-23% over 30 min, 23-35% over 10 min, 35-80% over 10 min and held at 80% for 10 min at 400 nL/min. MS1 scans were acquired from 100-1700 m/z and 1/K0=1.70 Vs/cm² to 0.60 Vs/cm² in DDA-PASEF mode. Ten PASEF ramps were acquired with an accumulation and ramp time of 200.0 ms. Precursors above the minimum intensity threshold of 1000 were isolated with 2 Th at <700 m/z or 3 Th at >800 m/z and re-sequenced until a target intensity of 20,000 cts/s followed by a dynamic exclusion of 20 s. The collision energy was lowered linearly as a function of increasing ion mobility from 55.00 eV at 1/K0=1.60 Vs/cm² to 15.00 eV at 1/K0=0.60 Vs/cm². Standard tryptic precursor isolation polygon placement was optimized for HLA-I peptide species by extending the polygon to include singly charged precursors with >600 m/z⁴⁷.

The second batch of multiple myeloma samples were acquired on a modified timsTOF SCP, with a higher capacity tims cartridge (Bruker, Billerica, Massachusetts, USA). Peptides were loaded onto a 25 cm Aurora Ultimate CSI nanoflow analytical column with 1.7 μm particle size (IonOpticks, Fitzroy, Victoria, Australia). Peptides were eluted using a stepped gradient on a Vanquish Neo UHPLC System (Thermo Fisher Scientific, Waltham, Massachusetts, USA, VN-S10-A-01). The gradient ranged from 0-15% Solvent B (0.1% FA in ACN) over 60 min, 15-23% over 30 min, 23-35% over 10 min, 35-85% over 10 min and held at 85% for 10 min at 200 nL/min. DDA-PASEF data acquisition parameters are identical to the timsTOF SCP HLA-I parameters described above.

### HLA-I Peptide Database Search

Raw mass spectra were interpreted with the Spectrum Mill (SM) software package, version 8.01 (Broad Institute; proteomics.broadinstitute.org). Only MS/MS spectra with a precursor sequence MH+ in the range of 700-2000, a precursor charge of +1 to +3, and a minimum of <5 detected peaks were extracted. Similar spectra with the same precursor m/z acquired in the same chromatographic peak were merged. MS/MS spectra with a sequence tag length >1 (i.e. minimum of three masses separated by the in-chain masses of two amino acids) were searched with no-enzyme specificity. MS/MS spectra were searched against a database comprised of human reference proteome Gencode 42 (ftp.ebi.ac.uk/pub/databases/gencode/Gencode_human/release_34/42) with 50,872 non-redundant protein coding transcript biotypes mapped to the human reference genome GRCh38, 602 common laboratory contaminants, 2043 curated smORFs (lncRNA and uORFs), 237,427 nuORF DB v1.037, and the JPT iRT peptides (JPT Peptide Technologies, Berlin, Germany, RTK-1-10 pmol) for a total of 290984 entries³³. Patient-specific predicted neoepitope peptides (see Methods: NeoEpitope Prediction) were appended to the sequence databases used for searches that are included in the MassIVE deposit. Parameters for SM MS/MS HLA-I search module for the first batch of samples included: ESI-QEXACTIVE-HCD-HLA-v3 30 scoring; fixed modifications: carbamidomethylation of cysteine; variable modifications: protein N-terminal acetylation and deamidation, oxidized methionine, pyroglutamic acid at peptide N-terminal glutamine, deamidation of asparagine, cysteinylation; precursor mass shift range of −18 to 81 Da; precursor mass tolerance of ±15 ppm; product mass tolerance of ±15 ppm; and a minimum matched peak intensity of 40%. For the second batch of samples, parameters for SM MS/MS HLA-I search module differed in: variable modifications: protein N-terminal acetylation and deamidation, oxidized methionine, pyroglutamic acid at peptide N-terminal glutamine, deamidation of asparagine; precursor mass shift range of −18 to 33 Da. Peptide spectrum matches (PSMs) within <1% false discovery rate (% FDR) were confidently assigned for individual spectra via the target decoy estimation of the SM Autovalidation module. PSMs were filtered for precursor charges of +1 to +3, sequence lengths ranging between 8 to 11 amino acids, and a minimum backbone cleavage score (BCS) of 5. BCS is a metric of peptide sequence coverage to enforce uniformly higher minimum sequence coverage for each PSM. The score is a sum after assigning a 1 or 0 between each pair of adjacent amino acids in the sequence (maximum score is peptide length−1) given all ion types, with the goal of decreasing false positive spectra having fragmentation in a limited portion of the peptide by multiple ion types. PSMs were consolidated to peptides through the SM Protein/Peptide Summary module in the file case sensitive mode. A distinct peptide is determined by the highest scoring PSM of a peptide detected for each sample. If different modification states were observed for a peptide, each were reported with a lowercase letter indicating a variable modification (i.e. “C”-carbamidomethylated, “c”-cysteinylated).

Subset-Specific FDR Filtering for nuORFs

Subset-specific FDR was performed as previously described⁹⁰. The aggregate FDR was set to <1% as described above. FDR for the subset of nuORF peptides requires more stringent score thresholding to reach a suitable subset specific FDR of <1%. Subsets of nuORF types were thresholded independently from the HLA dataset through a two-step approach. First, PSM scoring metrics thresholds were tightened on the nuORF subset: minimum SM score of 7, minimum percent scored peak intensity (SPI) of 50%, precursor mass error of +5 ppm, minimum backbone cleavage score (BCS) of 5. This allows nuORF distributions for each metric to meet or exceed the aggregate distributions. Second, remaining nuORF type subsets with FDR estimate above 1% were further subjected to a grid search to determine the lowest values of BCS and SM score that improved the FDR to <1% for each ORF type in the dataset.

### Identifying Cancer Testis Antigens (CTAs, or Cancer Associated Antigens, CAAs)

Each HLA-peptide maps to a parent protein, with an associated gene symbol. From preexisting lists of known CTAs^{4,46}, the gene symbols of the identified peptides were cross-referenced to identify CTAs in the immunopeptidome.

### Bone Marrow Reactivity Screenings Against Personalized and Shared Antigens Identified in Multiple Myeloma Immunopeptidomes by MHC Tetramer Staining

The following patient HLA-haplotype-matching tetramers were used: Flex-T™ HLA-A*01:01 Monomer UVX, 280001, Biolegend; Flex-T™ HLA-A*02:01 Monomer UVX, 280004, Biolegend; Flex-T™ HLA-A*03:01 Monomer UVX, 280005, Biolegend; Flex-T™ HLA-A*11:01 Monomer UVX, 280007, Biolegend. Tetramer:peptide complexes were prepared according to the manufacturer's instructions. 20 μl Flex-T™ monomer UVX (200 μg/ml, Biolegend) was mixed with 20 μl peptide solution (400 μM, GenScript Biotech) in PBS. Monomers were illuminated with UV light (365 nm, 8 Watts, 5 cm) for 30 min on ice. Monomers were subsequently incubated for 30 min at 37° C. in the dark. 4.4 μl of PE-streptavidin or APC-streptavidin solution (0.2 mg/ml, Biolegend) were added, mixed and incubated for 30 min on ice in the dark. A blocking solution was prepared by mixing 1.6 μl 50 mM D-Biotin (Biolegend) and 192.4 μl PBS. After incubation, 2.4 μl of blocking solution was added and tetramers were incubated on ice for at least 30 min in the dark. Cells were washed twice with staining buffer (PBS, 3% FBS, 2 mM EDTA), incubated with human Fc Block™ (BD Biosciences) for 10 min at RT and stained with 100 μl 1:100 diluted Flex-T™ complex for 30 min on ice in the dark. Cells were co-stained with anti-CD3-Pacific Blue™ and anti-CD8-FITC antibodies (both from Biolegend) as well as 1:1000 diluted Live/Dead™ fixable orange dye (Life Technologies) for 30 min on ice in the dark. Compensation was performed using OneComp eBeads™ (ThermoFisher Scientific). Fixed cells were acquired on the BD Canto II (BD Biosciences) running BD FACSDiva Software. Data were analyzed using FlowJo (v.10.8.1; Tree Star).

### Quantification and Statistical Analysis

Data are represented as individual values or as mean±SEM, as indicated. Group sizes (n) and applied statistical tests are indicated in figure legends. Significance was assessed by either unpaired t-test analysis, paired t-test analysis, or two-way ANOVA analysis with multiple hypothesis testing correction as indicated in figure legends. All reported p values are two-tailed. All analyses were performed using either R v4.3.2 (www.R-project.org) or GraphPad Prism 10.0.

For functional experiments, bone marrow samples were blinded to the experiment performers. Shannon and all others indices were calculated using diversity( ) vegan R package or Gini( ) as stated in methods above.

Due to the nature of this study, sample size determination was not applicable, as all available samples were included in this study. All cells passing QC (FIGS. 13 & 14) were included in downstream analyses on a single-cell basis.

### Data Visualization

Tabular data from single-cell sequencing analyses above were processed using the tidyverse suite of packages [CRAN.R-project.org/package=tidyverse] and visualized in the R programming environment using the ggplot2 package or the Python programming environment using the matplotlib package. Data from all other analyses were visualized using GraphPad Prism 10.0. Figures were produced using Adobe Illustrator 2024.

### Data Availability

Raw TCR sequencing and WGS data used in this study have been deposited through the European Genome-phenome Archive (EGA) in compliance with patient data privacy laws.

All annotated single-cell RNA/VDJ-sequencing data from newly diagnosed multiple myeloma patients (NDMM; patients 01-20) can be accessed at Gene Expression Omnibus (GEO; GSE242883).

Longitudinal TCRA/B sequencing data from patients enrolled in NCT02315716 (UCL cohort; patients 21-34) can be accessed via Zenodo (DOI: 10.5281/zenodo.8329336).

All annotated single-cell RNA/VDJ-sequencing data from patients enrolled in NCT03269136 (bsAb; patients 35-52) can be accessed at Gene Expression Omnibus (GEO; GSE216571).

The original mass spectra, peptide spectrum matches, and the protein sequence database used for searches have been deposited in the public proteomics repository MassIVE (massive.ucsd.edu) with the associated identifier MSV000094359 and are accessible at MSV000094359@massive.ucsd.edu with username: MSV000094359, password: TCRatlas.

### Code Availability

The annotated computer code used to generate the analyses can be accessed via Zenodo (DOI: 10.5281/zenodo.8337398).

TCR identification, error correction and CDR3 was performed using the Decombinator pipeline (academic.oup.com/bioinformatics/article/29/5/542/249065), available at https://github.com/innate2adaptive/Decombinator. The code used for data analysis included Seurat v5.0.1 (for single-cell sequencing analysis), Harmony v1.0 (for single-cell data integration), scVelo v0.2.5, velocyto.py v.0.17, CellRank v.1.5.1 (for RNA velocity analyses), Scanpy v1.8.2 and Matplotlib v3.8, which are each publicly available.

### Example 2—Expansion of Tumor Reactive BMR-T Underlies Immunotherapy in AML Patients

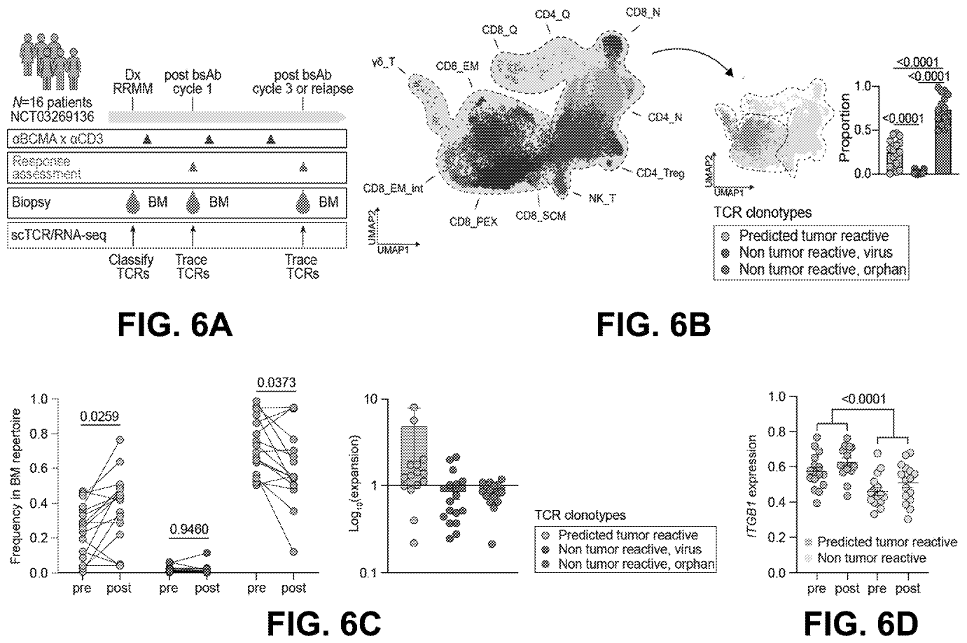

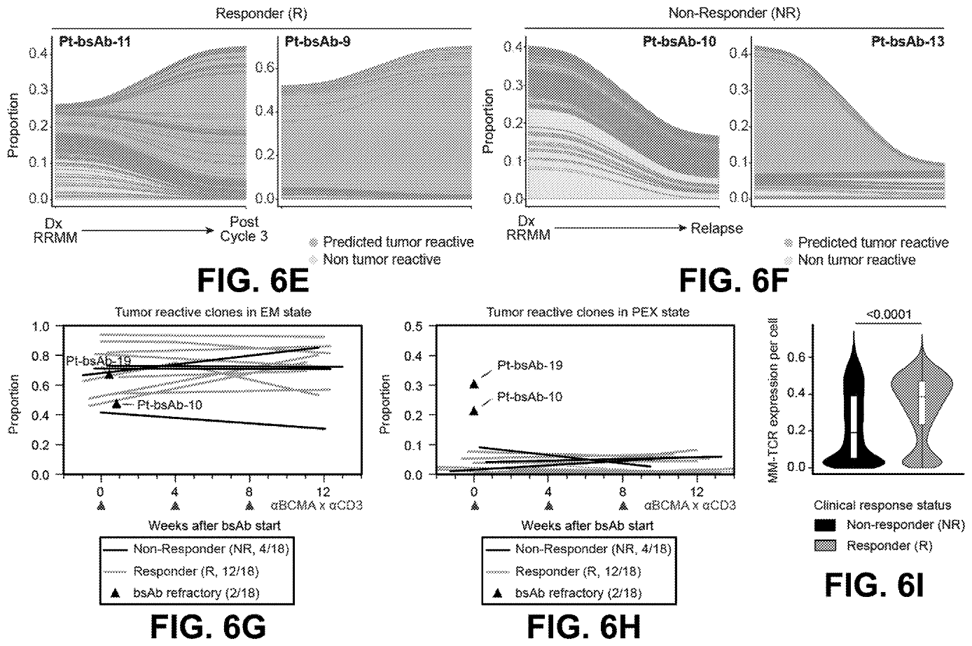

Previous studies on single cell phenotyping of solid tumors have shown that tumor reactive T cells are predominately found within differentiated cellular states³¹, limited stemlike TILs¹ present. However, dysfunctional tumor reactive T cells were less abundant in the diseased bone marrow microenvironment than in solid tumors, which might reflect different microenvironmental cues^{31,33,34,35} Accordingly, a TCR_BM signature was invested to determine if it can isolate clinically relevant clonal T cell expansion in other hematological cancers. AML frequently disrupts the bone marrow microenvironment and suppresses healthy lymphopoiesis, thereby evading tumor immune surveillance³⁶. Allogeneic stem cell transplantation (alloSCT) represents a curative approach to AML due to the powerful T cell-driven graft-versus-leukemia (GVL) effect; however, many patients are not eligible for alloSCT due to comorbidities or unsuccessful donor investigation³⁷. Immune checkpoint inhibition (ICI) to amplify autologous T cell responses therefore represents an attractive off-the-shelf alternative for these patients. Moreover, unlike alloSCT, ICI therapy facilitates longitudinal assessment of patient-autologous tumor reactive TCRs targeting AML cells in the bone marrow. Applicants conducted our analysis on an independent scRNA/TCR dataset of 22 (8 pre- and 14 post-treatment) bone marrow aspirates from 8 R/R AML patients treated with azacytidine and nivolumab therapy on NCT0239772038 (FIG. 7A). Prior to receiving ICI-based therapy, 7 of 8 patients progressed on hypomethylating agents. While on ICI-based treatment, 3/8 patients (PT1-3) responded, while 3/8 (PT4-6) were non-responders (NR) and 2/8 patients had stable disease (SD). Bone marrow biopsies were performed before treatment start, at remission or best response and at relapse on azacytidine and nivolumab. The AML TCR dataset was integrated into a CITE-seq mapped reference and found that the T cell subset composition of the AML dataset on-treatment is similar to the multiple myeloma bone marrow microenvironment, with the exception of a noticeable lack of quiescent CD8+ and CD4+ T cells (FIG. 7B). The frequencies of tumor reactive TCRs, predicted by the TCR_BM classifier (STAR methods), were measured among BMR-T through longitudinal mapping of TCRA/β chains. It was found that in AML, the tumor reactivity signature was highest in clones that persisted in patient bone marrow throughout treatment (FIG. 7D) and that most tumor reactive clones stably expressed ITGB1 transcripts on therapy (FIG. 7E). Following treatment, 4 patients (2 responders and 2 SD) had expansion in their TCR repertoire (FIG. 6F-6G). Conversely, non-responder (3/3) patients had contraction of their most abundant clonotypes (FIG. 6F-6G). Notably, TCRs classified as tumor reactive largely demonstrated clonal expansion between baseline and post-treatment and were associated with superior clinical response (CR/PR) while many clones found in NR could not be detected a relapse (FIG. 6H). In line with previous findings in NDMM, ASCT and bispecific antibody treatment, clinical response to ICI in AML patients was isolated based on the clonal expansion patterns of distinct TCRs (FIG. 7I-7K): Clinical responders demonstrated significant expansion of clonotypes classified as tumor reactive (FIG. 7I; FIG. 7L left), while (2/2) patients with stable TCR dynamics demonstrated SD at response assessment (FIG. 7J, FIG. 7L middle). Conversely, non-responders demonstrated a significant contraction of tumor reactive TCRs at follow-up, that remained stable at time of relapse (FIG. 7K, FIG. 7L right). Without being bound by theory, it is suspected that T cells responding to multiple myeloma and AML demonstrate a conserved transcriptional signature that is independent of the targeted malignancy and associated with clinical response to multiple immunotherapy regimens. Taken together, these data indicate that the multiplexed optical barcoding workflow developed herein can identify tumor reactive TCRs targeting hematological malignancies with high sensitivity and specificity. Identified tumor reactive TCRs demonstrated a conserved transcriptional signature that predicts clinically relevant clonal T cell expansions in the bone marrow. Throughout disease evolution, it was found that clonal expansion of a small subset of these tumor reactive TCRs mirrors the bone marrow immune response to multiple myeloma and AML. Lastly, the abundance of these tumor-recognizing cells is associated with clinical response to autologous stem cell transplantation and immunotherapy in these bone marrow cancers.

### Discussion

Here, a single-cell profiling of thousands of tumor-associated T cells and reverse phenotyping were integrated to define antigen specificities, phenotypes, and clonal dynamics of bone marrow resident T cells in multiple myeloma and acute myeloid leukemia as use cases for highly prevalent and low immunogenic hematological malignancies. A conserved gene signature of these T cells in the bone marrow was revealed. The fate of tumor reactive clonotypes was demonstrated to be linked to clinical immunotherapy response.

T cell receptors (TCRs) play a pivotal role in orchestrating cellular immunity in health and disease. Endogenous or synthetically amplified T cell responses can either control or eliminate tumors. Although frequent clinical responses to immunotherapy and stem cell transplantation have been observed in hematological cancers, the antigen specificities and phenotypes of tumor reactive TCRs have not been characterized in these largely low immunogenic entities. Here, issue was addressed by developing and applying a multiplexed optical barcoding workflow for TCR specificity. It allows for rapid profiling of single bone marrow-resident T cells and mapping of TCR specificity to the original cell state in situ. Importantly, all experiments were performed in patients that were treatment-naïve at the time of biopsy. This emphasizes the generalizability of this approach and the derived gene signatures in contrast to previous studies that have examined TCR specificities and phenotypes exclusively in patients that have received immune checkpoint blockade^{32,33,38,39,40}.

The capabilities of the disclosed platform to identify bona fide tumor reactive T cells in an antigen-agnostic fashion have been challenged by cross-validating it with established antigen expansion assays and functional testing of cloned TCR fragments in a proof-of-concept case. Although the disclosed platform theoretically allows for testing TCR specificities against all tumor types, the required purification of viable clonal tumor cells and tumor-infiltrating lymphocytes, as well as the adherence of solid tumor cells to reaction chambers, may present challenges in workflow for solid tumor analysis. Nonetheless, one could theoretically identify conserved transcriptional or CDR3 sequence features of TILs reactive against solid tumors in a small subset of patients per tumor type. This data could then inform current approaches to identify TCRs for personalized cell therapy from scratch.

The frequency of tumor reactive clones in the bone marrow correlated with gene expression profiles: smaller tumor reactive clones expressed markers and resembled genuine memory T cells, whereas larger clones expressed NK cell-like cytotoxic genes. It is tempting to speculate which microenvironmental cues caused these small tumor reactive clones to be either unable to clonally expand ab initio, or alternatively contract, given their memory state. Although longitudinal bone marrow biopsies in all patients have been performed, this question remains open due to potential binomial sampling error and lack of real-time resolution.

Tumor-specific T cells further exhibited clonal expansion and were more likely to persist long-term in patient bone marrow. These results support prior single cell phenotyping studies of solid tumors showing that tumor reactive T cells are enriched within differentiated cellular states³¹ with very few stemlike TILs¹. However, dysfunctional tumor reactive T cells were less abundant in the bone marrow microenvironment of multiple myeloma than in solid tumors, which might reflect different microenvironmental cues^{31,33,34,35}. Accordingly, the TCR_BM signature was derived to classify patient individual tumor reactive TCRs in multiple myeloma and acute myeloid leukemia. However, the TCR_BM signature might potentially also be applicable in other hematological cancers, as these more closely resemble the transcriptional profiles of BMR-T.

In solid tumors, somatic mutational and neoantigen burden have been shown to correlate with long-term benefit to immune checkpoint inhibition^{41,42}. This rationale predicts that immune recognition of neoantigens in multiple myeloma with its relatively low mutational load would be unlikely, thus limiting the relevance of tumor reactive T cells. However, a similar correlation was found between the detection frequency of TCR_BM+ cells and TMB and recent studies in several cancers with low mutational load have demonstrated the relevance of immune responses against alternative epitopes, including cancer testis antigens, a large family of tumor-associated antigens expressed in human tumors, that we too found as MHC class I-presented antigens by means of ligandome analyses in our patients and members of which have been described to be expressed in multiple hematological cancers^{43,44}. Non-privately expressed cancer testis antigens between individuals might further explain our observation of recurrent TCR motifs in patients affected with multiple myeloma.

Lastly, evidence was found that tumor reactive TCRs selectively expand on autologous stem cell transplantation and are associated with clinical response to bispecific T-cell engaging antibodies and immune checkpoint inhibitors. In patients with relapsed-refractory MM or AML, clinical non-response was underlain by a failure of potential tumor reactive TCRs to expand upon immunotherapy-mediated stimulation. It was previously shown that in patients with progressive disease, chronic bsAb stimulation and persistent tumor burden resulted in an increased fraction of BMR-T locked in exhausted states⁴⁵. These therefore emphasize the importance of disentangling TCR specificity and exhaustion state to achieve a productive anti-tumor response, as not all tumor reactive T cells in bone marrow cancers might be in an exhausted state. Indeed, several studies in solid tumors have proposed that successful anti-tumor responses induced by immunotherapy may originate from novel specificities created systemically or may be the result from reactivated intratumoral tumor-specific T cells with stem-like properties⁴⁰. Notably, Pt-8, who achieved MRD-negative complete response and has not yet shown relapse following ASCT, demonstrated the presence of an expanded bona fide tumor reactive clone with member cells primarily exhibiting an effector-memory phenotype and no tumor-specific TCRs in an exhausted state. Further studies could identify TCRs with similar properties using serial bone marrow biopsies to generate ex vivo reinvigorated or clones TCR-transgenic cell therapy products. The evidence of tumor reactive clones with distinct transcriptional profiles even before treatment, ASCT or immunotherapy establishes the possibility of future patient-individualized cell therapies in hematological malignancies. As this approach was able to identify tumor reactive TCRs in treatment-naïve tissue, it might be incorporated into workflows to generate personalized cell therapy products from initial diagnosis on.

Together, these data suggest clinically relevant endogenous anti-tumor reactivity in a subset of bone marrow-resident T cells and provide the rationale for identifying and monitoring tumor reactive T cell responses targeting hematological cancers.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### BIBLIOGRAPHY

1. Yong, K. et al. Upfront autologous haematopoietic stem-cell transplantation versus carfilzomib-cyclophosphamide-dexamethasone consolidation with carfilzomib maintenance in patients with newly diagnosed multiple myeloma in England and Wales (CARDAMON): a randomised, phase 2, non-inferiority trial. Lancet Haematol 10, e93-e106 (2023).
2. Nizar J. Bahlis et al. Efficacy and safety of elranatamab (PF-06863135), a B-cell maturation antigen (BCMA)-CD3 bispecific antibody, in patients with relapsed or refractory multiple myeloma (MM). J. Clin. Oncol. 39, 8006-8006 (2021).
3. Andrzej J. Jakubowiak et al. Elranatamab, a BCMA-targeted T-cell redirecting immunotherapy, for patients with relapsed or refractory multiple myeloma: Updated results from MagnetisMM-1. J. Clin. Oncol. 40, 8014-8014 (2022).
4. Danilova, L. et al. The Mutation-Associated Neoantigen Functional Expansion of Specific T Cells (MANAFEST) Assay: A Sensitive Platform for Monitoring Antitumor Immunity. Cancer Immunol Res 6, 888-899 (2018).
5. Alrasheed, N. et al. Marrow-Infiltrating Regulatory T Cells Correlate with the Presence of Dysfunctional CD4(+)PD-1(+) Cells and Inferior Survival in Patients with Newly Diagnosed Multiple Myeloma. Clin Cancer Res 26, 3443-3454 (2020).
6. Almeida, L. G. et al. CTdatabase: a knowledge-base of high-throughput and curated data on cancer-testis antigens. Nucleic Acids Res 37, D816-9 (2009).
7. Ouspenskaia, T. et al. Unannotated proteins expand the MHC-I-restricted immunopeptidome in cancer. Nat. Biotechnol. 40, 209-217 (2022).
8. Medina, A. et al. Molecular profiling of immunoglobulin heavy-chain gene rearrangements unveils new potential prognostic markers for multiple myeloma patients. Blood Cancer J. 10, 14 (2020).
9. Khodadoust, M. S. et al. B-cell lymphomas present immunoglobulin neoantigens. Blood 133, 878-881 (2019).
10. Kwak, L. W. et al. Induction of Immune Responses in Patients with B-Cell Lymphoma against the Surface-Immunoglobulin Idiotype Expressed by Their Tumors. N. Engl. J. Med. 327, 1209-1215 (1992).
11. Trojan, A. et al. Immunoglobulin framework-derived peptides function as cytotoxic T-cell epitopes commonly expressed in B-cell malignancies. Nat. Med. 6, 667-672 (2000).
12. Xiaoling, G. et al. Induction of anti B-cell malignance CTL response by subfamily-shared peptides derived from variable domain of immunoglobulin heavy chain. Cancer Immunol. Immunother. 54, 1106-1114 (2005).
13. Weng, J. et al. Targeting B-cell malignancies through human B-cell receptor specific CD4⁺ T cells. OncoImmunology 5, e1232220 (2016).
14. Cetin, M. et al. T-FINDER: A highly sensitive, pan-HLA platform for functional T cell receptor and ligand discovery. Sci. Adv. 10, eadk3060 (2024).
15. Yarchoan, M., Hopkins, A. & Jaffee, E. M. Tumor Mutational Burden and Response Rate to PD-1 Inhibition. N Engl J Med 377, 2500-2501 (2017).
16. Shugay, M. et al. VDJdb: a curated database of T-cell receptor sequences with known antigen specificity. Nucleic Acids Res 46, D419-D427 (2018).
17. Okhrimenko, A. et al. Human memory T cells from the bone marrow are resting and maintain long-lasting systemic memory. Proc. Natl. Acad. Sci. 111, 9229-9234 (2014).
18. Mazo, I. B. et al. Bone Marrow Is a Major Reservoir and Site of Recruitment for Central Memory CD8+ T Cells. Immunity 22, 259-270 (2005).
19. Wu, T. D. et al. Peripheral T cell expansion predicts tumour infiltration and clinical response. Nature 579, 274-278 (2020).
20. Li, H. et al. Dysfunctional CD8 T Cells Form a Proliferative, Dynamically Regulated Compartment within Human Melanoma. Cell 176, 775-789 e18 (2019).
21. Joshi, K. et al. Spatial heterogeneity of the T cell receptor repertoire reflects the mutational landscape in lung cancer. Nat Med 25, 1549-1559 (2019).
22. Venturi, V. et al. TCR β-Chain Sharing in Human CD8+ T Cell Responses to Cytomegalovirus and EBV. J. Immunol. 181, 7853-7862 (2008).
23. Parameswaran, P. et al. Convergent Antibody Signatures in Human Dengue. Cell Host Microbe 13, 691-700 (2013).
24. Dash, P. et al. Quantifiable predictive features define epitope-specific T cell receptor repertoires. Nature 547, 89-93 (2017).
25. Meeuwsen, M. H. et al. A broad and systematic approach to identify B cell malignancy-targeting TCRs for multi-antigen-based T cell therapy. Mol. Ther. 30, 564-578 (2022).
26. Dhodapkar, K. & Dhodapkar, M. Harnessing shared antigens and T-cell receptors in cancer: Opportunities and challenges. Proc. Natl. Acad. Sci. 113, 7944-7945 (2016).
27. Hosokawa, H. & Rothenberg, E. V. How transcription factors drive choice of the T cell fate. Nat Rev Immunol 21, 162-176 (2021).
28. Cortez, J. T. et al. CRISPR screen in regulatory T cells reveals modulators of Foxp3. Nature 582, 416-420 (2020).
29. Tirosh, I. et al. Dissecting the multicellular ecosystem of metastatic melanoma by single-cell RNA-seq. Science 352, 189-96 (2016).
30. Jansen, C. S. et al. An intra-tumoral niche maintains and differentiates stem-like CD8 T cells. Nature 576, 465-470 (2019).
31. Simoni, Y. et al. Bystander CD8 (+) T cells are abundant and phenotypically distinct in human tumour infiltrates. Nature 557, 575-579 (2018).
32. Oliveira, G. et al. Phenotype, specificity and avidity of antitumour CD8 (+) T cells in melanoma. Nature 596, 119-125 (2021).
33. Miller, B. C. et al. Subsets of exhausted CD8 (+) T cells differentially mediate tumor control and respond to checkpoint blockade. Nat Immunol 20, 326-336 (2019).
34. de Jong, M. M. E. et al. The multiple myeloma microenvironment is defined by an inflammatory stromal cell landscape. Nat. Immunol. 22, 769-780 (2021).
35. Kawano, Y. et al. Targeting the bone marrow microenvironment in multiple myeloma. Immunol Rev 263, 160-72 (2015).
36. Binnewies, M. et al. Understanding the tumor immune microenvironment (TIME) for effective therapy. Nat. Med. 24, 541-550 (2018).
37. Horowitz, M. et al. Graft-versus-leukemia reactions after bone marrow transplantation. Blood 75, 555-562 (1990).
38. Caushi, J. X. et al. Transcriptional programs of neoantigen-specific TIL in anti-PD-1-treated lung cancers. Nature 596, 126-132 (2021).
39. Lowery, F. J. et al. Molecular signatures of anti-tumor neoantigen-reactive T cells from metastatic human cancers. Science 375, 877-884 (2022).
40. Yost, K. E. et al. Clonal replacement of tumor-specific T cells following PD-1 blockade. Nat Med 25, 1251-1259 (2019).
41. Klempner, S. J. et al. Tumor Mutational Burden as a Predictive Biomarker for Response to Immune Checkpoint Inhibitors: A Review of Current Evidence. Oncologist 25, e147-e159 (2020).
42. Schrock, A. B. et al. Tumor mutational burden is predictive of response to immune checkpoint inhibitors in MSI-high metastatic colorectal cancer. Ann Oncol 30, 1096-1103 (2019).
43. Gjerstorff, M. F., Andersen, M. H. & Ditzel, H. J. Oncogenic cancer/testis antigens: prime candidates for immunotherapy. Oncotarget 6, 15772-87 (2015).
44. van Duin, M. et al. Cancer testis antigens in newly diagnosed and relapse multiple myeloma: prognostic markers and potential targets for immunotherapy. Haematologica 96, 1662-9 (2011).
45. Friedrich, M. J. et al. The pre-existing T cell landscape determines the response to bispecific T cell engagers in multiple myeloma patients. Cancer Cell (2023) doi: 10.1016/j.ccell.2023.02.008.
46. Klaeger, S. et al. Optimized Liquid and Gas Phase Fractionation Increases HLA-Peptidome Coverage for Primary Cell and Tissue Samples. Mol. Cell. Proteomics 20, 100133 (2021).
47. Phulphagar, K. M. et al. Sensitive, high-throughput HLA-I and HLA-II immunopeptidomics using parallel accumulation-serial fragmentation mass spectrometry. Mol. Cell. Proteomics 100563 (2023) doi: 10.1016/j.mcpro.2023.100563.
48. Djureinovic, D. et al. Profiling cancer testis antigens in non-small-cell lung cancer. JCI Insight 1, (2016).
49. Moreau, P. et al. Bortezomib, thalidomide, and dexamethasone with or without daratumumab before and after autologous stem-cell transplantation for newly diagnosed multiple myeloma (CASSIOPEIA): a randomised, open-label, phase 3 study. The Lancet 394, 29-38 (2019).
50. Nicolet, B. P. et al. CD29 identifies IFN-γ-producing human CD8⁺ T cells with an increased cytotoxic potential. Proc. Natl. Acad. Sci. 117, 6686-6696 (2020).
51. Martens, R. et al. Efficient homing of T cells via afferent lymphatics requires mechanical arrest and integrin-supported chemokine guidance. Nat. Commun. 11, 1114 (2020).
52. Kilian, M. et al. MHC class II-restricted antigen presentation is required to prevent dysfunction of cytotoxic T cells by blood-borne myeloids in brain tumors. Cancer Cell 41, 235-251 e9 (2023).
53. Durie, B. G. M. et al. International uniform response criteria for multiple myeloma. Leukemia 20, 1467-1473 (2006).
54. Rajkumar, S. V. et al. Consensus recommendations for the uniform reporting of clinical trials: report of the International Myeloma Workshop Consensus Panel 1. Blood 117, 4691-4695 (2011).
55. Hudson, D., Fernandes, R. A., Basham, M., Ogg, G. & Koohy, H. Can we predict T cell specificity with digital biology and machine learning? Nat Rev Immunol 1-11 (2023) doi: 10.1038/s41577-023-00835-3.
56. Waldman, A. D., Fritz, J. M. & Lenardo, M. J. A guide to cancer immunotherapy: from T cell basic science to clinical practice. Nat Rev Immunol 20, 651-668 (2020).
57. Attal, M. et al. Lenalidomide, Bortezomib, and Dexamethasone with Transplantation for Myeloma. N. Engl. J. Med. 376, 1311-1320 (2017).
58. Kumar, S. et al. Carfilzomib, lenalidomide, and dexamethasone (KRd) versus bortezomib, lenalidomide, and dexamethasone (VRd) for initial therapy of newly diagnosed multiple myeloma (NDMM): Results of ENDURANCE (E1A11) phase III trial. J. Clin. Oncol. 38, LBA3-LBA3 (2020).
59. van der Leun, A. M., Thommen, D. S. & Schumacher, T. N. CD8 (+) T cell states in human cancer: insights from single-cell analysis. Nat Rev Cancer 20, 218-232 (2020).
60. Dimopoulos, M. A. et al. Multiple myeloma: EHA-ESMO Clinical Practice Guidelines for diagnosis, treatment and follow-upt. Ann. Oncol. 32, 309-322 (2021).
61. Cavo, M. et al. Autologous haematopoietic stem-cell transplantation versus bortezomib-melphalan-prednisone, with or without bortezomib-lenalidomide-dexamethasone consolidation therapy, and lenalidomide maintenance for newly diagnosed multiple myeloma (EMN02/HO95): a multicentre, randomised, open-label, phase 3 study. Lancet Haematol. 7, e456-e468 (2020).
62. Kauer, J. et al. Stem cell collection after lenalidomide, bortezomib and dexamethasone plus elotuzumab or isatuximab in newly diagnosed multiple myeloma patients: a single centre experience from the GMMG-HD6 and -HD7 trials. BMC Cancer 23, 1132 (2023).
63. Zamora, A. E. et al. Pediatric patients with acute lymphoblastic leukemia generate abundant and functional neoantigen-specific CD8+ T cell responses. Sci. Transl. Med. 11, eaat8549 (2019).
64. Chapuis, A. G. et al. T cell receptor gene therapy targeting WT1 prevents acute myeloid leukemia relapse post-transplant. Nat. Med. 25, 1064-1072 (2019).
65. Biernacki, M. A. et al. CBFB-MYH11 fusion neoantigen enables T cell recognition and killing of acute myeloid leukemia. J. Clin. Invest. 130, 5127-5141 (2020).
66. Simoni, Y. et al. Bystander CD8(+) T cells are abundant and phenotypically distinct in human tumour infiltrates. Nature 557, 575-579 (2018).
67. Jansen, C. S. et al. An intra-tumoral niche maintains and differentiates stem-like CD8 T cells. Nature 576, 465-470 (2019).
68. Gjerstorff, M. F., Andersen, M. H. & Ditzel, H. J. Oncogenic cancer/testis antigens: prime candidates for immunotherapy. Oncotarget 6, 15772-87 (2015).
69. van Duin, M. et al. Cancer testis antigens in newly diagnosed and relapse multiple myeloma: prognostic markers and potential targets for immunotherapy. Haematologica 96, 1662-9 (2011).
70. Hafemeister, C. & Satija, R. Normalization and variance stabilization of single-cell RNA-seq data using regularized negative binomial regression. Genome Biol. 20, 296 (2019).
71. Hao, Y. et al. Dictionary learning for integrative, multimodal and scalable single-cell analysis. Nat. Biotechnol. 42, 293-304 (2024).
72. Korsunsky, I. et al. Fast, sensitive and accurate integration of single-cell data with Harmony. Nat Methods 16, 1289-1296 (2019).
73. Alquicira-Hernandez, J. & Powell, J. E. Nebulosa recovers single cell gene expression signals by kernel density estimation. Bioinformatics (2021) doi: 10.1093/bioinformatics/btab003.
74. Tickotsky, N., Sagiv, T., Prilusky, J., Shifrut, E. & Friedman, N. McPAS-TCR: a manually curated catalogue of pathology-associated T cell receptor sequences. Bioinformatics 33, 2924-2929 (2017).
75. Levin, N. et al. Identification and Validation of T-cell Receptors Targeting RAS Hotspot Mutations in Human Cancers for Use in Cell-based Immunotherapy. Clin. Cancer Res. 27, 5084-5095 (2021).
76. Kim, S. P. et al. Adoptive Cellular Therapy with Autologous Tumor-Infiltrating Lymphocytes and T-cell Receptor-Engineered T Cells Targeting Common p53 Neoantigens in Human Solid Tumors. Cancer Immunol. Res. 10, 932-946 (2022).
77. Malekzadeh, P. et al. Neoantigen screening identifies broad TP53 mutant immunogenicity in patients with epithelial cancers. J. Clin. Invest. 129, 1109-1114 (2019).
78. Aibar, S. et al. SCENIC: single-cell regulatory network inference and clustering. Nat Methods 14, 1083-1086 (2017).
79. Robin, X. et al. pROC: an open-source package for R and S+ to analyze and compare ROC curves. BMC Bioinformatics 12, 77 (2011).
80. Yokota, R., Kaminaga, Y. & Kobayashi, T. J. Quantification of Inter-Sample Differences in T-Cell Receptor Repertoires Using Sequence-Based Information. Front. Immunol. 8, 1500 (2017).
81. Thakkar, N. & Bailey-Kellogg, C. Balancing sensitivity and specificity in distinguishing TCR groups by CDR sequence similarity. BMC Bioinformatics 20, 241 (2019).
82. Vujovic, M. et al. T cell receptor sequence clustering and antigen specificity. Comput. Struct. Biotechnol. J. 18, 2166-2173 (2020).
83. Huang, Y., McCarthy, D. J. & Stegle, O. Vireo: Bayesian demultiplexing of pooled single-cell RNA-seq data without genotype reference. Genome Biol. 20, 273 (2019).
84. Heaton, H. et al. Souporcell: robust clustering of single-cell RNA-seq data by genotype without reference genotypes. Nat. Methods 17, 615-620 (2020).
85. Borcherding, N., Bormann, N. L. & Kraus, G. scRepertoire: An R-based toolkit for single-cell immune receptor analysis. F1000Research 9, 47 (2020).
86. Horak, P. et al. Comprehensive Genomic and Transcriptomic Analysis for Guiding Therapeutic Decisions in Patients with Rare Cancers. Cancer Discov. 11, 2780-2795 (2021).
87. Szolek, A. et al. OptiType: precision HLA typing from next-generation sequencing data. Bioinforma. Oxf. Engl. 30, 3310-3316 (2014).
88. Reynisson, B., Alvarez, B., Paul, S., Peters, B. & Nielsen, M. NetMHCpan-4.1 and NetMHCIIpan-4.0: improved predictions of MHC antigen presentation by concurrent motif deconvolution and integration of MS MHC eluted ligand data. Nucleic Acids Res. 48, W449-W454 (2020).
89. Chen, C. et al. A fast Peptide Match service for UniProt Knowledgebase. Bioinforma. Oxf. Engl. 29, 2808-2809 (2013).
90. Abelin, J. G. et al. Workflow enabling deepscale immunopeptidome, proteome, ubiquitylome, phosphoproteome, and acetylome analyses of sample-limited tissues. Nat. Commun. 14, 1851 (2023).

TABLE 1
Markers for newly diagnosed multiple myeloma bone marrow reference.

| | | | | PMID |
|---|---|---|---|---|
| | | | mRNA | Reference |
| Cell Type | Abbreviation | Protein Marker (CITE-seq) | marker | (optional) |
| CD4 T cells | | | | |
| CD4+ FOXP1+ quiescent T-cell | T_CD4_Q | CD3+, CD4+, CD8− | FOXP1 | 27861791 |
| CD4+ naive T-cells | T_CD4_N | CD3+, CD4+, CD45RA+, CD27+ | TCF7, | |
| | | | CCR7 | |
| CD4+ memory T-cells 1 | T_CD4_Mem_1 | CD3+, CD4+, CD45RO+, CD95+, | TCF7 | |
| | | CD278+, CD25− | | |
| CD4+ memory T-cells 2 | T_CD4_Mem_2 | CD3+, CD4+, CD45RO+, CD95+, | GATA3 | |
| | | CD26hi | | |
| CD4+ cytotoxic T-cells | T_CD4_tox | CD3+, CD4+, CD244+, KLRG1+ | GZMA | |
| CD4+ regulatory T-cells | T_CD4_reg | CD3+, CD4+, CD45RO+, CD25+, | FOXP3 | 31278120 |
| | | CD95hi | | |
| CD8 T cells | | | | |
| CD8+ FOXP1+ quiescent T-cell | T_CD8_Q | CD3+, CD8+, CD4- | FOXP1 | 27001958 |
| CD8+ naive T-cells | T_CD8_N | CD3+, CD8+, CD45RA+, CD28+ | CCR7 | 28060797 |
| CD8+ effector memory T-cells | T_CD8_EM | CD3+, CD8+, CD45RO+, CD57+, | GZMH, | 28060797 |
| | | CX3CR1+, CD16+ | GZMB | |
| CD8+ effector memory | T_CD8_EM_Int | CD3+, CD8+, CD45RA+, CD57dim, | GZMH, | 28060797 |
| intermediate T-cells | | CD360+, CX3CR1+, CD16+ | GZMB | |
| CD8+ terminally differentiated | T_CD8_TE | CD3+, CD8+, CD45RA+, CD360+, | EOMES | 28060797 |
| effector T-cells | | CD158b+ | | |
| CD8+ exhausted-like progenitor | T_CD8_PEX | CD3+, CD8+, CD279+, CD81+ | GZMK, | 33046887 |
| T-cell | | | JUNB | |
| CD8+ stem-like memory T-cells | T_CD8_SM | CD3+, CD8+, CD45RAdim, | IL7R | 28060797 |
| | | CD45ROdim, CD183+(CXCR3), | | |
| | | CD49f+ | | |
| CD8+ CD103+ tissue resident | T_CD8_TRM | CD3+, CD8+, CD103+, | IL7R | 25725111 |
| T-cells | | CD183+(CXCR3), CD49f− | | |
| CD8+ CCR4+ memory T-cells | T_CD8_Mem_CCR4 | CD49f+ | IL7R | 19261691 |
| | | CD3+, CD8+, CD194+(CCR4), CD82+, | | |
| CD8+ CD69+ T cells | T_CD8_CD69 | CD3+, CD8+, CD69+ | CD69 | |
| CD8+ KIR3DL1+ T-cell | T_CD8_KIR3DL1 | CD3+, CD8+, CD158e1+ (KIR3DL1) | KIR3DL1 | 30147699 |
| gamma/delta T-cells 1 | gdT | CD3+, TCR-Vdelta2+, TCR-Vgamma9+ | GZMB | |

TABLE 2
Overview of study population and clinical parameters part 1

| | | | antigen- | | | | |
|---|---|---|---|---|---|---|---|
| | | | agnostic | antigen- | | | |
| | | | forward TCR | dependent | | | |
| patient | | patient | screening | TCR screening | TCRA/B- | scRNA- | |
| ID | malignant disease | cohort | (microfluidics) | (MANAFEST) | seq | seq | scTCR-seq |
| Pt-01 | multiple myeloma | retrospective | x | | x | x | x |
| | | cohort | | | | | |
| Pt-02 | multiple myeloma | retrospective | x | | x | x | x |
| | | cohort | | | | | |
| Pt-03 | multiple myeloma | retrospective | x | | x | x | x |
| | | cohort | | | | | |
| Pt-04 | multiple myeloma | retrospective | x | x | x | x | x |
| | | cohort | | | | | |
| Pt-05 | multiple myeloma | retrospective | x | x | x | x | x |
| | | cohort | | | | | |
| Pt-06 | multiple myeloma | retrospective | x | x | x | x | x |
| | | cohort | | | | | |
| Pt-07 | multiple myeloma | prospective | x | x | x | x | x |
| | | cohort | | | | | |
| Pt-08 | multiple myeloma | prospective | x | x | x | x | x |
| | | cohort | | | | | |
| Pt-09 | multiple myeloma | prospective | x | x | x | x | x |
| | | cohort | | | | | |
| Pt-10 | multiple myeloma | prospective | x | x | x | x | x |
| | | cohort | | | | | |
| Pt-11 | multiple myeloma | prospective | x | x | x | x | x |
| | | cohort | | | | | |
| Pt-12 | multiple myeloma | prospective | x | x | x | x | x |
| | | cohort | | | | | |
| Pt-13 | multiple myeloma | prospective | x | | x | x | x |
| | | cohort | | | | | |
| Pt-14 | multiple myeloma | prospective | x | | x | x | x |
| | | cohort | | | | | |
| Pt-15 | multiple myeloma | prospective | x | | x | x | x |
| | | cohort | | | | | |
| Pt-16 | multiple myeloma | prospective | | | x | x | x |
| | | cohort | | | | | |
| Pt-17 | multiple myeloma | prospective | | | x | x | x |
| | | cohort | | | | | |
| Pt-18 | multiple myeloma | prospective | | | x | x | x |
| | | cohort | | | | | |
| Pt-19 | multiple myeloma | prospective | | | x | x | x |
| | | cohort | | | | | |
| Pt-20 | multiple myeloma | prospective | | | x | x | x |
| | | cohort | | | | | |
| Pt-21 | multiple myeloma | UCL cohort | | | x | x | x |
| Pt-22 | multiple myeloma | UCL cohort | | | x | | |
| Pt-23 | multiple myeloma | UCL cohort | | | x | | |
| Pt-24 | multiple myeloma | UCL cohort | | | x | | |
| Pt-25 | multiple myeloma | UCL cohort | | | x | | |
| Pt-26 | multiple myeloma | UCL cohort | | | x | | |
| Pt-27 | multiple myeloma | UCL cohort | | | x | | |
| Pt-28 | multiple myeloma | UCL cohort | | | x | | |
| Pt-29 | multiple myeloma | UCL cohort | | | x | | |
| Pt-30 | multiple myeloma | UCL cohort | | | x | | |
| Pt-31 | multiple myeloma | UCL cohort | | | x | | |
| Pt-32 | multiple myeloma | UCL cohort | | | x | | |
| Pt-33 | multiple myeloma | UCL cohort | | | x | | |
| Pt-34 | multiple myeloma | UCL cohort | | | x | | |
| Pt-35 | multiple myeloma | bsAb cohort | | | | x | x |
| Pt-36 | multiple myeloma | bsAb cohort | | | | x | x |
| Pt-37 | multiple myeloma | bsAb cohort | | | | x | x |
| Pt-38 | multiple myeloma | bsAb cohort | | | | x | x |
| Pt-39 | multiple myeloma | bsAb cohort | | | | x | x |
| Pt-40 | multiple myeloma | bsAb cohort | | | | x | x |
| Pt-41 | multiple myeloma | bsAb cohort | | | | x | x |
| Pt-42 | multiple myeloma | bsAb cohort | | | | x | x |
| Pt-43 | multiple myeloma | bsAb cohort | | | | x | x |
| Pt-44 | multiple myeloma | bsAb cohort | | | | x | x |
| Pt-45 | multiple myeloma | bsAb cohort | | | | x | x |
| Pt-46 | multiple myeloma | bsAb cohort | | | | x | x |
| Pt-47 | multiple myeloma | bsAb cohort | | | | x | x |
| Pt-48 | multiple myeloma | bsAb cohort | | | | x | x |
| Pt-49 | multiple myeloma | bsAb cohort | | | | x | x |
| Pt-50 | multiple myeloma | bsAb cohort | | | | x | x |
| Pt-51 | multiple myeloma | bsAb cohort | | | | x | x |
| Pt-52 | multiple myeloma | bsAb cohort | | | | x | x |
| | | | | | HLA | MHC | |
| patient | WGS tumor | WGS | RNA-seq | RNA-seq | haplo- | immuno- | |
| ID | cells | germline | tumor cells | germline | typing | peptidomics | diagnosis |
| Pt-01 | x | x | x | x | x | x | C90. (multiple myeloma) |
| Pt-02 | x | x | x | x | x | | C90. (multiple myeloma) |
| Pt-03 | x | x | x | x | x | x | C90. (multiple myeloma) |
| Pt-04 | x | x | x | x | x | x | C90. (multiple myeloma) |
| Pt-05 | x | x | x | x | x | x | C90. (multiple myeloma) |
| Pt-06 | x | x | x | x | x | x | C90. (multiple myeloma) |
| Pt-07 | x | x | x | x | x | x | C90. (multiple myeloma) |
| Pt-08 | x | x | x | x | x | x | C90. (multiple myeloma) |
| Pt-09 | x | x | x | x | x | x | C90. (multiple myeloma) |
| Pt-10 | x | x | x | x | x | x | C90. (multiple myeloma) |
| Pt-11 | x | x | x | x | x | x | C90. (multiple myeloma) |
| Pt-12 | x | x | x | x | x | x | C90. (multiple myeloma) |
| Pt-13 | x | x | x | x | x | | C90. (multiple myeloma) |
| Pt-14 | x | x | | | x | | C90. (multiple myeloma) |
| Pt-15 | x | x | x | x | x | | C90. (multiple myeloma) |
| Pt-16 | x | x | x | x | x | | C90. (multiple myeloma) |
| Pt-17 | x | x | x | x | x | | C90. (multiple myeloma) |
| Pt-18 | x | x | x | x | x | | C90. (multiple myeloma) |
| Pt-19 | x | x | x | x | x | | C90. (multiple myeloma) |
| Pt-20 | x | x | x | x | x | | C90. (multiple myeloma) |
| Pt-21 | | | | | | | C90. (multiple myeloma) |
| Pt-22 | | | | | | | C90. (multiple myeloma) |
| Pt-23 | | | | | | | C90. (multiple myeloma) |
| Pt-24 | | | | | | | C90. (multiple myeloma) |
| Pt-25 | | | | | | | C90. (multiple myeloma) |
| Pt-26 | | | | | | | C90. (multiple myeloma) |
| Pt-27 | | | | | | | C90. (multiple myeloma) |
| Pt-28 | | | | | | | C90. (multiple myeloma) |
| Pt-29 | | | | | | | C90. (multiple myeloma) |
| Pt-30 | | | | | | | C90. (multiple myeloma) |
| Pt-31 | | | | | | | C90. (multiple myeloma) |
| Pt-32 | | | | | | | C90. (multiple myeloma) |
| Pt-33 | | | | | | | C90. (multiple myeloma) |
| Pt-34 | | | | | | | C90. (multiple myeloma) |
| Pt-35 | | | | | | | C90.02 (multiple myeloma |
| | | | | | | | in relapse) |
| Pt-36 | | | | | | | C90.02 (multiple myeloma |
| | | | | | | | in relapse) |
| Pt-37 | | | | | | | C90.02 (multiple myeloma |
| | | | | | | | in relapse) |
| Pt-38 | | | | | | | C90.02 (multiple myeloma |
| | | | | | | | in relapse) |
| Pt-39 | | | | | | | C90.02 (multiple myeloma |
| | | | | | | | in relapse) |
| Pt-40 | | | | | | | C90.02 (multiple myeloma |
| | | | | | | | in relapse) |
| Pt-41 | | | | | | | C90.02 (multiple myeloma |
| | | | | | | | in relapse) |
| Pt-42 | | | | | | | C90.02 (multiple myeloma |
| | | | | | | | in relapse) |
| Pt-43 | | | | | | | C90.02 (multiple myeloma |
| | | | | | | | in relapse) |
| Pt-44 | | | | | | | C90.02 (multiple myeloma |
| | | | | | | | in relapse) |
| Pt-45 | | | | | | | C90.02 (multiple myeloma |
| | | | | | | | in relapse) |
| Pt-46 | | | | | | | C90.02 (multiple myeloma |
| | | | | | | | in relapse) |
| Pt-47 | | | | | | | C90.02 (multiple myeloma |
| | | | | | | | in relapse) |
| Pt-48 | | | | | | | C90.02 (multiple myeloma |
| | | | | | | | in relapse) |
| Pt-49 | | | | | | | C90.02 (multiple myeloma |
| | | | | | | | in relapse) |
| Pt-50 | | | | | | | C90.02 (multiple myeloma |
| | | | | | | | in relapse) |
| Pt-51 | | | | | | | C90.02 (multiple myeloma |
| | | | | | | | in relapse) |
| Pt-52 | | | | | | | C90.02 (multiple myeloma |
| | | | | | | | in relapse) |
| | | | | | | tumor | |
| | | | | | | reactive | |
| | | cytogenetic | | | | TCRs in | TCRs |
| patient | tumor | risk | primary | | | forward | tested for |
| ID | subtype | category | treatment | best response | PFS | screening* | specificity |
| Pt-01 | IgG | SR | 4x Dara-VTd | VGPR | not reached | 5 | 1 |
| | lambda | | | | | | |
| Pt-02 | LC | SR | 4x Dara-VTd | CR | not reached | 13.6666667 | 1 |
| | kappa | | | | | | |
| Pt-03 | IgG | SR | 4x VCD | VGPR | not reached | 6.5 | 3 |
| | kappa | | | | | | |
| Pt-04 | IgG | SR | 4x Dara-VCD | PR | 32 | 11.5 | 10 |
| | kappa | | | | | | |
| Pt-05 | LC | SR | 1x Dara-VCD, | sCR | not reached | 22 | 2 |
| | lambda | | 3x Dara-VTD | | | | |
| Pt-06 | IgG | SR | 4x Dara-VCD | VGPR, MRD | not reached | 9.5 | 7 |
| | kappa | | | neg. | | | |
| Pt-07 | IgG | SR | 6x Dara-VTd | VGPR, MRD | not reached | 13.5 | 3 |
| | kappa | | | neg. | | | |
| Pt-08 | IgG | HR | 5x VCD | n/a | 34 | 24 | 2 |
| | lambda | | | | | | |
| Pt-09 | IgG | SR | 4x Dara-VRd | CR | not reached | 27 | 12 |
| | kappa | | | | | | |
| Pt-10 | IgA | HR | 4x Dara-VRd | VGPR, MRD | not reached | 9 | 1 |
| | kappa | | | neg. | | | |
| Pt-11 | LC | SR | 4x Dara-RD | sCR | not reached | 27 | 12 |
| | kappa | | | | | | |
| Pt-12 | LC | HR | 4x Dara-VRd | VGPR | not reached | 7 | 11 |
| | lambda | | | | | | |
| Pt-13 | IgG | SR | 4x Dara-RD | CR | not reached | 28 | |
| | kappa | | | (radiographical) | | | |
| Pt-14 | LC | HR | 4x Dara-VRd | VGPR | not reached | 9.5 | |
| | lambda | | | | | | |
| Pt-15 | LC | SR | 4x Dara-VRd | CR | not reached | 11.5 | |
| | lambda | | | | | | |
| Pt-16 | IgG | SR | 3x Isa-VRd | sCR, MRD | not reached | 41 | |
| | Kappa | | | neg. | | | |
| Pt-17 | IgG | SR | 3x Isa-VRd | CR, MRD | not reached | 11.5 | |
| | lambda | | | neg. | | | |
| Pt-18 | IgG | SR | 3x Isa-VRd | sCR, MRD | not reached | 10.5 | |
| | lambda | | | neg. | | | |
| Pt-19 | IgG | SR | 3x Isa-VRd | SCR, MRD | not reached | | |
| | kappa | | | neg. | | | |
| Pt-20 | LC | HR | 4x Isa-VRd | SCR, MRD | not reached | | |
| | lambda | | | neg. | | | |
| Pt-21 | LC | HR | KCD | nCR, MRD | not reached | | |
| | lambda | | | neg. | | | |
| Pt-22 | LC | SR | KCD | nCR, MRD | not reached | | |
| | kappa | | | neg. | | | |
| Pt-23 | IgG | SR | KCD | nCR, MRD | not reached | | |
| | kappa | | | neg. | | | |
| Pt-24 | IgG | SR | KCD | VGPR | not reached | | |
| | kappa | | | | | | |
| Pt-25 | IgG | SR | KCD | VGPR | not reached | | |
| | kappa | | | | | | |
| Pt-26 | IgG | SR | KCD | nCR | not reached | | |
| | lambda | | | | | | |
| Pt-27 | LC | SR | KCD | nCR | not reached | | |
| | lambda | | | | | | |
| Pt-28 | LC | SR | KCD | PR | not reached | | |
| | lambda | | | | | | |
| Pt-29 | IgG | SR | KCD | PR | not reached | | |
| | kappa | | | | | | |
| Pt-30 | IgG | SR | KCD | nCR, MRD | not reached | | |
| | lambda | | | neg. | | | |
| Pt-31 | LC | SR | KCD | nCR, MRD | not reached | | |
| | lambda | | | neg. | | | |
| Pt-32 | LC | SR | KCD | nCR, MRD | not reached | | |
| | lambda | | | neg. | | | |
| Pt-33 | IgG | SR | KCD | nCR, MRD | not reached | | |
| | kappa | | | neg. | | | |
| Pt-34 | IgG | SR | KCD | nCR, MRD | not reached | | |
| | kappa | | | neg. | | | |
| Pt-35 | LC | HR | Elranatamab | CR | 23 | | |
| | lambda | | | | | | |
| Pt-36 | IgG | SR | Elranatamab | PD | 1 | | |
| | kappa | | | | | | |
| Pt-37 | IgG | SR | Elranatamab | CR | 13 | | |
| | lambda | | | | | | |
| Pt-38 | LC | SR | Elranatamab | MR | 11 | | |
| | kappa | | | | | | |
| Pt-39 | IgG | HR | Elranatamab | MR | 5 | | |
| | kappa | | | | | | |
| Pt-40 | IgA | SR | Elranatamab | SD | 2 | | |
| | lambda | | | | | | |
| Pt-41 | IgG | HR | Elranatamab | VGPR | 13 | | |
| | lambda | | | | | | |
| Pt-42 | LC | HR | Elranatamab | VGPR | 7 | | |
| | kappa | | | | | | |
| Pt-43 | IgG | HR | Elranatamab | PD | 1 | | |
| | kappa | | | | | | |
| Pt-44 | IgG | HR | Elranatamab | MR | 1 | | |
| | kappa | | | | | | |
| Pt-45 | IgG | SR | Elranatamab | CR | 21 | | |
| | kappa | | | | | | |
| Pt-46 | IgG | HR | Elranatamab | VGPR | 19 | | |
| | lambda | | | | | | |
| Pt-47 | LC | SR | Elranatamab | SD | 9 | | |
| | lambda | | | | | | |
| Pt-48 | LC | HR | Elranatamab | VGPR | 17 | | |
| | lambda | | | | | | |
| Pt-49 | IgG | SR | Elranatamab | CR | 14 | | |
| | kappa | | | | | | |
| Pt-50 | LC | HR | Elranatamab | CR | 14 | | |
| | kappa | | | | | | |
| Pt-51 | IgG | HR | Elranatamab | VGPR | 6 | | |
| | kappa | | | | | | |
| Pt-52 | IgG | HR | Elranatamab | PR | 17 | | |

| | | private | |
|---|---|---|---|
| | public | peptides | |
| patient ID | peptides tested | tested | Ig (BCR)-derived peptides tested |
| Pt-01 | 87 | 8 | |
| Pt-02 | 87 | | |
| Pt-03 | 87 | 17 | |
| Pt-04 | 87 | 11 | 1 |
| Pt-05 | 87 | 12 | 2 |
| Pt-06 | 87 | 14 | |
| Pt-07 | 87 | 8 | 2 |
| Pt-08 | 87 | 40 | 5 |
| Pt-09 | 87 | 13 | |
| Pt-10 | 87 | 11 | |
| Pt-11 | 87 | 24 | 10 |
| Pt-12 | 87 | 55 | 10 |

Overview of study population and clinical parameters part 2

| | | | antigen- | | | | | | |
|---|---|---|---|---|---|---|---|---|---|
| | | | agnostic | | | | | | |
| | | | TCR | | | | | | |
| | | | screening | | | HLA | MHC | | tumor |
| | malignant | patient | (micro- | scRNA- | scTCR- | haplo- | immuno- | | reactive |
| patient ID | disease | cohort | fluidics) | seq | seq | typing | peptidomics | diagnosis | TCRs* |
| AML-24 | acute | AML | x | x | x | x | x | C92.00 | 4 |
| | myeloid | PoC | | | | | | (acute | |
| | leukemia | | | | | | | myeloblastic | |
| | | | | | | | | leukemia) | |
| AML-37 | acute | AML | x | x | x | x | x | C92.00 | 12.5 |
| | myeloid | PoC | | | | | | (acute | |
| | leukemia | | | | | | | myeloblastic | |
| | | | | | | | | leukemia) | |
| AML-92 | acute | AML | x | x | x | x | x | C92.00 | 5 |
| | myeloid | PoC | | | | | | (acute | |
| | leukemia | | | | | | | myeloblastic | |
| | | | | | | | | leukemia) | |
| AML-108 | acute | AML | x | x | x | x | x | C92.00 | 9 |
| | myeloid | PoC | | | | | | (acute | |
| | leukemia | | | | | | | myeloblastic | |
| | | | | | | | | leukemia) | |
| AML-113 | acute | AML | x | x | x | x | x | C92.00 | 2 |
| | myeloid | PoC | | | | | | (acute | |
| | leukemia | | | | | | | myeloblastic | |
| | | | | | | | | leukemia) | |
| AML-116 | acute | AML | x | x | x | x | x | C92.00 | 3 |
| | myeloid | PoC | | | | | | (acute | |
| | leukemia | | | | | | | myeloblastic | |
| | | | | | | | | leukemia) | |

Overview of study population and clinical parameters part 3

| | | | antigen- | | | | | |
|---|---|---|---|---|---|---|---|---|
| | | | agnostic | | | | | |
| | | | TCR | | | | | |
| | | | screening | | HLA | | | tumor |
| | malignant | patient | (micro- | TCRA/B- | haplo- | | tumor | reactive |
| patient ID | disease | cohort | fluidics) | seq | typing | diagnosis | subtype | TCRs* |
| CLL-23626 | chronic | CLL | x | x | x | C91.1 | CD19+, | 3.5 |
| | lymphocytic | PoC | | | | (chronic | CD5+, CD20 | |
| | leukemia | | | | | lymphocytic | low, CD23+, | |
| | | | | | | leukemia of | CD79b−, | |
| | | | | | | B-cell type) | IgM+, CD22−, | |
| | | | | | | | CD200+ | |
| CLL-12977 | chronic | CLL | x | x | x | C91.1 | n.a. | 3 |
| | lymphocytic | PoC | | | | (chronic | | |
| | leukemia | | | | | lymphocytic | | |
| | | | | | | leukemia of | | |
| | | | | | | B-cell type) | | |
| CLL-13611 | chronic | CLL | x | x | x | C91.1 | CD20−, | 9 |
| | lymphocytic | PoC | | | | (chronic | CD5+, | |
| | leukemia | | | | | lymphocytic | CD22low, | |
| | | | | | | leukemia of | CD23+, | |
| | | | | | | B-cell type) | CD24+, | |
| | | | | | | | FMC7low, | |

| *average from all screening runs, normalized by % T cells successfully penned | |
|---|---|
| Definitions: | |
| bsAb | Bispecific antibody |
| C | Cyclophosphamide |
| CR | Complete response |
| d | Dexamethasone |
| Dara | Daratumumab |
| HR | High risk |
| | cytogenetics |
| | (one or more of |
| | t(4;14), t(14;16), |
| | or del 17p |
| | identified from FISH |
| | test results) |
| Isa | Isatuximab |
| K | Carfilzomib |
| LC | Light chain |
| MR | Minimal response |
| MRD | minimal residual |
| | disease |
| PD | Progressive disease |
| PFS | Progression-free |
| | survival |
| PR | Partial response |
| SD | S disease |
| SR | Standard risk |
| | cytogenetics |
| | (absense of HR) |
| T | Thalidomide |
| V | Bortezomib |
| VGPR | Very good partial |
| | response |

TABLE 3
Penning efficiencies and detected events for antigen-agnostic TCR tumor reactivity
screening - CLL part 1

| | | total detected events | | | | | % assayed BMR T cells | | |
|---|---|---|---|---|---|---|---|---|---|
| Exp. | Patient | CD137 | Mono cytokine | Poly- | | % T cells | CD137 | Mono cytokine | Poly- |
| No. | ID | only | secretion only | functional | Total | penned | only | secretion only | functional |
| 39 | AML- | 2 | 2 | 0 | 4 | 94 | 0.16 | 0.16 | 0 |
| | 24 | | | | | | | | |
| 40 | AML- | 0 | 4 | 1 | 5 | 96 | 0 | 0.32 | 0.08 |
| | 37 | | | | | | | | |
| 41 | AML- | 4 | 0 | 1 | 5 | 87 | 0.32 | 0 | 0.08 |
| | 92 | | | | | | | | |
| 42 | AML- | 1 | 3 | 1 | 5 | 96 | 0.08 | 0.24 | 0.08 |
| | 108 | | | | | | | | |
| 43 | AML- | 2 | 0 | 0 | 2 | 96 | 0.16 | 0 | 0 |
| | 113 | | | | | | | | |
| 44 | AML- | 2 | 1 | 0 | 3 | 96 | 0.16 | 0.08 | 0 |
| | 116 | | | | | | | | |
| 45 | AML- | 2 | 9 | 2 | 13 | 85 | 0.16 | 0.72 | 0.16 |
| | 108 | | | | | | | | |
| 46 | AML- | 2 | 2 | 0 | 4 | 91 | 0.16 | 0.16 | 0 |
| | 24 | | | | | | | | |
| 47 | AML- | 2 | 15 | 3 | 20 | 96 | 0.16 | 1.21 | 0.24 |
| | 37 | | | | | | | | |

| | |
|---|---|
| Total number of reactive T cells identified: | 61 |

Penning efficiencies and detected events for antigen-agnostic TCR tumor reactivity
screening - CLL part 2

| | | CD8+ | | | | | CD8− | | | | |
|---|---|---|---|---|---|---|---|---|---|---|---|
| Exp. | Patient | | | | | Poly- | | | | | Poly- |
| No. | ID | CD137+ | IFN_ [symbol] | IL-2 | TNF_ [symbol] | functional | CD137+ | IFN_ [symbol] | IL-2 | TNF_ [symbol] | functional |
| 39 | AML- | 2 | 0 | 0 | 1 | 0 | 0 | 1 | 0 | 0 | 0 |
| | 24 | | | | | | | | | | |
| 40 | AML- | 0 | 1 | 0 | 0 | 1 | 0 | 1 | 0 | 2 | 0 |
| | 37 | | | | | | | | | | |
| 41 | AML- | 1 | 0 | 0 | 0 | 0 | 3 | 0 | 0 | 0 | 1 |
| | 92 | | | | | | | | | | |
| 42 | AML- | 1 | 1 | 0 | 1 | 0 | 0 | 1 | 0 | 0 | 1 |
| | 108 | | | | | | | | | | |
| 43 | AML- | 0 | 0 | 0 | 0 | 0 | 2 | 0 | 0 | 0 | 0 |
| | 113 | | | | | | | | | | |
| 44 | AML- | 2 | 0 | 0 | 1 | 0 | 0 | 0 | 0 | 0 | 0 |
| | 116 | | | | | | | | | | |
| 45 | AML- | 1 | 3 | 0 | 1 | 2 | 1 | 0 | 0 | 5 | 0 |
| | 108 | | | | | | | | | | |
| 46 | AML- | 1 | 1 | 0 | 1 | 0 | 1 | 0 | 0 | 0 | 0 |
| | 24 | | | | | | | | | | |
| 47 | AML- | 1 | 9 | 0 | 2 | 1 | 1 | 1 | 0 | 3 | 2 |
| | 37 | | | | | | | | | | |
| Total | | 9 | 15 | 0 | 7 | 4 | 8 | 4 | 0 | 10 | 4 |
| Ratio | | 0.53 | 0.79 | #DIV/0! | 0.41 | 0.5 | 0.47 | 0.21 | #DIV/0! | 0.59 | 0.5 |

[symbol]

indicates data missing or illegible when filed

TABLE 4
Tumor-reactive TCR alpha and beta chains used for specificity testing part 1

| | | total detected events | | | | | | | |
|---|---|---|---|---|---|---|---|---|---|
| | | | Mono | | | | % assayed BMR T cells | | |
| | | | cytokine | | | | CD13 | | |
| Exp. | | CD13 | secretion | Poly- | | % T cells | 7 | Mono cytokine | |
| No. | Patient ID | 7 only | only | functional | Total | penned | only | secretion only | Polyfunctional |
| 53 | CLL- | 3 | 0 | 1 | 4 | 94 | 0.24 | 0 | 0.08 |
| | 23626 | | | | | | | | |
| 54 | CLL- | 3 | 0 | 0 | 3 | 86 | 0.24 | 0 | 0 |
| | 12977 | | | | | | | | |
| 55 | CLL- | 8 | 1 | 0 | 9 | 94 | 0.64 | 0.08 | 0 |
| | 13611 | | | | | | | | |
| 56 | CLL- | 1 | 2 | 0 | 3 | 95 | 0.08 | 0.16 | 0 |
| | 23626 | | | | | | | | |

| | |
|---|---|
| Total number of reactive T cells identified: | 19 |

Tumor-reactive TCR alpha and beta chains used for specificity testing part 2

| | | CD8+ | | | | | CD8− | | | | |
|---|---|---|---|---|---|---|---|---|---|---|---|
| Exp. | | CD137 | | | | Poly- | | | IL- | | Poly- |
| No. | Patient ID | + | IFN__ [symbol] | IL-2 | TNF_ [symbol] | functional | CD137+ | IFN_ [symbol] | 2 | TNF_ [symbol] | functional |
| 53 | CLL- | 3 | 0 | 0 | 0 | 0 | 0 | 0 | 0 | 0 | 1 |
| | 23626 | | | | | | | | | | |
| 54 | CLL- | 3 | 0 | 0 | 0 | 0 | 0 | 0 | 0 | 0 | 0 |
| | 12977 | | | | | | | | | | |
| 55 | CLL- | 5 | 1 | 0 | 0 | 0 | 3 | 0 | 0 | 0 | 0 |
| | 13611 | | | | | | | | | | |
| 56 | CLL- | 1 | 0 | 1 | 0 | 0 | 0 | 0 | 0 | 1 | 0 |
| | 23626 | | | | | | | | | | |
| Total | | 12 | 1 | 1 | 0 | 0 | 3 | 0 | 0 | 1 | 1 |
| Ratio | | 0.8 | 1 | 1 | 0 | 0 | 0.2 | 0 | 0 | 1 | 1 |

[symbol]

indicates data missing or illegible when filed

TABLE 5
HLA haplotypes in multiple myeloma patients.

| | allele | Pt-01 | Pt-02 | Pt-03 | Pt-04 | Pt-05 | Pt-06 | Pt-07 | Pt-08 | Pt-09 | Pt-10 | Pt-11 | Pt-12 | Pt-13 | Pt-14 | Pt-15 | Pt-16 | Pt-17 | Pt-18 | Pt-19 | Pt-20 |
|---|---|---|---|---|---|---|---|---|---|---|---|---|---|---|---|---|---|---|---|---|---|
| 1 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | AHL | A*03 | A*02 | A*02 | A*02 | A*02 | A*02 | A*03 | A*01 | A*03 | A*02 | A*01 | A*11 | A*03 | A*03 | A*01 | A*02 | A*26 | A*01 | A*02 | A*01 |
| 2 | A- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | A | A*33 | A*02 | A*31 | A*26 | A*03 | A*03 | A*25 | A*02 | A*03 | A*02 | A*23 | A*26 | A*23 | A*24 | A*02 | A*01 | A*02 | B*35 | A*26 | B*40 |
| 3 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | B | B*07 | B*27 | B*27 | B*15 | B*07 | B*07 | B*15 | B*07 | B*07 | B*27 | B*50 | B*27 | B*49 | B*51 | B*44 | B*37 | B*44 | B*44 | B*27 | B*08 |
| 4 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | B | B*58 | B*57 | B*44 | B*44 | B*55 | B*27 | B*35 | B*08 | B*07 | B*40 | B*52 | B*35 | B*51 | B*55 | B*57 | B*07 | B*51 | C*07 | B*57 | C*07 |
| 5 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | C | C*03 | C*02 | C*01 | C*05 | C*03 | C*02 | C*03 | C*07 | C*07 | C*02 | C*06 | C*01 | C*02 | C*01 | C*05 | C*07 | C*05 | C*06 | C*06 | C*02 |
| 6 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | C | C*07 | C*06 | C*05 | C*12 | C*07 | C*07 | C*04 | C*07 | C*07 | C*03 | C*12 | C*04 | C*07 | C*15 | C*06 | C*06 | C*15 | DQA1 | C*01 | DQA1 |
| | | | | | | | | | | | | | | | | | | | *05 | | *05 |
| 7 | HLA- | DRB1 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | DR | HLA- | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 |
| | B1 | *04 | *01 | *08 | *01 | *14 | *11 | *01 | *13 | *15 | *13 | *08 | *01 | *11 | *04 | *04 | *01 | *16 | *11 | *07 | *08 |
| 8 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | DR | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 | DRB1 |
| | B1 | *13 | *07 | *11 | *13 | *15 | *15 | *13 | *15 | *15 | *15 | *15 | *01 | *11 | *13 | *07 | *10 | *01 | *11 | *01 | *08 |
| 9 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | DQ | DQA1 | DQA1 | DQA1 | DQA1 | DQA1 | DQA1 | DQA1 | DQA1 | DQA1 | DQA1 | DQA1 | DQA1 | DQA1 | DQA1 | DQA1 | DQA1 | DQA1 | DQA1 | DQA1 | DQA1 |
| | A1 | *01 | *01 | *04 | *01 | *01 | *01 | *01 | *01 | *01 | *01 | *01 | *01 | *05 | *01 | *02 | *01 | *01 | *01 | *01 | *04 |
| 10 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | DQ | DQA1 | DQA1 | DQA1 | DQA1 | DQA1 | DQAI | DQA1 | DQA1 | DQA1 | DQAI | DQA1 | DQA1 | DQA1 | DQA1 | DQA1 | DQA1 | DQAI | DQB1 | DQA1 | DQB1 |
| | A1 | *03 | *02 | *05 | *01 | *01 | *05 | *05 | *01 | *01 | *01 | *06 | *01 | *05 | *03 | *03 | *01 | *01 | *05 | *02 | *02 |
| 11 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | DQ | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 |
| | B1 | *03 | *02 | *03 | *05 | *05 | *03 | *03 | *06 | *06 | *06 | *03 | *05 | *03 | *03 | *02 | *05 | *05 | *03 | *05 | *04 |
| 12 | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | DQ | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 | DQB1 | DRB1 | DQB1 | DRB1 |
| | B1 | *06 | *05 | *04 | *06 | *06 | *06 | *05 | *06 | *06 | *06 | *05 | *05 | *03 | *06 | *03 | *05 | *05 | *16 | *03 | *03 |
| DRA | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- | HLA- |
| | DRA | DRA* | DRA* | DRA* | DRA* | DRA* | DRA* | DRA* | DRA* | DRA* | DRA* | DRA* | DRA* | DRA* | DRA* | DRA* | DRA* | DRA* | DRA* | DRA* | DRA* |
| | | 01 | 01 | 01 | 01 | 01 | 01 | 01 | 01 | 01 | 01 | 01 | 01 | 01 | 01 | 01 | 01 | 01 | 01 | 01 | 01 |
| B2M | B2M | B2M | B2M | B2M | B2M | B2M | B2M | B2M | B2M | B2M | B2M | B2M | B2M | B2M | B2M | B2M | B2M | B2M | B2M | B2M | B2M |

TABLE 6
transcriptional signatures of TILS.

| | sigMM | sigMM_2 | sigMM_3 | sigMM_4 | sigMM_5 | sigMM_6 | sigMM_7 | sigMM_8 |
|---|---|---|---|---|---|---|---|---|
| 1 | GNLY | GNLY | GNLY | GNLY | GNLY | GNLY | GNLY | EFHD2 |
| 2 | ZNF683 | ZNF683 | ZNF683 | ZNF683 | ZNF683 | ZNF683 | ZNF683 | SH3BGRL3 |
| 3 | GZMH | GZMH | GZMH | GZMH | GZMH | GZMH | GZMH | CD52 |
| 4 | FGFBP2 | FGFBP2 | FGFBP2 | FGFBP2 | FGFBP2 | FGFBP2 | FGFBP2 | ZNF683 |
| 5 | GZMB | GZMB | GZMB | GZMB | GZMB | GZMB | GZMB | S100A10 |
| 6 | NKG7 | NA | NKG7 | NKG7 | NKG7 | NKG7 | NKG7 | S100A6 |
| 7 | CCL5 | NA | CCL5 | CCL5 | CCL5 | CCL5 | CCL5 | S100A4 |
| 8 | HOPX | NA | HOPX | HOPX | HOPX | HOPX | HOPX | FCRL6 |
| 9 | KLRD1 | NA | KLRD1 | KLRD1 | KLRD1 | KLRD1 | KLRD1 | TAGLN2 |
| 10 | EFHD2 | NA | EFHD2 | EFHD2 | EFHD2 | EFHD2 | EFHD2 | C1orf21 |
| 11 | CD8A | NA | NA | CD8A | CD8A | CD8A | CD8A | PLEK |
| 12 | CTSW | NA | NA | CTSW | CTSW | CTSW | CTSW | GNLY |
| 13 | CST7 | NA | NA | CST7 | CST7 | CST7 | CST7 | CD8A |
| 14 | ITGB1 | NA | NA | ITGB1 | ITGB1 | ITGB1 | ITGB1 | ZEB2 |
| 15 | BHLHE40 | NA | NA | BHLHE40 | BHLHE40 | BHLHE40 | BHLHE40 | ITGA4 |
| 16 | NA | NA | NA | LYAR | LYAR | LYAR | LYAR | BHLHE40 |
| 17 | NA | NA | NA | S100A4 | S100A4 | S100A4 | S100A4 | LYAR |
| 18 | NA | NA | NA | GZMA | GZMA | GZMA | GZMA | FGFBP2 |
| 19 | NA | NA | NA | MXRA7 | MXRA7 | MXRA7 | MXRA7 | HOPX |
| 20 | NA | NA | NA | KLRK1 | KLRK1 | KLRK1 | KLRK1 | GZMA |
| 21 | NA | NA | NA | NA | SH3BGRL3 | SH3BGRL3 | SH3BGRL3 | CLIC1 |
| 22 | NA | NA | NA | NA | ITGA4 | ITGA4 | ITGA4 | HLA-DPA1 |
| 23 | NA | NA | NA | NA | FCRL6 | FCRL6 | FCRL6 | HLA-DPB1 |
| 24 | NA | NA | NA | NA | TGFB1 | TGFB1 | TGFB1 | TNFAIP3 |
| 25 | NA | NA | NA | NA | CCL4 | CCL4 | CCL4 | AOAH |
| 26 | NA | NA | NA | NA | ZEB2 | ZEB2 | ZEB2 | ANXA1 |
| 27 | NA | NA | NA | NA | AOAH | AOAH | AOAH | KLF6 |
| 28 | NA | NA | NA | NA | AHNAK | AHNAK | AHNAK | ITGB1 |
| 29 | NA | NA | NA | NA | S100A10 | S100A10 | S100A10 | PRF1 |
| 30 | NA | NA | NA | NA | LGALS1 | LGALS1 | LGALS1 | AHNAK |
| 31 | NA | NA | NA | NA | NA | PRF1 | PRF1 | CTSW |
| 32 | NA | NA | NA | NA | NA | ITGB2 | ITGB2 | PRSS23 |
| 33 | NA | NA | NA | NA | NA | CD52 | CD52 | KLRD1 |
| 34 | NA | NA | NA | NA | NA | TPST2 | TPST2 | KLRK1 |
| 35 | NA | NA | NA | NA | NA | PRSS23 | PRSS23 | LINC02446 |
| 36 | NA | NA | NA | NA | NA | ANXA1 | ANXA1 | RPS26 |
| 37 | NA | NA | NA | NA | NA | CYBA | CYBA | C12orf75 |
| 38 | NA | NA | NA | NA | NA | C12orf75 | C12orf75 | RGCC |
| 39 | NA | NA | NA | NA | NA | LAIR2 | LAIR2 | GZMH |
| 40 | NA | NA | NA | NA | NA | MATK | MATK | GZMB |
| 41 | NA | NA | NA | NA | NA | NA | S100A6 | NFKBIA |
| 42 | NA | NA | NA | NA | NA | NA | TNFAIP3 | SYNE2 |
| 43 | NA | NA | NA | NA | NA | NA | CLIC1 | FOS |
| 44 | NA | NA | NA | NA | NA | NA | KLF6 | PPP2R5C |
| 45 | NA | NA | NA | NA | NA | NA | C1orf21 | CRIP1 |
| 46 | NA | NA | NA | NA | NA | NA | SYNE2 | AKAP13 |
| 47 | NA | NA | NA | NA | NA | NA | HLA-DPB1 | CYBA |
| 48 | NA | NA | NA | NA | NA | NA | HLA-DPA1 | CCL5 |
| 49 | NA | NA | NA | NA | NA | NA | DSTN | CCL4 |
| 50 | NA | NA | NA | NA | NA | NA | CD99 | MXRA7 |
| 51 | NA | NA | NA | NA | NA | NA | NA | GADD45B |
| 52 | NA | NA | NA | NA | NA | NA | NA | MATK |
| 53 | NA | NA | NA | NA | NA | NA | NA | ZFP36 |
| 54 | NA | NA | NA | NA | NA | NA | NA | TGFB1 |
| 55 | NA | NA | NA | NA | NA | NA | NA | NKG7 |
| 56 | NA | NA | NA | NA | NA | NA | NA | LAIR2 |
| 57 | NA | NA | NA | NA | NA | NA | NA | DSTN |
| 58 | NA | NA | NA | NA | NA | NA | NA | CST7 |
| 59 | NA | NA | NA | NA | NA | NA | NA | ITGB2 |
| 60 | NA | NA | NA | NA | NA | NA | NA | TPST2 |
| 61 | NA | NA | NA | NA | NA | NA | NA | LGALS1 |
| 62 | NA | NA | NA | NA | NA | NA | NA | CD99 |
| 63 | NA | NA | NA | NA | NA | NA | NA | FLNA |

TABLE 7
TCR classification benchmarking of MM-TCR
signature versus published signatures of TILs.

| gene_signature | AUROC_score |
|---|---|
| sigMM | 0.979454992 |
| sigMM_4 | 0.97825265 |
| sigMM_3 | 0.977027274 |
| sigMM_5 | 0.976573055 |
| sigMM_8 | 0.976445192 |
| sigMM_6 | 0.975252678 |
| sigMM_7 | 0.974456769 |
| sigMM_2 | 0.970222503 |
| Caushi.CD8-TRM(1) | 0.965857885 |
| Wu.8Trm.2 | 0.963973051 |
| Wu.8Trm.3 | 0.962482105 |
| Wu.8EFF | 0.962091176 |
| Oh.CD8.FGFBP2 | 0.961591086 |
| Oh.CD4.GZMB | 0.958639525 |
| Yost_CD8_Eff_mem | 0.956471813 |
| Caushi.CD8.Effector(III) | 0.954626133 |
| Lowery.Cluster_4 | 0.953521044 |
| Caushi.CD8-TRM(2) | 0.946018058 |
| Caushi.CD8-Effector(3) | 0.94536059 |
| Caushi.CD8.TRM(VI) | 0.936673922 |
| Caushi.CD8.TRM(III) | 0.93130301 |
| Wu.8chrom | 0.930739786 |
| Caushi.MANA | 0.926558745 |
| Oliveira.TTE | 0.921782674 |
| Lowery.Cluster_7 | 0.919537395 |
| Caushi.CD8.TRM(II) | 0.91133982 |
| Lowery.Cluster_6 | 0.910658187 |
| B16_TERMINAL.EX_Miller | 0.909042593 |
| Wu.8Trm.1 | 0.897183813 |
| Oliveira.Tumor-spec_TTE | 0.888652784 |
| Lowery.NeoTCR8 | 0.882253741 |
| Oh.CD4.GZMK | 0.874614979 |
| Wu.8EM | 0.861569889 |
| Wu.CD4.TCF7 | 0.860036074 |
| Caushi.CD8-Proliferating | 0.851600302 |
| Caushi.CD8-Effector(2) | 0.850382568 |
| Caushi.CD8.Effector(II) | 0.849950957 |
| Caushi.CD8-Effector(1) | 0.849903508 |
| Yost_CD8_Act/Exh | 0.847673402 |
| Wu.CD4.RPL32 | 0.843210254 |
| Caushi.CD4-Tfh(1) | 0.836392469 |
| Caushi.CD8.TRM(V) | 0.835458721 |
| Yost_CD8_Exh | 0.832391621 |
| Caushi.CD4CD8(I) | 0.824603467 |
| Caushi.CD8.CD4CD8(I) | 0.824603467 |
| Oh.CD4.TH17 | 0.823925071 |
| Caushi.CD4CD8(II) | 0.819895647 |
| Caushi.CD8.CD4CD8(II) | 0.819895647 |
| TOX_Scott | 0.813513627 |
| LCMV_PROG.EX_Miller | 0.812302205 |
| Yost_CD8_Memory | 0.802817308 |
| Caushi.Stem-like_memory | 0.801629529 |
| LCMV_TERMN.EX_Miller | 0.796909759 |
| Mem_Eff_6_Feldman | 0.786568365 |
| Caushi.CD8.Proliferating | 0.779251239 |
| Caushi.CD8.Stem-like_memory | 0.776130767 |
| Caushi.CD8.TRM(IV) | 0.773939609 |
| Yost_CD8_Activated | 0.767836261 |
| Caushi.CD4-Treg | 0.763451457 |
| Oh.CD8.MAIT | 0.760585889 |
| Lowery.Cluster_10 | 0.758340222 |
| Lowery.Cluster_9 | 0.757561379 |
| Oh.CD4.CM | 0.753216059 |
| Oh.CD8.XCL | 0.749609026 |
| B16_PROG.EX_Miller | 0.748717794 |
| Caushi.CD8-MHCII | 0.748653918 |
| Caushi.CD4-Th(3) | 0.747601115 |
| Oliveira.Tumor-spec_TEM | 0.729612107 |
| Wu.CD4.IL6ST | 0.722844324 |
| Lowery.Cluster_11 | 0.719261111 |
| Oh.CD4IL2RAHI | 0.715804021 |
| Lowery.Cluster_8 | 0.714186108 |
| Mel_Exhaust_Tirosh | 0.713250972 |
| Oh.CD8.RPL | 0.712896018 |
| Lowery.Cluster_5 | 0.705373455 |
| Wu.CD4.Trm | 0.702253049 |
| Oliveira.Tumor_spec-TAct | 0.698966368 |
| Yost_Naive | 0.692240661 |
| Wu.Treg.1 | 0.681945049 |
| Oh.CD4.Activate | 0.678526886 |
| Oliveira.TEM | 0.678039811 |
| Lowery.Cluster_1 | 0.673277892 |
| CD3.MT | 0.670303142 |
| Oh.CD4IL2RALO | 0.665216585 |
| Lowery.Cluster_2 | 0.650013134 |
| Oh.CD8.CD39 | 0.646849544 |
| Lowery.Cluster_3 | 0.646456911 |
| Oh.TIL_CD4IL2RALO | 0.641949683 |
| Caushi.CD4-Th(2) | 0.637265595 |
| Oliveira.TAct | 0.636068098 |
| Wu.8KLRB1 | 0.636058056 |
| Mem_Eff_4_Feldman | 0.630368423 |
| Caushi.CD8.Effector(I) | 0.629191376 |
| Krishna.ACT.Term.Diff | 0.624564565 |
| Wu.CD4.FOS | 0.614532189 |
| Oh.CD8.HSP | 0.61345735 |
| Caushi.CD4-Th(1) | 0.603378803 |
| Oh.CD4.MITO | 0.602747121 |
| Caushi.CD8.TRM(I) | 0.598832311 |
| Wu.8Mit | 0.593224942 |
| Early.Act_5_Feldman | 0.589249162 |
| Oh.CD8.CM | 0.579846992 |
| Caushi.MAIT | 0.572690012 |
| Oliveira.TProl | 0.57143871 |
| Oliveira.Tumor-spec_TProl | 0.571344877 |
| Oh.CD8.MITO | 0.568205048 |
| Oh.CD4.CXCL13 | 0.567987492 |
| Oh.TIL_CD4.GZMK | 0.56665576 |
| Oh.CD8.Naive | 0.566106541 |
| Oliveira.Tumor_spec-TPE | 0.561477785 |
| Krishna.ACT.Stem.Like | 0.550087941 |
| Jansen_Term_diff | 0.546452112 |
| CD8_G_Feldman | 0.544868236 |
| Lowery.Cluster_0 | 0.543120984 |
| CD8_B_Feldman | 0.534295236 |
| Caushi.CD8.MAIT | 0.531460197 |
| Oliveira.TPE | 0.523735185 |
| Lowery.NeoTCR4 | 0.523125231 |
| Caushi.CD4-Tfh(2) | 0.521000013 |
| Exhaust_2_Feldman | 0.520219093 |
| Oh.TIL_CD4.GZMB | 0.519965598 |
| Jansen_Stem_like | 0.51448825 |
| Oh.CD4.HSP | 0.50955146 |
| Oh.CD8.PRO | 0.508060074 |
| Li.CD8.DYS | 0.507012248 |
| Exhaust_3_Feldman | 0.506105234 |
| Oh.TIL_CD4IL2RAHI | 0.503070807 |
| Wu.Treg.2 | 0.501952616 |
| Oh.CD4.PROLIF | 0.500933755 |
| Exhaust_1_Feldman | 0.45011086 |
| sigMM | 0.895321107 |
| sigMM_2 | 0.89200986 |
| sigMM_3 | 0.89084223 |
| sigMM_4 | 0.886855553 |
| sigMM_5 | 0.879040188 |
| sigMM_6 | 0.877255934 |
| Caushi.CD8.Effector(III) | 0.876905719 |
| Yost_CD8_Eff_mem | 0.876206465 |
| Oh.CD4.GZMB | 0.872849902 |
| sigMM_7 | 0.871678595 |
| Wu.8EFF | 0.867950713 |
| Oh.CD8.FGFBP2 | 0.866798347 |
| Caushi.CD8-Effector(3) | 0.865309068 |
| Wu.8Trm.3 | 0.863285375 |
| sigMM_8 | 0.855620529 |
| Oliveira.TTE | 0.835686862 |
| Lowery.Cluster_4 | 0.83392276 |
| B16_TERMINAL.EX_Miller | 0.832919222 |
| Caushi.CD8.TRM(III) | 0.832852709 |
| Lowery.Cluster_6 | 0.832054604 |
| Caushi.CD8-TRM(1) | 0.825871237 |
| Wu.8Trm.2 | 0.822175109 |
| Caushi.CD8.TRM(II) | 0.820603134 |
| Caushi.CD8-TRM(2) | 0.819991659 |
| Lowery.NeoTCR8 | 0.812040947 |
| Caushi.MANA | 0.809978105 |
| Lowery.Cluster_7 | 0.808723495 |
| Oh.CD4.TH17 | 0.80242745 |
| Caushi.CD8.Stem-like_memory | 0.794494135 |
| Caushi.Stem-like_memory | 0.791127778 |
| Oliveira.Tumor-spec_TTE | 0.787931189 |
| Yost_CD8_Memory | 0.786671143 |
| Yost_CD8_Exh | 0.78347168 |
| Caushi.CD4-Tfh(1) | 0.782500691 |
| LCMV_PROG.EX_Miller | 0.782008241 |
| Caushi.CD8.TRM(VI) | 0.781689838 |
| Caushi.CD8.TRM(V) | 0.779968755 |
| Wu.CD4.TCF7 | 0.77908797 |
| Yost_CD8_Act/Exh | 0.777576045 |
| Caushi.CD8.Effector(II) | 0.771949463 |
| Oh.CD8.MAIT | 0.771723003 |
| Caushi.CD4CD8(I) | 0.77137746 |
| Caushi.CD8.CD4CD8(I) | 0.77137746 |
| Caushi.CD8-Effector(2) | 0.768905843 |
| CD8_G_Feldman | 0.761561778 |
| TOX_Scott | 0.757165839 |
| Oh.CD4.CM | 0.753146989 |
| LCMV_TERMN.EX_Miller | 0.751075011 |
| Wu.CD4.RPL32 | 0.750810227 |
| Oh.CD4.GZMK | 0.748102806 |
| Caushi.CD8.TRM(IV) | 0.74691325 |
| Mem_Eff_6_Feldman | 0.745301775 |
| Wu.8EM | 0.742993182 |
| Lowery.Cluster_8 | 0.742370039 |
| Oh.CD8.CM | 0.736478757 |
| Caushi.CD8.Proliferating | 0.732388877 |
| Caushi.CD8-Proliferating | 0.730870206 |
| B16_PROG.EX_Miller | 0.72388888 |
| Mel_Exhaust_Tirosh | 0.719372862 |
| Yost_Naive | 0.7161194 |
| Oh.CD8.RPL | 0.711816729 |
| Caushi.CD8-Effector(1) | 0.709895534 |
| Mem_Eff_4_Feldman | 0.709096034 |
| Caushi.CD4CD8(II) | 0.704312959 |
| Caushi.CD8.CD4CD8(II) | 0.704312959 |
| Wu.8chrom | 0.700517783 |
| Oliveira.TAct | 0.700299473 |
| Wu.CD4.Trm | 0.686767929 |
| Wu.CD4.FOS | 0.686725033 |
| Lowery.Cluster_3 | 0.678979858 |
| Lowery.Cluster_0 | 0.678827261 |
| Caushi.CD4-Th(3) | 0.677564698 |
| Oh.CD4.MITO | 0.675642927 |
| Caushi.CD4-Treg | 0.674306391 |
| Oh.CD4.Activate | 0.669866495 |
| Exhaust_3_Feldman | 0.669382355 |
| Caushi.CD4-Th(1) | 0.653897821 |
| Caushi.CD8-MHCII | 0.652511861 |
| Early.Act_5_Feldman | 0.651327134 |
| Lowery.Cluster_1 | 0.646503694 |
| Wu.CD4.IL6ST | 0.642064171 |
| Oh.TIL_CD4.GZMK | 0.640560264 |
| Oh.CD8.Naive | 0.627393217 |
| Oh.CD8.MITO | 0.623004843 |
| Oh.CD8.XCL | 0.621299162 |
| Caushi.CD4-Th(2) | 0.620594859 |
| Oh.TIL_CD4IL2RALO | 0.618680903 |
| Oh.CD8.HSP | 0.616426033 |
| Oh.CD8.CD39 | 0.613287031 |
| Lowery.Cluster_2 | 0.610904499 |
| Krishna.ACT.Term.Diff | 0.605721789 |
| Lowery.Cluster_9 | 0.603682172 |
| Yost_CD8_Activated | 0.599372728 |
| Caushi.CD4-Tfh(2) | 0.594834131 |
| Oh.CD4IL2RALO | 0.588156416 |
| Oliveira.TPE | 0.586165804 |
| CD8_B_Feldman | 0.582970755 |
| Oh.CD4.HSP | 0.582642063 |
| Caushi.MAIT | 0.579864337 |
| Lowery.Cluster_10 | 0.577680933 |
| Krishna.ACT.Stem.Like | 0.577370183 |
| Exhaust_1_Feldman | 0.576565735 |
| Caushi.CD8.Effector(I) | 0.566070734 |
| Oh.CD4.CXCL13 | 0.565621653 |
| Caushi.CD8.MAIT | 0.564943864 |
| Lowery.NeoTCR4 | 0.562740335 |
| Wu.Treg.1 | 0.560476652 |
| Oh.CD8.PRO | 0.554169365 |
| Wu.8Trm.1 | 0.553886362 |
| Lowery.Cluster_11 | 0.55316134 |
| Oliveira.Tumor_spec-TAct | 0.552789929 |
| Lowery.Cluster_5 | 0.550097849 |
| CD3.MT | 0.547864702 |
| Wu.8Mit | 0.546204065 |
| Caushi.CD8.TRM(I) | 0.546140583 |
| Oh.CD4IL2RAHI | 0.544037281 |
| Oliveira.Tumor-spec_TProl | 0.537211146 |
| Oliveira.TProl | 0.532943403 |
| Exhaust_2_Feldman | 0.529859223 |
| Jansen_Term_diff | 0.529520219 |
| Jansen_Stem_like | 0.52799653 |
| Oliveira.TEM | 0.524864859 |
| Oliveira.Tumor-spec_TEM | 0.521055058 |
| Oliveira.Tumor_spec-TPE | 0.515428311 |
| Li.CD8.DYS | 0.51459892 |
| Oh.TIL_CD4.GZMB | 0.514111849 |
| Oh.CD4.PROLIF | 0.503144703 |
| Oh.TIL_CD4IL2RAHI | 0.501817968 |
| Wu.Treg.2 | 0.500104326 |
| Wu.8KLRB1 | 0.490105141 |

TABLE 8
Tumor-reactive TCR alpha and beta chains

| TCFR | | | | |
|---|---|---|---|---|
| No. | patient | TRA | TRB | CD4_8 |
| 11 | PT07 | CAMREGPLDDMRF | CASSTAGTSGAVNEQFF | CD8 |
| 22 | PT07 | NA | CASSTAGTSGAVNEQFF | CD8 |
| 2975 | PT07 | NA | CATSILGGDYNEQFF | CD4 |
| 5545 | PT07 | CAGQGGTSYGKLTF | CASMRNTGELFF | CD4 |
| 6116 | PT07 | CALSEDMNRDDKIIF | CASSLVEQIGSEQFF | CD4 |
| 6620 | PT07 | CAVGSDMRF | CASSLSPTNYGYTF | CD4 |
| 6949 | PT07 | CAVNEGYGNKLVF | CASSFGGGEQFF | CD4 |
| 7008 | PT07 | CAASDTGRRALTF | CASSLVSSYNEQFF | CD4 |
| 9126 | PT08 | CIVQGSSGDKLTF | CSVLQAFIYGYTF | CD8 |
| 9721 | PT08 | CAAVWRNQGGKLIF | CASSLGVYEQYF | CD8 |
| 11729 | PT08 | CLVGDPKAAGNKLTF | CASTRTYSSTDTQYF | CD8 |
| 12383 | PT08 | NA | CSVLQAFIYGYTF | CD8 |
| 13985 | PT11 | CAYRSYNNNDMRF | CASSPIAGVENEQFF | CD8 |
| 14023 | PT11 | CAVRGASIKDTDKLIF | CASSPIAGVENEQFF | CD8 |
| 14036 | PT11 | NA | CASSQVAGFPDTQYF | CD8 |
| 15090 | PT11 | CAVRPYGKLTF | CASSPIAGVENEQFF | CD4 |
| 15434 | PT11 | CAVLDSNYQLIW | CASSQVAGFPDTQYF | CD8 |
| 17594 | PT11 | NA | CASSPIAGVENEQFF | CD8 |
| 23197 | PT09 | NA | CASSSGSGEQFF | CD8 |
| 27668 | PT09 | NA | CASSPTGVGSPLHF | CD8 |
| 31195 | PT10 | CAVSFSGTYKYIF | CASSVAGGYEQYF | CD8 |
| 31482 | PT10 | CVVSDRGSTLGRLYF | CASSERDQQALDEQYF | NKT |
| 31836 | PT10 | CAVRSFTGGGADGLTF | CASSQDRGHSYNEQFF | CD8 |
| 32021 | PT10 | NA | CASSERDQQALDEQYF | NKT |
| 32084 | PT10 | CAVMDSNYQLIW | CSARDGTSGETQYF | NKT |
| 32627 | PT10 | NA | CASSQDRGHSYNEQFF | CD8 |
| 33541 | PT10 | CAVSGSARQLTF | CASSPGTGYEQYF | CD8 |
| 35650 | PT10 | CAAPVGGSGNTPLVF | CASSPDSYEQYF | CD8 |
| 35786 | PT10 | CAVKGPKQAKIIF | CASWSRRTKGEQFF | CD8 |
| 36263 | PT10 | CLVGDIILTGGGNKLTF | CASSYGQGYEQYF | CD8 |
| 36298 | PT10 | CAYQGGSEKLVF | CSARDGTSGETQYF | NKT |
| 36389 | PT10 | CAVRGLQTGANNLFF | CSARDGTSGETQYF | NKT |
| 37804 | PT10 | CAVRDPLTSGTYKYIF | CASSLNTEAFF | CD8 |
| 40075 | PT10 | CATDAGNDMRF | CASSPGLAYEQYF | CD8 |
| 40463 | PT10 | CAVRSSYNTDKLIF | CASSFWGSSSTDTQYF | CD8 |
| 40471 | PT10 | CALSGRDSNYQLIW | CASSSNLGVEYEQYF | CD8 |
| 41046 | PT10 | CLVGDDRYSGGGADGLTF | CSARDGTSGETQYF | CD8 |
| 41233 | PT10 | CAEGHLASGGSYIPTF | CASSYSGQGYTF | CD8 |
| 42108 | PT10 | CLVGDTDDYGSGNTGKLIF | CSARDGTSGETQYF | CD8 |
| 42229 | PT10 | CAGRTDSWGKLQF | CASSLASEQYF | CD8 |
| 42446 | PT10 | CAEMYSGGGADGLTF | CASSSGTGAYEQYF | CD8 |
| 42573 | PT10 | CIVRVEMDSSYKLIF | CSARDGTSGETQYF | CD8 |
| 44685 | PT10 | CAGDRDDKIIF | CASSPGQGYEQYF | CD8 |
| 44741 | PT10 | CAGDRGAQKLVF | CASSLGNTEAFF | CD8 |
| 45040 | PT10 | NA | CASSSNLGVEYEQYF | CD8 |
| 45092 | PT10 | CAVSDLDTGNQFYF | CASSLGQSNQPQHF | CD8 |
| 45904 | PT10 | CAVEDPGYALNF | CASSDSYEQYF | CD8 |
| 46135 | PT10 | CAYRSANFGNEKLTF | CASSQDRGHSYNEQFF | CD8 |
| 46687 | PT12 | CAVRGSGGSYIPTF | CAIGTGDSNQPQHF | CD8 |
| 54695 | PT06 | CAGEEAGTALIF | CASRRTSGSYNEQFF | CD8 |
| 58447 | PT06 | CAATGNQFYF | CASSPGQGYEQYF | CD8 |
| 60713 | PT06 | CVGGVNDYKLSF | CASSPGTAYEQYF | CD8 |
| 67186 | PT05 | CAGMNYGGSQGNLIF | CASSLRSSGDGTQYF | CD4 |
| 69037 | PT04 | CAVMEAGTALIF | CSARDRNYGYTF | CD8 |
| 69571 | PT04 | CAGQETSGSRLTF | CASSLGGNTGELFF | CD8 |
| 70860 | PT04 | CVVNNNDMRF | CASSLSGGNQPQHF | CD8 |
| 71977 | PT04 | CAAWRNTPLVF | CASSLSGGNQPQHF | CD8 |
| 72612 | PT03 | CAFMRSIDMRF | CASSFSLAGNEQFF | CD8 |
| 72780 | PT03 | NA | CASSFSLAGNEQFF | CD8 |
| 80720 | PT03 | CAFIPFGNEKLTF | CASSLVNTEAFF | CD4 |
| 89478 | PT02 | CLVGDMGLGGYNKLIF | CASSPGTAYEQYF | CD8 |
| 92241 | PT02 | CAEGSGGYNKLIF | CASSPGGTEAFF | CD8 |
| 93876 | PT02 | CAGPLF | CASSPAGTDYGYTF | CD8 |
| 94879 | PT02 | CAESTPGGGNKLTF | CASSLGNTEAFF | CD8 |
| 102954 | PT01 | NA | CASRLKREGSYNEQFF | CD4 |
| 113247 | PT15 | CAVRDNEEGSYIPTF | CASSPGQGYEQYF | CD8 |
| 113412 | PT15 | CVVNVRSNYQLIW | CASSEDRREDTQYF | CD4 |
| 116156 | PT13 | NA | CASTRLAGANNEQFF | CD8 |
| 116169 | PT13 | CAASASSGTSYGKLTF | CASTRLAGANNEQFF | CD8 |
| 116260 | PT13 | CAVKAHNNDMRF | CASTRLAGANNEQFF | CD8 |
| 116341 | PT13 | CALSWLHGSSNTGKLIF | CASTRLAGANNEQFF | CD8 |
| 116654 | PT13 | CAVKAHNNDMRF | CASSEDRREDTQYF | CD4 |
| 116809 | PT13 | CIDGGSQGNLIF | CASSEDRREDTQYF | CD4 |
| 117395 | PT13 | CAASTRNQFYF | CASTRLAGANNEQFF | CD8 |
| 118435 | PT13 | CAASIVMIGSSNTGKLIF | CASSLVNTEAFF | CD4 |
| 118775 | PT13 | CAALSNTGKLIF | CASTRLAGANNEQFF | CD4 |
| 119131 | PT13 | CAVGANQAGTALIF | CASTRLAGANNEQFF | CD8 |
| 119467 | PT13 | CALATGNQFYF | CASTRLAGANNEQFF | CD4 |
| 119497 | PT13 | CAATVNTGTASKLTF | CASTRLAGANNEQFF | CD4 |
| 119678 | PT13 | CAMSGSGGYQKVTF | CASTRLAGANNEQFF | CD4 |
| 121265 | PT13 | NA | CASSEDRREDTQYF | CD4 |
| 121653 | PT13 | CAVGALNDYKLSF | CASTRLAGANNEQFF | CD8 |
| 122872 | PT13 | CIVRVAVGGGSNYKLTF | CASTRLAGANNEQFF | CD8 |
| 124954 | PT13 | CAVKYGQNFVF | CASSDPLGAGSTDTQYF | CD4 |
| 125647 | PT14 | CAVGANQAGTALIF | CSVFAVVASGSRYEQYF | CD8 |
| 125730 | PT14 | CAVRDLTPGSGNTPLVF | CSVFAVVASGSRYEQYF | CD4 |
| 125770 | PT14 | CAVRRGSNYQLIW | CSVFAVVASGSRYEQYF | CD8 |
| 125825 | PT14 | NA | CATSHSNQPQHF | CD8 |
| 125952 | PT14 | CAVKEGNTPLVF | CSVFAVVASGSRYEQYF | CD4 |
| 125996 | PT14 | CAVMETSGSRLTF | CSVFAVVASGSRYEQYF | CD8 |
| 126119 | PT14 | CAATLYNFNKFYF | CASSPTGTSSTDTQYF | CD8 |
| 126200 | PT14 | CAVSDRDMRF | CASSVGRGRDTEAFF | CD8 |
| 126399 | PT14 | NA | CASSVGRGRDTEAFF | CD8 |
| 126405 | PT14 | CAARQGGSEKLVF | CSVFAVVASGSRYEQYF | CD8 |
| 126466 | PT14 | NA | CSVFAVVASGSRYEQYF | CD8 |
| 126479 | PT14 | CALSSWGNEKLTF | CSVFAVVASGSRYEQYF | CD4 |
| 126502 | PT14 | CVVNIDSWGKLQF | CSARGRLAGEFSEQYF | CD8 |
| 126598 | PT14 | CAVYSSASKIIF | CASSVGRGRDTEAFF | CD8 |
| 126628 | PT14 | CAYKKDTGRRALTF | CSVFAVVASGSRYEQYF | CD4 |
| 126931 | PT14 | CAASIRGNTGKLIF | CSVFAVVASGSRYEQYF | CD4 |
| 127031 | PT14 | CAVSDRDMRF | CASSPTGTSSTDTQYF | CD8 |
| 127059 | PT14 | CAVSETNFGNEKLTF | CASSPDRGTEAFF | CD8 |
| 127247 | PT14 | CIQDNNNDMRF | CSVFAVVASGSRYEQYF | CD8 |
| 127371 | PT14 | CAVGSNQAGTALIF | CSVFAVVASGSRYEQYF | CD8 |
| 127378 | PT14 | CAGGATNKLIF | CASSVGRGRDTEAFF | CD8 |
| 127649 | PT14 | NA | CSARGRLAGEFSEQYF | CD8 |
| 127813 | PT14 | CALSAGGTSYGKLTF | CASSQLAGATDTQYF | CD8 |
| 127973 | PT14 | CAASWAGADGLTF | CATSHSNQPQHF | CD8 |
| 128279 | PT14 | CAPRGRDYKLSF | CATSHSNQPQHF | CD8 |
| 11 | PT07 | CAMREGPLDDMRF | CASSTAGTSGAVNEQFF | CD8 |
| 22 | PT07 | NA | CASSTAGTSGAVNEQFF | CD8 |
| 2975 | PT07 | NA | CATSILGGDYNEQFF | CD4 |
| 5545 | PT07 | CAGQGGTSYGKLTF | CASMRNTGELFF | CD4 |
| 6116 | PT07 | CALSEDMNRDDKIIF | CASSLVEQIGSEQFF | CD4 |
| 6620 | PT07 | CAVGSDMRF | CASSLSPTNYGYTF | CD4 |
| 6949 | PT07 | CAVNEGYGNKLVF | CASSFGGGEQFF | CD4 |
| 7008 | PT07 | CAASDTGRRALTF | CASSLVSSYNEQFF | CD4 |
| 9126 | PT08 | CIVQGSSGDKLTF | CSVLQAFIYGYTF | CD8 |
| 9721 | PT08 | CAAVWRNQGGKLIF | CASSLGVYEQYF | CD8 |
| 11729 | PT08 | CLVGDPKAAGNKLTF | CASTRTYSSTDTQYF | CD8 |
| 12383 | PT08 | NA | CSVLQAFIYGYTF | CD8 |
| 13985 | PT11 | CAYRSYNNNDMRF | CASSPIAGVENEQFF | CD8 |
| 14023 | PT11 | CAVRGASIKDTDKLIF | CASSPIAGVENEQFF | CD8 |
| 14036 | PT11 | NA | CASSQVAGFPDTQYF | CD8 |
| 15090 | PT11 | CAVRPYGKLTF | CASSPIAGVENEQFF | CD4 |
| 15434 | PT11 | CAVLDSNYQLIW | CASSQVAGFPDTQYF | CD8 |
| 17594 | PT11 | NA | CASSPIAGVENEQFF | CD8 |
| 23197 | PT09 | NA | CASSSGSGEQFF | CD8 |
| 27668 | PT09 | NA | CASSPTGVGSPLHF | CD8 |
| 31195 | PT10 | CAVSFSGTYKYIF | CASSVAGGYEQYF | CD8 |
| 31482 | PT10 | CVVSDRGSTLGRLYF | CASSERDQQALDEQYF | NKT |
| 31836 | PT10 | CAVRSFTGGGADGLTF | CASSQDRGHSYNEQFF | CD8 |
| 32021 | PT10 | NA | CASSERDQQALDEQYF | NKT |
| 32084 | PT10 | CAVMDSNYQLIW | CSARDGTSGETQYF | NKT |
| 32627 | PT10 | NA | CASSQDRGHSYNEQFF | CD8 |
| 33541 | PT10 | CAVSGSARQLTF | CASSPGTGYEQYF | CD8 |
| 35650 | PT10 | CAAPVGGSGNTPLVF | CASSPDSYEQYF | CD8 |
| 35786 | PT10 | CAVKGPKQAKIIF | CASWSRRTKGEQFF | CD8 |
| 36263 | PT10 | CLVGDIILTGGGNKLTF | CASSYGQGYEQYF | CD8 |
| 36298 | PT10 | CAYQGGSEKLVF | CSARDGTSGETQYF | NKT |
| 36389 | PT10 | CAVRGLQTGANNLFF | CSARDGTSGETQYF | NKT |
| 37804 | PT10 | CAVRDPLTSGTYKYIF | CASSLNTEAFF | CD8 |
| 40075 | PT10 | CATDAGNDMRF | CASSPGLAYEQYF | CD8 |
| 40463 | PT10 | CAVRSSYNTDKLIF | CASSFWGSSSTDTQYF | CD8 |
| 40471 | PT10 | CALSGRDSNYQLIW | CASSSNLGVEYEQYF | CD8 |
| 41046 | PT10 | CLVGDDRYSGGGADGLTF | CSARDGTSGETQYF | CD8 |
| 41233 | PT10 | CAEGHLASGGSYIPTF | CASSYSGQGYTF | CD8 |
| 42108 | PT10 | CLVGDTDDYGSGNTGKLIF | CSARDGTSGETQYF | CD8 |
| 42229 | PT10 | CAGRTDSWGKLQF | CASSLASEQYF | CD8 |
| 42446 | PT10 | CAEMYSGGGADGLTF | CASSSGTGAYEQYF | CD8 |
| 42573 | PT10 | CIVRVEMDSSYKLIF | CSARDGTSGETQYF | CD8 |
| 44685 | PT10 | CAGDRDDKIIF | CASSPGQGYEQYF | CD8 |
| 44741 | PT10 | CAGDRGAQKLVF | CASSLGNTEAFF | CD8 |
| 45040 | PT10 | NA | CASSSNLGVEYEQYF | CD8 |
| 45092 | PT10 | CAVSDLDTGNQFYF | CASSLGQSNQPQHF | CD8 |
| 45904 | PT10 | CAVEDPGYALNF | CASSDSYEQYF | CD8 |
| 46135 | PT10 | CAYRSANFGNEKLTF | CASSQDRGHSYNEQFF | CD8 |
| 46687 | PT12 | CAVRGSGGSYIPTF | CAIGTGDSNQPQHF | CD8 |
| 54695 | PT06 | CAGEEAGTALIF | CASRRTSGSYNEQFF | CD8 |
| 58447 | PT06 | CAATGNQFYF | CASSPGQGYEQYF | CD8 |
| 60713 | PT06 | CVGGVNDYKLSF | CASSPGTAYEQYF | CD8 |
| 67186 | PT05 | CAGMNYGGSQGNLIF | CASSLRSSGDGTQYF | CD4 |
| 69037 | PT04 | CAVMEAGTALIF | CSARDRNYGYTF | CD8 |
| 69571 | PT04 | CAGQETSGSRLTF | CASSLGGNTGELFF | CD8 |
| 70860 | PT04 | CVVNNNDMRF | CASSLSGGNQPQHF | CD8 |
| 71977 | PT04 | CAAWRNTPLVF | CASSLSGGNQPQHF | CD8 |
| 72612 | PT03 | CAFMRSIDMRF | CASSFSLAGNEQFF | CD8 |
| 72780 | PT03 | NA | CASSFSLAGNEQFF | CD8 |
| 80720 | PT03 | CAFIPFGNEKLTF | CASSLVNTEAFF | CD4 |
| 89478 | PT02 | CLVGDMGLGGYNKLIF | CASSPGTAYEQYF | CD8 |
| 92241 | PT02 | CAEGSGGYNKLIF | CASSPGGTEAFF | CD8 |
| 93876 | PT02 | CAGPLF | CASSPAGTDYGYTF | CD8 |
| 94879 | PT02 | CAESTPGGGNKLTF | CASSLGNTEAFF | CD8 |
| 102954 | PT01 | NA | CASRLKREGSYNEQFF | CD4 |
| 113247 | PT15 | CAVRDNEEGSYIPTF | CASSPGQGYEQYF | CD8 |
| 113412 | PT15 | CVVNVRSNYQLIW | CASSEDRREDTQYF | CD4 |
| 116156 | PT13 | NA | CASTRLAGANNEQFF | CD8 |
| 116169 | PT13 | CAASASSGTSYGKLTF | CASTRLAGANNEQFF | CD8 |
| 116260 | PT13 | CAVKAHNNDMRF | CASTRLAGANNEQFF | CD8 |
| 116341 | PT13 | CALSWLHGSSNTGKLIF | CASTRLAGANNEQFF | CD8 |
| 116654 | PT13 | CAVKAHNNDMRF | CASSEDRREDTQYF | CD4 |
| 116809 | PT13 | CIDGGSQGNLIF | CASSEDRREDTQYF | CD4 |
| 117395 | PT13 | CAASTRNQFYF | CASTRLAGANNEQFF | CD8 |
| 118435 | PT13 | CAASIVMIGSSNTGKLIF | CASSLVNTEAFF | CD4 |
| 118775 | PT13 | CAALSNTGKLIF | CASTRLAGANNEQFF | CD4 |
| 119131 | PT13 | CAVGANQAGTALIF | CASTRLAGANNEQFF | CD8 |
| 119467 | PT13 | CALATGNQFYF | CASTRLAGANNEQFF | CD4 |
| 119497 | PT13 | CAATVNTGTASKLTF | CASTRLAGANNEQFF | CD4 |
| 119678 | PT13 | CAMSGSGGYQKVTF | CASTRLAGANNEQFF | CD4 |
| 121265 | PT13 | NA | CASSEDRREDTQYF | CD4 |
| 121653 | PT13 | CAVGALNDYKLSF | CASTRLAGANNEQFF | CD8 |
| 122872 | PT13 | CIVRVAVGGGSNYKLTF | CASTRLAGANNEQFF | CD8 |
| 124954 | PT13 | CAVKYGQNFVF | CASSDPLGAGSTDTQYF | CD4 |
| 125647 | PT14 | CAVGANQAGTALIF | CSVFAVVASGSRYEQYF | CD8 |
| 125730 | PT14 | CAVRDLTPGSGNTPLVF | CSVFAVVASGSRYEQYF | CD4 |
| 125770 | PT14 | CAVRRGSNYQLIW | CSVFAVVASGSRYEQYF | CD8 |
| 125825 | PT14 | NA | CATSHSNQPQHF | CD8 |
| 125952 | PT14 | CAVKEGNTPLVF | CSVFAVVASGSRYEQYF | CD4 |
| 125996 | PT14 | CAVMETSGSRLTF | CSVFAVVASGSRYEQYF | CD8 |
| 126119 | PT14 | CAATLYNFNKFYF | CASSPTGTSSTDTQYF | CD8 |
| 126200 | PT14 | CAVSDRDMRF | CASSVGRGRDTEAFF | CD8 |
| 126399 | PT14 | NA | CASSVGRGRDTEAFF | CD8 |
| 126405 | PT14 | CAARQGGSEKLVF | CSVFAVVASGSRYEQYF | CD8 |
| 126466 | PT14 | NA | CSVFAVVASGSRYEQYF | CD8 |
| 126479 | PT14 | CALSSWGNEKLTF | CSVFAVVASGSRYEQYF | CD4 |
| 126502 | PT14 | CVVNIDSWGKLQF | CSARGRLAGEFSEQYF | CD8 |
| 126598 | PT14 | CAVYSSASKIIF | CASSVGRGRDTEAFF | CD8 |
| 126628 | PT14 | CAYKKDTGRRALTF | CSVFAVVASGSRYEQYF | CD4 |
| 126931 | PT14 | CAASIRGNTGKLIF | CSVFAVVASGSRYEQYF | CD4 |
| 127031 | PT14 | CAVSDRDMRF | CASSPTGTSSTDTQYF | CD8 |
| 127059 | PT14 | CAVSETNFGNEKLTF | CASSPDRGTEAFF | CD8 |
| 127247 | PT14 | CIQDNNNDMRF | CSVFAVVASGSRYEQYF | CD8 |
| 127371 | PT14 | CAVGSNQAGTALIF | CSVFAVVASGSRYEQYF | CD8 |
| 127378 | PT14 | CAGGATNKLIF | CASSVGRGRDTEAFF | CD8 |
| 127649 | PT14 | NA | CSARGRLAGEFSEQYF | CD8 |
| 127813 | PT14 | CALSAGGTSYGKLTF | CASSQLAGATDTQYF | CD8 |
| 127973 | PT14 | CAASWAGADGLTF | CATSHSNQPQHF | CD8 |
| 128279 | PT14 | CAPRGRDYKLSF | CATSHSNQPQHF | CD8 |
| TCFR | | global | tumor | tumor_ |
| No. | HLA haplotype | reactivity | reactivity | antigen |
| 11 | HLA-A*03; HLA-A*25; HLA-B*15; HLA-B*35; | tumor_rea_antigen_agnostic | MM_cell_ | NA |
| | HLA-C*03; HLA-C*04; B2M | | reactive | |
| 22 | HLA-A*03; HLA-A*25; HLA-B*15; HLA-B*35; | tumor_rea_antigen_agnostic | MM_cell_ | NA |
| | HLA-C*03; HLA-C*04; B2M | | reactive | |
| 2975 | HLA-DRB1*01; HLA-DRB1*13; HLA-DRB3*01; | tumor_rea_antigen_agnostic | MM_cell_ | NA |
| | HLA-DRB3*NN; HLA-DRB4*NN; HLA- | | reactive | |
| | DRB5*NN; HLA-DQA1*01; HLA-DQA1*05; | | | |
| | HLA-DQB1*03; HLA-DQB1*05; HLA-DPA1*01; | | | |
| | HLA-DPA1*02; HLA-DPB1*04; HLA-DPB1*10; | | | |
| | HLA-DRA*01 | | | |
| 5545 | HLA-DRB1*01; HLA-DRB1*13; HLA-DRB3*01; | tumor_rea_antigen_agnostic | MM_cell_ | NA |
| | HLA-DRB3*NN; HLA-DRB4*NN; HLA- | | reactive | |
| | DRB5*NN; HLA-DQA1*01; HLA-DQA1*05; | | | |
| | HLA-DQB1*03; HLA-DQB1*05; HLA-DPA1*01; | | | |
| | HLA-DPA1*02; HLA-DPB1*04; HLA-DPB1*10; | | | |
| | HLA-DRA*01 | | | |
| 6116 | HLA-DRB1*01; HLA-DRB1*13; HLA-DRB3*01; | tumor_rea_antigen_agnostic | MM_cell_ | NA |
| | HLA-DRB3*NN; HLA-DRB4*NN; HLA- | | reactive | |
| | DRB5*NN; HLA-DQA1*01; HLA-DQA1*05; | | | |
| | HLA-DQB1*03; HLA-DQB1*05; HLA-DPA1*01; | | | |
| | HLA-DPA1*02; HLA-DPB1*04; HLA-DPB1*10; | | | |
| | HLA-DRA*01 | | | |
| 6620 | HLA-DRB1*01; HLA-DRB1*13; HLA-DRB3*01; | tumor_rea_antigen_agnostic | MM_cell_ | NA |
| | HLA-DRB3*NN; HLA-DRB4*NN; HLA- | | reactive | |
| | DRB5*NN; HLA-DQA1*01; HLA-DQA1*05; | | | |
| | HLA-DQB1*03; HLA-DQB1*05; HLA-DPA1*01; | | | |
| | HLA-DPA1*02; HLA-DPB1*04; HLA-DPB1*10; | | | |
| | HLA-DRA*01 | | | |
| 6949 | HLA-DRB1*01; HLA-DRB1*13; HLA-DRB3*01; | tumor_rea_antigen_agnostic | MM_cell_ | NA |
| | HLA-DRB3*NN; HLA-DRB4*NN; HLA- | | reactive | |
| | DRB5*NN; HLA-DQA1*01; HLA-DQA1*05; | | | |
| | HLA-DQB1*03; HLA-DQB1*05; HLA-DPA1*01; | | | |
| | HLA-DPA1*02; HLA-DPB1*04; HLA-DPB1*10; | | | |
| | HLA-DRA*01 | | | |
| 7008 | HLA-DRB1*01; HLA-DRB1*13; HLA-DRB3*01; | tumor_rea_antigen_agnostic | MM_cell_ | NA |
| | HLA-DRB3*NN; HLA-DRB4*NN; HLA- | | reactive | |
| | DRB5*NN; HLA-DQA1*01; HLA-DQA1*05; | | | |
| | HLA-DQB1*03; HLA-DQB1*05; HLA-DPA1*01; | | | |
| | HLA-DPA1*02; HLA-DPB1*04; HLA-DPB1*10; | | | |
| | HLA-DRA*01 | | | |
| 9126 | HLA-A*01; HLA-A*02; HLA-B*07; HLA-B*08; | tumor_rea_antigen_derived | shared_peptides | NA |
| | HLA-C*07; B2M | | | |
| 9721 | HLA-A*01; HLA-A*02; HLA-B*07; HLA-B*08; | ambiguous_vir_tumor | public_antigen | AR |
| | HLA-C*07; B2M | | | |
| 11729 | HLA-A*01; HLA-A*02; HLA-B*07; HLA-B*08; | tumor_rea_antigen_agnostic | MM_cell_ | CTAG2 |
| | HLA-C*07; B2M | | reactive | |
| 12383 | HLA-A*01; HLA-A*02; HLA-B*07; HLA-B*08; | tumor_rea_antigen_derived | MM_cell_ | NA |
| | HLA-C*07; B2M | | reactive | |
| 13985 | HLA-A*01; HLA-A*23; HLA-B*50; HLA-B*52; | tumor_rea_antigen_agnostic | MM_cell_ | NA |
| | HLA-C*06; HLA-C*12; B2M | | reactive | |
| 14023 | HLA-A*01; HLA-A*23; HLA-B*50; HLA-B*52; | tumor_rea_antigen_agnostic | MM_cell_ | NA |
| | HLA-C*06; HLA-C*12; B2M | | reactive | |
| 14036 | HLA-A*01; HLA-A*23; HLA-B*50; HLA-B*52; | tumor_rea_antigen_agnostic | MM_cell_ | IGKV |
| | HLA-C*06; HLA-C*12; B2M | | reactive | |
| 15090 | HLA-DRB1*08; HLA-DRB1*15; HLA-DRB3*NN; | tumor_rea_antigen_agnostic | MM_cell_ | NA |
| | HLA-DRB4*NN; HLA-DRB5*01; HLA- | | | |
| | DRB5*NN; HLA-DQA1*01; HLA-DQA1*06; | | | |
| | HLA-DQB1*03; HLA-DQB1*05; HLA-DPA1*01; | | | |
| | HLA-DPA1*02; HLA-DPB1*04; HLA-DRA*01 | | | |
| 15434 | HLA-A*01; HLA-A*23; HLA-B*50; HLA-B*52; | tumor_rea_antigen_agnostic | MM_cell_ | IGKV |
| | HLA-C*06; HLA-C*12; B2M | | reactive | |
| 17594 | HLA-A*01; HLA-A*23; HLA-B*50; HLA-B*52; | tumor_rea_antigen_agnostic | MM_cell_ | NA |
| | HLA-C*06; HLA-C*12; B2M | | reactive | |
| 23197 | HLA-A*03; HLA-B*07; HLA-C*07; B2M | tumor_rea_antigen_derived | personalized_ | NA |
| | | | peptides | |
| 27668 | HLA-A*03; HLA-B*07; HLA-C*07; B2M | tumor_rea_antigen_agnostic | MM_cell_reactive | NA |
| 31195 | HLA-A*02; HLA-B*27; HLA-B*40; HLA-C*02; | tumor_rea_antigen_derived | public_antigen | IGH (JH) |
| | HLA-C*03; B2M | | | |
| 31482 | HLA-A*02; HLA-B*27; HLA-B*40; HLA-C*02; | tumor_rea_antigen_derived | shared_peptides | NA |
| | HLA-C*03; B2M | | | |
| 31836 | HLA-A*02; HLA-B*27; HLA-B*40; HLA-C*02; | tumor_rea_antigen_derived | personalized_ | NA |
| | HLA-C*03; B2M | | peptides | |
| 32021 | HLA-A*02; HLA-B*27; HLA-B*40; HLA-C*02; | tumor_rea_antigen_derived | shared_peptides | NA |
| | HLA-C*03; B2M | | | |
| 32084 | HLA-A*02; HLA-B*27; HLA-B*40; HLA-C*02; | tumor_rea_antigen_derived | shared_peptides | NA |
| | HLA-C*03; B2M | | | |
| 32627 | HLA-A*02; HLA-B*27; HLA-B*40; HLA-C*02; | tumor_rea_antigen_derived | personalized_ | NA |
| | HLA-C*03; B2M | | peptides | |
| 33541 | HLA-A*02; HLA-B*27; HLA-B*40; HLA-C*02; | tumor_rea_antigen_derived | public_antigen | IGH (JH) |
| | HLA-C*03; B2M | | | |
| 35650 | HLA-A*02; HLA-B*27; HLA-B*40; HLA-C*02; | tumor_rea_antigen_derived | public_antigen | AR |
| | HLA-C*03; B2M | | | |
| 35786 | HLA-A*02; HLA-B*27; HLA-B*40; HLA-C*02; | tumor_rea_antigen_derived | personalized_ | NA |
| | HLA-C*03; B2M | | peptides | |
| 36263 | HLA-A*02; HLA-B*27; HLA-B*40; HLA-C*02; | tumor_rea_antigen_derived | public_antigen | IGH (JH) |
| | HLA-C*03; B2M | | | |
| 36298 | HLA-A*02; HLA-B*27; HLA-B*40; HLA-C*02; | tumor_rea_antigen_derived | shared_peptides | NA |
| | HLA-C*03; B2M | | | |
| 36389 | HLA-A*02; HLA-B*27; HLA-B*40; HLA-C*02; | tumor_rea_antigen_derived | shared_peptides | NA |
| | HLA-C*03; B2M | | | |
| 37804 | HLA-A*02; HLA-B*27; HLA-B*40; HLA-C*02; | ambiguous_vir_tumor | public_antigen | IGL (JH) |
| | HLA-C*03; B2M | | | |
| 40075 | HLA-A*02; HLA-B*27; HLA-B*40; HLA-C*02; | tumor_rea_antigen_derived | public_antigen | p53 |
| | HLA-C*03; B2M | | | |
| 40463 | HLA-A*02; HLA-B*27; HLA-B*40; HLA-C*02; | tumor_rea_antigen_derived | personalized_ | NA |
| | HLA-C*03; B2M | | peptides | |
| 40471 | HLA-A*02; HLA-B*27; HLA-B*40; HLA-C*02; | tumor_rea_antigen_derived | shared_peptides | NA |
| | HLA-C*03; B2M | | | |
| 41046 | HLA-A*02; HLA-B*27; HLA-B*40; HLA-C*02; | tumor_rea_antigen_derived | shared_peptides | NA |
| | HLA-C*03; B2M | | | |
| 41233 | HLA-A*02; HLA-B*27; HLA-B*40; HLA-C*02; | tumor_rea_antigen_derived | public_antigen | GANAB |
| | HLA-C*03; B2M | | | |
| 42108 | HLA-A*02; HLA-B*27; HLA-B*40; HLA-C*02; | tumor_rea_antigen_derived | shared_peptides | NA |
| | HLA-C*03; B2M | | | |
| 42229 | HLA-A*02; HLA-B*27; HLA-B*40; HLA-C*02; | tumor_rea_antigen_derived | public_antigen | KAT6A |
| | HLA-C*03; B2M | | | |
| 42446 | HLA-A*02; HLA-B*27; HLA-B*40; HLA-C*02; | tumor_rea_antigen_derived | public_antigen | FLNA |
| | HLA-C*03; B2M | | | |
| 42573 | HLA-A*02; HLA-B*27; HLA-B*40; HLA-C*02; | tumor_rea_antigen_derived | shared_peptides | NA |
| | HLA-C*03; B2M | | | |
| 44685 | HLA-A*02; HLA-B*27; HLA-B*40; HLA-C*02; | ambiguous_vir_tumor | public_antigen | IGH (JH) |
| | HLA-C*03; B2M | | | |
| 44741 | HLA-A*02; HLA-B*27; HLA-B*40; HLA-C*02; | ambiguous_vir_tumor | public_antigen | SEC24A |
| | HLA-C*03; B2M | | | |
| 45040 | HLA-A*02; HLA-B*27; HLA-B*40; HLA-C*02; | tumor_rea_antigen_derived | shared_peptides | NA |
| | HLA-C*03; B2M | | | |
| 45092 | HLA-A*02; HLA-B*27; HLA-B*40; HLA-C*02; | tumor_rea_antigen_derived | shared_peptides | NA |
| | HLA-C*03; B2M | | | |
| 45904 | HLA-A*02; HLA-B*27; HLA-B*40; HLA-C*02; | tumor_rea_antigen_derived | public_antigen | Melan- |
| | HLA-C*03; B2M | | | A/MART-1 |
| 46135 | HLA-A*02; HLA-B*27; HLA-B*40; HLA-C*02; | tumor_rea_antigen_derived | personalized_pep | NA |
| | HLA-C*03; B2M | | tides | |
| 46687 | HLA-A*11; HLA-A*26; HLA-B*27; HLA-B*35; | ambiguous_vir_tumor | public_antigen | NY-ESO-If |
| | HLA-C*01; HLA-C*04; B2M | | | |
| 54695 | HLA-A*02; HLA-A*03; HLA-B*07; HLA-B*27; | tumor_rea_antigen_agnostic | MM_cell_ | NA |
| | HLA-C*02; HLA-C*07; B2M | | reactive | |
| 58447 | HLA-A*02; HLA-A*03; HLA-B*07; HLA-B*27; | ambiguous_vir_tumor | public_antigen | IGH (JH) |
| | HLA-C*02; HLA-C*07; B2M | | | |
| 60713 | HLA-A*02; HLA-A*03; HLA-B*07; HLA-B*27; | tumor_rea_antigen_derived | public_antigen | IGH (JH) |
| | HLA-C*02; HLA-C*07; B2M | | | |
| 67186 | HLA-DRB1*14; HLA-DRB1*15; HLA-DRB3*02; | tumor_rea_antigen_agnostic | MM_cell_ | NA |
| | HLA-DRB3*NN; HLA-DRB4*NN; HLA- | | reactive | |
| | DRB5*01; HLA-DRB5*NN; HLA-DQA1*01; | | | |
| | HLA-DQB1*05; HLA-DQB1*06; HLA-DPA1*01; | | | |
| | HLA-DPA1*02; HLA-DPB1*02; HLA-DPB1*05; | | | |
| | HLA-DRA*01 | | | |
| 69037 | HLA-A*02; HLA-A*26; HLA-B*15; HLA-B*44; | tumor_rea_antigen_derived | public_antigen | ETNK-1 |
| | HLA-C*05; HLA-C*12; B2M | | | |
| 69571 | HLA-A*02; HLA-A*26; HLA-B*15; HLA-B*44; | ambiguous_vir_tumor | public_antigen | Melan- |
| | HLA-C*05; HLA-C*12; B2M | | | A/MART-1 |
| 70860 | HLA-A*02; HLA-A*26; HLA-B*15; HLA-B*44; | tumor_rea_antigen_derived | personalized_ | NA |
| | HLA-C*05; HLA-C*12; B2M | | peptides | |
| 71977 | HLA-A*02; HLA-A*26; HLA-B*15; HLA-B*44; | tumor_rea_antigen_derived | personalized_ | NA |
| | HLA-C*05; HLA-C*12; B2M | | peptides | |
| 72612 | HLA-A*02; HLA-A*31; HLA-B*27; HLA-B*44; | tumor_rea_antigen_agnostic | MM_cell_ | NA |
| | HLA-C*01; HLA-C*05; B2M | | reactive | |
| 72780 | HLA-A*02; HLA-A*31; HLA-B*27; HLA-B*44; | tumor_rea_antigen_agnostic | MM_cell_ | NA |
| | HLA-C*01; HLA-C*05; B2M | | reactive | |
| 80720 | HLA-DRB1*08; HLA-DRB1*11; HLA-DRB3*02; | tumor_rea_antigen_derived | public_antigen | p53 |
| | HLA-DRB3*NN; HLA-DRB4*NN; HLA- | | | |
| | DRB5*NN; HLA-DQA1*04; HLA-DQA1*05; | | | |
| | HLA-DQB1*03; HLA-DQB1*04; HLA-DPA1*01; | | | |
| | HLA-DPB1*04; HLA-DRA*01 | | | |
| 89478 | HLA-A*02; HLA-B*27; HLA-B*57; HLA-C*02; | tumor_rea_antigen_derived | public_antigen | IGH (JH) |
| | HLA-C*06; B2M | | | |
| 92241 | HLA-A*02; HLA-B*27; HLA-B*57; HLA-C*02; | tumor_rea_antigen_derived | public_antigen | Melan- |
| | HLA-C*06; B2M | | | A/MART-1 |
| 93876 | HLA-A*02; HLA-B*27; HLA-B*57; HLA-C*02; | tumor_rea_antigen_derived | public_antigen | CHD8 |
| | HLA-C*06; B2M | | | |
| 94879 | HLA-A*02; HLA-B*27; HLA-B*57; HLA-C*02; | ambiguous_vir_tumor | public_antigen | SEC24A |
| | HLA-C*06; B2M | | | |
| 102954 | HLA-DRB1*04; HLA-DRB1*13; HLA-DRB3*03; | tumor_rea_antigen_agnostic | MM_cell_ | NA |
| | HLA-DRB3*NN; HLA-DRB4*01; HLA- | | reactive | |
| | DRB4*NN; HLA-DRB5*NN; HLA-DQA1*01; | | | |
| | HLA-DQA1*03; HLA-DQB1*03; HLA-DQB1*06; | | | |
| | HLA-DPA1*01; HLA-DPB1*02; HLA-DPB1*04; | | | |
| | HLA-DRA*01 | | | |
| 113247 | HLA-A*01; HLA-A*02; HLA-B*44; HLA-B*57; | ambiguous_vir_tumor | public_antigen | IGH (JH) |
| | HLA-C*05; HLA-C*06; B2M | | | |
| 113412 | HLA-DRB1*04; HLA-DRB1*07; HLA-DRB3*NN; | tumor_rea_antigen_agnostic | MM_cell_ | NA |
| | HLA-DRB4*01; HLA-DRB5*NN; HLA-DQA1*02; | | reactive | |
| | HLA-DQA1*03; HLA-DQB1*02; HLA-DQB1*03; | | | |
| | HLA-DPA1*01; HLA-DPA1*02; HLA-DPB1*04; | | | |
| | HLA-DPB1*05; HLA-DRA*01 | | | |
| 116156 | HLA-A*03; HLA-A*23; HLA-B*49; HLA-B*51; | tumor_rea_antigen_agnostic | MM_cell_ | NA |
| | HLA-C*02; HLA-C*07; B2M | | reactive | |
| 116169 | HLA-A*03; HLA-A*23; HLA-B*49; HLA-B*51; | tumor_rea_antigen_agnostic | MM_cell_ | NA |
| | HLA-C*02; HLA-C*07; B2M | | reactive | |
| 116260 | HLA-A*03; HLA-A*23; HLA-B*49; HLA-B*51; | tumor_rea_antigen_agnostic | MM_cell_ | NA |
| | HLA-C*02; HLA-C*07; B2M | | reactive | |
| 116341 | HLA-A*03; HLA-A*23; HLA-B*49; HLA-B*51; | tumor_rea_antigen_agnostic | MM_cell_ | NA |
| | HLA-C*02; HLA-C*07; B2M | | reactive | |
| 116654 | HLA-DRB1*11; HLA-DRB3*02; HLA-DRB4*NN; | tumor_rea_antigen_agnostic | MM_cell_ | NA |
| | HLA-DRB5*NN; HLA-DQA1*05; HLA-DQB1*03; | | reactive | |
| | HLA-DPA1*01; HLA-DPB1*02; HLA-DPB1*04; | | | |
| | HLA-DRA*01 | | | |
| 116809 | HLA-DRB1*11; HLA-DRB3*02; HLA-DRB4*NN; | tumor_rea_antigen_agnostic | MM_cell_ | NA |
| | HLA-DRB5*NN; HLA-DQA1*05; HLA-DQB1*03; | | reactive | |
| | HLA-DPA1*01; HLA-DPB1*02; HLA-DPB1*04; | | | |
| | HLA-DRA*01 | | | |
| 117395 | HLA-A*03; HLA-A*23; HLA-B*49; HLA-B*51; | tumor_rea_antigen_agnostic | MM_cell_ | NA |
| | HLA-C*02; HLA-C*07; B2M | | reactive | |
| 118435 | HLA-DRB1*11; HLA-DRB3*02; HLA-DRB4*NN; | tumor_rea_antigen_derived | public_antigen | p53 |
| | HLA-DRB5*NN; HLA-DQA1*05; HLA-DQB1*03; | | | |
| | HLA-DPA1*01; HLA-DPB1*02; HLA-DPB1*04; | | | |
| | HLA-DRA*01 | | | |
| 118775 | HLA-DRB1*11; HLA-DRB3*02; HLA-DRB4*NN; | tumor_rea_antigen_agnostic | MM_cell_ | NA |
| | HLA-DRB5*NN; HLA-DQA1*05; HLA-DQB1*03; | | reactive | |
| | HLA-DPA1*01; HLA-DPB1*02; HLA-DPB1*04; | | | |
| | HLA-DRA*01 | | | |
| 119131 | HLA-A*03; HLA-A*23; HLA-B*49; HLA-B*51; | tumor_rea_antigen_agnostic | MM_cell_ | NA |
| | HLA-C*02; HLA-C*07; B2M | | reactive | |
| 119467 | HLA-DRB1*11; HLA-DRB3*02; HLA-DRB4*NN; | tumor_rea_antigen_agnostic | MM_cell_ | NA |
| | HLA-DRB5*NN; HLA-DQA1*05; HLA-DQB1*03; | | reactive | |
| | HLA-DPA1*01; HLA-DPB1*02; HLA-DPB1*04; | | | |
| | HLA-DRA*01 | | | |
| 119497 | HLA-DRB1*11; HLA-DRB3*02; HLA-DRB4*NN; | tumor_rea_antigen_agnostic | MM_cell_ | NA |
| | HLA-DRB5*NN; HLA-DQA1*05; HLA-DQB1*03; | | reactive | |
| | HLA-DPA1*01; HLA-DPB1*02; HLA-DPB1*04; | | | |
| | HLA-DRA*01 | | | |
| 119678 | HLA-DRB1*11; HLA-DRB3*02; HLA-DRB4*NN; | tumor_rea_antigen_agnostic | MM_cell_ | NA |
| | HLA-DRB5*NN; HLA-DQA1*05; HLA-DQB1*03; | | reactive | |
| | HLA-DPA1*01; HLA-DPB1*02; HLA-DPB1*04; | | | |
| | HLA-DRA*01 | | | |
| 121265 | HLA-DRB1*11; HLA-DRB3*02; HLA-DRB4*NN; | tumor_rea_antigen_agnostic | MM_cell_ | NA |
| | HLA-DRB5*NN; HLA-DQA1*05; HLA-DQB1*03; | | reactive | |
| | HLA-DPA1*01; HLA-DPB1*02; HLA-DPB1*04; | | | |
| | HLA-DRA*01 | | | |
| 121653 | HLA-A*03; HLA-A*23; HLA-B*49; HLA-B*51; | tumor_rea_antigen_agnostic | MM_cell_ | NA |
| | HLA-C*02; HLA-C*07; B2M | | reactive | |
| 122872 | HLA-A*03; HLA-A*23; HLA-B*49; HLA-B*51; | tumor_rea_antigen_agnostic | MM_cell_ | NA |
| | HLA-C*02; HLA-C*07; B2M | | reactive | |
| 124954 | HLA-DRB1*11; HLA-DRB3*02; HLA-DRB4*NN; | tumor_rea_antigen_agnostic | MM_cell_ | NA |
| | HLA-DRB5*NN; HLA-DQA1*05; HLA-DQB1*03; | | reactive | |
| | HLA-DPA1*01; HLA-DPB1*02; HLA-DPB1*04; | | | |
| | HLA-DRA*01 | | | |
| 125647 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 125730 | HLA-DRB1*04; HLA-DRB1*13; HLA-DRB3*01; | tumor_rea_antigen_agnostic | MM_cell_ | NA |
| | HLA-DRB3*NN; HLA-DRB4*01; HLA- | | reactive | |
| | DRB4*NN; HLA-DRB5*NN; HLA-DQA1*01; | | | |
| | HLA-DQA1*03; HLA-DQB1*03; HLA-DQB1*06; | | | |
| | HLA-DPA1*02; HLA-DPB1*05; HLA-DPB1*13; | | | |
| | HLA-DRA*01 | | | |
| 125770 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 125825 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 125952 | HLA-DRB1*04; HLA-DRB1*13; HLA-DRB3*01; | tumor_rea_antigen_agnostic | MM_cell_ | NA |
| | HLA-DRB3*NN; HLA-DRB4*01; HLA- | | reactive | |
| | DRB4*NN; HLA-DRB5*NN; HLA-DQA1*01; | | | |
| | HLA-DQA1*03; HLA-DQB1*03; HLA-DQB1*06; | | | |
| | HLA-DPA1*02; HLA-DPB1*05; HLA-DPB1*13; | | | |
| | HLA-DRA*01 | | | |
| 125996 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 126119 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 126200 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 126399 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 126405 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 126466 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 126479 | HLA-DRB1*04; HLA-DRB1*13; HLA-DRB3*01; | tumor_rea_antigen_agnostic | MM_cell_ | NA |
| | HLA-DRB3*NN; HLA-DRB4*01; HLA- | | reactive | |
| | DRB4*NN; HLA-DRB5*NN; HLA-DQA1*01; | | | |
| | HLA-DQA1*03; HLA-DQB1*03; HLA-DQB1*06; | | | |
| | HLA-DPA1*02; HLA-DPB1*05; HLA-DPB1*13; | | | |
| | HLA-DRA*01 | | | |
| 126502 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 126598 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 126628 | HLA-DRB1*04; HLA-DRB1*13; HLA-DRB3*01; | tumor_rea_antigen_agnostic | MM_cell_ | NA |
| | HLA-DRB3*NN; HLA-DRB4*01; HLA- | | reactive | |
| | DRB4*NN; HLA-DRB5*NN; HLA-DQA1*01; | | | |
| | HLA-DQA1*03; HLA-DQB1*03; HLA-DQB1*06; | | | |
| | HLA-DPA1*02; HLA-DPB1*05; HLA-DPB1*13; | | | |
| | HLA-DRA*01 | | | |
| 126931 | HLA-DRB1*04; HLA-DRB1*13; HLA-DRB3*01; | tumor_rea_antigen_agnostic | MM_cell_ | NA |
| | HLA-DRB3*NN; HLA-DRB4*01; HLA- | | reactive | |
| | DRB4*NN; HLA-DRB5*NN; HLA-DQA1*01; | | | |
| | HLA-DQA1*03; HLA-DQB1*03; HLA-DQB1*06; | | | |
| | HLA-DPA1*02; HLA-DPB1*05; HLA-DPB1*13; | | | |
| | HLA-DRA*01 | | | |
| 127031 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 127059 | HLA-A*03; HLA-A*24; HLA-B*51; HLA-B*55; | tumor_rea_antigen_derived | MM_cell_ | Tax-1 |
| | HLA-C*01; HLA-C*15; B2M | | reactive | |
| 127247 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 127371 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 127378 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 127649 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 127813 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 127973 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |
| 128279 | HLA-A*03; HLA-A*24; HLA-B*51; HLA-B*55; | tumor_rea_antigen_agnostic | MM_cell_ | NA |
| | HLA-C*01; HLA-C*15; B2M | | reactive | |

1. An isolated engineered immune cell comprising a T cell receptor (TCR) capable of recognizing a disease-associated antigen.

2. The cell of claim 1, wherein the disease-associated antigen is a virus-associated antigen or a cancer-associated antigen.

3. (canceled)

4. The cell of claim 3, wherein the cancer-associated antigens are associated with one or more hematological malignancies, and wherein the hematological malignancy is MM, AML, or CLL.

5. (canceled)

6. (canceled)

7. (canceled)

8. The cell of claim 1, wherein the disease-associated antigen is selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

9. The cell of claim 1, wherein the TCR comprises SEQ ID NOs: 1-121, and/or a TCR alpha chain CDR3 sequence selected from SEQ ID NO: 1-62, 41855-41902 or TCR beta chain CDR3 sequence selected from SEQ ID NO: 63-121 or 41903-41948.

10. The cell of claim 1, wherein the cell is a CD8 T cell.

11. The cell of claim 10, wherein the CD8 T cell is isolated from a subject to be treated, and wherein the cell comprises one or more modifications to one or more genes that modify an immune reactivity of the cell.

12. (canceled)

13. The cell of claim 1, wherein the cell is a CAR T cell.

14. A method of treating cancer comprising delivering the cell of claim 1.

15. The method of claim 14, wherein the cancer is a hematological malignancy, and wherein the hematological malignancy is MM, AML, or CLL.

16. (canceled)

17. A method of bone marrow transplant for use in treating hematological malignancies comprising transfusing a composition comprising the cells of claim 1 into a subject suffering from a hematological malignancy, and wherein the hematological malignancy is MM, AML, or CLL.

18. (canceled)

19. A vaccine comprising a cancer-associated antigen, wherein the antigen is recognized by a TCR selected from SEQ ID NOs: 1-121 and/or a TCR alpha chain CDR3 sequence selected from SEQ ID NO: 1-62, or 41855-41902 or TCR beta chain CDR3 sequence selected from SEQ ID NO: 63-121 or 41903-41948.

20. (canceled)

21. The vaccine of claim 19, wherein the antigen is selected from SEQ ID NO: 325-41854, and/or TATGATAGC, CAGGCGTCT, TTGGCTTCT, GGTGCATCC, AGTGCATCC, AAAGACAGT, GCTGCATCT, TGGGCATCA, AGTACTTAT, GCTGCGTCC, GAGGTCACC.

22. The vaccine of claim 19, wherein the vaccine comprises a polynucleotide encoding the conserved cancer antigen, wherein the polynucleotide is mRNA, and wherein the vaccine comprises the antigen and optionally a carrier or adjuvant.

23. (canceled)

24. (canceled)

25. A method of treating cancer in a subject comprising administering the cancer vaccine of claim 19.

26. A method for detecting tumor-reactive T-cell receptors (TCRs):
(a) characterizing the phenotype and clonality of a population of isolated T cells to define a baseline transcriptional state;
(b) segregating single isolated T cells from the population of isolated T cells into individual discrete volumes and exposing the single isolated T cells to a tumor cell;
(c) identifying and retrieving single isolated T cells from the individual discrete volumes and conducting TCR alpha and beta chain sequencing; and
(d) identifying antigen-reactive T cells by matching each TCR to its baseline transcriptional state using the CDR3 amino acid sequence as an endogenous barcode of each TCR.

27. The method of claim 26, wherein step (b) further comprises capture beads to detect T cell-derived cytokines and wherein single isolated T cells are retrieved for step (c) if T cell cytokines are detected, wherein the T cell-derived cytokines comprise interleukin-2 (IL-2), interferon-gamma, and tumor necrosis factor (TNF), and wherein step (b) further comprises assaying for expression of surface 4-IBB as an indicator of an antigen-activated T cell.

28. (canceled)

29. (canceled)

30. (canceled)

31. (canceled)

32. The method of claim 26, wherein characterizing the phenotype and clonality of the cells comprises using high-throughput single-cell RNA sequencing (scRNA-seq), single-cell TCR sequencing (scTCR-seq) coupled with the detection of surface proteins using cellular indexing of transcriptomes and epitopes by sequencing (CITE-seq), wherein determining one or more epitopes on the cells to define the clonotype comprises using high-throughput single-cell RNA sequencing (scRNA) and single-cell TCR sequencing, and/or using cellular indexing of the transcriptomes and epitopes by sequencing (CITE-seq), further comprising exposing a subset of the population of isolated T cells to stimulation with tumor or viral antigens and obtaining TCR sequencing TCRs using TCRV (Beta)-seq, and integrating the TCRV (beta)-seq with the baseline transcriptional state using the CDR3 amino acid sequence, and further comprising defining an antigen-reactive TCR signature based on the identified baseline transcriptional state.

33. (canceled)

34. (canceled)

35. (canceled)

36. The method of claim 26, wherein step (b) further comprises optical screening to quantify T cell activation and cytokine production, wherein the TCR detected comprises a sequence corresponding to TCR11729 or TCR15434 as shown in Table 8 with TCR11729 recognizing the CTAG2 antigen and TCR15434 recognizing a patient-specific mutation in the IGKV gene, and further comprising expanding the identified antigen-specific T cells in a cell population and delivering the cell population to a subject in need thereof.

37. (canceled)

38. (canceled)

39. The method of claim 26, wherein the TCR detected comprises a sequence corresponding to TCR11729 or TCR15434 as shown in Table 8 with TCR11729 recognizing the CTAG2 antigen and TCR15434 recognizing a patient-specific mutation in the IGKV gene.

40. (canceled)

41. (canceled)

42. (canceled)

43. (canceled)
